SEQUENCE LISTING (105 sequences)

SEQ ID NO: 1 - protein, 239 aa, Artificial sequence
  synthetic sequence
MAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWDAFVELYGPSMRPLFDFSWLSLKTLLSLALVGACITLGAYLGHK

SEQ ID NO: 2 - protein, 233 aa, Artificial sequence
  synthetic sequence
MSQSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNAAAESRKGQERFNRWFLTGMTVAGVVLLGSLFSRK

SEQ ID NO: 3 - protein, 193 aa, Artificial sequence
  synthetic sequence
MATPASAPDTRALVADFVGYKLRQKGYVCGAGPGEGPAADPLHQAMRAAGDEFETRFRRTFSDLAAQLHVTPGSAQQRFTQVSDELFQGGPNWGRLVAFFVFGAALCAESVNKEMEPLVGQVQEWMVAYLETQLADWIHSSGGWAEFTALYGDGALEEARRLREGNWASVRTVLTGAVALGALVTVGAFFASK

SEQ ID NO: 4 - protein, 350 aa, Artificial sequence
  synthetic sequence
MFGLKRNAVIGLNLYCGGAGLGAGSGGATRPGGRLLATEKEASARREIGGGEAGAVIGGSAGASPPSTLTPDSRRVARPPPIGAEVPDVTATPARLLFFAPTRRAAPLEEMEAPAADAIMSPEEELDGYEPEPLGKRPAVLPLLELVGESGNNTSTDGSLPSTPPPAEEEEDELYRQSLEIISRYLREQATGAKDTKPMGRSGATSRKALETLRRVGDGVQRNHETAFQGMLRKLDIKNEDDVKSLSRVMIHVFSDGVTNWGRIVTLISFGAFVAKHLKTINQESCIEPLAESITDVLVRTKRDWLVKQRGWDGFVEFFHVEDLEGGIRNVLLAFAGVAGVGAGLAYLIR

SEQ ID NO: 5 - protein, 194 aa, Artificial sequence
  synthetic sequence
MADPLRERTELLLADYLGYCAREPGTPEPAPSTPEAAVLRSAAARLRQIHRSFFSAYLGYPGNRFELVALMADSVLSDSPGPTWGRVVTLVTFAGTLLERGPLVTARWKKWGFQPRLKEQEGDVARDCQRLVALLSSRLMGQHRAWLQAQGGWDGFCHFFRTPFPLAFWRKQLVQAFLSCLLTTAFIYLWTRLL

SEQ ID NO: 6 - protein, 175 aa, Artificial sequence
  synthetic sequence
MTDCEFGYIYRLAQDYLQCVLQIPQPGSGPSKTSRVLQNVAFSVQKEVEKNLKSCLDNVNVVSVDTARTLFNQVMEKEFEDGIINWGRIVTIFAFEGILIKKLLRQQIAPDVDTYKEISYFVAEFIMNNTGEWIRQNGGWENGFVKKFEPKSGWMTFLEVTGKICEMLSLLKQYC

SEQ ID NO: 7 - protein, 192 aa, Artificial sequence
  synthetic sequence
MDGSGEQPRGGGPTSSEQIMKTGALLLQGFIQDRAGRMGGEAPELALDPVPQDASTKKLSECLKRIGDELDSNMELQRMIAAVDTDSPREVFFRVAADMFSDGNFNWGRVVALFYFASKLVLKALCTKVPELIRTIMGWTLDFLRERLLGWIQDQGGWDGLLSYFGTPTWQTVTIFVAGVLTASLTIWKKMG

SEQ ID NO: 8 - protein, 211 aa, Artificial sequence
  synthetic sequence
MASGQGPGPPRQECGEPALPSASEEQVAQDTEEVFRSYVFYRHQQEQEAEGVAAPADPEMVTLPLQPSSTMGQVGRQLAIIGDDINRRYDSEFQTMLQHLQPTAENAYEYFTKIATSLFESGINWGRVVALLGFGYRLALHVYQHGLTGFLGQVTRFVVDFMLHHCIARWIAQRGGWVAALNLGNGPILNVLVVLGVVLLGQFVVRRFFKS

SEQ ID NO: 9 - protein, 212 aa, Artificial sequence
  synthetic sequence
MEVLRRSSVFAAEIMDAFDRSPTDKELVAQAKALGREYVHARLLRAGLSWSAPERAAPVPGRLAEVCAVLLRLGDELEMIRPSVYRNVARQLHISLQSEPVVTDAFLAVAGHIFSAGITWGKVVSLYAVAAGLAVDCVRQAQPAMVHALVDCLGEFVRKTLATWLRRRGGWTDVLKCVVSTDPGLRSHWLVAALCSFGRFLKAAFFVLLPER

SEQ ID NO: 10 - protein, 199 aa, Artificial sequence
  synthetic sequence
MGRSDDAVIGRGLNSPDPLVREAFLMAYDYISYVTAKPGVPLCPAPSRASAALRHAGDELLIRFPIFFRRWPRVFQDVTEHTACPTLLSILDEHFAPTRRRDLAWSAVLSVFVLAGQLALHCQDRGMEDITPQIQECVGSYVERVICPEIRDKGGWSGFISRFGEKQNLEDHVVKVCCWSLLLLCVGILSYFIWTRRKT

SEQ ID NO: 11 - protein, 195 aa, Artificial sequence
  synthetic sequence
MDCEVNNGSSLRDECITNLLVFGFLQSCSDNSFRRELDALGHELPVLAPQWEGYDELQTDGNRSSHSRLGRIEADSESQEDIIRNIARHLAQVGDSMDRSIPPGLVNGLALQLRNTSRSEEDRNRDLATALEQLLQAYPRDMEKEKTMLVLALLLAKKVASHTPSLLRDVFHTTVNFINQNLRTYVRSLARNGMD

SEQ ID NO: 12 - protein, 334 aa, Artificial sequence
  synthetic sequence
MGRPAGLFPPLCPFLGFRPEACWERHMQIERAPSVPPFLRWAGYRPGPVRRRGKVELIKFVRVQWRRPQVEWRRRRWGPGPGASMAGSEELGLREDTLRVLAAFLRRGEAAGSPVPTPPRSPAQEEPTDFLSRLRRCLPCSLGRGAAPSESPRPCSLPIRPCYGLEPGPATPDFYALVAQRLEQLVQEQLKSPPSPELQGPPSTEKEAILRRLVALLEEEAEVINQKLASDPALRSKLVRLSSDSFARLVELFCSRDDSSRPSRACPGPPPPSPEPLARLALAMELSRRVAGLGGTLAGLSVEHVHSFTPWIQAHGGWEGILAVSPVDLNLPLD

SEQ ID NO: 13 - protein, 485 aa, Artificial sequence
  synthetic sequence
MASSSTVPLGFHYETKYVVLSYLGLLSQEKLQEQHLSSPQGVQLDIASQSLDQEILLKVKTEIEEELKSLDKEISEAFTSTGFDRHTSPVFSPANPESSMEDCLAHLGEKVSQELKEPLHKALQMLLSQPVTYQAFRECTLETTVHASGWNKILVPLVLLRQMLLELTRRGQEPLSALLQFGVTYLEDYSAEYIIQQGGWGTVFSLESEEEEYPGITAEDSNDIYILPSDNSGQVSPPESPTVTTSWQSESLPVSLSASQSWHTESLPVSLGPESWQQIAMDPEEVKSLDSNGAGEKSENNSSNSDIVHVEKEEVPEGMEEAAVASVVLPARELQEALPEAPAPLLPHITATSLLGTREPDTEVITVEKSSPATSLFVELDEEEVKAATTEPTEVEEVVPALEPTETLLSEKEINAREESLVEELSPASEKKPVPPSEGKSRLSPAGEMKPMPLSEGKSILLFGGAAAVAILAVAIGVALALRKK

SEQ ID NO: 14 - protein, 327 aa, Artificial sequence
  synthetic sequence
MCSTSGCDLEEIPLDDDDLNTIEFKILAYYTRHHVFKSTPALFSPKLLRTRSLSQRGLGNCSANESWTEVSWPCRNSQSSEKAINLGKKKSSWKAFFGVVEKEDSQSTPAKVSAQGQRTLEYQDSHSQQWSRCLSNVEQCLEHEAVDPKVISIANRVAEIVYSWPPPQATQAGGFKSKEIFVTEGLSFQLQGHVPVASSSKKDEEEQILAKIVELLKYSGDQLERKLKKDKALMGHFQDGLSYSVFKTITDQVLMGVDPRGESEVKAQGFKAALVIDVTAKLTAIDNHPMNRVLGFGTKYLKENFSPWIQQHGGWEKILGISHEEVD

SEQ ID NO: 15 - protein, 163 aa, Artificial sequence
  synthetic sequence
MKSSQTFEEQTECIVNTLLMDFLSPTLQVASRNLCCVDEVDSGEPCSFDVAIIAGRLRMLGDQFNGELEASAKNVIAETIKGQTGAILQDTVESLSKTWCAQDSSLAYERAFLAVSVKLLEYMAHIAPEVVGQVAIPMTGMINGNQAIREFIQGQGGWENLES

SEQ ID NO: 16 - protein, 280 aa, Artificial sequence
  synthetic sequence
MTRCTADNSLTNPAYRRRTMATGEMKEFLGIKGTEPTDFGINSDAQDLPSPSRQASTRRMSIGESIDGKINDWEEPRLDIEGFVVDYFTHRIRQNGMEWFGAPGLPCGVQPEHEMMRVMGTIFEKKHAENFETFCEQLLAVPRISFSLYQDVVRTVGNAQTDQCPMSYGRLIGLISFGGFVAAKMMESVELQGQVRNLFVYTSLFIKTRIRNNWKEHNRSWDDFMTLGKQMKEDYERAEAEKVGRRKQNRRWSMIGAGVTAGAIGIVGVVVCGRMMFSLK

SEQ ID NO: 17 - protein, 299 aa, Artificial sequence
  synthetic sequence
MPGTSYPTNNDNFSNGFPMATTQSERLLQAQNRRKFSFPATLHSASLLEVGGGPKETTRRRLSNVSDAVTRKLSYTIGWKAAQIPAQDIISQGRCLCGHYIKRRLRRSGLFNKKLGLQRIRSILGSTSMGIVRDVFPAVQVLGDELERMHPRIYNGVARQICRNPGGEFHTPDAVSLLLGAVGRELFRVEITWSKVISLFAIAGGLSVDCVRQGHPEYLPKLMESVSEVIEDELVPWINENGGWSGINTHVLPTTNSLNPLEWTTLVIGVVFGLILVFMILRFIFNLIVPKIYQRFTNS

SEQ ID NO: 18 - protein, 300 aa, Artificial sequence
  synthetic sequence
MAPTTSPPPKLAKFKSSSLDHEIYTANRRGTIATASSDWKALRGGVGGGAGGPGSVPNPSNGRSLHAGGPMTRAASTSSLASSTRTMTNYQEYKMDIINQGKCLCGQYIRARLRRAGVLNRKVTQRLRNILDPGSSHVVYEVFPALNSMGEELERMHPRVYTNISRQLSRAPFGELEDSDMAPMLLNLVAKDLFRSSITWGKIISIFAVCGGFAIDCVRQGHFDYLQCLIDGLAEIIEDDLVYWLIDNGGWLGLSRHIRPRVGEFTFLGWLTLFVTISAGAYMVSNVCRRIGGQLYSLLF

SEQ ID NO: 19 - protein, 235 aa, Artificial sequence
  synthetic sequence
MAARGSAAPGGRANGRFHSRLYLQNTAVMEELYRRNLSEDLVRDNGLSCGGREYWREPASTVGAASDGLSEEERRTAADAAERMTAVIAGTPGIAVERNVRDFRRGGWDVTPDNVESEFREVERRTFSDGVHWGRVIAFLAFSMSFAAYVNSRGIDGGAYSVFNWTLRVLNDSLADFIQRENGWRGFIVYADTLLRAQGSTPPQHQTRGVWDAVAGIGVIGVGTLLALGMRQAFS

SEQ ID NO: 20 - protein, 176 aa, Artificial sequence
  synthetic sequence
MEAWECLEDFSAVRNLLEQSSNSTSWFWRFLWGSSQAKLVCRIKEDYKWEFEELLKSCGELFDSLNLGHQALFQEKVIKTLDFSTPGRAAAAVAFLSFIKDKWSEETHLSGGYLLDFLAMHLWRAVVRHKNRLLLLSSVRPAIIPTEEQQQQQEEARRRRQEQSPWNPRAGLDPRE

SEQ ID NO: 21 - protein, 179 aa, Artificial sequence
  synthetic sequence
MEGEELIYHNIINEILVGYIKYYMNDISEHELSPYQQQIKKILTYYDECLNKQVTITFSLTNAQEIKTQFTGVVTELFKDLINWGRICGFIVFSARMAKYCKDANNHLESTVITTAYNFMKHNLLPWMISHGGQEEFLAFSLHSDIYSVIFNIKYFLSKFCNHMFLRSCVQLLRNCNLI

SEQ ID NO: 22 - protein, 168 aa, Artificial sequence
  synthetic sequence
MKMLGEPEFKENILYYSFLNELFLILIRNGFSCSHAKLILDETRKRGLECSGQFEVISNSVEAPEPESLERIAKTLFTPRPHWGRLVAFLAYLAYLQKNSTEKLFWNDHLKKLKQIVKCHIVPWTLGPRDPKPKQRPFDKLPSAFYFLTAAASCLTLLLLYFRTTQTK

SEQ ID NO: 23 - protein, 220 aa, Artificial sequence
  synthetic sequence
MNLAIALDSPHPGLASYTILPRPFYHISLKPVSWPDETMRPAKSTDSVFVRTPVEAWVAPSPPDDKVAESSYLMFRAMYAVFTRDEKDLPLPALVLCRLIKASLRKDRKLYAELACRTADIGGKDTHVRLIISVLRAVYNDHYDYWSRLRVVLCYTVVFAVRNYLDDHKSAAFVLGAIAHYLALYRRLWFARLGGMPRSLRRQFPVTWALASLTDFLKSL

SEQ ID NO: 24 - protein, 191 aa, Artificial sequence
  synthetic sequence
MAYSTREILLALCIRDSRVHGNGTLHPVLELAARETPLRLSPEDTVVLRYHVLLEEIIERNSETFTETWNRFITHTEHVDLDFNSVFLEIFHRGDPSLGRALAWMAWCMHACRTLCCNQSTPYYVVDLSVRGMLEASEGLDGWIHQQGGWSTLIEDNIPGSRRFSWTLFLAGLTLSLLVICSYLFISRGRH

SEQ ID NO: 25 - protein, 226 aa, Artificial sequence
  synthetic sequence
MSLFFVVWYWVNYITKVCSGEVYIPSVLKFQYHSDTEHEPYSNLCKNLITMAEQDMDEVVSTIRRLLVECGMGLEEYLEHPVTAPIKVAVQDVIRTKQDIFSNFLTNINSVEDLETLGHAITTLNDYPSPNMGRVVCGIAFSVYVVQTVCKRKPLLVRCCLDIFTRATVQALNVNWFLQEGGWPALASFCKVVNSPSPRSRWLFPMFAISGLVLTVGVARNMVHFT

SEQ ID NO: 26 - protein, 171 aa, Artificial sequence
  synthetic sequence
MSHKKSGTYWATLITAFLKTVSKVEELDCVDSAVLVDVSKIITLTQEFRRHYDSVYRADYGPALKNWKRDLSKLFTSLFVDVINSGRIVGFFDVGRYVCEEVLCPGSWTEDHELLNDCMTHFFIENNLMNHFPLEDIFLAQRKFQTTGFTFLLHALAKVLPRIYSGNVIYV

SEQ ID NO: 27 - protein, 175 aa, Artificial sequence
  synthetic sequence
MDEDVLPGEVLAIEGIFMACGLNEPEYLYHPLLSPIKLYITGLMRDKESLFEAMLANVRFHSTTGINQLGLSMLQVSGDGNMNWGRALAILTFGSFVAQKLSNEPHLRDFALAVLPVYAYEAIGPQWFRARGGWRGLKAYCTQVLTRRRGRRMTALLGSIALLATILAAVAMSRR

SEQ ID NO: 28 - protein, 162 aa, Artificial sequence
  synthetic sequence
MADSLKEETALLLEDYFQHCCGKEGPPPSPTAAELRRAAAELERRERPFFRSCAPLASGGTQAALSALQSVVSELNSGSGFNWGRCLATIVLGGSLATALYENGCEEGPSRLAAALAAYLAEEQGEWLREHGGWVSTARAGMSGSFGSPVSARSEEHRARRW

SEQ ID NO: 29 - protein, 175 aa, Artificial sequence
  synthetic sequence
MASSNMKDETYYIALNMIQNYIIEYNTNKPRKSFVIDSISYDVLKAACKSVIKTNYNEFDIIISRNIDFNVIVTQVLEDKINWGRIITIIAFCAYYSKKVKQDTSPQYYDGIISEAITDAILSKYRSWFIDQDYWNGIRIYKNYSYIFNTASYCIFTASLIIASLAVFKICSFYM

SEQ ID NO: 30 - protein, 173 aa, Artificial sequence
  synthetic sequence
MANREEIDASAVMAAYLAREYAAAVEEQLTPRERDALEALRVSGEEVRSPLLQELSNAGEHRANPENSHIPAALVSALLEAPTSPGRMVTAIELCAQMGRVWTRGRQLVEFMRLVYVLLDRLPPTADEDLSTWLQAVARVHGTRRRLHRVLGVGAVMAGVGMLLLGVRVLRRT

SEQ ID NO: 31 - protein, 174 aa, Artificial sequence
  synthetic sequence
MMSNKFETDTKYLIDAFFKEYFNSQESNDIILKTIKKEVNILFDKHRLVYSNMINDISITTEIDILVKKTAESIFSDGLVNWGRIISLITFGILIVEYLKTINNTDKITSVSTIISSYLIEHQKHWLIKNNAWIGLVDFFTVQTYTSPVKSLLTFFIVFMGTGAMLYSAFNLTY

SEQ ID NO: 32 - protein, 137 aa, Artificial sequence
  synthetic sequence
MDVRQFLSDCEAPEEMVALRAAADAVGVDNRACAHLYTMLWEGVNLEEVHASLLGDGVVNWGRVAAFMHICRYIVRTFPSSMDRTEVALTKFIQDPKIDKQLREWTDRLGTVGLIGRCLEWLGAGVITGVVLSLLFY

SEQ ID NO: 33 - protein, 152 aa, Artificial sequence
  synthetic sequence
MTNINFSALLRGERMCPLTREIHSQMLIVTKSYSLVETFRAFPRLPNILEIGNNIVSDGNLNWGRILILLGISQLYFTKSESESERTQITEQLERFFRQDAISNWIVSNGGWVTCASLDLRNYSSVTNALQAMCFFGALFGTIAVIAYYLLP

SEQ ID NO: 34 - protein, 179 aa, Artificial sequence
  synthetic sequence
MEAAIEFDEIVKKLLNIYINDICTMGEKRLLNNYEKSILDRIYKSCEYIKKNYELDFNSMYNQININDITTSDIKSKIIESLLIDSRPSVKLATLSFISLIAEKWGEKNRTKIMEILSNEIVEKISNNGKDFIDFIDRDDDDIVDDYVLITNYLKITIFGAILGITAYYICKYLLKSIF

SEQ ID NO: 35 - protein, 222 aa, Artificial sequence
  synthetic sequence
MLSMFMCNNIVDYVDGIVQDIEDEASNNVDHDYVYPLPENMVYRFDKSTNILDYLSTERDHVMMAVRYYMSKQRLDDLYRQLPTKTRSYIDIINIYCDKVSNDYNRDMNIMYDMASTKSFTVYDINNEVNTILMDNKGLGVRLATISFITELGRRCMNPVKTIKMFTLLSHTICDDCFVDYITDISPPDNTIPNTSTREYLKLIGITAIMFATYKTLKYMIG

SEQ ID NO: 36 - protein, 166 aa, Artificial sequence
  synthetic sequence
MMSRLKTAVYDYLNDVDITECTEMDLLCQLSNCCDFINETYAKNYDTLYDIMERDILSYNIVNIKNTLTFALRDASPSVKLATLTLLASVIKKLNKIQHTDAAMFSEVIDGIVAEEQQVIGFIQKKCKYNTTYYNVRSGGCKISVYLTAAVVGFVAYGILKWYRGT

SEQ ID NO: 37 - protein, 117 aa, Artificial sequence
  synthetic sequence
MRTLLIRYILWRNDNDQTYYNDNFKKLMLLDELVDDGDVCTLIKNMRMTLSDGPLLDRLNQPVNNIEDAKRMIAISAKVARDIGERSEIRWEESFTILFRMIETYFDDLMIDLYGEK

SEQ ID NO: 38 - protein, 176 aa, Artificial sequence
  synthetic sequence
MDNCNYNIEKVLNVYLRDLRIESLNNNELAILIMIRECCEVIKKDYKTEFNEICNFILHNNVKSCYDINDVKNIIIETINSDFRPSVILASISLLSIIIKKKKNENNEVVNDDLALNELINTFSSYQKDIISFVEKNKKNNEHNDFIFSIINFFVMVGSIIIAYYLLKIIGRIRWK

SEQ ID NO: 39 - protein, 240 aa, Artificial sequence
  synthetic sequence
MAFDISVNASKTINALVYFSTQQNKLVIRNEVNDTHYTVEFDRDKVVDTFISYNRHNDTIEIRGVLPEETNIGCAVNTPVSMTYLYNKYSFKLILAEYIRHRNTISGNIYSALMTLDDLAIKQYGDIDLLFNEKLKVDSDSGLFDFVNFVKDMICCDSRIVVALSSLVSKHWELTNKKYRCMALAEHISDSIPISELSRLRYNLCKYLRGHTESIEDKFDYFEDDDSSTCSAVTDRETDV

SEQ ID NO: 40 - protein, 162 aa, Artificial sequence
  synthetic sequence
MDEAYYSGNLESVLGYVSDMHTELASISQLVIAKIETIDNDILNKDIVNFIMCRSNLDNPFISFLDTVYTIIDQENYQTELINSLDDNEIIDCIVNKFMSFYKDNLENIVDAIITLKYIMNNPDFKTTYAEVLGSRIADIDIKQVIRKNILQLSNDIRERYL

SEQ ID NO: 41 - protein, 190 aa, Artificial sequence
  synthetic sequence
MDIKIDISISGDKFTVTTRRENEERKKYLPLQKEKTTDVIKPDYLEYDDLLDRDEMFTILEEYFMYRGLLGLRIKYGRLFNEIKKFDNDAEEQFGTIEELKQKLRLNSEEGADNFIDYIKVQKQDIVKLTVYDCISMIGLCACVVDVWRNEKLFSRWKYCLRAIKLFINDHMLDKIKSILQNRLVYVEMS

SEQ ID NO: 42 - protein, 149 aa, Artificial sequence
  synthetic sequence
MTANFSTHVFSPQHCGCDRLTSIDDVRQCLTEYIYWSSYAYRNRQCAGQLYSTLLSFRDDAELVFIDIRELVKNMPWDDVKDCAEIIRCYIPDEQKTIREISAIIGLCAYAATYWGGEDHPTSNSLNALFVMLEMLNYVDYNIIFRRMN

SEQ ID NO: 43 - protein, 229 aa, Artificial sequence
  synthetic sequence
MVKNNKIQKNKISNSCRMIMSTDPNNILMRHLKNLTDDEFKCIIHRSSDFLYLSDSDYTSITKETLVSEIVEEYPDDCNKILAIIFLVLDKDIDVDIKTKLKPKPAVRFAILDKMTEDIKLTDLVRHYFRYIEQDIPLGPLFKKIDSYRTRAINKYSKELGLATEYFNKYGHLMFYTLPIPYNRFFCRNSIGFLAVLSPTIGHVKAFYKFIEYVSIDDRRKFKKELMSK

SEQ ID NO: 44 - protein, 149 aa, Artificial sequence
  synthetic sequence
MATKLDYEDAVFYFVDDDKICSRDSIIDLIDEYITWRNHVIVFNKDITSCGRLYKELMKFDDVAIRYYGIDKINEIVEAMSEGDHYINFTKVHDQESLFATIGICAKITEHWGYKKISESRFQSLGNITDLMTDDNINILILFLEKKLN

SEQ ID NO: 45 - protein, 175 aa, Artificial sequence
  synthetic sequence
MTSSAMDNNEPKVLEMVYDATILPEGSSMDPNIMDCINRHINMCIQRTYSSSIIAILDRFLMMNKDELNNTQCHIIKEFMTYEQMAIDHYGGYVNAILYQIRKRPNQHHTIDLFKRIKRTRYDTFKVDPVEFVKKVIGFVSILNKYKPVYSYVLYENVLYDEFKCFINYVETKYF

SEQ ID NO: 46 - protein, 151 aa, Artificial sequence
  synthetic sequence
MNAYNKADSFSLESDSIKDVIHDYICWLSMTDEMRPSIGNVFKAMETFKIDAVRYYDGNIYELAKDINAMSFDGFIRSLQTIASKKDKLTVYGTMGLLSIVVDINKGCDISNIKFAAGIIILMEYIFDDTDMSHLKVALYRRIQRRDDVDR

SEQ ID NO: 47 - protein, 181 aa, Artificial sequence
  synthetic sequence
MISLSFLIHNPLKKWKLKPSISINGYRSTFTMAFPCAQFRPCHCHATKDSLNTVADVRHCLTEYILWVSHRWTHRESAGSLYRLLISFRTDATELFGGELKDSLPWDNIDNCVEIIKCFIRNDSMKTAEELRAIIGLCTQSAIVSGRVFNDKYIDILLMLRKILNENDYLTLLDHIRTAKY

SEQ ID NO: 48 - protein, 198 aa, Artificial sequence
  synthetic sequence
MAKQPSDVSSECDREGRQLQPAERPPQLRPGAPTSLQTEPQGNPEGNHGGEGDSCPHGSPQGPLAPPASPGPFATRSPLFIFMRRSSLLSRSSSGYFSFDTDRSPAPMSCDKSTQTPSPPCQAFNHYLSAMASMRQAEPADMRPEIWIAQELRRIGDEFNAYYARRVFLNNYQAAEDHPRMVILRLLRYIVRLVWRMH

SEQ ID NO: 49 - protein, 193 aa, Artificial sequence
  synthetic sequence
MARARQEGSSPEPVEGLARDGPRPFPLGRLVPSAVSCGLCEPGLAAAPAAPTLLPAAYLCAPTAPPAVTAALGGSRWPGGPRSRPRGPRPDGPQPSLSLAEQHLESPVPSAPGALAGGPTQAAPGVRGEEEQWAREIGAQLRRMADDLNAQYERRRQEEQQRHRPSPWRVLYNLIMGLLPLPRGHRAPEMEPN

SEQ ID NO: 50 - protein, 168 aa, Artificial sequence
  synthetic sequence
MFQIPEFEPSEQEDSSSAERGLGPSPAGDGPSGSGKHHRQAPGLLWDASHQQEQPTSSSHHGGAGAVEIRSRHSSYPAGTEDDEGMGEEPSPFRGRSRSAPPNLWAAQRYGRELRRMSDEFVDSFKKGLPRPKSAGTATQMRQSSSWTRVFQSWWDRNLGRGSSAPSQ

SEQ ID NO: 51 - protein, 160 aa, Artificial sequence
  synthetic sequence
MSEVRPLSRDILMETLLYEQLLEPPTMEVLGMTDSEEDLDPMEDFDSLECMEGSDALALRLACIGDEMDVSLRAPRLAQLSEVAMHSLGLAFIYDQTEDIRDVLRSFMDGFTTLKENIMRFWRSPNPGSWVSCEQVLLALLLLLALLLPLLSGGLHLLLK

SEQ ID NO: 52 - protein, 184 aa, Artificial sequence
  synthetic sequence
MEPSQCVEELEDDVFQPEDGEPVTQPGSLLSADLFAQSLLDCPLSRLQLFPLTHCCGPGLRPTSQEDKATQTLSPASPSQGVMLPCGVTEEPQRLFYGNAGYRLPLPASFPAVLPIGEQPPEGQWQHQAEVQIARKLQCIADQFHRLHVQQHQQNQNRVWWQILLFLHNLALNGEENRNGAGPR

SEQ ID NO: 53 - protein, 106 aa, Artificial sequence
  synthetic sequence
MLMLTFASTSSDLLPMSNVFDVQSSVFYNEKNMFYSSSQDFSSCEDSSQFADDSGFFDDSEISSIGYEIGSKLAAMCDDFDAQMMSYSAHASDRSLFHRLLDFFAF

SEQ ID NO: 54 - protein, 91 aa, Artificial sequence
  synthetic sequence
MCPCPLHRGRGPPAVCACSAGRLGLRSSAAQLTAARLKALGDELHQRTMWRRRARSRRAPAPGALPTYWPWLCAAAQVAALAAWLLGRRNL

SEQ ID NO: 55 - protein, 54 aa, Artificial sequence
  synthetic sequence
MPGKKARKNAQPSPARAPAELEVECATQLRRFGDKLNFRQKLLNLISKLFCSGT

SEQ ID NO: 56 - protein, 343 aa, Artificial sequence
  synthetic sequence
MDNQAERESEAGVGLQRDEDDAPLCEDVELQDGDLSPEEKIFLREFPRLKEDLKGNIDKLRALADDIDKTHKKFTKANMVATSTAVISGVMSLLGLALAPATGGGSLLLSTAGQGLATAAGVTSIVSGTLERSKNKEAQARAEDILPTYDQEDREDEEEKADYVTAAGKIIYNLRNTLKYAKKNVRAFWKLRANPRLANATKRLLTTGQVSSRSRVQVQKAFAGTTLAMTKNARVLGGVMSAFSLGYDLATLSKEWKHLKEGARTKFAEELRAKALELERKLTELTQLYKSLQQKVRSRARGVGKDLTGTCETEAYWKELREHVWMWLWLCVCLCVCVYVQFT

SEQ ID NO: 57 - protein, 140 aa, Artificial sequence
  synthetic sequence
MAEEPQSVLQLPTSIAAGGEGLTDVSPETTTPEPPSSAAVSPGTEEPAGDTKKKIDILLKAVGDTPIMKTKKWAVERTRTIQGLIDFIKKFLKLVASEQLFIYVNQSFAPSPDQEVGTLYECFGSDGKLVLHYCKSQAWG

SEQ ID NO: 58 - protein, 98 aa, Artificial sequence
  synthetic sequence
MMSYKRDGYFSIVSSCLIFCLHFLPLLSIRTKLSSLFITSLANNACNSNTVEYNIGRKLTVMCDEFDSELMSYKEEKSFVKFLGSGFKTYASIVRRVF

SEQ ID NO: 59 - protein, 4374 aa, Artificial sequence
  synthetic sequence
MKVDRTKLKKTPTEAPADCRALIDKLKVCNDEQLLLELQQIKTWNIGKCELYHWVDLLDRFDGILADAGQTVENMSWMLVCDRPEREQLKMLLLAVLNFTALLIEYSFSRHLYSSIEHLTTLLASSDMQVVLAVLNLLYVFSKRSNYITRLGSDKRTPLLTRLQHLAESWGGKENGFGLAECCRDLHMMKYPPSATTLHFEFYADPGAEVKIEKRTTSNTLHYIHIEQLDKISESPSEIMESLTKMYSIPKDKQMLLFTHIRLAHGFSNHRKRLQAVQARLHAISILVYSNALQESANSILYNGLIEELVDVLQITDKQLMEIKAASLRTLTSIVHLERTPKLSSIIDCTGTASYHGFLPVLVRNCIQAMIDPSMDPYPHQFATALFSFLYHLASYDAGGEALVSCGMMEALLKVIKFLGDEQDQITFVTRAVRVVDLITNLDMAAFQSHSGLSIFIYRLEHEVDLCRKECPFVIKPKIQRPNTTQEGEEMETDMDGVQCIPQRAALLKSMLNFLKKAIQDPAFSDGIRHVMDGSLPTSLKHIISNAEYYGPSLFLLATEVVTVFVFQEPSLLSSLQDNGLTDVMLHALLIKDVPATREVLGSLPNVFSALCLNARGLQSFVQCQPFERLFKVLLSPDYLPAMRRRRSSDPLGDTASNLGSAVDELMRHQPTLKTDATTAIIKLLEEICNLGRDPKYICQKPSIQKADGTATAPPPRSNHAAEEASSEDEEEEEVQAMQSFNSTQQNETEPNQQVVGTEERIPIPLMDYILNVMKFVESILSNNTTDDHCQEFVNQKGLLPLVTILGLPNLPIDFPTSAACQAVAGVCKSILTLSHEPKVLQEGLLQLDSILSSLEPLHRPIESPGGSVLLRELACAGNVADATLSAQATPLLHALTAAHAYIMMFVHTCRVGQSEIRSISVNQWGSQLGLSVLSKLSQLYCSLVWESTVLLSLCTPNSLPSGCEFGQADMQKLVPKDEKAGTTQGGKRSDGEQDGAAGSMDASTQGLLEGIGLDGDTLAPMETDEPTASDSKGKSKITPAMAARIKQIKPLLSASSRLGRALAELFGLLVKLCVGSPVRQRRSHHAASTTTAPTPAARSTASALTKLLTKGLSWQPPPYTPTPRFRLTFFICSVGFTSPMLFDERKYPYHLMLQKFLCSGGHNALFETFNWALSMGGKVPVSEGLEHSDLPDGTGEFLDAWLMLVEKMVNPTTVLESPHSLPAKLPGGVQNFPQFSALRFLVVTQKAAFTCIKNLWNRKPLKVYGGRMAESMLAILCHILRGEPVIRERLSKEKEGSRGEEDTGQEEGGSRREPQVNQQQLQQLMDMGFTREHAMEALLNTSTMEQATEYLLTHPPPIMGGVVRDLSMSEEDQMMRAIAMSLGQDIPMDQRAESPEEVACRKEEEERKAREKQEEEEAKCLEKFQDADPLEQDELHTFTDTMLPGCFHLLDELPDTVYRVCDLIMTAIKRNGADYRDMILKQVVNQVWEAADVLIKAALPLTTSDTKTVSEWISQMATLPQASNLATRILLLTLLFEELKLPCAWVVESSGILNVLIKLLEVVQPCLQAAKEQKEVQTPKWITPVLLLIDFYEKTAISSKRRAQMTKYLQSNSNNWRWFDDRSGRWCSYSASNNSTIDSAWKSGETSVRFTAGRRRYTVQFTTMVQVNEETGNRRPVMLTLLRVPRLNKNSKNSNGQELEKTLEESKEMDIKRKENKGNDTPLALESTNTEKETSLEETKIGEILIQGLTEDMVTVLIRACVSMLGVPVDPDTLHATLRLCLRLTRDHKYAMMFAELKSTRMILNLTQSSGFNGFTPLVTLLLRHIIEDPCTLRHTMEKVVRSAATSGAGSTTSGVVSGSLGSREINYILRVLGPAACRNPDIFTEVANCCIRIALPAPRGSGTASDDEFENLRIKGPNAVQLVKTTPLKPSPLPVIPDTIKEVIYDMLNALAAYHAPEEADKSDPKPGVMTQEVGQLLQDMGDDVYQQYRSLTRQSSDFDTQSGFSINSQVFAADGASTETSASGTSQGEASTPEESRDGKKDKEGDRASEEGKQKGKGSKPLMPTSTILRLLAELVRSYVGIATLIANYSYTVGQSELIKEDCSVLAFVLDHLLPHTQNAEDKDTPALARLFLASLAAAGSGTDAQVALVNEVKAALGRALAMAESTEKHARLQAVMCIISTIMESCPSTSSFYSSATAKTQHNGMNNIIRLFLKKGLVNDLARVPHSLDLSSPNMANTVNAALKPLETLSRIVNQPSSLFGSKSASSKNKSEQDAQGASQDSSSNQQDPGEPGEAEVQEEDHDVTQTEVADGDIMDGEAETDSVVIAGQPEVLSSQEMQVENELEDLIDELLERDGGSGNSTIIVSRSGEDESQEDVLMDEAPSNLSQASTLQANREDSMNILDPEDEEEHTQEEDSSGSNEDEDDSQDEEEEEEEDEEDDQEDDEGEEGDEDDDDDGSEMELDEDYPDMNASPLVRFERFDREDDLIIEFDNMFSSATDIPPSPGNIPTTHPLMVRHADHSSLTLGSGSSTTRLTQGIGRSQRTLRQLTANTGHTIHVHYPGNRQPNPPLILQRLLGPSAAADILQLSSSLPLQSRGRARLLVGNDDVHIIARSDDELLDDFFHDQSTATSQAGTLSSIPTALTRWTEECKVLDAESMHDCVSVVKVSIVNHLEFLRDEELEERREKRRKQLAEEETKITDKGKEDKENRDQSAQCTASKSNDSTEQNLSDGTPMPDSYPTTPSSTDAATSESKETLGTLQSSQQQPTLPTPPALGEVPQELQSPAGEGGSSTQLLMPVEPEELGPTRPSGEAETTQMELSPAPTITSLSPERAEDSDALTAVSSQLEGSPMDTSSLASCTLEEAVGDTSAAGSSEQPRAGSSTPGDAPPAVAEVQGRSDGSGESAQPPEDSSPPASSESSSTRDSAVAISGADSRGILEEPLPSTSSEEEDPLAGISLPEGVDPSFLAALPDDIRREVLQNQLGIRPPTRTAPSTNSSAPAVVGNPGVTEVSPEFLAALPPAIQEEVLAQQRAEQQRRELAQNASSDTPMDPVTFIQTLPSDLRRSVLEDMEDSVLAVMPPDIAAEAQALRREQEARQRQLMHERLFGHSSTSALSAILRSPAFTSRLSGNRGVQYTRLAVQRGGTFQMGGSSSHNRPSGSNVDTLLRLRGRLLLDHEALSCLLVLLFVDEPKLNTSRLHRVLRNLCYHAQTRHWVIRSLLSILQRSSESELCIETPKLTTSEEKGKKSSKSCGSSSHENRPLDLLHKMESKSSNQLSWLSVSMDAALGCRTNIFQIQRSGGRKHTEKHASGGSTVHIHPQAAPVVCRHVLDTLIQLAKVFPSHFTQQRTKETNCESDRERGNKACSPCSSQSSSSGICTDFWDLLVKLDNMNVSRKGKNSVKSVPVSAGGEGETSPYSLEASPLGQLMNMLSHPVIRRSSLLTEKLLRLLSLISIALPENKVSEAQANSGSGASSTTTATSTTSTTTTTAASTTPTPPTAPTPVTSAPALVAATAISTIVVAASTTVTTPTTATTTVSISPTTKGSKSPAKVSDGGSSSTDFKMVSSGLTENQLQLSVEVLTSHSCSEEGLEDAANVLLQLSRGDSGTRDTVLKLLLNGARHLGYTLCKQIGTLLAELREYNLEQQRRAQCETLSPDGLPEEQPQTTKLKGKMQSRFDMAENVVIVASQKRPLGGRELQLPSMSMLTSKTSTQKFFLRVLQVIIQLRDDTRRANKKAKQTGRLGSSGLGSASSIQAAVRQLEAEADAIIQMVREGQRARRQQQAATSESSQSEASVRREESPMDVDQPSPSAQDTQSIASDGTPQGEKEKEERPPELPLLSEQLSLDELWDMLGECLKELEESHDQHAVLVLQPAVEAFFLVHATERESKPPVRDTRESQLAHIKDEPPPLSPAPLTPATPSSLDPFFSREPSSMHISSSLPPDTQKFLRFAETHRTVLNQILRQSTTHLADGPFAVLVDYIRVLDFDVKRKYFRQELERLDEGLRKEDMAVHVRRDHVFEDSYRELHRKSPEEMKNRLYIVFEGEEGQDAGGLLREWYMIISREMFNPMYALFRTSPGDRVTYTINPSSHCNPNHLSYFKFVGRIVAKAVYDNRLLECYFTRSFYKHILGKSVRYTDMESEDYHFYQGLVYLLENDVSTLGYDLTFSTEVQEFGVCEVRDLKPNGANILVTEENKKEYVHLVCQMRMTGAIRKQLAAFLEGFYEIIPKRLISIFTEQELELLISGLPTIDIDDLKSNTEYHKYQSNSIQIQWFWRALRSFDQADRAKFLQFVTGTSKVPLQGFAALEGMNGIQKFQIHRDDRSTDRLPSAHTCFNQLDLPAYESFEKLRHMLLLAIQECSEGFGLA

SEQ ID NO: 60 - protein, 364 aa, Artificial sequence
  synthetic sequence
MDSSRAIGLYFDSALPSSSLLAFPIVLQDTGDGKKQITPQYRIQRLDSWTDSKEDSVFITTYGFIFQVGNEEVTVGMINDNPGHELLSSAMLCLGSVPNDGDLVELARACLTMVVTCKKSATNTERIVFSVVQAPRVLQSCMVVANRYSSVNAVNHVKAPEKIPGSGTLEYKVNFVSLTVVPRKDVYRIPTAALKSIWLKPVQSCAQCHYDCGGGPEEPVSQIPFQVDSWILCNSFLAYRGMVHCRKEGKESDIDQARGEDKETQSICRAQDCARTFRACEGERCTDKAVGTFLLSQWDSLLSYSKCLSPGGKILWSQTAHLRSVKIVIQAGTQRAVAVTADHEVTSTKIEKRHTIAKYNPFKK

SEQ ID NO: 61 - protein, 2204 aa, Artificial sequence
  synthetic sequence
MASSGPERAEHQIILPESHLSSPLVKHKLLYYWKLTGLPLPDECDFDHLIVSRQWKKILESAAPDTERMIKLGRAVHQTLNHNSKITGVLHPRCLEKLASIEVPDSTNKFRKIEKKIQIHNTRYGELFTRLCTNVEKKLLGSSWSNNVSRSEEFNSIRTDPAFWFHSKWSKAKFGWAHFKQVPRHLIVAARTRSAVTKFVTLTHKLGQVFGTPELVMGNHTNENKSPCLTQELVLMYADMMEGRDMVNIISSTATHLRSLSEKIDDILRLVDALAKDLGNQVYDVVALMEGFAYGAVQLLEPSGTFAGDFFAFNLQELKDTLIELLPNDVAELVTHAIATVFSGLEQNQAAEMLCLLRLWGHPLLESRIAAKAVRSQMCAPKMIDFDMILQVLSFFKGTIINGYRKKNSGVWPHVKMDTIYGKVIGQLHADSAEISHDVMLREYKSLSALEFEPCIEYDPVTNLSMFLKDKAIAHPRDNWLASFRRNLLSEDQKRHIKEATSTNRLLIEFLESNDFDPYKEMEYLTTLEYLRDDNVAVSYSLKEKEVKVNGRIFAKLTKKLRNCQVMAEGILADQIAPFFQGNGVIQDSISLTKSMLAMSQLSFNSNKKRITDCKERVSSNRNHDPKSKNRRRVATFITTDLQKYCLNWRYQTVKLFAHAINQLMGLPHFFEWIHLRLMDTTMFVGDPFNPPSDPTDCDLSRVPNDDIYIVSARGGIEGLCQKLWTMISIAAIQLAAARSHCRVACMVQGDNQVIAVTREVRSDDSPEMVWTQWLQASDNFFKEMIHVNHLNGHNLKDRETIRSDTFFLYSKRIFKDGAILSQVLKNSSKLVLISGDLSENTVMSCANIASTIARLCENGLPKDFCYYLNYIMSCVQTYFDSEFSITHSSQPDSNQSWFEDISFVHSYVLTPAQLGGLSNLQYSRLYTRNIGDPGTTAFAEVKRLEAVGLLSPSIMTNILTRPPGNGDWASLCNDPYSFNFETVASPNIVLKKHTQKVLFETCSNPLLSGVHTEDNEAEEKALVEFLLNQEVVHPRVAHAIMESSSVGRRKQIQGLVDTTNTVINIALTRRPLGIKRLMRIINYSSMHAMLFTDDVFLSNRPNHPLVSSNMCSLTLADYARNRSWSPLTGGRKILGVSNPDTIEPVEGEILSVSGGCKKCDSGDEQFTWFHLPSNIQLTDDTSKNPPMRVPYLGSKTQERRAASLAKIAHMSPHVKAALRASSVLIWAYGDNEVNWTAALKIARSRCNISSEYLRLLSPLPTAGNLQHRLDDGITQMTFTPASLYRVSPYIHISNDSQRLFTEEGIKEGNVVYQQIMLLGLSLIESLFPMTTTKTYDEITLHLHSKFSCCIREAPVAVPFELLGLAPELRAVTSNKFMYDPSPVSERDFARLDLAIFKSYELNLESYPTIELMNILSISSGKLIGQSVVSYDEDTSIKNDAIIVYDNTRNWISEAQNSDVVRLFEYAALEVLLDCSYQLYYLRVRGLNNIVLYMSDLYKNMPGILLSNIAATISHPIIHSRLNAVGLVNHDGSHQLADTDFIEMSAKLLVSCTRRVVSGLYAGNKYDLLFPSVLDDNLSEKMLQLISRLCCLYTVLFATTREIPKIRGLSAEEKCSVLTEYLLSDAVKPLLRSEQVSCIMSPNIITFPANLYYMSRKSLNLIREREDRDAILALLFPQEPLLEFRPVQDIGVRVKDPFTRQPAALLQELDLSAPARYDAFTLNEVRSEHTLPNPEEDYLVRYLFRGIGTASSSWYKASHLLSVPEVRCARYGNSLYLAEGSGAIMSLLELHVPHETIYYNTLFSNEMNPPQRHFGPTPTQFLNSVVYRNLQAEVPCKDGFIQEFRPLWRENAEESDLTSDKAVGYITSAVPYRSVSLLHCDIEIPPGSNQSLLDQLAINLSLIAMHSVREGGVVIIKVLYAMGYYFHLLMNLFTPCSTKGYILSNGYACRGDMECYLIFVMGYLGGPTFVHEVVRMAKTLVQRHGTLLSKSDEITLTRLFTSQQHRVTDILSSPLPRLMKFLRENIDAALIEAGGQPVRPFCAESLVSTLTDMTQMTQIIASHIDTVIRSVIYMEAEGDLADTVFLFTPYNLSTDGKKRTSLKQCTRQILEVTILGLRVKDLNKVGDVIGLVLRGMVSLEDLIPLRTYLRRSTCPKYLKAVLGITKLKEMFTDTSLLYLTLAQQKFYMKTIGNAAKGYYSNCDS

SEQ ID NO: 62 - protein, 553 aa, Artificial sequence
  synthetic sequence
MGSRPFTKNPAPMMLTIRVALVLSCICPANSIDGRPFAAAGIVVTGDKAVNIYTSSQTGSIIVKLLPNLPKDKEACAKAPLDAYNRTLTTLLTPLGDSIRRIQESVTTSGGGRQGRLIGAIIGGVALGVATAAQITAAAALIQAKQNAANILRLKESIAATNEAVHEVTDGLSQLAVAVGKMQQFVNDQFNKTAQELDCIKIAQQVGVELNLYLTELTTVFGPQITSPALNKLTIQALYNLAGGNMDYLLTKLGIGNNQLSSLIGSGLITGNPILYDSQTQLLGIQVTLPSVGNLNNMRATYLETLSVSTTRGFASALVPKVVTQVGSVIEELDTSYCIETDLDLYCTRIVTFPMSPGIYSCLSGNTSACMYSKTEGALTTPYMTIKGSVIANCKMTTCRCVNPPGIISQNYGEAVSLIDKQSCNVLSLGGITLRLSGEFDVTYQKNISIQDSQVIITGNLDISTELGNVNNSISNALNKLEESNRKLDKVNVKLTSTSALITYIVLTIISLVFGILSLILACYLMYKQKAQQKTLLWLGNNTLDQMRATTKM

SEQ ID NO: 63 - protein, 450 aa, Artificial sequence
  synthetic sequence
MEGSKTSNNSTMQVSFVCQRCSQPLKLDTSFKILDRVTIQELTAPLLTTAQAKPGETQEEETNSGEEPFIETPRQDGVSRRFIPPARMMSTESANSFTLIGEASDGGTMENLSRRLKVTGDLFDIMSGQTDVDHPLCEECTDTLLDQLDTQLNVTENECQNYKRCLEILEQMNEDDSEQLQMELKELALEEERLIQELEDVEKNRKIVAENLEKVQAEAERLDQEEAQYQREYSEFKRQQLELDDELKSVENQMRYAQTQLDKLKKTNVFNATFHIWHSGQFGTINNFRLGRLPSVPVEWNEINAAWGQTVLLLHALANKMGLKFQRYRLVPYGNHSYLESLTDKSKELPLYCSGGLRFFWDNKFDHAMVAFLDCVQQFKEEVEKGETRFCLPYRMDVEKGKIEDTGGSGGSYSIKTQFNSEEQWTKALKFMLTNLKWGLAWVSSQFYNK

SEQ ID NO: 64 - protein, 228 aa, Artificial sequence
  synthetic sequence
MAAPQDVHVRICNQEIVKFDLEVKALIQDIRDCSGPLSALTELNTKVKEKFQQLRHRIQDLEQLAKEQDKESEKQLLLQEVENHKKQMLSNQASWRKANLTCKIAIDNLEKAELLQGGDLLRQRKTTKESLAQTSSTITESLMGISRMMAQQVQQSEEAMQSLVTSSRTILDANEEFKSMSGTIQLGRKLITKYNRRELTDKLLIFLALALFLATVLYIVKKRLFPFL

SEQ ID NO: 65 - protein, 314 aa, Artificial sequence
  synthetic sequence
MEGVELKEEWQDEDFPIPLPEDDSIEADILAITGPEDQPGSLEVNGNKVRKKLMAPDISLTLDPSDGSVLSDDLDESGEIDLDGLDTPSENSNEFEWEDDLPKPKTTEVIRKGSITEYTAAEEKEDGRRWRMFRIGEQDHRVDMKAIEPYKKVISHGGYYGDGLNAIVVFAVCFMPESSQPNYRYLMDNLFKYVIGTLELLVAENYMIVYLNGATTRRKMPSLGWLRKCYQQIDRRLRKNLKSLIIVHPSWFIRTLLAVTRPFISSKFSQKIRYVFNLAELAELVPMEYVGIPECIKQVDQELNGKQDEPKNEQ

SEQ ID NO: 66 - protein, 259 aa, Artificial sequence
  synthetic sequence
MGDAAADPPGPALPCEFLRPGCGAPLSPGAQLGRGAPTSAFPPPAAEAHPAARRGLRSPQLPSGAMSQNGAPGMQEESLQGSWVELHFSNNGNGGSVPASVSIYNGDMEKILLDAQHESGRSSSKSSHCDSPPRSQTPQDTNRASETDTHSIGEKNSSQSEEDDIERRKEVESILKKNSDWIWDWSSRPENIPPKEFLFKHPKRTATLSMRNTSVMKKGGIFSAEFLKVFLPSLLLSHLLAIGLGIYIGRRLTTSTSTF

SEQ ID NO: 67 - protein, 219 aa, Artificial sequence
  synthetic sequence
MSSHLVEPPPPLHNNNNNCEENEQSLPPPAGLNSSWVELPMNSSNGNDNGNGKNGGLEHVPSSSSIHNGDMEKILLDAQHESGQSSSRGSSHCDSPSPQEDGQIMFDVEMHTSRDHSSQSEEEVVEGEKEVEALKKSADWVSDWSSRPENIPPKEFHFRHPKRSVSLSMRKSGAMKKGGIFSAEFLKVFIPSLFLSHVLALGLGIYIGKRLSTPSASTY

SEQ ID NO: 68 - protein, 108 aa, Artificial sequence
  synthetic sequence
MVTLLPIEGQEIHFFEILESECVLYTGWIERASGSSIYPEAKARLPLEALLGSNKEPMLPKETVLSLKRYNLGSSAMKRNVPGHVLQRPSYLTRIQVTLLCNSSAEAL

SEQ ID NO: 69 - protein, 221 aa, Artificial sequence
  synthetic sequence
MSSFLEFAKPKMLDIKRKINFASGEKTDESVQPQQQTEQSSAQQTTPSAKAVSNPFITPLTESTPGMSESWVELAPSRTSLCSSVDINMVIIDEKDKDSRLSPVSIAQSPHVEFESLEQVKYKLVREMLPPGKNTDWIWDWSSRPENTPPKTVRMVQYGSNLTTPPNSPEPELYQYLPCESDSLFNVRVVFGFLVTNIFSFVVGAAVGFAVCRKLIKHHRQ

SEQ ID NO: 70 - protein, 449 aa, Artificial sequence
  synthetic sequence
MMKTLLLFVGLLLTWESGQVLGDQTVSDNELQEMSNQGSKYVNKEIQNAVNGVKQIKTLIEKTNEERKTLLSNLEEAKKKKEDALNETRESETKLKELPGVCNETMMALWEECKPCLKQTCMKFYARVCRSGSGLVGRQLEEFLNQSSPFYFWMNGDRIDSLLENDRQQTHMLDVMQDHFSRASSIIDELFQDRFFTREPQDTYHYLPFSLPHRRPHFFFPKSRIVRSLMPFSPYEPLNFHAMFQPFLEMIHEAQQAMDIHFHSPAFQHPPTEFIREGDDDRTVCREIRHNSTGCLRMKDQCDKCREILSVDCSTNNPSQAKLRRELDESLQVAERLTRKYNELLKSYQWKMLNTSSLLEQLNEQFNWVSRLANLTQGEDQYYLRVTTVASHTSDSDVPSGVTEVVVKLFDSDPITVTVPVEVSRKNPKFMETVAEKALQEYRKKHREE

SEQ ID NO: 71 - protein, 3010 aa, Artificial sequence
  synthetic sequence
MSTNPKPQRKTKRNTYRRPQDVKFPGGGQIVGGVYVLPRRGPTLGVRATRKTSERSQPRGRRQPIPKARRPEGRAWAQPGYPWPLYGNEGLGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTIPASAYQVRNASGLYHVTNDCSNSSIVYEAAGMIMHTPGCVPCVRENNASRCWVALTPTLAARNTSIPTTTIRRHVDLLVGAAAFCSAMYVGDLCGSVFLVSQLFTFSPRRYETVQDCNCSIYPGHVSGHRMAWDMMMNWSPTTALVVSQLLRIPQAVVDMVAGAHWGVLAGLAYYSMVGNWAKVLIVMLLFAGVDGVTYTTGGSQARHTQSVTSFFTQGPAQRIQLINTNGSWHINRTALNCNESLNTGFFAALFYAHKFNSSGCPERMASCSSIDKFAQGWGPITYTEPRDLDQRPYCWHYAPRQCGIVPASQVCGPVYCFTPSPVVVGTTDRSGAPTYNWGANETDVLLLNNTRPPQGNWFGCTWMNSTGFTKTCGGPPCNIGGVGNLTLTCPTDCFRKHPEATYTKCGSGPWLTPRCIVDYPYRLWHYPCTVNFTIFKVRMYVGGVEHRLSAACNWTRGERCDLEDRDRSELSPLLLSTTEWQTLPCSFTTLPALSTGLIHLHQNIVDVQYLYGIGSAVVSFVIKWEYIVLLFLLLADARVCACLWMMLLIAQAEAALENLVVLNAASLAGADGILSFLVFFCAAWYIKGRLVPGAAYALYGVWPLLLLLLALPPRAYAMDREMAASCGGVVFVGLILLTLSPHYKVFLARLIWWLQYFITRAEAHLCVWVPPLNVRGGRDAIILLTCAAHPELIFDITKLLLAILGPLMVLQAAITAMPYFVRAQGLIRACMLVRKVAGGHYVQMAFMKLAALTGTYVYDHLTPLQDWAHAGLRDLAVAVEPVVFSDMETKIITWGADTAACGDIILGLPVSARRGREILLGPADSIEGQGWRLLAPITAYAQQTRGLLGCIVTSLTGRDKNQVEGEVQVVSTATQSFLATCVNGVCWTVFHGAGSKTLAGPKGPITQMYTNVDQDLVGWHAPPGARSLTPCTCGSSDLYLVTRHADVIPVRRRGDGRGSLLSPRPVSYLKGSSGGPLLCPSGHAVGIFRAAVCTRGVAKAVDFIPVESMETTMRSPVFTDNSSPPAVPQTFQVAHLHAPTGSGKSTKVPAAYAAQGYKVLVLNPSVAATLGFGAYMSKAHGTDPNIRTGVRTITTGAPITYSTYGKFLADGGCSGGAYDIIICDECHSTDSTTILGIGTVLDQAETAGARLVVLATATPPGSVTVPHPNIEEVALSNTGEIPFYGKAIPLEAIKGGRHLIFCHSKKKCDELAAKLSGLGINAVAYYRGLDVSVIPTSGDVVIVATDALMTGYTGDFDSVIDCNTCVTQTVDFSLDPTFTIETTTVPQDAVSRSQRRGRTGRGRGGIYRFVTPGERPSGMFDSSVLCECYDAGCAWYELTPAETTVRLRAYLNTPGLPVCQDHLEFWESVFTGLTHIDAHFLSQTKQAGDNFPYLVAYQATVCARAQAPPPSWDQMWKCLIRLKPTLHGPTPLLYRLGAVQNEITLTHPITKFIMACMSADLEVVTSTWVLVGGVLAALAAYCLTTGSVVIVGRIILSGRPAVVPDREVLYREFDEMEECASHLPYIEQGMQLAEQFKQKALGLLQTATKQAEAAAPVVESRWRALEAFWAKHMWNFISGIQYLAGLSTLPGNPAIASLMAFTASITSPLTTQNTLLFNILGGWVAAQLAPPSAASAFVGAGIAGAAIGSIGLGKVLVDILAGYGAGVAGALVAFKVMSGEAPSAEDLVNLLPAILSPGALVVGVVCAAILRRHVGPGEGAVQWMNRLIAFASRGNHVSPTHYVPESDAAARVTQILSSLTITQLLKRLHQWINEDCSTPCSGSWLKDVWDWICTVLTDFKTWLQSKLLPKLPGVPFFSCQRGYKGVWRGDGIMQTTCPCGAQITGHVKNGSMRIVGPKTCSNTWHGTFPINAYTTGPCTPSPAPNYSRALWRVAAEEYVEITRVGDFHYVTGMTTDNVKCPCQVPAPEFFTELDGVRLHRYAPACRPLLREDVTFQVGLNQYLVGSQLPCEPEPDVAVLTSMLTDPSHITAETAKRRLARGSPPSLASSSASQLSAPSLKATCTTHHDSPDADLIEANLLWRQEMGGNITRVESENKVVILDSFDPLRAEEDEREVSVAAEILRKSKKFPPALPIWARPDYNPPLLESWKSPDYVPPAVHGCPLPPTTGPPIPPPRKKRTVVLTESTVSSALAELATKTFGSSGSSAVDSGTATAPPDQTSDDGDKESDVESYSSMPPLEGEPGDPDLSDGSWSTVSGEASDDIVCCSMSYTWTGALITPCAAEESKLPINALSNSLLRHHNMVYATTSRSASLRQKKVTFDRLQVLDDHYRDVLKEMKAKASTVKAKLLSVEEACKLTPPHSAKSKFGYGAKDVRNLSSKAINHIRSVWKDLLEDTETPIDTTIMAKSEVFCVQPEKGGRKPARLIVFPDLGVRVCEKMALYDVVSTLPQAVMGSSYGFQYSPGQRVEFLVNAWKSKKSPMGFSYDTRCFDSTVTESDIRVEESIYQCCDLAPEARQAIKSLTERLYIGGPLTNSKGQNCGYRRCRASGVLTTSCGNTLTCYLKATAACRAAKLQDCTMLVNGDDLVVICESAGTQEDAASLRVFTEAMTRYSAPPGDPPQPEYDLELITSCSSNVSVAHDASGKRVYYLTRDPTTPLARAAWETARHTPVNSWLGNIIMYAPTLWARMILMTHFFSILLAQEQLEKALDCQIYGACYSIEPLDLPQIIQRLHGLSAFSLHSYSPGEINRVASCLRKLGVPPLRVWRHRARSVRARLLSQGGRAATCGKYLFNWAVRTKLKLTPIPAASQLDLSSWFVAGYSGGDIYHSLSRARPRWFMWCLLLLSVGVGIYLLPNR

SEQ ID NO: 72 - protein, 1689 aa, Artificial sequence
  synthetic sequence
MVGELRYREFRVPLGPGLHAYPDELIRQRVGHNGHPEYQIRWLILRRGDDGDRDSTVDCKAEHILLWMSDDEIYANCHKMLGENGQVIAPSRESTEAGALDKSVLGEMETDVKSLIQRALRQLEECVGTVPPAPLLHTVHVLSAYASIEPLTGIFKDRRVVNLLMHMLSSPDYQIRWSAGRMIQALSSHDAGTRTQILLSLSQQEAIEKHLDFDSRCALLALFAQATLTEHPMSFEGVQLPQVPGRLLFSLVKRYLHVTFLLDRLNGDAGDQGAQNNFIPEELNVGRGRLELEFSMAMGTLISELVQAMRWDGASSRPESSSSSTFQPRPAQFRPYTQRFRRSRRFRPRASFASFNTYALYVRDTLRPGMRVRMLENYEEIAAGDEGQFRQSNDGVPPAQVLWDSTGHTYWVHWHMLEILGFEEDIEDVIDIEELQELGANGALSIVPPSQRWKPITQLFAEPYVVPEEEDREESENLTQAEWWELLFFIRQLSEAERLHIVDLLQDHLEEERVLDYDMLPELTVPVDLAQDLLLSLPQQLEDSALRDLFSCSVYRKYGPEVLVGHLSYPFVPGAQPNLFGANEESEAKDPPLQSASPALQRLVESLGPEGEVLVELEQALGSEAPQETEVKSCLLQLQEQPQPFLALMRSLDTSASNKTLHLTVLRILMQLVNFPEALLLPWHEAMDACVTCLRSPNTDREVLQELIFFLHRLTTTSRDYAVILNQLGARDAISKVLEKHRGKLELAQELRDMVSKCEKHAHLYRKLTTNILGGCIQMVLGQIEDHRRTHRPIQIPFFDVFLRYLCQGSSEEMKKNRYWEKVEVSSNPQRASRLTDRNPKTYWESSGRAGSHFITLHMRPGVIIRQLTLLVAGEDSSYMPAWVVVCGGNSIKSVNKELNTVNVMPSASRVTLLENLTRFWPIIQIRIKRCQQGGINTRIRGLEVLGPKPTFWPVFREQLCRHTRLFYMVRAQAWSQDIAEDRRSLLHLSSRLNGALRHEQNFAERFLPDMEAAQALSKTCWEALVSPLVQNITSPDEDSTSSLGWLLDQYLGCREAAYNPQSRAAAFSSRVRRLTHLLVHVEPREAAPPVVAIPRSKGRNRIHDWSYLITRGLPSSIMKNLTRCWRSVVEEQMNKFLTASWKDDDFVPRYCERYYVLQKSSSELFGPRAAFLLAMRNGCADAVLRLPFLRAAHVSEQFARHIDQRIQGSRMGGARGMEMLAQLQRCLESVLIFSPLEIATTFEHYYQHYMADRLLSVGSSWLEGAVLEQIGPCFPSRLPQQMLQSLNVSEELQRQFHVYQLQQLDQELLKLEDTEKKIQVAHEDSGREDKSKKEEAIGEAAAVAMAEEEDQGKKEEGEEEGEGEDEEEERYYKGTMPEVCVLVVTPRFWPVASVCQMLNPATCLPAYLRGTINHYTNFYSKSQSRSSLEKEPQRRLQWTWQGRAEVQFGGQILHVSTVQMWLLLHLNNQKEVSVESLQAISELPPDVLHRAIGPLTSSRGPLDLQEQKNVPGGVLKIRDDSEEPRPRRGNVWLIPPQTYLQAEAEEGRNMEKRRNLLNCLVVRILKAHGDEGLHVDRLVYLVLEAWEKGPCPARGLVSSLGRGATCRSSDVLSCILHLLVKGTLRRHDDRPQVLYYAVPVTVMEPHMESLNPGSAGPNPPLTFHTLQIRSRGVPYASCTDNHTFSTFR

SEQ ID NO: 73 - protein, 76 aa, Artificial sequence
  synthetic sequence
MYSFVSEETGTLIVNSVLLFLAFVVFLLVTLAILTALRLCAYCCNIVNVSLVKPTVYVYSRVKNLNSSEGVPDLLV

SEQ ID NO: 74 - protein, 1255 aa, Artificial sequence [sequence omitted]

SEQ ID NO: 75 - protein, 1308 aa, Artificial sequence
  synthetic sequence
MKPATGLWVWVSLLVAAGTVQPSDSQSVCAGTENKLSSLSDLEQQYRALRKYYENCEVVMGNLEITSIEHNRDLSFLRSVREVTGYVLVALNQFRYLPLENLRIIRGTKLYEDRYALAIFLNYRKDGNFGLQELGLKNLTEILNGGVYVDQNKFLCYADTIHWQDIVRNPWPSNLTLVSTNGSSGCGRCHKSCTGRCWGPTENHCQTLTRTVCAEQCDGRCYGPYVSDCCHRECAGGCSGPKDTDCFACMNFNDSGACVTQCPQTFVYNPTTFQLEHNFNAKYTYGAFCVKKCPHNFVVDSSSCVRACPSSKMEVEENGIKMCKPCTDICPKACDGIGTGSLMSAQTVDSSNIDKFINCTKINGNLIFLVTGIHGDPYNAIEAIDPEKLNVFRTVREITGFLNIQSWPPNMTDFSVFSNLVTIGGRVLYSGLSLLILKQQGITSLQFQSLKEISAGNIYITDNSNLCYYHTINWTTLFSTINQRIVIRDNRKAENCTAEGMVCNHLCSSDGCWGPGPDQCLSCRRFSRGRICIESCNLYDGEFREFENGSICVECDPQCEKMEDGLLTCHGPGPDNCTKCSHFKDGPNCVEKCPDGLQGANSFIFKYADPDRECHPCHPNCTQGCNGPTSHDCIYYPWTGHSTLPQHARTPLIAAGVIGGLFILVIVGLTFAVYVRRKSIKKKRALRRFLETELVEPLTPSGTAPNQAQLRILKETELKRVKVLGSGAFGTVYKGIWVPEGETVKIPVAIKILNETTGPKANVEFMDEALIMASMDHPHLVRLLGVCLSPTIQLVTQLMPHGCLLEYVHEHKDNIGSQLLLNWCVQIAKGMMYLEERRLVHRDLAARNVLVKSPNHVKITDFGLARLLEGDEKEYNADGGKMPIKWMALECIHYRKFTHQSDVWSYGVTIWELMTFGGKPYDGIPTREIPDLLEKGERLPQPPICTIDVYMVMVKCWMIDADSRPKFKELAAEFSRMARDPQRYLVIQGDDRMKLPSPNDSKFFQNLLDEEDLEDMMDAEEYLVPQAFNIPPPIYTSRARIDSNRSEIGHSPPPAYTPMSGNQFVYRDGGFAAEQGVSVPYRAPTSTIPEAPVAQGATAEIFDDSCCNGTLRKPVAPHVQEDSSTQRYSADPTVFAPERSPRGELDEEGYMTPMRDKPKQEYLNPVEENPFVSRRKNGDLQALDNPEYHNASNGPPKAEDEYVNEPLYLNTFANTLGKAEYLKNNILSMPEKAKKAFDNPDYWNHSLPPRSTLQHPDYLQEYSTKYFYKQNGRIRPIVAENPEYLSEFSLKPGTVLPPPPYRHRNTVV

SEQ ID NO: 76 - protein, 266 aa, Artificial sequence
  synthetic sequence
MVKVTFNSALAQKEAKKDEPKSGEEALIIPPDAVAVDCKDPDDVVPVGQRRAWCWCMCFGLAFMLAGVILGGAYLYKYFALQPDDVYYCGIKYIKDDVILNEPSADAPAALYQTIEENIKIFEEEEVEFISVPVPEFADSDPANIVHDFNKKLTAYLDLNLDKCYVIPLNTSIVMPPRNLLELLINIKAGTYLPQSYLIHEHMVITDRIENIDHLGFFIYRLCHDKETYKLQRRETIKGIQKREASNCFAIRHFENKFAVETLICS

SEQ ID NO: 77 - protein, 351 aa, Artificial sequence
  synthetic sequence
MTLRLLEDWCRGMDMNPRKALLIAGISQSCSVAEIEEALQAGLAPLGEYRLLGRMFRRDENRKVALVGLTAETSHALVPKEIPGKGGIWRVIFKPPDPDNTFLSRLNEFLAGEGMTVGELSRALGHENGSLDPEQGMIPEMWAPMLAQALEALQPALQCLKYKKLRVFSGRESPEPGEEEFGRWMFHTTQMIKAWQVPDVEKRRRLLESLRGPALDVIRVLKINNPLITVDECLQALEEVFGVTDNPRELQVKYLTTYQKDEEKLSAYVLRLEPLLQKLVQRGAIERDAVNQARLDQVIAGAVHKTIRRELNLPEDGPAPGFLQLLVLIKDYEAAEEEEALLQAILEGNFT

SEQ ID NO: 78 - protein, 2929 aa, Artificial sequence
  synthetic sequence
MASISKDFTNLLNTLIDGQIGAASRQTEWFNMSPDERTDYIKQVDERLQEMQQSTLSVLAAQHFQMQDNPVSVGDQLQTLQKRRQQMTDVPGTPAINAYKQQLDRDILLYRRQQTAMTHFDSTWRKVLVMLGPDDSKPLNATTLRENAVDKQAKLDTEIKRLEQQLTIQVADSTFSQKYVTLFSELQAYKDVNARYNALLKASATEEAAALGALTKVPQASDDLPVNISLLMMEERPGYIRMNVALVNASTDGRFKDFFLENGRLVVLTDGVLNFSFGTAARSLAWQQQYRLKSEPPSFRSPTYTPIRSVLVKTEFVEKYFANYLVSESTLRGGFKAQLLGNGRKMLLTSVDRKVPNQIGIQVSGQAPNTTITREVPLASALSDLINQNADIASFRTIGLEGFRQSSYHPDRDGLFVNIHELERSVGFAGRQYLLEMPQDNDYLSATPFGVMSVDGDKVSSSHLSKAQTDTLYQYNAAFFEKLEQLRSGGMKASRLFEGSIERTAFVQQLVRLLERNHITPAGVLAPEYPRDNMRDIKGNNLNKVLWEQAFAASVWRSRDNDPLLFRLATRLVKNPAVVKVLQNGYVQSDIAQARELLAPLYEQWRTRAVEAETQRVASANAAQHPSNPKVHVFDQAEVERSLDDKLLILLLTGPQSLEGTDVQLRPMVEAALLSNEGRSLRKQILFHALRPVADSFSKAAAPVNPHAELGVGKIMINNRLNQPDPYLILNTSSEEQAYRDGSYLIKDDKYRSYNQFRPDFKNDATRYMNDLDTPFVGGISGTTQTVSNVLTELFGGALSVKQYWQFQMANAAFMIRNGYHSFFETFYVAARYEPEGADSIGKEMLQMFDKYRVEGSKKALQGKLYDGVMARVLPIINQGLSAADEFHPPRFTRIGPRPALLGQAVKDLELKAGLTSVGDGFEPRQGSADIHQFVTDPVLFAKTHTVSAEALVRSGRLPAEGSAQLVKVGSGLYELEYTEQSANDISSSSIPAYFLGYNGPNQANAVPAYVDIPKRTIAGNFLFTGTLSGGSLVVTSLDANTFRVYHDGRVNSSLLYDNVVMAVDYKDYQIAGTAEGLAAAYMQYVNHEWQLVLQRQEYQRDGQMLRLRLRDDEEPLSIQVADSQVVERNQAQFVAYREQIHQQLKKVATQFEVSISGVSDGVYTEGEFSPDHPAIAAWAKLCAEVYDRINADTKQLVDKRNKLYENRRNTIRRDLINQQIKQLNITLEYYKAQYDTVLREAGFVEQSWLWQQIKAKNGSAAVVRIDDTAIQGGGKQRTDSVGERYAISEAYQRGARGTGFSDGLRNFREIEIPGVDDKMSALEMKRLFLEGKLTSEQQGALSGRITETSRAEYIDKVLRQTAVFSEDFHDAGSVFDRLVPQDFYLSLVGDRSGGRCYPLVRAMTVALASGGEAGINSLVQKLFFASADPQAGSSTLLRNSLIKLHSNVEAVQASTELGQFGLSEVVSRLAATTGTSMFALNTQNHSMMVGSTVTTEGRRYYFYDPNVGIFAFDNTKSLSRAMEQHLVGRRLAVHYGSFGSKSAPAFNLIEIDTGKMAEVPVGNGLNVADLTRFEELSSVIGQRRQVEQVMSAQERITEDLQLSTALQAFDAEQWGARFEAASTRLAQEHQLDSRWLPIIATTEEQGEGRYRVQFINRDQPEQTRWLDTDDSTFVEFRRFVDEHMSVLNEHFTLESGRMRPRGGVGEAAPVDGLNAGFAVQALIQWFSDKNRHDAANGMASPDLATALKVHSYLNFVQMVHGGVQDVIKVTALVRTALRGEVVAAQTSFKEFALSLGHTVNEGVGVLFGGAMIGLDAYELAHAENDVQKAVFGTQLAFDSASFVTGAAGIGAGLVGASTAGAVLGGAGVILGGLAVGFTALAQAFGAVAEDAKAVGRYFDTVDKAYKGNGYRYDNEKQVLVPLAGAVIKTLDLSKNQIDFDSQYIYRTHSGSTGSGKINYFFWVGDFPRMVHDRGQAIEVRSGIGYKDVSRPLEHGDSNVVILPGTPKSYISYEYMLLPGATTRHDAGFDVIRRLEEDKRFDYDFYIFPGEETIRRIHHEYVDTPIEVVLDQRNRQLVAPELPKELHGFLCYEIKGAGGEYLIGLNEGAKVNLTSDVASTWIIDSSQLASDSISVSKDQLLVGEKGKEVVVKLYLAQNSQVLVVNGKGEVRKVDFTSLTAQVISEDASKWQVPGQQIEQHLSDLAKAHQLHGQYVVVENYRHQGRDVGRAFYDVTKDRMLFTDTTNEQAKRAQLGAVMGDYAYFYDADNAVAWRVDIATGQVDAQFEPWFNQNAGHISRFWQEGDVVYLARRYRLKEREAELGYRIIGDRMELVSAVGDDALLQLSARIGRHGDELEAILQGYRSNSTQRGTLMYTLGARLIQPTSAALVTVFGVDAAGVPHRYWIRTSDGTLIKPNLAPPADQTLHFEAHEQTRSAWQIPADLVLAGSMPLLGGKEVFFFYSKEQKTLFRQEGPGQEVLDANQPSALRVTTPALTNVINLNGHLVVVTEDGRVARLDALGQLSYAAVNEHWLKGRIHWWQDLTSVTDGRATLAVFGVKDTDGKSLLPVWYHNGQVVVASAALQDKHPQFLGFEVDGSSARLFEPASGKLYRQPAMTADALAAAFGTDEVLEASAQLPAANELEPELHLKAAEQVDAGLRLTTVKGEILLRTHDGKLQLVAVDKDWQQDNLVRLSQALAEVAGQWRVKGVLTLQGDDTQGWFDVGSGQVFSIGGIPATDNLRFIGIAVGKKGAYVYNPTDQMLYQVKESGAQKLNHYADVERIGSSLLLQDGGKGDLSPMLIAGVDSVVLHGGAGSDTYRLSQTMWSYYRTVVIDNDDPNQVLDRLIILAVDAEKIFVSRHEDDLMLTDSVNGTVLVIRKVFGSQAVTHRHLQIDLEGSSSVISVDHLVKGFTRLGTANIGLFELPWAI

SEQ ID NO: 79 - protein, 51 aa, Artificial sequence
  synthetic sequence
MQLRHIGDSVNHRVIQEHLAQEVGDVLAPFVALVFVRGQVLLRFFWNNHLL

SEQ ID NO: 80 - protein, 391 aa, Artificial sequence
  synthetic sequence
MKCLVTGGNVKVLGKAVHSLSRIGDELYLEPLEDGLSLRTVNSSRSAYACFLFAPLFFQQYQAATPGQDLLRCKILMKSFLSVFRSLAMLEKTVEKCCISLNGRSSRLVVQLHCKFGVRKTHNLSFQDCESLQAVFDPASCPHMLRAPARVLGEAVLPFSPALAEVTLGIGRGRRVILRSYHEEEADSTAKAMVTEMCLGEEDFQQLQAQEGVAITFCLKEFRGLLSFAESANLNLSIHFDAPGRPAIFTIKDSLLDGHFVLATLSDTDSHSQDLGSPERHQPVPQLQAHSTPHPDDFANDDIDSYMIAMETTIGNEGSRVLPSISLSPGPQPPKSPGPHSEEEDEAEPSTVPGTPPPKKFRSLFFGSILAPVRSPQGPSPVLAEDSEGEG

SEQ ID NO: 81 - protein, 426 aa, Artificial sequence
  synthetic sequence
MEFTVSNVNLRDLARIFTNLSRIDDAVNWEINKNQIEITCLNSSRSGFSMVTLKKAFFDKYIFQPDSVLLTGLMTPTIRIRTQVKPILSVFRNKIFDFIPTVVTTNSKNGYGSESASRKDVIVENVQISISTGSECRIIFKFLCKHGVIKTYKISYEQTQTLHAVFDKSLSHNNFQINSKILKDLTEHFGQRTEELTIQPLQERVLLTSFTEEVVHNRDILKQPTQTTVSIDGKEFERVALNEGVSVTLSLREFRAAVILAEALGSSICAYYGVPGKPILLTFAKGKNSEIEAQFILATVVGSDEQEVSSMMGNRWQHSSTPASLFNSVERNNSLTAVAHNPPGSIGWQTDQSDSSRMFNSALDRSDETNGIKEPSTTNDAGQSLFLDGIPNESELAAFNNDVNDDAEFGPTQAEQSYHGIFSQED

SEQ ID NO: 82 - protein, 205 aa, Artificial sequence
  synthetic sequence
MAEPLQPDPGAAEDAAAQAVETPGWKAPEDAGPQPGSYEIRHYGPAKWVSTSVESMDWDSAIQTGFTKLNSYIQGKNEKEMKIKMTAPVTSYVEPGSGPFSESTITISLYIPSEQQFDPPRPLESDVFIEDRAEMTVFVRSFDGFSSAQKNQEQLLTLASILREDGKVFDEKVYYTAGYNSPVKLLNRNNEVWLIQKNEPTKENE

SEQ ID NO: 83 - protein, 654 aa, Artificial sequence
  synthetic sequence
MNGHLEAEEQQDQRPDQELTGSWGHGPRSTLVRAKAMAPPPPPLAASTPLLHGEFGSYPARGPRFALTLTSQALHIQRLRPKPEARPRGGLVPLAEVSGCCTLRSRSPSDSAAYFCIYTYPRGRRGARRRATRTFRADGAATYEENRAEAQRWATALTCLLRGLPLPGDGEITPDLLPRPPRLLLLVNPFGGRGLAWQWCKNHVLPMISEAGLSFNLIQTERQNHARELVQGLSLSEWDGIVTVSGDGLLHEVLNGLLDRPDWEEAVKMPVGILPCGSGNALAGAVNQHGGFEPALGLDLLLNCSLLLCRGGGHPLDLLSVTLASGSRCFSFLSVAWGFVSDVDIQSERFRALGSARFTLGTVLGLATLHTYRGRLSYLPATVEPASPTPAHSLPRAKSELTLTPDPAPPMAHSPLHRSVSDLPLPLPQPALASPGSPEPLPILSLNGGGPELAGDWGGAGDAPLSPDPLLSSPPGSPKAALHSPVSEGAPVIPPSSGLPLPTPDARVGASTCGPPDHLLPPLGTPLPPDWVTLEGDFVLMLAISPSHLGADLVAAPHARFDDGLVHLCWVRSGISRAALLRLFLAMERGSHFSLGCPQLGYAAARAFRLEPLTPRGVLTVDGEQVEYGPLQAQMHPGIGTLLTGPPGCPGREP

SEQ ID NO: 84 - protein, 219 aa, Artificial sequence
  synthetic sequence
MNRLFGKAKPKAPPPSLTDCIGTVDSRAESIDKKISRLDAELVKYKDQIKKMREGPAKNMVKQKALRVLKQKRMYEQQRDNLAQQSFNMEQANYTIQSLKDTKTTVDAMKLGVKEMKKAYKQVKIDQIEDLQDQLEDMMEDANEIQEALSRSYGTPELDEDDLEAELDALGDELLADEDSSYLDEAASAPAIPEGVPTDTKNKDGVLVDEFGLPQIPAS

SEQ ID NO: 85 - protein, 687 aa, Artificial sequence
  synthetic sequence
MAEELVLERCDLELETNGRDHHTADLCREKLVVRRGQPFWLTLHFEGRNYEASVDSLTFSVVTGPAPSQEAGTKARFPLRDAVEEGDWTATVVDQQDCTLSLQLTTPANAPIGLYRLSLEASTGYQGSSFVLGHFILLFNAWCPADAVYLDSEEERQEYVLTQQGFIYQGSAKFIKNIPWNFGQFEDGILDICLILLDVNPKFLKNAGRDCSRRSSPVYVGRVVSGMVNCNDDQGVLLGRWDNNYGDGVSPMSWIGSVDILRRWKNHGCQRVKYGQCWVFAAVACTVLRCLGIPTRVVTNYNSAHDQNSNLLIEYFRNEFGEIQGDKSEMIWNFHCWVESWMTRPDLQPGYEGWQALDPTPQEKSEGTYCCGPVPVRAIKEGDLSTKYDAPFVFAEVNADVVDWIQQDDGSVHKSINRSLIVGLKISTKSVGRDEREDITHTYKYPEGSSEEREAFTRANHLNKLAEKEETGMAMRIRVGQSMNMGSDFDVFAHITNNTAEEYVCRLLLCARTVSYNGILGPECGTKYLLNLNLEPFSEKSVPLCILYEKYRDCLTESNLIKVRALLVEPVINSYLLAERDLYLENPEIKIRILGEPKQKRKLVAEVSLQNPLPVALEGCTFTVEGAGLTEEQKTVEIPDPVEAGEEVKVRMDLLPLHMGLHKLVVNFESDKLKAVKGFRNVIIGPA

SEQ ID NO: 86 - protein, 297 aa, Artificial sequence
  synthetic sequence
MSGPPPPYEEQSSHLYGQPASSQDGNAFIPEDFKYSTVVISCEPIIRQRFMHKVYSLLSCQLLASLSFCYWASVSTSLQNFIMSHIALFYICMVVSLVSCIWLAVSPRPEDYEASVPEPLLTGSSEEPAQEQRRLPWYVLSSYKQKLTLLSIFTLSEAYCLSLVTLAYDKDTVLSALLITTIVVVGVSLTALSERFENVLNSATSIYYWLNWGLWIMIGMGLTALLFGWNTHSSKFNLLYGWLGAILFTAYLFIDTQLIFRKVYPDEEVRCAMMLYLDIVNLFLSILRILANSNDDN

SEQ ID NO: 87 - protein, 843 aa, Artificial sequence
  synthetic sequence
MPLSYQHFRKLLLLDDEAGPLEEELPRLADEGLNRRVAEDLNLGNPNVSIPWTHKVGNFTGLYSSTVPVFNPEWQTPSFPDIHLQEDIVDRCKQFVGPLTVNENRRLKLIMPARFYPNVTKYLPLDKGIKPYYPEHVVNHYFQARHYLHTLWKAGILYKRESTHSASFCGSPYSWEQDLQHGRLVFQTSKRHGDKSFCPQSPGILPRSSVGPCIQSQLRKSRLGPQPPQGQLAGRPQGGSGSIRARVHPSPWGTVGVEPSGSGHTHICASSSSSCLHQSAVRKAAYSLISTSKGHSSSGRAVELHHFPPNSSRSQSQGSVPSCWWLQFRNSKPCSEYCLCHIVNLIDDWGPCAEHGEHRIRTPRTPARVTGGVFLVDKNPHNTTESRLVVDFSQFSRGNTRVSWPKFAVPNLQSLTNLLSSNLSWLSLDVSAAFYHLPLHPAAMPHLLVGSSGLSRYVARLSSNSRIINHQHGTMQDLHNSCSRNLYVSLMLLYKTYGRKLHLYSHPIILGFRKIPMGVGLSPFLLAQFTSAICSVVRRAFPHCLAFSYMDDVVLGAKSVQHLESLYAAVTNFLVSLGIHVNPHKTKRWGYSLNFMGYVIGSWGTLPQEHIRQKIKLCFRKLPVNRPIDWKVCQRIVGLLGFAAPFTQCGYPALMPLYACISAKQAFTFSPTYKAFLSQQYLNLYPVARQRSGLCQVFADATPTGWGLAIGHQRMRGTFVSPLPIHTAELLAACFARSRSGAKLIGTDNSVVLSRKYTSFPWLLGCAANWILRGTSFVYVPSALNPADDPSRGRLGLYRPLLRLPYRPTTGRTSLYADSPSVPSHLPDRVHFASPLHVAWRPP

SEQ ID NO: 88 - protein, 154 aa, Artificial sequence
  synthetic sequence
MAARMCCKLDPARDVLCLRPIGAESRGRPLPGPLGAVPPSSPSAVPADDGSHLSLRGLPVCSFSSAGPCALRFTSARRMETTVNAPWSLPTVLHKRTLGLSGWSMTWIEEYIKDCVFKDWEELGEEIRLKVFVLGGCRHKLVCSPAPCNFFTSA

SEQ ID NO: 89 - protein, 1298 aa, Artificial sequence
  synthetic sequence
MKVVPEKNAVRILWGRERGARAMGAQRLLQELVEDKTRWMKWEGKRVELPDSPRSTFLLAFSPDRTLLASTHVNHNIYITEVKTGKCVHSLIGHRRTPWCVTFHPTISGLIASGCLDGEVRIWDLHGGSESWFTDSNNAIASLAFHPTAQLLLIATANEIHFWDWSRREPFAVVKTASEMERVRLVRFDPLGHYLLTAIVNPSNQQGDDEPEIPIDGTELSHYRQRALLQSQPVRRTPLLHNFLHMLSSRSSGIQVGEQSTVQDSATPSPPPPPPQPSTERPRTSAYIRLRQRVSYPTAECCQHLGILCLCSRCSGTRVPSLLPHQDSVPPASARATTPSFSFVQTEPFHPPEQASSTQQDQGLLNRPSAFSTVQSSTAGNTLRNLSLGPTRRSLGGPLSSHPSRYHREIAPGLTGSEWTRTVLSLNSRSEAESMPPPRTSASSVSLLSVLRQQEGGSQASVYTSATEGRGFPASGLATESDGGNGSSQNNSGSIRHELQCDLRRFFLEYDRLQELDQSLSGEAPQTQQAQEMLNNNIESERPGPSHQPTPHSSENNSNLSRGHLNRCRACHNLLTFNNDTLRWERTTPNYSSGEASSSWQVPSSFESVPSSGSQLPPLERTEGQTPSSSRLELSSSASPQEERTVGVAFNQETGHWERIYTQSSRSGTVSQEALHQDMPEESSEEDSLRRRLLESSLISLSRYDGAGSREHPIYPDPARLSPAAYYAQRMIQYLSRRDSIRQRSMRYQQNRLRSSTSSSSSDNQGPSVEGTDLEFEDFEDNGDRSRHRAPRNARMSAPSLGRFVPRRFLLPEYLPYAGIFHERGQPGLATHSSVNRVLAGAVIGDGQSAVASNIANTTYRLQWWDFTKFDLPEISNASVNVLVQNCKIYNDASCDISADGQLLAAFIPSSQRGFPDEGILAVYSLAPHNLGEMLYTKRFGPNAISVSLSPMGRYVMVGLASRRILLHPSTEHMVAQVFRLQQAHGGETSMRRVFNVLYPMPADQRRHVSINSARWLPEPGLGLAYGTNKGDLVICRPEALNSGVEYYWDQLNETVFTVHSNSRSSERPGTSRATWRTDRDMGLMNAIGLQPRNPATSVTSQGTQTLALQLQNAETQTEREVPEPGTAASGPGEGEGSEYGASGEDALSRIQRLMAEGGMTAVVQREQSTTMASMGGFGNNIIVSHRIHRSSQTGTEPGAAHTSSPQPSTSRGLLPEAGQLAERGLSPRTASWDQPGTPGREPTQPTLPSSSPVPIPVSLPSAEGPTLHCELTNNNHLLDGGSSRGDAAGPRGEPRNR

SEQ ID NO: 90 - protein, 364 aa, Artificial sequence
  synthetic sequence
MPRGRCRQQGPRIPIWAAANYANAHPWQQMDKASPGVAYTPLVDPWIERPCCGDTVCVRTTMEQKSTASGTCGGKPAERGPLAGHMPSSRPHRVDFCWVPGSDPGTFDGSPWLLDRFLAQLGDYMSFHFEHYQDNISRVCEILRRLTGRAQAWAAPYLDGDLPLPDDYELFCQDLKEVVQDPNSFAEYHAVVTCPLPLASSQLPVAPQLPVVRQYLARFLEGLALDMGTAPRSLPAAMATPAVSGSNSVSRSALFEQQLTKESTPGPKEPPVLPSSTCSSKPGPVEPASSQPEEAAPTPVPRLSESANPPAQRPDPAHPGGPKPQKTEEEVLETEGDQEVSLGTPQEVVEAPETPGEPPLSPGF

SEQ ID NO: 91 - protein, 171 aa, Artificial sequence
  synthetic sequence
MLLLMMNVKWDVKEIMSQHNIYVDALLKEFEQFNKRLNEVSKRVRIPLPVSNILWEHCIRLANRTIVEGYANVKKCSNEGRALMQLDFQQFLMKLEKLTDIRPIPDKEFVETYIKAYYLTENDMERWIKEHREYSTKQLTNLVNVCLGSHINKKARQKLLAAIDEIDRPKR

SEQ ID NO: 92 - protein, 2758 aa, Artificial sequence
  synthetic sequence
MSDKMSSFLHIGDICSLYAEGSTNGFISTLGLVDDRCVVQPETGDLNNPPKKFRDCLFKLCPMNRYSAQKQFWKAAKPGANSTTDAVLLNKLHHAADLEKKQNETENRKLLGTVIQYGNVIQLLHLKSNKYLTVNKRLPALLEKNAMRVTLDEAGNEGSWFYIQPFYKLRSIGDSVVIGDKVVLNPVNAGQPLHASSHQLVDNPGCNEVNSVNCNTSWKIVLFMKWSDNKDDILKGGDVVRLFHAEQEKFLTCDEHRKKQHVFLRTTGRQSATSATSSKALWEVEVVQHDPCRGGAGYWNSLFRFKHLATGHYLAAEVDPDFEEECLEFQPSVDPDQDASRSRLRNAQEKMVYSLVSVPEGNDISSIFELDPTTLRGGDSLVPRNSYVRLRHLCTNTWVHSTNIPIDKEEEKPVMLKIGTSPVKEDKEAFAIVPVSPAEVRDLDFANDASKVLGSIAGKLEKGTITQNERRSVTKLLEDLVYFVTGGTNSGQDVLEVVFSKPNRERQKLMREQNILKQIFKLLQAPFTDCGDGPMLRLEELGDQRHAPFRHICRLCYRVLRHSQQDYRKNQEYIAKQFGFMQKQIGYDVLAEDTITALLHNNRKLLEKHITAAEIDTFVSLVRKNREPRFLDYLSDLCVSMNKSIPVTQELICKAVLNPTNADILIETKLVLSRFEFEGVSSTGENALEAGEDEEEVWLFWRDSNKEIRSKSVRELAQDAKEGQKEDRDVLSYYRYQLNLFARMCLDRQYLAINEISGQLDVDLILRCMSDENLPYDLRASFCRLMLHMHVDRDPQEQVTPVKYARLWSEIPSEIAIDDYDSSGASKDEIKERFAQTMEFVEEYLRDVVCQRFPFSDKEKNKLTFEVVNLARNLIYFGFYNFSDLLRLTKILLAILDCVHVTTIFPISKMAKGEENKGNNDVEKLKSSNVMRSIHGVGELMTQVVLRGGGFLPMTPMAAAPEGNVKQAEPEKEDIMVMDTKLKIIEILQFILNVRLDYRISCLLCIFKREFDESNSQTSETSSGNSSQEGPSNVPGALDFEHIEEQAEGIFGGSEENTPLDLDDHGGRTFLRVLLHLTMHDYPPLVSGALQLLFRHFSQRQEVLQAFKQVQLLVTSQDVDNYKQIKQDLDQLRSIVEKSELWVYKGQGPDETMDGASGENEHKKTEEGNNKPQKHESTSSYNYRVVKEILIRLSKLCVQESASVRKSRKQQQRLLRNMGAHAVVLELLQIPYEKAEDTKMQEIMRLAHEFLQNFCAGNQQNQALLHKHINLFLNPGILEAVTMQHIFMNNFQLCSEINERVVQHFVHCIETHGRNVQYIKFLQTIVKAEGKFIKKCQDMVMAELVNSGEDVLVFYNDRASFQTLIQMMRSERDRMDENSPLMYHIHLVELLAVCTEGKNVYTEIKCNSLLPLDDIVRVVTHEDCIPEVKIAYINFLNHCYVDTEVEMKEIYTSNHMWKLFENFLVDICRACNNTSDRKHADSILEKYVTEIVMSIVTTFFSSPFSDQSTTLQTRQPVFVQLLQGVFRVYHCNWLMPSQKASVESCIRVLSDVAKSRAIAIPVDLDSQVNNLFLKSHSIVQKTAMNWRLSARNAARRDSVLAASRDYRNIIERLQDIVSALEDRLRPLVQAELSVLVDVLHRPELLFPENTDARRKCESGGFICKLIKHTKQLLEENEEKLCIKVLQTLREMMTKDRGYGEKLISIDELDNAELPPAPDSENATEELEPSPPLRQLEDHKRGEALRQVLVNRYYGNVRPSGRRESLTSFGNGPLSAGGPGKPGGGGGGSGSSSMSRGEMSLAEVQCHLDKEGASNLVIDLIMNASSDRVFHESILLAIALLEGGNTTIQHSFFCRLTEDKKSEKFFKVFYDRMKVAQQEIKATVTVNTSDLGNKKKDDEVDRDAPSRKKAKEPTTQITEEVRDQLLEASAATRKAFTTFRREADPDDHYQPGEGTQATADKAKDDLEMSAVITIMQPILRFLQLLCENHNRDLQNFLRCQNNKTNYNLVCETLQFLDCICGSTTGGLGLLGLYINEKNVALINQTLESLTEYCQGPCHENQNCIATHESNGIDIITALILNDINPLGKKRMDLVLELKNNASKLLLAIMESRHDSENAERILYNMRPKELVEVIKKAYMQGEVEFEDGENGEDGAASPRNVGHNIYILAHQLARHNKELQSMLKPGGQVDGDEALEFYAKHTAQIEIVRLDRTMEQIVFPVPSICEFLTKESKLRIYYTTERDEQGSKINDFFLRSEDLFNEMNWQKKLRAQPVLYWCARNMSFWSSISFNLAVLMNLLVAFFYPFKGVRGGTLEPHWSGLLWTAMLISLAIVIALPKPHGIRALIASTILRLIFSVGLQPTLFLLGAFNVCNKIIFLMSFVGNCGTFTRGYRAMVLDVEFLYHLLYLVICAMGLFVHEFFYSLLLFDLVYREETLLNVIKSVTRNGRSIILTAVLALILVYLFSIVGYLFFKDDFILEVDRLPNETAVPETGESLASEFLFSDVCRVESGENCSSPAPREELVPAEETEQDKEHTCETLLMCIVTVLSHGLRSGGGVGDVLRKPSKEEPLFAARVIYDLLFFFMVIIIVLNLIFGVIIDTFADLRSEKQKKEEILKTTCFICGLERDKFDNKTVTFEEHIKEEHNMWHYLCFIVLVKVKDSTEYTGPESYVAEMIKERNLDWFPRMRAMSLVSSDSEGEQNELRNLQEKLESTMKLVTNLSGQLSELKDQMTEQRKQKQRIGLLGHPPHMNVNPQQPA

SEQ ID NO: 93 - protein, 362 aa, Artificial sequence
  synthetic sequence
MQAERGARGGRGRRPGRGRPGGDRHSERPGAAAAVARGGGGGGGGDGGGRRGRGRGRGFRGARGGRGGGGAPRGSRREPGGWGAGASAPVEDDSDAETYGEENDEQGNYSKRKIVSNWDRYQDIEKEVNNESGESQRGTDFSVLLSSAGDSFSQFRFAEEKEWDSEASCPKQNSAFYVDSELLVRALQELPLCLRLNVAAELVQGTVPLEVPQVKPKRTDDGKGLGMQLKGPLGPGGRGPIFELKSVAAGCPVLLGKDNPSPGPSRDSQKPTSPLQSAGDHLEEELDLLLNLDAPIKEGDNILPDQTSQDLKSKEDGEVVQEEEVCAKPSVTEEKNMEPEQPSTSKNVTEEELEDWLDSMIS

SEQ ID NO: 94 - protein, 449 aa, Artificial sequence
  synthetic sequence
MDPGQRPNPFGAPGAIPKKPCLSQGSPGTSGSGAPCDEPSRSESPGEGPSGTGGSAAAGDITRQAVVAAITEWSRTRQLRISTGASEGKASIKDWIVCQVNSGKFPGVEWEDEERTRFRIPVTPLADPCFEWRRDGELGVVYIRERGNMPVDASFKGTRGRRRMLAALRRTRGLQEIGKGISQDGHHFLVFRVRKPEEEQCVECGVVAGAVHDFNNMARLLQEGFFSPGQCLPGEIVTPVPSCTTAEGQEAVIDWGRLFIRMYYNGEQVHELLTTSQSGCRISSALRRDPAVHYCAVGSPGQVWLPNVPNLACEIAKRELCDTLDACAKGILLTSSCNGIFCVCYHNGPVHFIGNTVPPDSGPLLLPQGKPTRIFNPNTFLVGLANSPLPAPSHVTCPLVKLWLGKPVAVGKLEPHAPSPRDFAARCSNFSDACVVLEIMPKPLWDAMQ

SEQ ID NO: 95 - protein, 2644 aa, Artificial sequence
  synthetic sequence
MGEHGLELASMIPALRELGSATPEEYNTVVQKPRQILCQFIDRILTDVNVVAVELVKKTDSQPTSVMLLDFIQHIMKSSPLMFVNVSGSHEAKGSCIEFSNWIITRLLRIAATPSCHLLHKKICEVICSLLFLFKSKSPAIFGVLTKELLQLFEDLVYLHRRNVMGHAVEWPVVMSRFLSQLDEHMGYLQSAPLQLMSMQNLEFIEVTLLMVLTRIIAIVFFRRQELLLWQIGCVLLEYGSPKIKSLAISFLTELFQLGGLPAQPASTFFSSFLELLKHLVEMDTDQLKLYEEPLSKLIKTLFPFEAEAYRNIEPVYLNMLLEKLCVMFEDGVLMRLKSDLLKAALCHLLQYFLKFVPAGYESALQVRKVYVRNICKALLDVLGIEVDAEYLLGPLYAALKMESMEIIEEIQCQTQQENLSSNSDGISPKRRRLSSSLNPSKRAPKQTEEIKHVDMNQKSILWSALKQKAESLQISLEYSGLKNPVIEMLEGIAVVLQLTALCTVHCSHQNMNCRTFKDCQHKSKKKPSVVITWMSLDFYTKVLKSCRSLLESVQKLDLEATIDKVVKIYDALIYMQVNSSFEDHILEDLCGMLSLPWIYSHSDDGCLKLTTFAANLLTLSCRISDSYSPQAQSRCVFLLTLFPRRIFLEWRTAVYNWALQSSHEVIRASCVSGFFILLQQQNSCNRVPKILIDKVKDDSDIVKKEFASILGQLVCTLHGMFYLTSSLTEPFSEHGHVDLFCRNLKATSQHECSSSQLKASVCKPFLFLLKKKIPSPVKLAFIDNLHHLCKHLDFREDETDVKAVLGTLLNLMEDPDKDVRVAFSGNIKHILESLDSEDGFIKELFVLRMKEAYTHAQISRNNELKDTLILTTGDIGRAAKGDLVPFALLHLLHCLLSKSASVSGAAYTEIRALVAAKSVKLQSFFSQYKKPICQFLVESLHSSQMTALPNTPCQNADVRKQDVAHQREMALNTLSEIANVFDFPDLNRFLTRTLQVLLPDLAAKASPAASALIRTLGKQLNVNRREILINNFKYIFSHLVCSCSKDELERALHYLKNETEIELGSLLRQDFQGLHNELLLRIGEHYQQVFNGLSILASFASSDDPYQGPRDIISPELMADYLQPKLLGILAFFNMQLLSSSVGIEDKKMALNSLMSLMKLMGPKHVSSVRVKMMTTLRTGLRFKDDFPELCCRAWDCFVRCLDHACLGSLLSHVIVALLPLIHIQPKETAAIFHYLIIENRDAVQDFLHEIYFLPDHPELKKIKAVLQEYRKETSESTDLQTTLQLSMKAIQHENVDVRIHALTSLKETLYKNQEKLIKYATDSETVEPIISQLVTVLLKGCQDANSQARLLCGECLGELGAIDPGRLDFSTTETQGKDFTFVTGVEDSSFAYGLLMELTRAYLAYADNSRAQDSAAYAIQELLSIYDCREMETNGPGHQLWRRFPEHVREILEPHLNTRYKSSQKSTDWSGVKKPIYLSKLGSNFAEWSASWAGYLITKVRHDLASKIFTCCSIMMKHDFKVTIYLLPHILVYVLLGCNQEDQQEVYAEIMAVLKHDDQHTINTQDIASDLCQLSTQTVFSMLDHLTQWARHKFQALKAEKCPHSKSNRNKVDSMVSTVDYEDYQSVTRFLDLIPQDTLAVASFRSKAYTRAVMHFESFITEKKQNIQEHLGFLQKLYAAMHEPDGVAGVSAIRKAEPSLKEQILEHESLGLLRDATACYDRAIQLEPDQIIHYHGVVKSMLGLGQLSTVITQVNGVHANRSEWTDELNTYRVEAAWKLSQWDLVENYLAADGKSTTWSVRLGQLLLSAKKRDITAFYDSLKLVRAEQIVPLSAASFERGSYQRGYEYIVRLHMLCELEHSIKPLFQHSPGDSSQEDSLNWVARLEMTQNSYRAKEPILALRRALLSLNKRPDYNEMVGECWLQSARVARKAGHHQTAYNALLNAGESRLAELYVERAKWLWSKGDVHQALIVLQKGVELCFPENETPPEGKNMLIHGRAMLLVGRFMEETANFESNAIMKKYKDVTACLPEWEDGHFYLAKYYDKLMPMVTDNKMEKQGDLIRYIVLHFGRSLQYGNQFIYQSMPRMLTLWLDYGTKAYEWEKAGRSDRVQMRNDLGKINKVITEHTNYLAPYQFLTAFSQLISRICHSHDEVFVVLMEIIAKVFLAYPQQAMWMMTAVSKSSYPMRVNRCKEILNKAIHMKKSLEKFVGDATRLTDKLLELCNKPVDGSSSTLSMSTHFKMLKKLVEEATFSEILIPLQSVMIPTLPSILGTHANHASHEPFPGHWAYIAGFDDMVEILASLQKPKKISLKGSDGKFYIMMCKPKDDLRKDCRLMEFNSLINKCLRKDAESRRRELHIRTYAVIPLNDECGIIEWVNNTAGLRPILTKLYKEKGVYMTGKELRQCMLPKSAALSEKLKVFREFLLPRHPPIFHEWFLRTFPDPTSWYSSRSAYCRSTAVMSMVGYILGLGDRHGENILFDSLTGECVHVDFNCLFNKGETFEVPEIVPFRLTHNMVNGMGPMGTEGLFRRACEVTMRLMRDQREPLMSVLKTFLHDPLVEWSKPVKGHSKAPLNETGEVVNEKAKTHVLDIEQRLQGVIKTRNRVTGLPLSIEGHVHYLIQEATDENLLCQMYLGWTPYM

SEQ ID NO: 96 - protein, 172 aa, Artificial sequence
  synthetic sequence
MIIYRDLISHDEMFSDIYKIREIADGLCLEVEGKMVSRTEGNIDDSLIGGNASAEGPEGEGTESTVITGVDIVMNHHLQETSFTKEAYKKYIKDYMKSIKGKLEEQRPERVKPFMTGAAEQIKHILANFKNYQFFIGENMNPDGMVALLDYREDGVTPYMIFFKDGLEMEKC

SEQ ID NO: 97 - protein, 398 aa, Artificial sequence
  synthetic sequence
MEGAALLRVSVLCIWMSALFLGVGVRAEEAGARVQQNVPSGTDTGDPQSKPLGDWAAGTMDPESSIFIEDAIKYFKEKVSTQNLLLLLTDNEAWNGFVAAAELPRNEADELRKALDNLARQMIMKDKNWHDKGQQYRNWFLKEFPRLKSELEDNIRRLRALADGVQKVHKGTTIANVVSGSLSISSGILTLVGMGLAPFTEGGSLVLLEPGMELGITAALTGITSSTMDYGKKWWTQAQAHDLVIKSLDKLKEVREFLGENISNFLSLAGNTYQLTRGIGKDIRALRRARANLQSVPHASASRPRVTEPISAESGEQVERVNEPSILEMSRGVKLTDVAPVSFFLVLDVVYLVYESKHLHEGAKSETAEELKKVAQELEEKLNILNNNYKILQADQEL

SEQ ID NO: 98 - protein, 337 aa, Artificial sequence
  synthetic sequence
MNPESSIFIEDYLKYFQDQVSRENLLQLLTDDEAWNGFVAAAELPRDEADELRKALNKLASHMVMKDKNRHDKDQQHRQWFLKEFPRLKRELEDHIRKLRALAEEVEQVHRGTTIANVVSNSVGTTSGILTLLGLGLAPFTEGISFVLLDTGMGLGAAAAVAGITCSVVELVNKLRARAQARNLDQSGTNVAKVMKEFVGGNTPNVLTLVDNWYQVTQGIGRNIRAIRRARANPQLGAYAPPPHIIGRISAEGGEQVERVVEGPAQAMSRGTMIVGAATGGILLLLDVVSLAYESKHLLEGAKSESAEELKKRAQELEGKLNFLTKIHEMLQPGQDQ

SEQ ID NO: 99 - protein, 198 aa, Artificial sequence; identical to SEQ ID NO: 48
  synthetic sequence

SEQ ID NO: 100 - protein, 195 aa, Artificial sequence; identical to SEQ ID NO: 11
  synthetic sequence

SEQ ID NO: 101 - protein, 135 aa, Artificial sequence
  synthetic sequence
GNRSSHSRLGRIEADSESQEDIIRNIARHLAQVGDSMDRSIPPGLVNGLALQLRNTSRSEEDRNRDLATALEQLLQAYPRDMEKEKTMLVLALLLAKKVASHTPSLLRDVFHTTVNFINQNLRTYVRSLARNGMD

SEQ ID NO: 102 - protein, 184 aa, Artificial sequence; identical to SEQ ID NO: 52
  synthetic sequence

SEQ ID NO: 103 - protein, 91 aa, Artificial sequence; identical to SEQ ID NO: 54
  synthetic sequence

SEQ ID NO: 104 - protein, 160 aa, Artificial sequence; identical to SEQ ID NO: 51
  synthetic sequence

SEQ ID NO: 105 - protein, 239 aa, Artificial sequence; identical to SEQ ID NO: 1
  synthetic sequence

(19) United States
(12) Patent Application Publication
(10) Pub. No.: US 2022/0204575 A1
(43) Pub. Date: Jun. 30, 2022

(54) MODULATING SURVIVAL OF THERAPEUTIC CELLS AND METHODS, CELLS AND NUCLEIC ACIDS RELATED THERETO

(71) Applicant: The Regents of the University of California, Oakland, CA (US)

(72) Inventors: Wendell A. Lim, San Francisco, CA (US); Jasper Z. Williams, San Francisco, CA (US); Greg Allen, San Francisco, CA (US)

(21) Appl. No.: 17/605,198

(22) PCT Filed: Apr. 22, 2020
(86) PCT No.: PCT/US2020/029246

§ 371 Date: Oct. 20, 2021

Related U.S. Application Data
(60) Provisional application No. 62/837,394, filed on Apr. 23, 2019.

(51) Int. Cl. C07K 14/47; A61K 35/17; A61P 35/00; C07K 14/725; A61K 48/00

(52) U.S. Cl. CPC C07K 14/4747; A61K 35/17; A61K 38/00; C07K 14/7051; A61K 48/005; A61P 35/00

(57) ABSTRACT

Provided are methods of modulating the survival of therapeutic cells as well as cells and nucleic acids and vectors useful in such methods. Such survival modulation may include enhancing survival and/or enhancing death of the therapeutic cells. The provided methods include administering a therapeutic cell, nucleic acid and/or vector to a subject, the administered therapeutic cells, nucleic acids and/or vectors including one or more heterologous apoptosis modulating agents and/or one or more encoding sequences thereof. Cells of the disclosure include or encode one or more heterologous apoptosis modulating agents.

### CROSS-REFERENCING

This application claims the benefit of U.S. provisional application Ser. No. 62/837,394, filed on Apr. 23, 2019, which application is incorporated by reference herein.

### STATEMENT REGARDING FEDERALLY SPONSORED RESEARCH

This invention was made with government support under Grant No. R01 CA196277 awarded by National Institutes of Health. The government has certain rights in the invention.

### INTRODUCTION

Cellular therapies, such as stem cell therapy and adoptive cell therapies including e.g., chimeric antigen receptor (CAR) and T cell receptor (TCR) T-cell therapies, involve the administration of therapeutic cells to a subject to treat the subject for a particular condition. In many instances these therapies are exquisitely targeted, e.g., to particular tissues and/or to target cells expressing a particular antigen. Although, cellular therapies have been investigated for decades, recently a number of cellular therapies have proved to be quite successful in the clinic.

For example, adoptive cell therapies for cancer have resulted in multiple cases of complete remission, even in subjects with refractory cancers. In 2017, the US Food and Drug Administration (FDA) approved the first anti-CD19 CAR T-cell therapies for the treatment of patients with relapsed/refractory lymphomas and leukemias and currently, two types of CAR T-cell therapies (tisagenlecleucel and axicabtagene-ciloleucel) are available in the United States and Europe. In addition, hematopoietic stem cell transplants utilizing cells harboring the CCR5-delta32 mutation have resulted in at least two reports of HIV-1 remission in subjects following treatment (Gupta et al., Nature 2019) and this approach is being further evaluated in several clinical trials. In addition to the many adult stem therapies currently moving through the clinic, embryonic stem cell (ESC) and induced pluripotent stem (iPS) cell therapies, including where such cells are used directly or to derive progenitors, have also shown some limited promise in treatments for various conditions such as spinal cord injury, eye diseases, and diabetes.

However, cellular therapies have the potential to cause severe side effects, in some cases resulting in a significant risk of adverse events. For example, subjects treated with CAR T-cell therapies frequently develop cytokine release syndrome (CRS) and are also at risk for certain neurological toxicities. CRS is a systemic response to the activation and proliferation of CAR-T cells causing high fever and flu-like symptoms, which can be life threatening. Neurotoxicities associated with CAR T-cell therapy encompass a wide range of neurological symptoms, including delirium, headache, problems speaking, a decrease in consciousness, seizures and coma, and can also be life threatening. Other cell therapies carry various risks, including neoplasm (e.g., teratoma), immune rejection (e.g., graft-vs-host disease, GVHD), unintended physiological and anatomical consequences (e.g. arrhythmia), blindness, off-target engraftment, toxicity, and others.

### SUMMARY

Provided are methods of modulating the survival of therapeutic cells as well as cells and nucleic acids and vectors useful in such methods. Such survival modulation may include enhancing survival and/or enhancing death of the therapeutic cells. The provided methods include administering a therapeutic cell, nucleic acid and/or vector to a subject, the administered therapeutic cells, nucleic acids and/or vectors including one or more heterologous apoptosis modulating agents and/or one or more encoding sequences thereof. Cells of the disclosure include or encode one or more heterologous apoptosis modulating agents.

### BRIEF DESCRIPTION OF THE DRAWINGS

FIG. 1 schematically depicts precision control of the size of a therapeutic cell population achieved through the implementation of synthetic systems for regulating the activity of BCL2 family proteins to control apoptosis of therapeutic cells.

FIG. 2 depicts differences in levels of anti- and pro-apoptotic reporter expression in cells expressing heterologous BIM, BID, or BAD pro-apoptotic proteins.

FIG. 3 schematically depicts a T cell configured to constitutively express both anti-apoptotic BCL-2 protein and pro-apoptotic BAD protein as described herein.

FIG. 4 demonstrates that constitutive expression of BAD in the presence of constitutive expression of BCL2 was insufficient to drive T cell death.

FIG. 5 schematically depicts a T cell configured for ligand-inducible expression of tBID and constitutive expression of BCL2, demonstrating at least partial BCL2 buffering against the pro-apoptotic effects of induced tBID expression.

FIG. 6 demonstrates buffering of pro-apoptotic effects, where increasing BCL2 expression resulted in less tBID-induced apoptosis.

FIG. 7 schematically depicts a dual-ligand-inducible system where anti-apoptotic and pro-apoptotic factors are separately inducible by two different antigen inputs separately influencing survival and apoptosis.

FIG. 8 schematically depicts an inducible BCL2 circuit employed to result in antigen-induced expression of BCL2 when, e.g., T cells are cultured in the absence of IL-2.

FIG. 9 provides a diagram depicting a synNotch NOT module engineered in primary human CD8 T cells where an anti-Her2 Gal4-VP64 synNotch induces expression of pro-apoptosis protein tBID.

FIG. 10 provides quantification of replicate T cell survival data gathered via flow cytometry after 1:1 E:T co-cultures for 48 hours between engineered CD8 T cells shown in FIG. 9 and K562 target cells with or without Her2 expression. While survival of untransduced control T cells did not depend on the antigen expression of co-cultured K562 cells, tBID/synNotch T cells showed reduced survival after co-culture with Her2 positive K562 target cells (n=3, error bars are SD).

FIG. 11 shows a schematic design of a 3-input circuit integrating an AND-gate with a NOT-gate to control T cell activation: the anti-GFP synNotch induces expression of the anti-CD19 41BB-zeta CAR, and the orthogonal anti-Her2 synNotch induces expression of pro-apoptosis protein tBID.

FIG. 12 provides a diagram depicting 3-receptor circuit engineered in primary human CD8 T cells to integrate both positive and negative regulation controlling T cell activation. These T cells must first bind GFP via the LexA-VP64 synNotch to induce the anti-CD19 41BB-zeta CAR, and only activate and kill target cells if they sense both GFP and CD19. The T cells also express the anti-Her2 Gal4-VP64 synNotch circuit that induces tBID expression and T cell apoptosis in response to Her2 antigen binding. SynNotch-driven T cell apoptosis depletes the effector T cell population and serves as an antigen-dependent NOT-gate to prevent target cell killing.

FIG. 13 provides quantification of replicate target cell killing data gathered via flow cytometry after 1:1 E:T co-cultures of varied times between engineered CD8 T cells shown in FIG. 12 and K562 target cells expressing different combinations of CD19, GFP, and Her2. As shown, while in the first 24 hours the T cells kill some of the Her2/GFP/CD19+ off-target cells, there is no further killing at later time points. In contrast, the T cells continue to kill the GFP/CD19+ on-target cells over the 96-hour time course (n=3, error bars are SD).

FIG. 14 provides forward scatter and side scatter flow cytometry plots corresponding to the 96-hour time point data shown in FIG. 13. K562 target cells fall within the indicated gate. The 3_{AND-NOT} circuit T cells specifically killed the GFP/CD19+ K562 cells and spared Her2/GFP/CD19+ K562 cells, as demonstrated by the selective reduction of cells in the K562 gate (representative of at least 3 independent experiments).

FIG. 15 provides quantification of T cell numbers via flow cytometry after 96 hours in the co-culture experiment described in FIG. 13. T cell counts show selective expansion when T cells were co-cultured with GFP/CD19+ target cells and NOT Her2/GFP/CD19+ target cells (n=3, error bars are SD).

FIG. 16 provides histograms of T cells stained with CellTrace Violet dye in the co-culture experiment described in FIG. 13 and processed via flow cytometry after 96 hours. The leftward shift of peaks in the histograms indicate T cell proliferation and dilution of the pre-labeled dye during increasing rounds of cell division. The T cells strongly proliferate only in the presence of GFP/CD19+ target cells and NOT Her2/GFP/CD19+ target cells (representative of at least 3 independent experiments).

FIG. 17 provides results for the same time-course experiments as provided in FIG. 13 for CD8 T cells as performed with primary human CD4 T cells engineered with the circuit shown in FIG. 12. Similar to the CD8 cells, these CD4 T cells specifically killed the GFP/CD19+K562 cells and spared Her2/GFP/CD19+K562 cells (n=3, error bars are SD).

FIG. 18 provides quantification of CD4 T cell numbers via flow cytometry after 96 hours in the coculture experiment described in FIG. 17. Similar to the CD8 cells, the CD4 T cell counts showed selective expansion when T cells were co-cultured with GFP/CD19+ target cells and NOT Her2/GFP/CD19+ target cells (n=3, error bars are SD).

FIG. 19 schematically depicts response element constructs, responsive to the released transcription factor (TF)-containing intracellular portion of a SynNotch receptor, driving expression of various BCL-2 family proteins.

FIG. 20 demonstrates different levels of basal T cell death from the inducible response element constructs depicted in FIG. 19 in the absence of SynNotch-triggering antigen.

FIG. 21 schematically depicts a ligand-inducible circuit configured with an alternative binding-triggered transcriptional switch (BTTS), namely a A2 force sensor receptor, to drive expression of a BCL-2 family protein in response to antigen binding.

FIG. 22 demonstrates antigen-induced T cell apoptosis, in comparison to survival in the absence of antigen, using the circuit depicted in FIG. 21 employing an alternative BTTS.

FIG. 23 provides schematic depictions of non-limiting examples of different antigen-input driven circuits providing cell-autonomous modulation of cell survival.

FIG. 24 provides a schematic depicting a T cell configured with a small molecule-inducible circuit driving expression of a pro-apoptotic BCL-2 family protein.

FIG. 25 demonstrates drug-titratable T cell apoptosis using the small molecule-inducible circuit depicted in FIG. 24.

FIG. 26 provides the raw flow cytometry data quantified in FIG. 25, further showing small molecule-titratable primary T cell apoptosis in the described system.

FIG. 27 schematically depicts a T cell configured for Her2 antigen-dependent induction of a pro-apoptotic BCL-2 family protein “X” using an anti-Her2 synNotch receptor.

FIG. 28 provides the resulting levels of T cell survival when various pro-apoptotic BCL-2 family members were employed as protein “X” in the T cell depicted in FIG. 27, demonstrating that tunable levels of T cell death may be achieved.

FIG. 29 schematically depicts a T cell configured for antigen-dependent induction of a BCL-2 family protein using BTTS regulation.

FIG. 30 schematically depicts a T cell configured for local stimulus regulated expression of a BCL-2 family protein using local stimulus, such as hypoxia, to regulate a corresponding transcription factor and regulatory element.

FIG. 31 schematically depicts a T cell configured for drug-regulated expression of a BCL-2 family protein using small molecule regulated transcription factor and a corresponding regulatory element.

FIG. 32 schematically depicts a T cell configured for drug-regulated activity of a BCL-2 family protein using a regulatory domain bound by a small molecule to control activity of a BCL-2 family protein.

FIG. 33 schematically depicts a T cell configured for stimulus regulated expression of a BCL-2 family protein using a stimulus, such as light, to regulate a corresponding transcription factor and regulatory element.

### DEFINITIONS

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to a mammal, including, but not limited to, murines (e.g., rats, mice), lagomorphs (e.g., rabbits), non-human primates, humans, canines, felines, ungulates (e.g., equines, bovines, ovines, porcines, caprines), etc. In some cases, the individual is a human.

A “therapeutically effective amount” or “efficacious amount” refers to the amount of an agent, or combined amounts of two agents, that, when administered to a mammal or other subject for treating a disease, is sufficient to effect such treatment for the disease. The “therapeutically effective amount” will vary depending on the agent(s), the disease and its severity and the age, weight, etc., of the subject to be treated.

By “specifically binds” or “selectively bind” is meant that the molecule binds preferentially to the target of interest or binds with greater affinity to the target than to other molecules. For example, a DNA molecule will bind to a substantially complementary sequence and not to unrelated sequences. Specific binding may refer to non-covalent or covalent preferential binding to a molecule relative to other molecules or moieties in a solution or reaction mixture (e.g., an antibody specifically binds to a particular polypeptide or epitope relative to other available polypeptides). In some embodiments, the affinity of one molecule for another molecule to which it specifically binds is characterized by a K_{D} (dissociation constant) of 10⁻⁵ M or less (e.g., 10⁻⁶ M or less, 10⁻⁷ M or less, 10⁻⁸ M or less, 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, or 10⁻¹⁶ M or less). “Affinity” refers to the strength of binding, increased binding affinity being correlated with a lower K_{D}.

The terms “antibody” and “immunoglobulin”, as used herein, are used interchangeably may generally refer to whole or intact molecules or fragments thereof and modified and/or conjugated antibodies or fragments thereof that have been modified and/or conjugated. The immunoglobulins can be divided into five different classes, based on differences in the amino acid sequences in the constant region of the heavy chains. All immunoglobulins within a given class will have very similar heavy chain constant regions. These differences can be detected by sequence studies or more commonly by serological means (i.e. by the use of antibodies directed to these differences). Immunoglobulin classes include IgG (Gamma heavy chains), IgM (Mu heavy chains), IgA (Alpha heavy chains), IgD (Delta heavy chains), and IgE (Epsilon heavy chains).

Antibody or immunoglobulin may refer to a class of structurally related glycoproteins consisting of two pairs of polypeptide chains, one pair of light (L) low molecular weight chains and one pair of heavy (H) chains, all four inter-connected by disulfide bonds. The structure of immunoglobulins has been well characterized, see for instance Fundamental Immunology Ch. 7 (Paul, W., ed., 2nd ed. Raven Press, N.Y. (1989)). Briefly, each heavy chain typically is comprised of a heavy chain variable region (abbreviated as V_{H}) and a heavy chain constant region (abbreviated as C_{H}). The heavy chain constant region typically is comprised of three domains, C_{H}1, C_{H}2, and C_{H}3. Each light chain typically is comprised of a light chain variable region (abbreviated as V_{L}) and a light chain constant region (abbreviated herein as C_{L}). The light chain constant region typically is comprised of one domain, C_{L}. The V_{H} and V_{L} regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs).

Whole or largely intact antibodies are generally multivalent, meaning they may simultaneously bind more than one molecule of antigen whereas antibody fragments may be monovalent. Antibodies produced by an organism as part of an immune response are generally monospecific, meaning they generally bind a single species of antigen. Multivalent monospecific antibodies, i.e. antibodies that bind more than one molecule of a single species of antigen, may bind a single antigen epitope (e.g., a monoclonal antibody) or multiple different antigen epitopes (e.g., a polyclonal antibody).

Multispecific (e.g., bispecific) antibodies, which bind multiple species of antigen, may be readily engineered by those of ordinary skill in the art and, thus, may be encompassed within the use of the term “antibody” used herein where appropriate. Also, multivalent antibody fragments may be engineered, e.g., by the linking of two monovalent antibody fragments. As such, bivalent and/or multivalent antibody fragments may be encompassed within the use of the term “antibody”, where appropriate, as the ordinary skilled artisan will be readily aware of antibody fragments, e.g., those described below, which may be linked in any convenient and appropriate combination to generate multivalent monospecific or polyspecific (e.g., bispecific) antibody fragments.

Antibody fragments include but are not limited to antigen-binding fragments (Fab or F(ab), including Fab′ or F(ab′), (Fab)₂, F(ab′)₂, etc.), single chain variable fragments (scFv or Fv), “third generation” (3G) molecules, etc. which are capable of binding the epitopic determinant. These antibody fragments retain some ability to selectively bind to the subject antigen, examples of which include, but are not limited to:

(1) Fab, the fragment which contains a monovalent antigen-binding fragment of an antibody molecule can be produced by digestion of whole antibody with the enzyme papain to yield an intact light chain and a portion of one heavy chain;

(2) Fab′, the fragment of an antibody molecule can be obtained by treating whole antibody with pepsin, followed by reduction, to yield an intact light chain and a portion of the heavy chain; two Fab′ fragments are obtained per antibody molecule;

(3) (Fab)₂, the fragment of the antibody that can be obtained by treating whole antibody with the enzyme pepsin without subsequent reduction;

(4) F(ab)₂ is a dimer of two Fab′ fragments held together by two disulfide bonds;

(5) Fv, defined as a genetically engineered fragment containing the variable region of the light chain and the variable region of the heavy chain expressed as two chains;

(6) Single chain antibody (“SCA”), defined as a genetically engineered molecule containing the variable region of the light chain, the variable region of the heavy chain, linked by a suitable polypeptide linker as a genetically fused single chain molecule; such single chain antibodies may be in the form of multimers such as diabodies, triabodies, tetrabodies, etc. which may or may not be polyspecific (see, for example, WO 94/07921 and WO 98/44001) and

(7) “3G”, including single domain (typically a variable heavy domain devoid of a light chain) and “miniaturized” antibody molecules (typically a full-sized Ab or mAb in which non-essential domains have been removed).

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as polynucleotides or polypeptides. The term “biological sample” encompasses a clinical sample, and also includes cells in culture, cell supernatants, cell lysates, serum, plasma, biological fluid, and tissue samples. The term “biological sample” includes urine, saliva, cerebrospinal fluid, interstitial fluid, ocular fluid, synovial fluid, blood fractions such as plasma and serum, and the like. The term “biological sample” also includes solid tissue samples, tissue culture samples, and cellular samples.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a cell” includes a plurality of such cells and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication. Further, the dates of publication provided may be different from the actual publication dates which may need to be independently confirmed.

### DETAILED DESCRIPTION

As summarized above, the present disclosure provides methods, cells, nucleic acids, and the like for, or involving, the modulation of survival of therapeutic cells. For example, methods of the present disclosure may involve modulating the survival of therapeutic cells, e.g., to enhance the survival (e.g., decrease death, promote proliferation, etc.) of therapeutic cells and/or to decrease the survival (e.g., increase death, prevent proliferation, etc.) of therapeutic cells, including populations thereof. Strategies for modulating the survival of therapeutic cells are described in more detail below, including strategies for modulating apoptosis in therapeutic cells employing BCL-2 family member proteins.

Also provided are therapeutic cells that are configured such that their survival may be modulated. Various means for enhancing the survival and/or decreasing the survival of such cells are provided, including e.g., molecular circuits that include nucleic acid sequences configured to allow for the desired control of therapeutic cell survival modulation. Various strategies employing BCL-2 family member proteins in such cells are described in more detail below.

In some embodiments, enhanced survival of therapeutic cells may result in increased efficacy of cellular therapies, e.g., through increasing the resulting population size of therapeutic cells administered to a subject and/or increasing the persistence in the subject of the administered therapeutic cells. In some embodiments, decreased survival of therapeutic cells may result in increased efficacy of cellular therapies, e.g., through decreasing toxic side effects of the administered cells. In some embodiments, decreased survival of therapeutic cells, e.g., through an inducible or cell-autonomous increase in apoptosis of the therapeutic cells, may prevent or reduce the occurrence of one or more adverse events attributable to the therapeutic cells and/or a cellular therapy employing the therapeutic cells. Toxic side effects of, and/or adverse events attributable to, therapeutic cells may be due to on-target activity, off-target activity, or a combination thereof. By “on-target activity”, as used herein in this context, is meant the activity of a therapeutic cell attributable or induced by an antigen targeted by the therapeutic cells. As will be readily understood, in some instances, a targeted antigen may be expressed by only both target cells (e.g., cancer cells) and non-target cells (e.g., non-cancer cells). By “off-target activity”, as used herein in this context, is meant an activity of a therapeutic cell not attributable or not induced by an antigen targeted by the therapeutic cells.

In some instances, strategies for both enhancing and decreasing therapeutic cell survival may be employed in the same method and/or cell population. The combined use of inducible enhancement of survival and inducible decreased survival in a therapeutic cell may be referred to herein as bi-directional control.

Bi-directional control of therapeutic cell survival may be employed for various purposes including but not limited to, to buffer pro- and/or anti-apoptotic effects. For example, in some instances, the effects of a pro-apoptotic agent may be buffered by an anti-apoptotic agent, including where the anti-apoptotic agent is constitutively or inducibly expressed. In particular contexts, any pro-apoptotic factor may be subject to buffering by an anti-apoptotic factor as desired. Buffered pro-apoptotic effect may be induced or uninduced or a combination thereof. By “uninduced pro-apoptotic effects” as used herein, is meant any pro-apoptotic effect attributable to a pro-apoptotic agent in the absence of induction of the agent, e.g., in the absence of the inducing antigen, small molecule, stimulus, etc. Uninduced pro-apoptotic effects may be due to various characteristics of a pro-apoptotic factor including e.g., high effectiveness of the factor to induce apoptosis, and/or characteristics of the system employed for induction of the factor, including e.g., leaky expression of an inducible system, such as a leaky inducible promoter or the like. As a non-limiting example, in some instances, the pro-apoptotic effects of uninduced BIM may be buffered by an anti-apoptotic factor, such as e.g., BCL-2.

In some instances, bi-directional control may be employed to preferentially increase and/or decrease the presence of therapeutic cells at a desired site and/or an undesired site, respectively. For example, a therapeutic cell may be configured with bi-directional control such that an anti-apoptotic agent is induced when the cell is present at a desired site and/or not present at an undesired site and a pro-apoptotic agent is induced when the cell is present at an undesired site and/or not present at the desired site. Desired and undesired sites (also referred to as targeted and non-targeted sites) may be sensed in various ways by a cell configured with a bi-directional control circuit of the present disclosure, including e.g., through the presence of an antigen expressed by a target cell or tissue, the absence of an antigen expressed by a target cell or tissue, the presence of an antigen expressed by a non-target cell or tissue, the absence of an antigen expressed by a non-target cell or tissue, the presence/absence of a delivered agent (e.g., a delivered bioorthogonal ligand, a delivered small molecule, etc.), the presence/absence of a stimulus (e.g., light, ultrasound, hypoxia, etc.), and the like. In some instances, strategies for enhancing and decreasing therapeutic cell survival may be employed separately.

In some embodiments, the methods, cells, nucleic acids, circuits, and the like, described herein may provide for enhanced or preferential survival of therapeutic cells at a target site (e.g., as compared to therapeutic cell survival at non-target sites), including where such enhanced or preferential survival is externally controlled (i.e., user-controlled), cell-autonomous, or a combination thereof. In some embodiments, the methods, cells, nucleic acids, circuits, and the like, described herein may provide for decreased survival of therapeutic cells at a non-target (i.e., off-target) sites (e.g., as compared to therapeutic cell survival at a target site), including where such decreased survival is externally controlled (i.e., user-controlled), cell-autonomous, or a combination thereof. Such differential survival at target versus non-target sites may, in some instances, provide one or more advantages, including e.g., increased efficacy of a cell therapy, decreased risk of adverse events attributable to administered therapeutic cells, decreased occurrence of side-effects from cell therapy, and the like.

Methods

As summarized above, methods are provided that involve modulating the survival of therapeutic cells, including enhancing survival of therapeutic cells and/or decreasing the survival of therapeutic cells. Methods of enhancing the survival of therapeutic cells will generally involve expressing one or more heterologous anti-apoptotic BCL-2 family proteins in the therapeutic cells. Methods of decreasing the survival of therapeutic cells will generally involve expressing one or more heterologous pro-apoptotic BCL-2 family proteins in the therapeutic cells. In some embodiments, methods may employ expressing a combination of one or more heterologous anti-apoptotic BCL-2 family proteins and one or more heterologous pro-apoptotic BCL-2 family proteins in the therapeutic cells.

Expression of the heterologous BCL-2 family members may be configured to be conditional or constitutive. For example, in some instances, a therapeutic cell may constitutively express a heterologous anti-apoptotic BCL-2 family member protein to constitutively enhance survival of the therapeutic cell. In some instances, a therapeutic cell may conditionally express a heterologous pro-apoptotic BCL-2 family member protein to conditionally decrease survival of the therapeutic cell.

Conditional expression may vary and may include e.g., cell-autonomous conditional expression, user-defined inducible expression, and the like. For example, in some instances, a therapeutic cell may be configured to cell-autonomously express a heterologous BCL-2 family protein in response to a cell encountered stimulus (e.g., an antigen, a ligand, hypoxia, etc.) that results in induction of the heterologous BCL-2 family protein in a cell-autonomous manner. In some instances, a therapeutic cell may be configured such that a stimulus (e.g., a small molecule, an antigen, a ligand, light, ultra sound, etc.) may be applied to the cell by a user to result in induction of a heterologous BCL-2 family protein. In some instances, expression of one or more, including all, heterologous BCL-2 family proteins in a subject method may be wholly constitutive or wholly conditional. In some instances, constitutive and conditional expression strategies may be combined such that, in a cell, one or more heterologous BCL-2 family proteins are expressed constitutively and one or more heterologous BCL-2 family proteins are expressed conditionally.

Various configurations of heterologous modulators of apoptosis and various BCL-2 family member proteins may be utilized in modulating survival of therapeutic cells in the methods of the present disclosure and the cells employed in such methods as described in more detail below.

Methods of the present disclosure may include administering a therapeutic cell that includes one or more heterologous apoptosis modulating agents to a subject in need thereof, including e.g., where one or more of the heterologous apoptosis modulating agents are constitutive, inducible, or a combination thereof. Useful heterologous apoptosis modulating agents include BCL-2 family pro- and anti-apoptotic proteins, including inducible BCL-2 family pro- and inducible anti-apoptotic proteins configured as described in more detail below.

Methods of the present disclosure may involve therapeutic cells, including administering therapeutic cells to a subject and/or modulating the survival of administered therapeutic cells. Useful therapeutic cells will vary and may include but are not limited to e.g., therapeutic immune cells, therapeutic stem cells, and the like. Suitable therapeutic cells may be autologous and allogenically derived. For example, in some instances, cells may be obtained from a subject and modified ex vivo, e.g., to include one or more heterologous apoptosis modulating BCL-2 family pro- and/or anti-apoptotic proteins, and subsequently administered to the subject as a cell therapy. In some instances, cells may be obtained from a first subject and modified ex vivo, e.g., to include one or more heterologous apoptosis modulating BCL-2 family pro- and/or anti-apoptotic proteins, and subsequently administered to a second subject in need thereof as a cell therapy. Such cells are described in more detail below.

Therapeutic cells may or may not express or encode a heterologous therapeutic agent. For example, in some instances, a therapeutic cell may be a cell, such as a stem cell or an immune cell, that has not been modified to encode a therapeutic agent. In some instances, a therapeutic cell may be a cell, such as a stem cell or an immune cell, that has been modified to encode a therapeutic agent. In some instances, therapeutic cells may be modified in other ways, including e.g., by mutating or deleting an endogenous gene of the cell, by introducing a sequence encoding a gene product other than a therapeutic agent, by a cell culture regimen designed to modify the cell, and the like.

Accordingly, in some instances, therapeutic cells of the methods of the present disclosure may have been modified to include at least one therapeutic agent and/or sequence encoding the therapeutic agent. The therapeutic agent may be endogenous, such as a cytokine or TCR in a therapeutic immune cell that endogenously produces the cytokine or TCR. The therapeutic agent may be heterologous, such as a CAR, a therapeutic antibody, an engineered TCR, or a cytokine in a cell that does not naturally express the CAR, the therapeutic antibody, the engineered TCR, or the cytokine.

Whether the therapeutic agent is endogenous or heterologous the therapeutic agent may be encoded from a heterologous nucleic acid. Put another way, the therapeutic cell may include a heterologous nucleic acid encoding a therapeutic agent. For example, a therapeutic immune cell that endogenously expresses a cytokine may further include a heterologous copy of a nucleic acid encoding the cytokine. Thus, a therapeutic cell may have been modified to include one or more additional copies of an endogenous therapeutic agent, including e.g., where the one or more additional copies are operably linked to a heterologous regulatory sequence. Accordingly, expression of a therapeutic agent may be driven non-endogenously (i.e., by a heterologous regulatory sequence). In some instances, the therapeutic agent may be entirely heterologous, partially or entirely synthetic, and/or recombinantly derived.

Useful therapeutic agents will vary. Therapeutic agents expressed by a therapeutic cell may be encoded and may include essentially any encoded therapeutic, including nucleic acid therapeutics (e.g., DNA, RNA, DNA/RNA hybrids, DNA analogs, RNA analogs, and the like) and polypeptide therapeutics. Useful polypeptide therapeutics include proteins of interest (POIs) as described in more detail below. In some instances, useful encoded therapeutic agents include but are not limited to e.g., therapeutic antibodies, chimeric antigen receptors (CARs), engineered T cell receptors (TCRs), cytokines, bispecific binding agents, and the like.

Therapeutic polypeptide agents may target, and be responsive to, one or more target antigens. Useful therapeutic agents for targeting one or more target antigens include but are not limited to antibodies, bispecific binding members, CARs, TCRs, and the like. A therapeutic polypeptide may target essentially any antigen, or combination of antigens, for which an antigen binding agent that binds to one or more epitopes of the antigen(s) are known or can be derived. In some instances, a therapeutic polypeptide may target an antigen expressed by cells of the subject, including e.g., tissue specific antigens, cancer antigens, and the like. In some instances, a therapeutic polypeptide may target an antigen that is not naturally expressed by cells of the subject, including e.g., bioorthogonal antigens, such as non-natural bioorthogonal ligands.

Useful antigens that may be targeted by a therapeutic agent, or in some cases by a BTTS, include but are not limited to e.g., cancer antigens, i.e., an antigen expressed by (synthesized by) a neoplasia or cancer cell, i.e., a cancer cell associated antigen or a cancer (or tumor) specific antigen.

A cancer cell associated antigen can be an antigen associated with, e.g., a breast cancer cell, a B cell lymphoma, a pancreatic cancer, a Hodgkin lymphoma cell, an ovarian cancer cell, a prostate cancer cell, a mesothelioma, a lung cancer cell (e.g., a small cell lung cancer cell), a non-Hodgkin B-cell lymphoma (B-NHL) cell, an ovarian cancer cell, a prostate cancer cell, a mesothelioma cell, a lung cancer cell (e.g., a small cell lung cancer cell), a melanoma cell, a chronic lymphocytic leukemia cell, an acute lymphocytic leukemia cell, a neuroblastoma cell, a glioma, a glioblastoma, a medulloblastoma, a colorectal cancer cell, etc. A cancer cell associated antigen may also be expressed by a non-cancerous cell.

Non-limiting examples of cancer associated antigens include but are not limited to e.g., CD19, CD20, CD38, CD30, Her2/neu, ERBB2, CA125, MUC-1, prostate-specific membrane antigen (PSMA), CD44 surface adhesion molecule, mesothelin, carcinoembryonic antigen (CEA), epidermal growth factor receptor (EGFR), EGFRvIII, vascular endothelial growth factor receptor-2 (VEGFR2), high molecular weight-melanoma associated antigen (HMW-MAA), MAGE-A1, IL-13R-a2, GD2, and the like. Cancer-associated antigens also include, e.g., 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD152, CD19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DRS, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, L1-CAM, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, and vimentin.

A cancer cell specific antigen can be an antigen specific for cancer and/or a particular type of cancer or cancer cell including e.g., a breast cancer cell, a B cell lymphoma, a pancreatic cancer, a Hodgkin lymphoma cell, an ovarian cancer cell, a prostate cancer cell, a mesothelioma, a lung cancer cell (e.g., a small cell lung cancer cell), a non-Hodgkin B-cell lymphoma (B-NHL) cell, an ovarian cancer cell, a prostate cancer cell, a mesothelioma cell, a lung cancer cell (e.g., a small cell lung cancer cell), a melanoma cell, a chronic lymphocytic leukemia cell, an acute lymphocytic leukemia cell, a neuroblastoma cell, a glioma, a glioblastoma, a medulloblastoma, a colorectal cancer cell, etc. A cancer (or tumor) specific antigen is generally not expressed by non-cancerous cells (or non-tumor cells). In some instances, a cancer (or tumor) specific antigen may be minimally expressed by one or more non-cancerous cell types (or non-tumor cell types). By “minimally expressed” is meant that the level of expression, in terms of either the per-cell expression level or the number of cells expressing, minimally, insignificantly or undetectably results in binding of a specific binding member to non-cancerous cells expressing the antigen.

Useful antigens also include surface expressed antigens. As used herein the term “surface expressed antigen” generally refers to antigenic proteins that are expressed at least partially extracellularly such that at least a portion of the protein is exposed outside the cell and available for binding with a binding partner. Essentially any surface expressed protein, for which a specific binding member is known or may be derived, may find use as a target of a BTTS or antigen-specific therapeutic.

Useful antigens also include intracellular antigen, e.g., intracellular antigens expressed in the context of MHC. For example, a specific binding member may be employed that specifically binds to a peptide-major histocompatibility complex (peptide-MHC). Non-limiting examples of antigens that may be targeted in the context of MHC, including e.g., by targeting a peptide-MHC, include WT1, KRAS and mutants thereof (e.g., G12V & G12C), EGFP and mutants thereof (e.g., L858R), PR1/Proteinase 3, MAGE-A1, MAGE3, P53, MART-1, gp100, CMV pp65, HIV Vpr, HA-1H, NY-ESO-1, EBNA3C, AFP, Her2, hCG-beta, HBV Env183-91, hTERT, MUC1, TARP, Tyrosinase, p68, MIF, PRAME, and the like, including but not limited to e.g., those described in PCT Pub. No. WO 2018/039247; the disclosure of which is incorporated herein by reference in its entirety.

Non-limiting examples of useful antigens include but are not limited to e.g., CD19, CD20, CD38, CD30, Her2/neu, ERBB2, CA125, MUC-1, prostate-specific membrane antigen (PSMA), CD44 surface adhesion molecule, mesothelin, carcinoembryonic antigen (CEA), epidermal growth factor receptor (EGFR), EGFRvIII, vascular endothelial growth factor receptor-2 (VEGFR2), high molecular weight-melanoma associated antigen (HMW-MAA), MAGE-A1, IL-13R-a2, GD2, and the like. In some instances, useful antigens may be selected from: AFP, BCMA, CD10, CD117, CD123, CD133, CD138, CD171, CD19, CD20, CD22, CD30, CD33, CD34, CD38, CD5, CD56, CD7, CD70, CD80, CD86, CEA, CLD18, CLL-1, cMet, EGFR, EGFRvIII, EpCAM, EphA2, GD-2, Glypican 3, GPC3, HER-2, kappa immunoglobulin, LeY, LMP1, mesothelin, MG7, MUC1, NKG2D-ligands, PD-L1, PSCA, PSMA, ROR1, ROR1R, TACI and VEGFR2 and may include, e.g., an antigen binding-domain of or derived from a CAR currently or previously under investigation in one or more clinical trials.

Useful targets of a therapeutic agent, or in some cases by a BTTS, include but are not limited to bioorthogonal ligands. By “orthogonal”, as used herein, is generally meant a component or system that may be present and/or function without interfering with another component or system. Thus, two components or systems may be orthogonal in that they may be used and/or function in the same cell, tissue, organ, organism, or subject without interfering with one another. Also, a component or system may be orthogonal in that it does not interfere with all or substantially all of the biological functions of the cell, tissue, organ, organism, or subject within which it is present and/or used.

By “bioorthogonal ligand”, as used herein, is generally meant a ligand that is biocompatible and does not interfere with and is not naturally present in the relevant cell, tissue, organ, organism, and/or subject. In addition, two ligands may be bioorthogonal in that they do not interfere with each other and/or one does not interfere with the processes and/or signaling attributable to the other.

Accordingly, in some instances, bioorthogonal ligands may be non-natural in the sense that they are naturally occurring in some unrelated context but not naturally present or produced or occurring in the relevant context, e.g., by the relevant cell, tissue, organ, organism, and/or subject. For example, GFP may serve as a bioorthogonal ligand in the context of a cell that is modified to include a GFP-binding partner (e.g., a synthetic receptor comprising an anti-GFP binding domain) where the cell does not naturally express GFP and the presence of GFP does not substantially interfere with any biological activity of the cell or the activity of another ligand employed in a signaling system of the cell. Systems may, in some instances, employ components from an evolutionarily distant organism or cell type to achieve an orthogonal activity. For example, components derived from prokaryotes or plants, such as e.g., plant hormone S-(+)-abscisic acid (ABA) and its binding proteins PYL1 and ABI1 or gibberellic acid 3 (GA₃) and its cognate binding proteins GAI and GID1, may be employed in eukaryotic or mammalian systems, respectively, to achieve bioorthogonal activity.

In some instances, bioorthogonal ligands may be non-natural in the sense that they are recombinant or partially or wholly synthetic and not produced in nature and thus, not naturally present or produced in the relevant context, e.g., by the relevant cell, tissue, organ, organism and/or subject. For example, a synthetic ligand may serve a bioorthogonal ligand in the context of a cell that includes or is contacted with a binding partner for the synthetic ligand (e.g., synthetic biotin/avidin mimetics, binding partners employing synthetic binding domains such as zippers (see e.g., Anderson et al., ACS Omega (2018) 3(5):4810-4815), chemically induced dimerization (CID) systems employing recombinant binding domains and/or synthetic analogs of chemical dimerizer (see e.g., DeRose et al., Pflugers Arch (2013) 465(3):409-417), etc. Synthetic and/or recombinant ligands may be employed that do not significantly interfere with natural biological activities of a cell, tissue, organ, organism, or subject in which they are employed.

A therapeutic agent expressed by therapeutic cells may or may not be regulatable. For examples, in some instances, a non-regulatable therapeutic agent may be expressed constitutively. In some instances, expression of a therapeutic agent may be regulatable through the use of an inducible promoter controlling expression of the therapeutic agent, where essentially any appropriate inducible promoter may be employed. In some instances, a tetracycline/doxycycline inducible system may be employed, including e.g., where expression of a CAR by a therapeutic cell is induced by administering doxycycline or an analog or derivative thereof. In some instances, expression of the therapeutic agent may be regulated by a binding-triggered transcriptional switch.

As summarized above, expression of heterologous inducible apoptosis modulating agent(s) in the methods of the present disclosure may vary and may include conditional expression, inducible expression, and combinations thereof. Methods of the present disclosure may therefore employ one or more sequences encoding BCL-2 family pro- and/or anti-apoptotic proteins operably linked to one or more regulatory sequences.

In some embodiments, the subject methods, or cells employed in such methods, may employ three or more different heterologous coding sequences. Accordingly, the actual number of heterologous coding sequences employed in a subject method or cell may vary and may range from 1 to 6 or more, including but not limited to e.g., 1 or more, 2 or more, 3 or more, 4 or more, 5 or more, 6 or more, 1, 2, 3, 4, 5, 6, etc. For example, in some embodiments a method or cell may include a first sequence encoding a heterologous pro-apoptotic agent, a second sequence encoding a heterologous anti-apoptotic agent, and at least a third sequence encoding a therapeutic polypeptide. In some instances, multiple pro-apoptotic agents may be employed. In some instances, multiple anti-apoptotic agents may be employed. In some instances, multiple therapeutic agents may be employed. Such sequences may individually be constitutive or inducible. Useful regulatory sequences include but are not limited to e.g., constitutive promoters, small molecule inducible regulatory sequences, stimuli inducible regulatory sequences, and the like.

In some instances, expression of a heterologous apoptosis modulating agent may be ligand inducible. As used herein, by “ligand inducible” is generally meant that the expression of the encoded heterologous apoptosis modulating agent is dependent upon whether the cell containing the coding sequence is bound to a particular ligand. For example, in some instances a binding triggered transcriptional switch (BTTS) that includes a binding domain specific for a ligand may be employed to drive expression of a heterologous apoptosis modulating agent. Accordingly, upon binding its ligand, the BTTS may release an intracellular domain that induces expression of the heterologous apoptosis modulating agent through a regulatory sequence operably linked to a sequence encoding the heterologous apoptosis modulating agent.

Essentially any ligand for which a binding domain, that specifically binds to one or more epitopes of the ligand, is known or can be derived may be targeted by a BTTS. In some instances, a binding domain of a BTTS may bind a ligand expressed by cells of the subject, including e.g., a ligand expressed by non-target cells, e.g., non-cancer cells, a ligand expressed by target cells, e.g., cancer cells, a ligand expressed tissue specifically, or the like. In some instances, a BTTS may bind to a ligand that is not naturally expressed by cells of the subject, including e.g., a non-natural bioorthogonal ligand. Accordingly, useful ligands include naturally occurring and recombinant and/or synthetic ligands. Cellular ligands may be endogenous or heterologous to a cell expressing the ligand and ligands may be present on a cell, expressed by cells (including target cells and non-target cells), present on a solid support, or otherwise presented.

In some instances, ligands may not be expressed by cells and may be provided by other non-cellular means. For example, in some instances, a ligand may be provided in solution or bound to a solid- or semi-solid support, e.g., a solid- or semi-solid non-cellular support. Any appropriate support may be employed including but not limited to e.g., a flat or substantially planar surface, a particle, a vessel surface, or the like. Useful particles include but are not limited to e.g., polymer particles, such as latex (e.g., polystyrene) beads, microspheres, polymer coated particles (e.g., polymer coated superparamagnetic particles, polymer coated (e.g., PEGylated) nanoparticles), and the like.

Methods of the present disclosure may include administering to a subject a nucleic acid, including a vector and/or expression cassette including a nucleic acid sequence, encoding one or more heterologous apoptosis modulators or a cell comprising such a nucleic acid, vector or expression cassette. Accordingly, in some instances, a subject may be administered a nucleic acid to genetically modify one or more cells of the subject to encode one or more heterologous apoptosis modulators. In some instances, a subject may be administered a cell that has been previously genetically modified to encode one or more heterologous apoptosis modulators. Cells may be genetically modified in various ways, including where such cells are modified in vitro, ex vivo, in vivo, and the like. Methods of the present disclosure may include genetically modifying a cell with a nucleic acid, including a vector and/or expression cassette including a nucleic acid sequence, encoding one or more heterologous apoptosis modulators.

Methods of treating a subject may include inducing one or more heterologous inducible apoptosis modulating agents. In some instances, one or more heterologous inducible pro-apoptosis agents may be induced. In some instances, one or more heterologous inducible anti-apoptosis agents may be induced. In some instances, both one or more heterologous inducible pro-apoptosis agents and one or more heterologous inducible anti-apoptosis agents may be induced.

Methods of treating a subject may include treating a subject for an adverse reaction to a therapeutic cell. For example, a subject may have been or may be administered therapeutic cells containing an inducible heterologous pro-apoptotic BCL-2 family protein and the heterologous pro-apoptotic BCL-2 family protein may be induced, e.g., to treat the subject for an adverse reaction to the administered therapeutic cells. In some instances, the heterologous pro-apoptotic BCL-2 family member may be a truncated BID (tBID), a BIM, a PUMA, a BMF, a HRK, or a BIK.

Induction of the heterologous pro-apoptotic BCL-2 family protein may be performed at various times, including e.g., before the subject displays symptoms of the adverse reaction, essentially when the subject displays symptoms of the adverse reaction, after the subject displays symptoms of the adverse reaction (including after one or more of symptoms have resolved or improved). The heterologous pro-apoptotic BCL-2 family protein may be induced consistent with the system employed for induction in the administered cells, including e.g., by administering a small molecule where a small molecule-inducible system is employed, by administering a stimulus where a stimuli-inducible system is employed, by administering a ligand where a ligand-inducible system is employed, etc.

Symptoms of an adverse response to a cell therapy may vary depending on the particular cell therapy employed. Non-limiting examples of symptoms of an adverse response to a cell therapy may include be are not limited to e.g., symptoms of cytokine release syndrome (CRS, e.g., high fever, flu-like symptoms, etc.), symptoms of neurotoxicity (e.g., delirium, headache, problems speaking, a decrease in consciousness, seizures, coma, etc.), symptoms of neoplasia (e.g., lump or growth, abnormal imaging, abnormal biopsy, abnormal cytology, etc.), symptoms of immune rejection (including e.g., symptoms of GVHD), atypical physiological conditions (e.g., arrhythmia, atypical blood pressure, atypical respiration, etc.), symptoms of chronic or acute toxicity, and the like.

Where a heterologous pro-apoptotic BCL-2 family protein is induced before the subject displays symptoms of the adverse reaction the timing of the induction may be determined in various ways. For example, in some instances, the induction may be performed at a predetermined time point after the start of treatment with the therapeutic cells (e.g., to reduce or terminate treatment with the therapeutic cells). In some instances, the induction may be performed when a predetermined treatment outcome is reached (e.g., to reduce or terminate treatment with the therapeutic cells). In some instances, the induction may be performed before starting another, new, and/or different course of therapy (e.g., to reduce or clear a prior dose of therapeutic cells prior to administering a new dose).

Methods of the present disclosure may enhance a cellular therapy administered to a subject. Such methods may include administering or having administered a therapeutic cell comprising an inducible heterologous anti-apoptotic agent comprising a BCL-2 family anti-apoptotic protein to a subject; and inducing the inducible heterologous anti-apoptotic agent. Such inducible heterologous anti-apoptotic agent comprising a BCL-2 family anti-apoptotic protein will vary and may include e.g., where the BCL-2 family anti-apoptotic protein is a BCL-2 protein.

The therapeutic cell administered or having been administered in such methods may vary and may include but are not limited to e.g., cells that include one or more therapeutic polypeptides, and/or one or more nucleic acid sequence(s) encoding such therapeutic polypeptides. Useful therapeutic polypeptides will vary and may include those responsive to a target antigen. Useful therapeutic polypeptides responsive to a target antigen include but are not limited to e.g., therapeutic antibodies, chimeric antigen receptors, engineered T cell receptors, and the like.

Heterologous Apoptosis Modulating Agents

As summarized above, the present disclosure employs heterologous apoptosis modulating agents in modulating the survival of therapeutic cells. Useful heterologous apoptosis modulating agents include BCL-2 family member proteins, as described in more detail below. Heterologous apoptosis modulating agents may be constitutive or inducible.

In some embodiments, heterologous inducible pro-apoptotic agents may include inducible BCL-2 family pro-apoptotic proteins. In some embodiments, heterologous anti-apoptotic agents may include BCL-2 family anti-apoptotic proteins. In some embodiments, combinations of pro- and anti-apoptotic proteins may be employed.

As summarized above, the activity and/or expression of heterologous apoptosis modulating agents may be induced by a variety of means. For example, in various embodiments, such agents may be ligand inducible, small molecule inducible, stimuli inducible, and the like.

In some instances, systems for small molecule induction of expression, or components of such systems, may be employed. Useful systems for small molecule induction of expression include but are not limited to e.g., Tet or Tet analog (e.g., doxycycline) regulated systems, estrogen receptor (ER)/tamoxifen regulated systems, the Rheoswitch system (e.g., as available from Ziopharm, Boston, Mass.), and the like.

In some instances, systems for regulated activity, or components of such systems, may be employed. Such systems may be induced by various means, including small molecules, polypeptide ligands, and the like. Useful systems for small molecule regulated activity include small molecule inducible dimerization systems such as but not limited to e.g., rapamycin-induced dimerization systems, iDimerize systems (Takara Bio Inc., Mountain View, Calif.), and the like. Useful systems for regulated activity also include autoinhibited synthetic switches, such as but not limited to e.g., those described in Dueber et al., Science. (2003) 301(5641):1904-8 and U.S. Pat. No. 7,604,805; the disclosures of which are incorporated herein by reference in their entirety. Useful systems for regulated activity also include ligand and small molecule controlled protein degradation such as, but not limited to e.g., those systems and components described in Rakhit et al., Chem Biol. (2014) 21(9):1238-52 and commercialized by Obsidian Therapeutics, Inc. (Boston, Mass.), as well as ligand induced degradation (LID) and destabilization domain (DD) systems (such as but not limited to e.g., those described in Bonger et al., Nat Chem Biol. 2012; 7(8): 531-537; Grimley et al., Bioorg. Med. Chem. Lett. (2008) 18: 759-761; and Chu et al. Bioorg. Med. Chem. Lett. (2008) 18: 5941-5944; Iwamoto et al., Chemistry & Biology (2010) 17: 981-988; the disclosures of which are incorporated herein by reference in their entirety).

Accordingly, methods of the present disclosure may include contacting a sample with an agent (e.g., a small molecule or ligand) or administering an agent (e.g., small molecule or ligand) to a subject containing cells that include the agent inducible system. In some instances, a contacted or administered small molecule binds a transcriptional activator of the regulatory sequence thereby inducing expression of the heterologous apoptosis modulating agent. In some instances, a contacted or administered small molecule competitively binds a transcriptional repressor of the regulatory sequence thereby inducing expression of the heterologous apoptosis modulating agent. In some instances, a contacted or administered small molecule or ligand regulates activity of a heterologous apoptosis modulating agent (e.g., reversing autoinhibition, promoting or inhibiting induced degradation, etc.) thereby inducing activity of the heterologous apoptosis modulating agent.

In some instances, split heterologous apoptosis modulating agents may be employed. By “split heterologous apoptosis modulating agent” is generally meant a heterologous apoptosis modulating agent that has been modified by splitting the heterologous apoptosis modulating agent into at least two part and configuring the split portions of the heterologous apoptosis modulating agent such that they may be controllably or inducibly associated. Accordingly, the unassociated portions of the split heterologous apoptosis modulating agent will generally be essentially non-functional, and when the split portions are re-associated the function of the heterologous apoptosis modulating agent may be restored.

Split heterologous apoptosis modulating agents may be configured to be re-associated by a variety of means. For example, in some instances, the split portions of a heterologous apoptosis modulating agent may be re-associated using a small molecule, e.g., a heterologous apoptosis modulating agent split into two portions may be dimerized by a small-molecule dimerizer. Useful systems for dimerizing split portions of a heterologous apoptosis modulating agent include small molecule regulated dimerization systems such as but not limited to e.g., rapamycin-induced dimerization systems, iDimerize systems (Takara Bio Inc., Mountain View, Calif.), and the like. In some instances, a dimerizer in such a system may be referred to as a re-associating agent and the portions of the split heterologous apoptosis modulating agent may be dimerized by the re-associating agent such that the dimerized (i.e., re-associated) split heterologous apoptosis modulating agent performs its respective function, e.g., promoting or inhibiting apoptosis.

The amount of agent (e.g., small molecule, polypeptide (e.g., polypeptide ligand), etc.) administered may vary and may be an amount effective to induce the heterologous apoptosis modulating agent, including where such induction includes induced expression, induced activity, and the like. Such effective amounts may be pre-determined or may be determined during treatment, e.g., by monitoring the subject or cells for the desired response, including e.g., a desired pro-apoptotic effect or a desired anti-apoptotic effect. In some instances, monitoring may be compared to an appropriate reference or control or may involve the use of a reporter or appropriate assay for apoptosis and/or survival. In some embodiments, the agent may be administered systemically to a subject or a sample. In some embodiments, the agent may be administered locally to, e.g., a portion of, a subject or a sample, including e.g., a target site such as a desired tissue or a tumor.

In some instances, systems for stimuli induced expression, or components of such systems, may be employed. Useful systems for stimuli induced expression include but are not limited to e.g., those systems that are induced by light, ultrasound or hypoxia. Non-limiting examples of stimuli inducible systems include systems employing light-responsive promoters and light-switchable transactivators, such as but not limited to e.g., those described in Horner et al. Methods Mol Biol. (2017) 1651:173-186; Chen et al. Nucleic Acids Res. (2016) 44(6): 2677-2690; Muller et al. Nucleic Acids Res. (2013) 41:e124; Muller et al. Nucleic Acids Res. (2013) 41:e77; Kennedy et al. Nat. Methods. (2010) 7:973-975; Ye et al. Science. (2011) 332:1565-1568; Motta-Mena et al. Nat. Chem. Biol. (2014) 10:196-202; Wang et al., Nat. Methods. (2012) 9:266-269; Polstein et al. Nat. Chem. Biol. (2015) 11:198-200; and Nihongaki et al. Chem. Biol. (2015) 22:169-174; as well as U.S. Pat. Nos. 10,221,422; 9,988,655; 9,540,653; 6,858,429; 6,294,714; and 5,639,952; the disclosures of which are incorporated herein by reference in their entirety.

Non-limiting examples of stimuli inducible systems include systems employing ultrasound-responsive promoters and ultrasound-switchable components, such as but not limited to e.g., those described in Ogawa et al., Methods Mol Biol. (2017) 1651:187-203; Watanabe et al., J Med Ultrasound. (2009) 36:9-17; Ogawa et al. Ultrason Sonochem. 2012; Vollmer et al. Appl Environ Microbiol. (1998) 64(10): 3927-3931; Wilson et al. J Dent Res. (2013) 92(5): 409-417; and Kagiya et al., Ultrasound Med. Bio. 35(1):155-64; as well as PCT Pub No. WO/1998/006864 and U.S. Pat. No. 7,056,897; the disclosures of which are incorporated herein by reference in their entirety.

Non-limiting examples of stimuli inducible systems include systems employing hypoxia-responsive promoters and hypoxia-switchable components, such as but not limited to e.g., those described in Javan & Shahbazi, Ecancermedicalscience. (2017) 11: 751; Lee et al., Gene Ther. (2006) 13(10):857-68; Gao et al., Toxicol Sci. (2013) 132(2): 379-389; and Luo & Zhu, Biomed Res Int. (2014) 2014: 751397; as well as PCT Pub. Nos. WO1999013067A2, WO1999048916A2 and WO2004076633A2 and U.S. Pat. Nos. 5,942,434; 10,155,795; 9,783,526; 7,973,156; 7,608,698; 7,524,935; 7,396,922; 6,555,667; 6,541,621; and 6,218,179; the disclosures of which are incorporated herein by reference in their entirety

Accordingly, methods of the present disclosure may include applying a stimulus to a sample or a subject containing the cells that include the stimuli inducible system or subjecting a sample or a subject containing the cells that include the stimuli inducible system to conditions that result in the cells being exposed to the stimulus.

As such, methods of the present disclosure may include applying light to a sample or a subject or exposing the sample or subject to light sufficient to induce the inducible system. Various different forms of light may be employed depending on the particular conditions and configuration of the inducible system. In some instances, broad spectrum light may be applied, including but not limited to e.g., broad spectrum light in the visible range, broad spectrum light in the ultraviolet (UV) range, broad spectrum light in the infrared (IR) range, or combinations thereof spanning one or more broad spectrum ranges. In some instances, light of a wavelength, or restricted or narrow range of wavelengths may be applied, including but not limited to e.g., a UV wavelength, or range of UV wavelengths, within e.g., the range of 10 nm to 380 nm; a violet wavelength, or range of violet wavelengths, within e.g., the range of 380 nm to 450 nm; a blue wavelength, or range of blue wavelengths, within e.g., the range of 450 nm to 495 nm; a green wavelength, or range of green wavelengths, within e.g., the range of 495 nm to 570 nm; a yellow wavelength, or range of yellow wavelengths, within e.g., the range of 570 nm to 590 nm; a orange wavelength, or range of orange wavelengths, within e.g., the range of 590 nm to 620 nm; a red wavelength, or range of red wavelengths, within e.g., the range of 620 nm to 750 nm; a far red wavelength, or range of far red wavelengths, within e.g., the range of 710 nm to 850 nm; an infrared wavelength, or range of infrared wavelengths, within e.g., the range of 700 nm to 1 mm; or overlapping ranges or combinations thereof. Ranges of wavelengths, also referred to as bands, may vary in size and in some instances, may span less than 5 nm to 100 nm or more, including but not limited to e.g., 5 nm to 10 nm, 10 nm to 20 nm, 20 nm to 30 nm, 30 nm to 40 nm, 40 nm to 50 nm, 50 nm to 60 nm, 60 nm to 70 nm, 70 nm to 80 nm, 80 nm to 90 nm, 90 nm to 100 nm, 5 nm to 100 nm, 10 to 100 nm, 10 to 50 nm, etc.

Methods of the present disclosure may include applying ultrasound waves to a sample or a subject or exposing the sample or subject to ultrasound waves sufficient to induce the inducible system. Various different forms of ultrasound may be employed depending on the particular conditions and configuration of the inducible system. In some instances, ultrasound may be applied ranging from 20 kHz up to several gigahertz, including but not limited to e.g., ultrasound in the 20 to 200 kHz range, ultrasound in the 200 to 400 kHz range; ultrasound in the 400 to 800 kHz range; ultrasound in the 800 kHz to 1 MHz range; ultrasound in the 1 to 10 MHz range; ultrasound in the 1 to 100 MHz range; ultrasound in the 1 to 200 MHz range; ultrasound in the 10 to 100 MHz range; ultrasound in the 200 to 400 MHz range; ultrasound in the 400 to 600 MHz range; ultrasound in the 600 to 800 MHz range; ultrasound in the 800 MHz to 1 gHz range; ultrasound in the 1 to 10 gHz range; and the like.

Methods of the present disclosure may include subjecting a sample or a subject to hypoxic conditions or exposing the sample or subject to a hypoxic environment sufficient to induce the inducible system. Various different methods of rendering a sample or a subject hypoxic may be employed depending on the particular conditions and configuration of the inducible system. In some instances, a subject or sample may be placed in a hypoxic environment. For example, a sample may be placed in a chamber containing a lower-than-atmospheric concentration of oxygen or a hypoxic session may be applied to a subject, e.g., where the subject breathes a gas mixture with reduced inspiratory oxygen fraction. In some instances, the oxygen concentration of a gas mixture administered to a subject may be continually adjusted to reach at target arterial oxygen saturation. Various target hypoxic levels may be applied, including but not limited to e.g., where the target level of oxygen in a tissue is at or below 3%, including e.g., 3% or less, 2.5% or less, 2% or less, 1.5% or less, 1% or less, etc. In some instances, a target hypoxic level of atmospheric/environmental oxygen applied, e.g., to a sample in a tissue culture incubator, may be at or below 18%, including but not limited to e.g., 18% or less, 16% or less, 14% or less, 12% or less, 10% or less, 8% or less, 6% or less, 5% or less, 4% or less, 3% or less, 2% or less, etc.

In some instances, a cell of the present disclosure may be exposed to hypoxic conditions by nature of being associated with, or within the proximity of, a solid tumor, or within a solid tumor microenvironment. Hypoxia is a feature of many solid tumors. The rapid growth of tumor cells and their inability to form normal blood vessels reduces the blood supply and oxygen transmission. Therefore, the oxygen concentration in the tumour microenvironment often drops to hypoxic levels, e.g., levels at or below 3%, including e.g., 3% or less, 2.5% or less, 2% or less, 1.5% or less, 1% or less, etc. Accordingly, in some instances, subjecting a cell to hypoxic conditions may include administering the cell to a subject having, or suspected of having, a solid tumor having, or expected to have, a hypoxic microenvironment. In such instances, the cell may be administered by a direct route to, or within the proximity of, the tumor microenvironment. In some instances, the cell may be administered indirectly and the cell may diffuse or actively migrate to the tumor microenvironment.

The amount of stimulus administered may vary and may be an amount effective to induce the heterologous apoptosis modulating agent. Such effective amounts may be pre-determined or may be determined during treatment, e.g., by monitoring the subject for the desired response, including e.g., a desired pro-apoptotic effect or a desired anti-apoptotic effect. In some instances, monitoring may be compared to an appropriate reference or control or may involve the use of a reporter or appropriate assay for apoptosis and/or survival. In some embodiments, the stimulus may be administered systemically to a subject or a sample. In some embodiments, the stimulus may be administered locally to, e.g., a portion of, a subject or a sample.

As summarized above, in some instances ligand inducible systems may be employed. As described above, ligand inducible systems generally involve where the expression of the encoded heterologous apoptosis modulating agent is dependent upon whether the cell containing the coding sequence is bound to a particular ligand. Ligand may be administered by a user, or naturally or synthetically present. For example, in some instances a BTTS that includes a binding domain specific for a ligand may be employed to drive expression of a heterologous apoptosis modulating agent, where the ligand is administered, naturally present on a cell, or synthetically present on a cell. Accordingly, upon binding the ligand, the BTTS may induce expression of the heterologous apoptosis modulating agent.

In some embodiments, the expression of a heterologous apoptosis modulator that includes a pro-apoptotic BCL-2 family protein may be regulated by a BTTS. For example, in some embodiments, BTTS-regulated expression of a heterologous apoptosis modulator that includes a pro-apoptotic BCL-2 family protein may be employed as a NOT-gate, e.g., as a NOT-gate in a combinatorial antigen sensing circuit. In such cases, a first antigen may be the target of an antigen-directed therapeutic and a second antigen may be the target of a BTTS driving expression of the heterologous apoptosis modulator that includes a pro-apoptotic BCL-2 family protein. Thus, a cell, that includes the subject combinatorial antigen sensing, may be activated in the presence of the first antigen. However, in the presence of the second antigen, expression of the pro-apoptotic BCL-2 family protein is triggered by the BTTS, thus promoting apoptosis of the cell. Accordingly, survival of the cell is reduced in the presence of the second antigen and the second antigen therefore serves as a NOT-gate for the functional outcome of the activated cell.

BTTS regulated expression of a heterologous apoptosis modulator that includes a pro-apoptotic BCL-2 family protein may therefore prevent therapeutic cells from contributing to an adverse event. For example, a T cell that includes a BTTS regulated heterologous apoptosis modulator that includes a pro-apoptotic BCL-2 family protein may be prevented from contributing an adverse immune response in the presence of the antigen to which the BTTS binds and/or prevented from damaging cells and/or tissue that expresses the antigen to which the BTTS binds (i.e., the NOT-gate antigen). Accordingly, in some instances, the BTTS of such a circuit may target an antigen of a non-target tissue, thus protecting the non-target tissue.

In some instances, a NOT-gate may prevent a therapeutic cell from engrafting in and/or delivering a payload (e.g., a therapeutic included in or expressed in the therapeutic cell) to a non-target tissue. For example, in some instances, a NOT-gate may prevent a T cell from delivering a therapeutic payload to the wrong (i.e., non-target or off-target) tissue.

In some embodiments, the expression of a heterologous apoptosis modulator that includes a pro-apoptotic BCL-2 family protein may be drug-regulated. Drug-regulated expression of a heterologous apoptosis modulator that includes a pro-apoptotic BCL-2 family protein in a cell may provide the ability to eliminate the cell as desired in a drug-dependent manner. For example, a subject treated with a therapeutic cell that includes a drug-regulated heterologous apoptosis modulator that includes a pro-apoptotic BCL-2 family protein may be administered the drug to reduce the function and/or population of therapeutic cells present in the subject. Accordingly, in some instances, administration of the drug may be employed to titrate down the function and/or size of the population of therapeutic cells present in a subject.

In some embodiments, administration of the drug may eliminate or substantially eliminate therapeutic cells administered to a subject. For example, a subject may be administered a population of therapeutic T cells (e.g., CAR T cells, TCR T cells, or the like) that include a heterologous apoptosis modulator that includes a pro-apoptotic BCL-2 family protein and the drug may be administered to eliminate a portion, or all or substantially all, of the therapeutic T cells. Depending on the context, elimination of the therapeutic cells may be localized or systemic, e.g., depending on the drug and/or the route of administration of the drug. In some instances, administering the drug to a subject treated with a population of therapeutic T cells that include a heterologous apoptosis modulator that includes a pro-apoptotic BCL-2 family protein may treat the subject for an adverse event associated with the administered therapeutic T cells.

In some embodiments, the activity of a heterologous apoptosis modulator that includes a pro-apoptotic BCL-2 family protein may be drug-regulated. Drug-regulated activity of a heterologous apoptosis modulator that includes a pro-apoptotic BCL-2 family protein in a cell may provide the ability to eliminate the cell as desired in a drug-dependent manner. For example, the drug may be administered to convert an inactive form of the pro-apoptotic BCL-2 family protein into an active form, thus rendering the pro-apoptotic BCL-2 family protein capable of inducing apoptosis in the cell in which it is expressed.

In some embodiment, a subject treated with a therapeutic cell configured for drug-regulated activity of heterologous apoptosis modulator that includes a pro-apoptotic BCL-2 family protein may be administered the drug to reduce the function and/or population of therapeutic cells present in the subject. Accordingly, in some instances, administration of the drug may be employed to titrate down the function and/or size of the population of therapeutic cells present in a subject.

In some embodiments, administration of the drug may eliminate or substantially eliminate therapeutic cells configured for drug-regulated activity of the heterologous apoptosis modulator. For example, a subject may be administered a population of therapeutic T cells (e.g., CAR T cells, TCR T cells, or the like) that have been configured for drug-regulated activity of a heterologous apoptosis modulator that includes a pro-apoptotic BCL-2 family protein and the drug may be administered to eliminate a portion, or all or substantially all, of the therapeutic T cells. Depending on the context, elimination of the therapeutic cells may be localized or systemic, e.g., depending on the drug and/or the route of administration of the drug. In some instances, administering the drug to a subject treated with a population of therapeutic T cells configured for drug-regulated activity of a heterologous apoptosis modulator may treat the subject for an adverse event associated with the administered therapeutic T cells.

In some instances, employing a drug to induce drug-regulated activity of a heterologous apoptosis modulator that includes a pro-apoptotic BCL-2 family protein may provide for a faster response as compared to mediating expression of a heterologous apoptosis modulator that includes a pro-apoptotic BCL-2 family protein in a drug-regulated manner. In some instances, employing a drug to induce expression of a heterologous apoptosis modulator that includes a pro-apoptotic BCL-2 family protein may provide for a more sustained response as compared to inducing activity of the heterologous apoptosis modulator that includes a pro-apoptotic BCL-2 family protein in a drug-regulated manner. Accordingly, various drug-regulated systems may be employed and may be selected, in some instances, based on various factors, including the desired timing of the response, the desired duration of the response, the desired robustness of the response, and the like.

In some embodiments, the expression of a heterologous apoptosis modulator that includes a pro-apoptotic BCL-2 family protein may be stimuli-regulated. Stimuli-regulated expression of a heterologous apoptosis modulator that includes a pro-apoptotic BCL-2 family protein in a cell may provide the ability to eliminate the cell as desired through the application of the stimulus, e.g., to a subject or a sample. For example, the stimuli may be administered to induce expression of the pro-apoptotic BCL-2 family protein, thus inducing apoptosis in the cell in which it is expressed. As described above, various stimuli may be administered, including e.g., light, ultrasound, and the like.

In some embodiments, stimuli-induced expression may provide for localized induction of a heterologous apoptosis modulator that includes a pro-apoptotic BCL-2 family protein. For example, in some instances, the stimulus may be focused on a desired area, site, tissue, organ, etc., such that expression of the heterologous apoptosis modulator is induced only, or essentially only, in therapeutic cells within the targeted area, site, tissue, organ, etc. In addition, in some instances, a stimulus may be selected for having certain physical properties that are beneficial for targeting of a particular area, site, tissue, organ, etc.

For example, where a surface of a tissue is to be targeted, e.g., to induce apoptosis in therapeutic cells near the surface of a tissue, a low-penetrating light stimulus (e.g., UV light, blue light, etc.) or an ultrasonic stimulation configured for shallow penetration may be selected. Where a deep tissue, or a deep portion of a tissue, is to be targeted, e.g., to induce apoptosis in therapeutic cells far from the surface of a tissue, a high-penetrating light stimulus (e.g., red light, IR light, etc.) or an ultrasonic stimulation configured for deep penetration may be selected. Application of a stimulus, such as light or ultrasound, may also provide for tight temporal control over induction and/or multiple rounds of induction that may be easily applied. Accordingly, stimuli-induced expression of a heterologous apoptosis modulator may, in some instances, provide for modulation of survival of therapeutic cells with specific spatiotemporally control of induction in a user-controlled manner.

In some embodiments, the expression of a heterologous apoptosis modulator that includes an anti-apoptotic BCL-2 family protein may be regulated by a BTTS. For example, in some embodiments, BTTS-regulated expression of a heterologous apoptosis modulator that includes an anti-apoptotic BCL-2 family protein may be employed to enhance survival of therapeutic cells in an antigen dependent manner. For example, a therapeutic T cell (e.g., a CAR T cell, a TCR T cell, etc.), that includes a BTTS-regulated heterologous apoptosis modulator that includes an anti-apoptotic BCL-2 family protein may be administered to enhance on-target effects of the administered cells, e.g., killing by therapeutic (e.g., CAR/TCR/etc.) T cells. For such purposes, the BTTS may be configured to target an antigen present on the target cells and/or within the target tissue. As such, survival of the therapeutic cells in or associated with the target cells or tissues may be enhanced relative to therapeutic cells in or associated with non-target cells or tissues.

In some instances, a circuit configured with a BTTS driving expression of a heterologous apoptosis modulator that includes an anti-apoptotic BCL-2 family protein may promote engraftment of the therapeutic cell in a target tissue and/or promote delivery of a payload (e.g., a therapeutic included in or expressed in the therapeutic cell) to a target tissue. the tumor microenvironment is often immunosuppressive. Thus, in some embodiments, a circuit configured with a BTTS driving expression of a heterologous apoptosis modulator that includes an anti-apoptotic BCL-2 family protein may enhance survival of a therapeutic cell within the immunosuppressive tumor microenvironment. In some embodiments, a circuit configured with a BTTS driving expression of a heterologous apoptosis modulator that includes an anti-apoptotic BCL-2 family protein may amplify the delivery of a therapeutic payload to a target tissue, e.g., by promoting survival, accumulation, and/or proliferation of therapeutic cells, thus resulting in more payload being delivered to target cells/tissues.

In some embodiments, the expression of a heterologous apoptosis modulator that includes an anti-apoptotic BCL-2 family protein may be drug-regulated. Drug-regulated expression of a heterologous apoptosis modulator that includes an anti-apoptotic BCL-2 family protein in a cell may provide the ability to enhance survival of the cell as desired in a drug-dependent manner. For example, a subject treated with a therapeutic cell that includes a drug-regulated heterologous apoptosis modulator that includes an anti-apoptotic BCL-2 family protein may be administered the drug to enhance the function and/or population of therapeutic cells present in the subject. Accordingly, in some instances, administration of the drug may be employed to titrate up the function and/or size of the population of therapeutic cells present in a subject.

In some embodiments, drug-regulated expression of a heterologous apoptosis modulator that includes an anti-apoptotic BCL-2 family protein may provide the ability to enhance survival of therapeutic cells in a drug-dependent manner in a way that is orthogonal to other cells in the body. For example, in comparison to conventional drugs employed to either boost or suppress the immune system, the drug employed for drug-regulated expression may be bioorthogonal. Accordingly, the survival and/or population of therapeutic cells may be enhanced without effecting, or without substantially effecting, the endogenous cells of the subject, including e.g., endogenous stem cells, endogenous immune cells, and the like.

In some instances, drug-regulated expression of a heterologous apoptosis modulator that includes an anti-apoptotic BCL-2 family protein to enhance survival of therapeutic cells in a subject may eliminate or reduce or substantially reduce the need for pre-treatment (i.e., preconditioning) of a subject conventionally employed in various cell therapies.

In some embodiments, the activity of a heterologous apoptosis modulator that includes an anti-apoptotic BCL-2 family protein may be drug-regulated. Drug-regulated activity of a heterologous apoptosis modulator that includes an anti-apoptotic BCL-2 family protein in a cell may provide the ability to enhance survival of the cell as desired in a drug-dependent manner. For example, the drug may be administered to convert an inactive form of the anti-apoptotic BCL-2 family protein into an active form, thus rendering the anti-apoptotic BCL-2 family protein capable of inhibiting apoptosis in the cell in which it is expressed.

In some embodiments, a subject treated with a therapeutic cell configured for drug-regulated activity of heterologous apoptosis modulator that includes an anti-apoptotic BCL-2 family protein may be administered the drug to enhance the function and/or population size of therapeutic cells present in the subject. Accordingly, in some instances, administration of the drug may be employed to titrate up the function and/or size of the population of therapeutic cells present in a subject.

In some instances, employing a drug to induce drug-regulated activity of a heterologous apoptosis modulator that includes an anti-apoptotic BCL-2 family protein may provide for a faster response as compared to mediating expression of a heterologous apoptosis modulator that includes an anti-apoptotic BCL-2 family protein in a drug-regulated manner. In some instances, employing a drug to induce expression of a heterologous apoptosis modulator that includes an anti-apoptotic BCL-2 family protein may provide for a more sustained response as compared to inducing activity of the heterologous apoptosis modulator that includes the anti-apoptotic BCL-2 family protein in a drug-regulated manner. Accordingly, various drug-regulated systems may be employed and may be selected, in some instances, based on various factors, including the desired timing of the response, the desired duration of the response, the desired robustness of the response, and the like.

In some embodiments, the expression of a heterologous apoptosis modulator that includes an anti-apoptotic BCL-2 family protein may be stimuli-regulated. Stimuli-regulated expression of a heterologous apoptosis modulator that includes an anti-apoptotic BCL-2 family protein in a cell may provide the ability to enhance survival of the cell as desired through the application of the stimulus, e.g., to a subject or a sample. For example, the stimuli may be administered to induce expression of the anti-apoptotic BCL-2 family protein, thus inhibiting apoptosis in the cell in which it is expressed. As described above, various stimuli may be administered, including e.g., light, ultrasound, and the like.

In some embodiments, stimuli-induced expression may provide for localized induction of a heterologous apoptosis modulator that includes an anti-apoptotic BCL-2 family protein. For example, in some instances, the stimulus may be focused on a desired area, site, tissue, organ, etc., such that expression of the heterologous apoptosis modulator is induced only, or essentially only, in therapeutic cells within the targeted area, site, tissue, organ, etc. Local induction through the use of a stimulus may provide for local maintenance/expansion of therapeutic cells in a user-controlled spatiotemporally specific manner. In addition, in some instances, a stimulus may be selected for having certain physical properties that are beneficial for targeting of a particular area, site, tissue, organ, etc.

In some embodiments, stimuli-induced expression may provide for cell-autonomous induction of a heterologous apoptosis modulator that includes an anti-apoptotic BCL-2 family protein. For example, in some embodiments, a hypoxia-induced system may be employed in a subject having a tumor. According, when the cell is present in or associated with the hypoxic tumor microenvironments the heterologous apoptosis modulator that includes an anti-apoptotic BCL-2 family protein may be induced by the hypoxia stimulus. Thus, in this context, the survival of the cells is preferentially enhanced within the tumor microenvironment.

In some embodiments, methods and circuits of the present disclosure may employ buffering of pro- and anti-apoptotic heterologous apoptosis modulators. For example, in some instances, basal expression of a first heterologous apoptosis modulator may be buffered by a second heterologous apoptosis modulator having the opposite effect as the first modulator. In some instances, an anti-apoptotic modulator may buffer basal expression of a pro-apoptotic modulator. In some instances, a pro-apoptotic modulator may buffer basal expression of an anti-apoptotic modulator. In some instances, buffering may be employed to reduce or eliminate or substantially eliminate the effects of a heterologous apoptosis modulator driven by a leaky transcriptional regulator, including e.g., those of inducible systems.

In some instances, a heterologous apoptosis modulator that includes an anti-apoptotic BCL-2 family protein may be employed to at least partially buffer basal induction of a heterologous apoptosis modulator that includes a pro-apoptotic BCL-2 family protein. In some instances, the pro-apoptotic BCL-2 family protein may be a truncated BID (tBID) protein. In some instances, the anti-apoptotic BCL-2 family protein may be BCL-2. In some instances, where an anti-apoptotic BCL-2 family protein is employed to buffer the effects of a pro-apoptotic BCL-2 family protein, the anti-apoptotic BCL-2 family protein may be constitutively expressed. In some instances, where an anti-apoptotic BCL-2 family protein is employed to buffer the effects of a pro-apoptotic BCL-2 family protein, the anti-apoptotic BCL-2 family protein may be inducible.

In some instances, the buffering-heterologous apoptosis modulator may be tuned, e.g., by selecting a more or less potent buffering-modulator, by selecting a more or less potent buffered-modulator, by modifying the level of expression of the buffering-modulator, by modulating the level of expression of the buffered-modulator, or the like.

Useful heterologous apoptosis modulators include proteins, and protein encoding sequences, derived from a variety of sources. In some instances, a heterologous apoptosis modulator is a eukaryotic protein, or derived from a eukaryote, including but not limited to e.g., where the heterologous apoptosis modulator protein is a mammalian protein, or mammalian encoding sequence, including but not limited to e.g., a human protein or encoding sequence, a non-human primate protein or encoding sequence, a rodent (e.g., a mouse, a rat, etc.) protein or encoding sequence, or the like.

BCL-2 Family Proteins

As summarized above, various BCL-2 family proteins may be employed as apoptosis modulating agents in the methods of the preset disclosure. As used herein, by “BCL-2 family proteins” is generally meant a class of BCL-2 homologs (which have a phylogenetic relationship) and collection of evolutionarily unrelated proteins that have pro- and anti-apoptotic functions and include Bcl-2 (i.e., B-cell lymphoma-2) homology (BH) domains. Members of the Bcl-2 family may include one or more of the four characteristic domains of homology entitled Bcl-2 homology (BH) domains named BH1, BH2, BH3 and BH4. The BH domains are known to be important for function, as deletion of these domains via molecular cloning affects survival/apoptosis rates. Anti-apoptotic Bcl-2 proteins, such as Bcl-2 and Bcl-xL, conserve all four BH domains. The BH domains also serve to subdivide the pro-apoptotic Bcl-2 proteins into those with several BH domains (e.g., Bax and Bak) or those proteins that have only the BH3 domain (e.g., Bim, Bid, and BAD). The three functionally important Bcl-2 homology regions (BH1, BH2 and BH3) may be in close spatial proximity, forming binding sites for other BCL-2 family members.

Bcl-2 proteins are central regulators of caspase activation and play a key role in cell death by regulating the integrity of the mitochondrial and endoplasmic reticulum (ER) membranes. Bcl-2 family proteins have a general structure that consists of a hydrophobic α-helix surrounded by amphipathic α-helices. Some members of the family have transmembrane domains at their c-terminus which primarily function to localize them to the mitochondrion.

In some classifications BCL-2 family proteins may be divided into three subgroups: BCL-2 homologs, viral BCL-2-related proteins and BH3-containing proteins with little to no homology to BCL-2. BCL-2 homologs include cellular and viral BCL-2 homologs and BCL-2 homologous proteins usually fall into one of three clades called the BCL-2-like, BAX-like and BID-like subfamilies. Cellular and viral BCL-2 homologs form the core BCL-2 family. Other viral proteins in the family, in addition to viral BCL-2 homologs, are classified as structurally related BCL-2-like fold proteins. BH3-containing proteins with little to no homology to BCL-2 include classical BH3-only members and others that suggest interaction with pro-survival BCL-2 homologs through a BH3-like region. In some instances, subgroups, such as BH3-containing proteins are further subdivided into activators and sensitizers.

Cellular BCL-2 Homologs

Non-limiting examples of BCL-2-like cellular BCL-2 homologs, all of which are BCL-2 family anti-apoptotic proteins, include:

Bcl-2 encoded by the BCL-2 gene and having e.g., the following amino acid sequence as set forth in UniProt Accession No. P10415:

(SEQ ID NO: 1)
MAHAGRTGYDNREIVMKYIHYKLSQRGYEWDAGDVGAAPPGAAPAPGIFS
SQPGHTPHPAASRDPVARTSPLQTPAAPGAAAGPALSPVPPVVHLTLRQA
GDDFSRRYRRDFAEMSSQLHLTPFTARGRFATVVEELFRDGVNWGRIVAF
FEFGGVMCVESVNREMSPLVDNIALWMTEYLNRHLHTWIQDNGGWDAFVE
LYGPSMRPLFDFSWLSLKTLLSLALVGACITLGAYLGHK;

Bcl-xL (aka Bcl211) encoded by the BCL2L1 gene and having e.g., the following amino acid sequence as set forth in UniProt Accession No. Q07817:

(SEQ ID NO: 2)
MSQSNRELVVDFLSYKLSQKGYSWSQFSDVEENRTEAPEGTESEMETPSA
INGNPSWHLADSPAVNGATGHSSSLDAREVIPMAAVKQALREAGDEFELR
YRRAFSDLTSQLHITPGTAYQSFEQVVNELFRDGVNWGRIVAFFSFGGAL
CVESVDKEMQVLVSRIAAWMATYLNDHLEPWIQENGGWDTFVELYGNNAA
AESRKGQERFNRWFLTGMTVAGVVLLGSLFSRK;

Bcl-w (aka Bcl212) encoded by the BCL2L2 gene and having e.g., the following amino acid sequence as set forth in UniProt Accession No. Q92843:

(SEQ ID NO: 3)
MATPASAPDTRALVADFVGYKLRQKGYVCGAGPGEGPAADPLHQAMRAAG
DEFETRFRRTFSDLAAQLHVTPGSAQQRFTQVSDELFQGGPNWGRLVAFF
VFGAALCAESVNKEMEPLVGQVQEWMVAYLETQLADWIHSSGGWAEFTAL
YGDGALEEARRLREGNWASVRTVLTGAVALGALVTVGAFFASK;

Mcl-1 (aka Bcl213) encoded by the MCL-1 gene and having e.g., the following amino acid sequence as set forth in UniProt Accession No. Q07820:

(SEQ ID NO: 4)
MFGLKRNAVIGLNLYCGGAGLGAGSGGATRPGGRLLATEKEASARREIGG
GEAGAVIGGSAGASPPSTLTPDSRRVARPPPIGAEVPDVTATPARLLFFA
PTRRAAPLEEMEAPAADAIMSPEEELDGYEPEPLGKRPAVLPLLELVGES
GNNTSTDGSLPSTPPPAEEEEDELYRQSLEIISRYLREQATGAKDTKPMG
RSGATSRKALETLRRVGDGVQRNHETAFQGMLRKLDIKNEDDVKSLSRVM
IHVFSDGVTNWGRIVTLISFGAFVAKHLKTINQESCIEPLAESITDVLVR
TKRDWLVKQRGWDGFVEFFHVEDLEGGIRNVLLAFAGVAGVGAGLAYLI
R;

Bcl2110 (aka Bcl-B, Nrh, Nr-13, Diva, Boo) encoded by the BCL2L10 gene and having e.g., the following amino acid sequence as set forth in UniProt Accession No. Q9HD36:

(SEQ ID NO: 5)
MADPLRERTELLLADYLGYCAREPGTPEPAPSTPEAAVLRSAAARLRQIH
RSFFSAYLGYPGNRFELVALMADSVLSDSPGPTWGRVVTLVTFAGTLLER
GPLVTARWKKWGFQPRLKEQEGDVARDCQRLVALLSSRLMGQHRAWLQAQ
GGWDGFCHFFRTPFPLAFWRKQLVQAFLSCLLTTAFIYLWTRLL;

and

Bfl1 (aka Bcl2a1, Bcl215) encoded by the BCL2A1 gene and having e.g., the following amino acid sequence as set forth in UniProt Accession No. Q16548:

(SEQ ID NO: 6)
MTDCEFGYIYRLAQDYLQCVLQIPQPGSGPSKTSRVLQNVAFSVQKEVEK
NLKSCLDNVNVVSVDTARTLFNQVMEKEFEDGIINWGRIVTIFAFEGILI
KKLLRQQIAPDVDTYKEISYFVAEFIMNNTGEWIRQNGGWENGFVKKFEP
KSGWMTFLEVTGKICEMLSLLKQYC.

Non-limiting examples of BAX-like cellular BCL-2 homologs, all of which are BCL-2 family pro-apoptotic proteins, include:

Bax (aka Bcl214) encoded by the BAX gene and having e.g., the following amino acid sequence as set forth in UniProt Accession No. Q07812:

(SEQ ID NO: 7)
MDGSGEQPRGGGPTSSEQ1MKTGALLLQGFIQDRAGRMGGEAPELALDPV
PQDASTKKLSECLKRIGDELDSNMELQRMIAAVDTDSPREVFFRVAADMF
SDGNFNWGRVVALFYFASKLVLKALCTKVPELIRTIMGWTLDFLRERLLG
WIQDQGGWDGLLSYFGTPTWQTVTIFVAGVLTASLTIWKKMG;

Bak1 (aka Bcl217) encoded by the BAK1 gene and having e.g., the following amino acid sequence as set forth in UniProt Accession No. Q16611:

(SEQ ID NO: 8)
MASGQGPGPPRQECGEPALPSASEEQVAQDTEEVFRSYVFYRHQQEQEAE
GVAAPADPEMVTLPLQPSSTMGQVGRQLAIIGDDINRRYDSEFQTMLQHL
QPTAENAYEYFTKIATSLFESGINWGRVVALLGFGYRLALHVYQHGLTGF
LGQVTRFVVDFMLHHCIARWIAQRGGWVAALNLGNGPILNVLVVLGVVLL
GQFVVRRFFKS;

Bok (aka Bcl219, Mtd) encoded by the BOK gene and having e.g., the following amino acid sequence as set forth in UniProt Accession No. Q9UMX3:

(SEQ ID NO: 9)
MEVLRRSSVFAAEIMDAFDRSPTDKELVAQAKALGREYVHARLLRAGLSW
SAPERAAPVPGRLAEVCAVLLRLGDELEMIRPSVYRNVARQLHISLQSEP
VVTDAFLAVAGHIFSAGITWGKVVSLYAVAAGLAVDCVRQAQPAMVHALV
DCLGEFVRKTLATWLRRRGGWTDVLKCVVSTDPGLRSHWLVAALCSFGRF
LKAAFFVLLPER;

and

Bcl-WAV encoded by the BCL-WAV gene and having e.g., the following amino acid sequence as set forth in UniProt Accession No. D2Y5Q2:

(SEQ ID NO: 10)
MGRSDDAVIGRGLNSPDPLVREAFLMAYDYISYVTAKPGVPLCPAPSRAS
AALRHAGDELLIRFPIFFRRWPRVFQDVTEHTACPTLLSILDEHFAPTRR
RDLAWSAVLSVFVLAGQLALHCQDRGMEDITPQIQECVGSYVERVICPEI
RDKGGWSGFISRFGEKQNLEDHVVKVCCWSLLLLCVGILSYFIWTRRKT.

Non-limiting examples of BID-like cellular BCL-2 homologs, all of which are BCL-2 family pro-apoptotic proteins, include:

Bid (aka Bid) encoded by the BID gene and having e.g., the following amino acid sequence as set forth in UniProt Accession No. P55957:

(SEQ ID NO: 11)
MDCEVNNGSSLRDECITNLLVFGFLQSCSDNSFRRELDALGHELPVLAPQ
WEGYDELQTDGNRSSHSRLGRIEADSESQEDIIRNIARHLAQVGDSMDRS
IPPGLVNGLALQLRNTSRSEEDRNRDLATALEQLLQAYPRDMEKEKTMLV
LALLLAKKVASHTPSLLRDVFHTTVNFINQNLRTYVRSLARNGMD;

Bcl2112 (aka Bpr) encoded by the BCL2L12 gene and having e.g., the following amino acid sequence as set forth in UniProt Accession No. Q9HB09:

(SEQ ID NO: 12)
MGRPAGLFPPLCPFLGFRPEACWERHMQIERAPSVPPFLRWAGYRPGPVR
RRGKVELIKFVRVQWRRPQVEWRRRRWGPGPGASMAGSEELGLREDTLRV
LAAFLRRGEAAGSPVPTPPRSPAQEEPTDFLSRLRRCLPCSLGRGAAPSE
SPRPCSLPIRPCYGLEPGPATPDFYALVAQRLEQLVQEQLKSPPSPELQG
PPSTEKEAILRRLVALLEEEAEVINQKLASDPALRSKLVRLSSDSFARLV
ELFCSRDDSSRPSRACPGPPPPSPEPLARLALAMELSRRVAGLGGTLAGL
SVEHVHSFTPWIQAHGGWEGILAVSPVDLNLPLD;

Bcl2113 (aka Bcl-rambo, Mill) encoded by the BCL2L13 gene and having e.g., the following amino acid sequence as set forth in UniProt Accession No. Q9BXK5:

(SEQ ID NO: 13)
MASSSTVPLGFHYETKYVVLSYLGLLSQEKLQEQHLSSPQGVQLDIASQS
LDQEILLKVKTEIEEELKSLDKEISEAFTSTGFDRHTSPVFSPANPESSM
EDCLAHLGEKVSQELKEPLHKALQMLLSQPVTYQAFRECTLETTVHASGW
NKILVPLVLLRQMLLELTRRGQEPLSALLQFGVTYLEDYSAEYIIQQGGW
GTVFSLESEEEEYPGITAEDSNDIYILPSDNSGQVSPPESPTVTTSWQSE
SLPVSLSASQSWHTESLPVSLGPESWQQIAMDPEEVKSLDSNGAGEKSEN
NSSNSDIVHVEKEEVPEGMEEAAVASVVLPARELQEALPEAPAPLLPHIT
ATSLLGTREPDTEVITVEKSSPATSLFVELDEEEVKAATTEPTEVEEVVP
ALEPTETLLSEKEINAREESLVEELSPASEKKPVPPSEGKSRLSPAGEMK
PMPLSEGKSILLFGGAAAVAILAVAIGVALALRKK;

Bcl2114 (aka Bcl-G) encoded by the BCL2L14 gene and having e.g., the following amino acid sequence as set forth in UniProt Accession No. Q9BZR8:

(SEQ ID NO: 14)
MCSTSGCDLEEIPLDDDDLNTIEFKILAYYTRHHVFKSTPALFSPKLLRT
RSLSQRGLGNCSANESWTEVSWPCRNSQSSEKAINLGKKKSSWKAFFGVV
EKEDSQSTPAKVSAQGQRTLEYQDSHSQQWSRCLSNVEQCLEHEAVDPKV
ISIANRVAEIVYSWPPPQATQAGGFKSKEIFVTEGLSFQLQGHVPVASSS
KKDEEEQILAKIVELLKYSGDQLERKLKKDKALMGHFQDGLSYSVFKTIT
DQVLMGVDPRGESEVKAQGFKAALVIDVTAKLTAIDNHPMNRVLGFGTKY
LKENFSPWIQQHGGWEKILGISHEEVD;

and

Bcl2115 (aka Bfk) encoded by the BCL2L15 gene and having e.g., the following amino acid sequence as set forth in UniProt Accession No. Q5TBC7:

(SEQ ID NO: 15)
MKSSQTFEEQTECIVNTLLMDFLSPTLQVASRNLCCVDEVDSGEPCSFDV
AIIAGRLRMLGDQFNGELEASAKNVIAETIKGQTGAILQDTVESLSKTWC
AQDSSLAYERAFLAVSVKLLEYMAHIAPEVVGQVAIPMTGMINGNQAIRE
FIQGQGGWENLES.

Non-limiting examples of other cellular BCL-2 homologs that are BCL-2 family anti-apoptotic proteins, include:

CED-9 encoded by the ced-9 gene and having e.g., the following amino acid sequence as set forth in UniProt Accession No. P41958:

(SEQ ID NO: 16)
MTRCTADNSLTNPAYRRRTMATGEMKEFLGIKGTEPTDFGINSDAQDLPS
PSRQASTRRMSIGESIDGKINDWEEPRLDIEGFVVDYFTHRIRQNGMEWF
GAPGLPCGVQPEHEMMRVMGTIFEKKHAENFETFCEQLLAVPRISFSLYQ
DVVRTVGNAQTDQCPMSYGRLIGLISFGGFVAAKMMESVELQGQVRNLFV
YTSLFIKTRIRNNWKEHNRSWDDFMTLGKQMKEDYERAEAEKVGRRKQNR
RWSMIGAGVTAGAIGIVGVVVCGRMMFSLK;

and

BUFFY (aka Drob-2, dBorg-2) encoded by the Buffy gene and having e.g., the following amino acid sequence as set forth in UniProt Accession No. Q8T8Y5:

(SEQ ID NO: 17)
MPGTSYPTNNDNFSNGFPMATTQSERLLQAQNRRKFSFPATLHSASLLEV
GGGPKETTRRRLSNVSDAVTRKLSYTIGWKAAQIPAQDIISQGRCLCGHY
IKRRLRRSGLFNKKLGLQRIRSILGSTSMGIVRDVFPAVQVLGDELERMH
PRIYNGVARQICRNPGGEFHTPDAVSLLLGAVGRELFRVEITWSKVISLF
AIAGGLSVDCVRQGHPEYLPKLMESVSEVIEDELVPWINENGGWSGINTH
VLPTTNSLNPLEWTTLVIGVVFGLILVFMILRFIFNLIVPKIYQRFTNS.

Non-limiting examples of other cellular BCL-2 homologs that are BCL-2 family pro-apoptotic proteins, include:

Debcl (aka Drob-1, dBorg-1, dBok) encoded by the debcl gene and having e.g., the following amino acid sequence as set forth in UniProt Accession No. Q9V9C8:

(SEQ ID NO: 18)
MAPTTSPPPKLAKFKSSSLDHEIYTANRRGTIATASSDWKALRGGVGGGA
GGPGSVPNPSNGRSLHAGGPMTRAASTSSLASSTRTMTNYQEYKMDIINQ
GKCLCGQYIRARLRRAGVLNRKVTQRLRNILDPGSSHVVYEVFPALNSMG
EELERMHPRVYTNISRQLSRAPFGELEDSDMAPMLLNLVAKDLFRSSITW
GKIISIFAVCGGFAIDCVRQGHFDYLQCLIDGLAEIIEDDLVYWLIDNGG
WLGLSRHIRPRVGEFTFLGWLTLFVTISAGAYMVSNVCRRIGGQLYSLL
F.

Non-limiting examples of other cellular BCL-2 homologs also include:

BHP2 encoded by the bhp2 gene and having e.g., the following amino acid sequence as set forth in UniProt Accession No. Q967D2:

(SEQ ID NO: 19)
MAARGSAAPGGRANGRFHSRLYLQNTAVMEELYRRNLSEDLVRDNGLSCG
GREYWREPASTVGAASDGLSEEERRTAADAAERMTAVIAGTPGIAVERNV
RDFRRGGWDVTPDNVESEFREVERRTFSDGVHWGRVIAFLAFSMSFAAYV
NSRGIDGGAYSVFNWTLRVLNDSLADFIQRENGWRGFIVYADTLLRAQGS
TPPQHQTRGVWDAVAGIGVIGVGTLLALGMRQAFS.

Viral BCL-2 Homologs

Non-limiting examples of viral BCL-2 homologs (including those from Adenoviridae, Asfarviridae, Herpesviridae, and Poxviridae families) that are BCL-2 family anti-apoptotic proteins, include:

E1B 19K (aka E1Bs) encoded by the E1B19K gene and having e.g., the following amino acid sequence as set forth in UniProt Accession No. P03246:

(SEQ ID NO: 20)
MEAWECLEDFSAVRNLLEQSSNSTSWFWRFLWGSSQAKLVCRIKEDYKWE
FEELLKSCGELFDSLNLGHQALFQEKVIKTLDFSTPGRAAAAVAFLSFIK
DKWSEETHLSGGYLLDFLAMHLWRAVVRHKNRLLLLSSVRPAIIPTEEQQ
QQQEEARRRRQEQSPWNPRAGLDPRE;

LMW5-HL (aka A179L) encoded by the LMW5-HL gene and having e.g., the following amino acid sequence as set forth in UniProt Accession No. Q07819:

(SEQ ID NO: 21)
MEGEELIYHNIINEILVGYIKYYMNDISEHELSPYQQQIKKILTYYDECL
NKQVTITFSLTNAQEIKTQFTGVVTELFKDLINWGRICGFIVFSARMAKY
CKDANNHLESTVITTAYNFMKHNLLPWMISHGGQEEFLAFSLHSDIYSVI
FNIKYFLSKFCNHMFLRSCVQLLRNCNLI;

A9 encoded by the A9 gene and having e.g., the following amino acid sequence as set forth in UniProt Accession No. 036423:

(SEQ ID NO: 22)
MKMLGEPEFKENILYYSFLNELFLILIRNGFSCSHAKLILDETRKRGLEC
SGQFEVISNSVEAPEPESLERIAKTLFTPRPHWGRLVAFLAYLAYLQKNS
TEKLFWNDHLKKLKQIVKCHIVPWTLGPRDPKPKQRPFDKLPSAFYFLTA
AASCLTLLLLYFRTTQTK;

BALF1 encoded by the BALF1 gene and having e.g., the following amino acid sequence as set forth in UniProt Accession No. POCK58:

(SEQ ID NO: 23)
MNLAIALDSPHPGLASYTILPRPFYHISLKPVSWPDETMRPAKSTDSVFV
RTPVEAWVAPSPPDDKVAESSYLMFRAMYAVFTRDEKDLPLPALVLCRLI
KASLRKDRKLYAELACRTADIGGKDTHVRLIISVLRAVYNDHYDYWSRLR
VVLCYTVVFAVRNYLDDHKSAAFVLGAIAHYLALYRRLWFARLGGMPRSL
RRQFPVTWALASLTDFLKSL;

BHRF1 encoded by the BHRF1 gene and having e.g., the following amino acid sequence as set forth in UniProt Accession No. P03182:

(SEQ ID NO: 24)
MAYSTREILLALCIRDSRVHGNGTLHPVLELAARETPLRLSPEDTVVLRY
HVLLEEIIERNSETFTETWNRFITHTEHVDLDFNSVFLEIFHRGDPSLGR
ALAWMAWCMHACRTLCCNQSTPYYVVDLSVRGMLEASEGLDGWIHQQGGW
STLIEDNIPGSRRFSWTLFLAGLTLSLLVICSYLFISRGRH;

v-Bcl-2 (aka BoHV4) encoded by the V-BCL-2 gene and having e.g., the following amino acid sequence as set forth in UniProt Accession No. Q9WH78:

(SEQ ID NO: 25)
MSLFFVVWYWVNYITKVCSGEVYIPSVLKFQYHSDTEHEPYSNLCKNLIT
MAEQDMDEVVSTIRRLLVECGMGLEEYLEHPVTAPIKVAVQDVIRTKQDI
FSNFLTNINSVEDLETLGHAITTLNDYPSPNMGRVVCGIAFSVYVVQTVC
KRKPLLVRCCLDIFTRATVQALNVNWFLQEGGWPALASFCKVVNSPSPRS
RWLFPMFAISGLVLTVGVARNMVHFT;

M11 (aka gammaHV68 Bcl-2) encoded by the M11 gene and having e.g., the following amino acid sequence as set forth in UniProt Accession No. P89884:

(SEQ ID NO: 26)
MSHKKSGTYWATLITAFLKTVSKVEELDCVDSAVLVDVSKIITLTQEFRR
HYDSVYRADYGPALKNWKRDLSKLFTSLFVDVINSGRIVGFFDVGRYVCE
EVLCPGSWTEDHELLNDCMTHFFIENNLMNHFPLEDIFLAQRKFQTTGFT
FLLHALAKVLPRIYSGNVIYV;

ORF16 (aka KsBcl-2) encoded by the ORF16 gene and having e.g., the following amino acid sequence as set forth in UniProt Accession No. Q76RI8:

(SEQ ID NO: 27)
MDEDVLPGEVLAIEGIFMACGLNEPEYLYHPLLSPIKLYITGLMRDKESL
FEAMLANVRFHSTTGINQLGLSMLQVSGDGNMNWGRALAILTFGSFVAQK
LSNEPHLRDFALAVLPVYAYEAIGPQWFRARGGWRGLKAYCTQVLTRRRG
RRMTALLGSIALLATILAAVAMSRR;

vNR13 encoded by the vNR13 gene and having e.g., the following amino acid sequence as set forth in UniProt Accession No. Q9DH00:

(SEQ ID NO: 28)
MADSLKEETALLLEDYFQHCCGKEGPPPSPTAAELRRAAAELERRERPFF
RSCAPLASGGTQAALSALQSVVSELNSGSGFNWGRCLATIVLGGSLATAL
YENGCEEGPSRLAAALAAYLAEEQGEWLREHGGWVSTARAGMSGSFGSPV
SARSEEHRARRW;

FPV039 encoded by the FPV039 gene and having e.g., the following amino acid sequence as set forth in UniProt Accession No. Q9J5G4:

(SEQ ID NO: 29)
MASSNMKDETYYIALNMIQNYIIEYNTNKPRKSFVIDSISYDVLKAACKS
VIKTNYNEFDIIISRNIDFNVIVTQVLEDKINWGRIITIIAFCAYYSKKV
KQDTSPQYYDGIISEAITDAILSKYRSWFIDQDYWNGIRIYKNYSYIFNT
ASYCIFTASLIIASLAVFKICSFYM;

and

ORFV125 encoded by the ORFV125 gene and having e.g., the following amino acid sequence as set forth in UniProt Accession No. Q80G30:

(SEQ ID NO: 30)
MANREEIDASAVMAAYLAREYAAAVEEQLTPRERDALEALRVSGEEVRSPL
LQELSNAGEHRANPENSHIPAALVSALLEAPTSPGRMVTAIELCAQMGRVW
TRGRQLVEFMRLVYVLLDRLPPTADEDLSTWLQAVARVHGTRRRLHRVLGV
GAVMAGVGMLLLGVRVLRRT.

Non-limiting examples of viral BCL-2 homologs from the Iridoviridae family include:

070L encoded by the 070L gene and having e.g., the following amino acid sequence as set forth in UniProt Accession No. Q677S5:

(SEQ ID NO: 31)
MMSNKFETDTKYLIDAFFKEYFNSQESNDIILKTIKKEVNILFDKHRLVYS
NMINDISITTEIDILVKKTAESIFSDGLVNWGRIISLITFGILIVEYLKTI
NNTDKITSVSTIISSYLIEHQKHWLIKNNAWIGLVDFFTVQTYTSPVKSLL
TFFIVFMGTGAMLYSAFNLTY;

097R encoded by the 097R gene and having e.g., the following amino acid sequence as set forth in UniProt Accession No. Q6GZM8:

(SEQ ID NO: 32)
MDVRQFLSDCEAPEEMVALRAAADAVGVDNRACAHLYTMLWEGVNLEEVHA
SLLGDGVVNWGRVAAFMHICRYIVRTFPSSMDRTEVALTKFIQDPKIDKQL
REWTDRLGTVGLIGRCLEWLGAGVITGVVLSLLFY;

and

ORF115R encoded by the ORF115R gene and having e.g., the following amino acid sequence as set forth in UniProt Accession No. Q5YFF0:

(SEQ ID NO: 33)
MTNINFSALLRGERMCPLTREIHSQMLIVTKSYSLVETFRAFPRLPNILEI
GNNIVSDGNLNWGRILILLGISQLYFTKSESESERTQITEQLERFFRQDAI
SNWIVSNGGWVTCASLDLRNYSSVTNALQAMCFFGALFGTIAVIAYYLLP.

Structurally Related Bcl-2-Like Fold Proteins

Non-limiting examples of viral proteins that are structurally related BCL-2-like fold proteins with low similarity to BCL-2, that are anti-apoptotic, include:

DPV022 encoded by the DPV022 gene and having e.g., the following amino acid sequence as set forth in UniProt Accession No. Q08FX8:

(SEQ ID NO: 34)
MEAAIEFDEIVKKLLNIYINDICTMGEKRLLNNYEKSILDRIYKSCEYIKK
NYELDFNSMYNQININDITTSDIKSKIIESLLIDSRPSVKLATLSFISLIA
EKWGEKNRTKIMEILSNEIVEKISNNGKDFIDFIDRDDDDIVDDYVLITNY
LKITIFGAILGITAYYICKYLLKSIF;

F1L encoded by the F1L gene and having e.g., the following amino acid sequence as set forth in UniProt Accession No. 057173:

(SEQ ID NO: 35)
MLSMFMCNNIVDYVDGIVQDIEDEASNNVDHDYVYPLPENMVYRFDKSTNI
LDYLSTERDHVMMAVRYYMSKQRLDDLYRQLPTKTRSYIDIINIYCDKVSN
DYNRDMNIMYDMASTKSFTVYDINNEVNTILMDNKGLGVRLATISFITELG
RRCMNPVKTIKMFTLLSHTICDDCFVDYITDISPPDNTIPNTSTREYLKLI
GITAIMFATYKTLKYMIG;

M11L encoded by the M11L gene and having e.g., the following amino acid sequence as set forth in UniProt Accession No. Q85295:

(SEQ ID NO: 36)
MMSRLKTAVYDYLNDVDITECTEMDLLCQLSNCCDFINETYAKNYDTLYDI
MERDILSYNIVNIKNTLTFALRDASPSVKLATLTLLASVIKKLNKIQHTDA
AMFSEVIDGIVAEEQQVIGFIQKKCKYNTTYYNVRSGGCKISVYLTAAVVG
FVAYGILKWYRGT;

N1L encoded by the N1L gene and having e.g., the following amino acid sequence as set forth in UniProt Accession No. P17361:

(SEQ ID NO: 37)
MRTLLIRYILWRNDNDQTYYNDNFKKLMLLDELVDDGDVCTLIKNMRMTLS
DGPLLDRLNQPVNNIEDAKRMIAISAKVARDIGERSEIRWEESFTILFRMI
ETYFDDLMIDLYGEK;

and

SPPV14 encoded by the SPPV14 gene and having e.g., the following amino acid sequence as set forth in UniProt Accession No. Q8JU19:

(SEQ ID NO: 38)
MDNCNYNIEKVLNVYLRDLRIESLNNNELAILIMIRECCEVIKKDYKTEFN
EICNFILHNNVKSCYDINDVKNIIIETINSDFRPSVILASISLLSIIIKKK
KNENNEVVNDDLALNELINTFSSYQKDIISFVEKNKKNNEHNDFIFSIINF
FVMVGSIIIAYYLLKIIGRIRWK.

Non-limiting examples of viral proteins that are structurally related BCL-2-like fold proteins with low similarity to BCL-2 also include:

A46 encoded by the A46 gene and having e.g., the following amino acid sequence as set forth in UniProt Accession No. P26672:

(SEQ ID NO: 39)
MAFDISVNASKTINALVYFSTQQNKLVIRNEVNDTHYTVEFDRDKVVDTFI
SYNRHNDTIEIRGVLPEETNIGCAVNTPVSMTYLYNKYSFKLILAEYIRHR
NTISGNIYSALMTLDDLAIKQYGDIDLLFNEKLKVDSDSGLFDFVNFVKDM
ICCDSRIVVALSSLVSKHWELTNKKYRCMALAEHISDSIPISELSRLRYNL
CKYLRGHTESIEDKFDYFEDDDSSTCSAVTDRETDV;

A49 encoded by the A49R gene and having e.g., the following amino acid sequence as set forth in UniProt Accession No. P21068:

(SEQ ID NO: 40)
MDEAYYSGNLESVLGYVSDMHTELASISQLVIAKIETIDNDILNKDIVNFI
MCRSNLDNPFISFLDTVYTIIDQENYQTELINSLDDNEIIDCIVNKFMSFY
KDNLENIVDAIITLKYIMNNPDFKTTYAEVLGSRIADIDIKQVIRKNILQL
SNDIRERYL;

A52 encoded by the A52 gene and having e.g., the following amino acid sequence as set forth in UniProt Accession No. Q01220:

(SEQ ID NO: 41)
MDIKIDISISGDKFTVTTRRENEERKKYLPLQKEKTTDVIKPDYLEYDDLL
DRDEMFTILEEYFMYRGLLGLRIKYGRLFNEIKKFDNDAEEQFGTIEELKQ
KLRLNSEEGADNFIDYIKVQKQDIVKLTVYDCISMIGLCACVVDVWRNEKL
FSRWKYCLRAIKLFINDHMLDKIKSILQNRLVYVEMS;

B14 encoded by the B14 gene and having e.g., the following amino acid sequence as set forth in UniProt Accession No. P24772:

(SEQ ID NO: 42)
MTANFSTHVFSPQHCGCDRLTSIDDVRQCLTEYIYWSSYAYRNRQCAGQLY
STLLSFRDDAELVFIDIRELVKNMPWDDVKDCAEIIRCYIPDEQKTIREIS
AIIGLCAYAATYWGGEDHPTSNSLNALFVMLEMLNYVDYNIIFRRMN;

C1 encoded by the C1 gene and having e.g., the following amino acid sequence as set forth in UniProt Accession No. P17368:

(SEQ ID NO: 43)
MVKNNKIQKNKISNSCRMIMSTDPNNILMRHLKNLTDDEFKCIIHRSSDFL
YLSDSDYTSITKETLVSEIVEEYPDDCNKILAIIFLVLDKDIDVDIKTKLK
PKPAVRFAILDKMTEDIKLTDLVRHYFRYIEQDIPLGPLFKKIDSYRTRAI
NKYSKELGLATEYFNKYGHLMFYTLPIPYNRFFCRNSIGFLAVLSPTIGHV
KAFYKFIEYVSIDDRRKFKKELMSK;

K7 encoded by the K7 gene and having e.g., the following amino acid sequence as set forth in UniProt Accession No. Q76ZX2:

(SQ ID NO: 44)
MATKLDYEDAVFYFVDDDKICSRDSIIDLIDEYITWRNHVIVFNKDITSCG
RLYKELMKFDDVAIRYYGIDKINEIVEAMSEGDHYINFTKVHDQESLFATI
GICAKITEHWGYKKISESRFQSLGNITDLMTDDNINILILFLEKKLN;

N2 encoded by the N2 gene and having e.g., the following amino acid sequence as set forth in UniProt Accession No. P14357:

(SEQ ID NO: 45)
MTSSAMDNNEPKVLEMVYDATILPEGSSMDPNIMDCINRHINMCIQRTYSS
SIIAILDRFLMMNKDELNNTQCHIIKEFMTYEQMAIDHYGGYVNAILYQIR
KRPNQHHTIDLFKRIKRTRYDTFKVDPVEFVKKVIGFVSILNKYKPVYSYV
LYENVLYDEFKCFINYVETKYF;

C6 encoded by the C6 gene and having e.g., the following amino acid sequence as set forth in UniProt Accession No. P17362:

(SEQ ID NO: 46)
MNAYNKADSFSLESDSIKDVIHDYICWLSMTDEMRPSIGNVFKAMETFKID
AVRYYDGNIYELAKDINAMSFDGFIRSLQTIASKKDKLTVYGTMGLLSIVV
DINKGCDISNIKFAAGIIILMEYIFDDTDMSHLKVALYRRIQRRDDVDR;

and

C16 encoded by the C16 gene and having e.g., the following amino acid sequence as set forth in UniProt Accession No. P21100:

(SEQ ID NO: 47)
MISLSFLIHNPLKKWKLKPSISINGYRSTFTMAFPCAQFRPCHCHATKDSL
NTVADVRHCLTEYILWVSHRWTHRESAGSLYRLLISFRTDATELFGGELKD
SLPWDNIDNCVEIIKCFIRNDSMKTAEELRAIIGLCTQSAIVSGRVFNDKY
IDILLMLRKILNENDYLTLLDHIRTAKY.

BH3-Containing Proteins

Non-limiting examples of classical BH3-only member proteins that contain BH-3 domains, and are all pro-apoptotic BLC-2 family proteins, include:

Bim (aka Bcl2111, Bod) encoded by the BCL2L11 gene and having e.g., the following amino acid sequence as set forth in UniProt Accession No. 043521:

(SEQ ID NO: 48)
MAKQPSDVSSECDREGRQLQPAERPPQLRPGAPTSLQTEPQGNPEGNHGGE
GDSCPHGSPQGPLAPPASPGPFATRSPLFIFMRRSSLLSRSSSGYFSFDTD
RSPAPMSCDKSTQTPSPPCQAFNHYLSAMASMRQAEPADMRPEIWIAQELR
RIGDEFNAYYARRVFLNNYQAAEDHPRMVILRLLRYIVRLVWRMH;

Puma (aka Bbc3) encoded by the BBC3 gene and having e.g., the following amino acid sequence as set forth in UniProt Accession No. Q9BXH1:

(SEQ ID NO: 49)
MARARQEGSSPEPVEGLARDGPRPFPLGRLVPSAVSCGLCEPGLAAAPAAP
TLLPAAYLCAPTAPPAVTAALGGSRWPGGPRSRPRGPRPDGPQPSLSLAEQ
HLESPVPSAPGALAGGPTQAAPGVRGEEEQWAREIGAQLRRMADDLNAQYE
RRRQEEQQRHRPSPWRVLYNLIMGLLPLPRGHRAPEMEPN;

Bad (aka Bcl218, Bbc2) encoded by the BAD gene and having e.g., the following amino acid sequence as set forth in UniProt Accession No. Q92934:

(SEQ ID NO: 50)
MFQIPEFEPSEQEDSSSAERGLGPSPAGDGPSGSGKHHRQAPGLLWDASHQ
QEQPTSSSHHGGAGAVEIRSRHSSYPAGTEDDEGMGEEPSPFRGRSRSAPP
NLWAAQRYGRELRRMSDEFVDSFKKGLPRPKSAGTATQMRQSSSWTRVFQS
WWDRNLGRGSSAPSQ;

Bik (aka Nbk, Blk) encoded by the BIK gene and having e.g., the following amino acid sequence as set forth in UniProt Accession No. Q13323:

(SEQ ID NO: 51)
MSEVRPLSRDILMETLLYEQLLEPPTMEVLGMTDSEEDLDPMEDFDSLECM
EGSDALALRLACIGDEMDVSLRAPRLAQLSEVAMHSLGLAFIYDQTEDIRD
VLRSFMDGFTTLKENIMRFWRSPNPGSWVSCEQVLLALLLLLALLLPLLSG
GLHLLLK;

Bmf encoded by the BMF gene and having e.g., the following amino acid sequence as set forth in UniProt Accession No. Q96LC9:

(SEQ ID NO: 52)
MEPSQCVEELEDDVFQPEDGEPVTQPGSLLSADLFAQSLLDCPLSRLQLFP
LTHCCGPGLRPTSQEDKATQTLSPASPSQGVMLPCGVTEEPQRLFYGNAGY
RLPLPASFPAVLPIGEQPPEGQWQHQAEVQIARKLQCIADQFHRLHVQQHQ
QNQNRVWWQILLFLHNLALNGEENRNGAGPR;

Egl-1 encoded by the egl-1 gene and having e.g., the following amino acid sequence as set forth in UniProt Accession No. 061667:

(SEQ ID NO: 53)
MLMLTFASTSSDLLPMSNVFDVQSSVFYNEKNMFYSSSQDFSSCEDSSQFA
DDSGFFDDSEISSIGYEIGSKLAAMCDDFDAQMMSYSAHASDRSLFHRLLD
FFAF;

Hrk (aka Dp5) encoded by the HRK gene and having e.g., the following amino acid sequence as set forth in UniProt Accession No. 000198:

(SEQ ID NO: 54)
MCPCPLHRGRGPPAVCACSAGRLGLRSSAAQLTAARLKALGDELHQRTMWR
RRARSRRAPAPGALPTYWPWLCAAAQVAALAAWLLGRRNL;

and

Noxa (aka Apr, Pmaip1) encoded by the PMAIP1 gene and having e.g., the following amino acid sequence as set forth in UniProt Accession No. Q13794:

(SEQ ID NO: 55)
MPGKKARKNAQPSPARAPAELEVECATQLRRFGDKLNFRQKLLNLISKLFC
SGT.

Non-limiting examples of other BH-3-containing proteins that are pro-apoptotic, include:

Apol6 (aka Apol-VI) encoded by the APOL6 gene and having e.g., the following amino acid sequence as set forth in UniProt Accession No. Q9BWW8:

(SEQ ID NO: 56)
MDNQAERESEAGVGLQRDEDDAPLCEDVELQDGDLSPEEKIFLREFPRLKE
DLKGNIDKLRALADDIDKTHKKFTKANMVATSTAVISGVMSLLGLALAPAT
GGGSLLLSTAGQGLATAAGVTSIVSGTLERSKNKEAQARAEDILPTYDQED
REDEEEKADYVTAAGKIIYNLRNTLKYAKKNVRAFWKLRANPRLANATKRL
LTTGQVSSRSRVQVQKAFAGTTLAMTKNARVLGGVMSAFSLGYDLATLSKE
WKHLKEGARTKFAEELRAKALELERKLTELTQLYKSLQQKVRSRARGVGKD
LTGTCETEAYWKELREHVWMWLWLCVCLCVCVYVQFT;

Atg12 (aka Apg12, Apg12L) encoded by the ATG12 gene and having e.g., the following amino acid sequence as set forth in UniProt Accession No. 094817:

(SEQ ID NO: 57)
MAEEPQSVLQLPTSIAAGGEGLTDVSPETTTPEPPSSAAVSPGTEEPAGDT
KKKIDILLKAVGDTPIMKTKKWAVERTRTIQGLIDFIKKFLKLVASEQLFI
YVNQSFAPSPDQEVGTLYECFGSDGKLVLHYCKSQAWG;

Ced-13 encoded by the CED-13 gene and having e.g., the following amino acid sequence as set forth in UniProt Accession No. Q9TY06:

(SEQ ID NO: 58)
MMSYKRDGYFSIVSSCLIFCLHFLPLLSIRTKLSSLFITSLANNACNSNTV
EYNIGRKLTVMCDEFDSELMSYKEEKSFVKFLGSGFKTYASIVRRVF;

Mule (aka Huwel) encoded by the HUWE gene and having e.g., the following amino acid sequence as set forth in UniProt Accession No. Q7Z6Z7:

(SEQ ID NO: 59)
MKVDRTKLKKTPTEAPADCRALIDKLKVCNDEQLLLELQQIKTWNIGKCELYHWVDL
LDRFDGILADAGQTVENMSWMLVCDRPEREQLKMLLLAVLNFTALLIEYSFSRHLYSSI
EHLTTLLASSDMQVVLAVLNLLYVFSKRSNYITRLGSDKRTPLLTRLQHLAESWGGKE
NGFGLAECCRDLHMMKYPPSATTLHFEFYADPGAEVKIEKRTTSNTLHYIHIEQLDKISE
SPSEIMESLTKMYSIPKDKQMLLFTHIRLAHGFSNHRKRLQAVQARLHAISILVYSNALQ
ESANSILYNGLIEELVDVLQITDKQLMEIKAASLRTLTSIVHLERTPKLSSIIDCTGTASYH
GFLPVLVRNCIQAMIDPSMDPYPHQFATALFSFLYHLASYDAGGEALVSCGMMEALLK
VIKFLGDEQDQITFVTRAVRVVDLITNLDMAAFQSHSGLSIFIYRLEHEVDLCRKECPFVI
KPKIQRPNTTQEGEEMETDMDGVQCIPQRAALLKSMLNFLKKAIQDPAFSDGIRHVMD
GSLPTSLKHIISNAEYYGPSLFLLATEVVTVFVFQEPSLLSSLQDNGLTDVMLHALLIKD
VPATREVLGSLPNVFSALCLNARGLQSFVQCQPFERLFKVLLSPDYLPAMRRRRSSDPL
GDTASNLGSAVDELMRHQPTLKTDATTAIIKLLEEICNLGRDPKYICQKPSIQKADGTAT
APPPRSNHAAEEASSEDEEEEEVQAMQSFNSTQQNETEPNQQVVGTEERIPIPLMDYILN
VMKFVESILSNNTTDDHCQEFVNQKGLLPLVTILGLPNLPIDFPTSAACQAVAGVCKSIL
TLSHEPKVLQEGLLQLDSILSSLEPLHRPIESPGGSVLLRELACAGNVADATLSAQATPLL
HALTAAHAYIMMFVHTCRVGQSEIRSISVNQWGSQLGLSVLSKLSQLYCSLVWESTVL
LSLCTPNSLPSGCEFGQADMQKLVPKDEKAGTTQGGKRSDGEQDGAAGSMDASTQGL
LEGIGLDGDTLAPMETDEPTASDSKGKSKITPAMAARIKQIKPLLSASSRLGRALAELFG
LLVKLCVGSPVRQRRSHHAASTTTAPTPAARSTASALTKLLTKGLSWQPPPYTPTPRFR
LTFFICSVGFTSPMLFDERKYPYHLMLQKFLCSGGHNALFETFNWALSMGGKVPVSEG
LEHSDLPDGTGEFLDAWLMLVEKMVNPTTVLESPHSLPAKLPGGVQNFPQFSALRFLV
VTQKAAFTCIKNLWNRKPLKVYGGRMAESMLAILCHILRGEPVIRERLSKEKEGSRGEE
DTGQEEGGSRREPQVNQQQLQQLMDMGFTREHAMEALLNTSTMEQATEYLLTHPPPI
MGGVVRDLSMSEEDQMMRAIAMSLGQDIPMDQRAESPEEVACRKEEEERKAREKQEE
EEAKCLEKFQDADPLEQDELHTFTDTMLPGCFHLLDELPDTVYRVCDLIMTAIKRNGA
DYRDMILKQVVNQVWEAADVLIKAALPLTTSDTKTVSEWISQMATLPQASNLATRILL
LTLLFEELKLPCAWVVESSGILNVLIKLLEVVQPCLQAAKEQKEVQTPKWITPVLLLIDF
YEKTAISSKRRAQMTKYLQSNSNNWRWFDDRSGRWCSYSASNNSTIDSAWKSGETSV
RFTAGRRRYTVQFTTMVQVNEETGNRRPVMLTLLRVPRLNKNSKNSNGQELEKTLEES
KEMDIKRKENKGNDTPLALESTNTEKETSLEETKIGEILIQGLTEDMVTVLIRACVSMLG
VPVDPDTLHATLRLCLRLTRDHKYAMMFAELKSTRMILNLTQSSGFNGFTPLVTLLLRH
IIEDPCTLRHTMEKVVRSAATSGAGSTTSGVVSGSLGSREINYILRVLGPAACRNPDIFTE
VANCCIRIALPAPRGSGTASDDEFENLRIKGPNAVQLVKTTPLKPSPLPVIPDTIKEVIYD
MLNALAAYHAPEEADKSDPKPGVMTQEVGQLLQDMGDDVYQQYRSLTRQSSDFDTQ
SGFSINSQVFAADGASTETSASGTSQGEASTPEESRDGKKDKEGDRASEEGKQKGKGSK
PLMPTSTILRLLAELVRSYVGIATLIANYSYTVGQSELIKEDCSVLAFVLDHLLPHTQNA
EDKDTPALARLFLASLAAAGSGTDAQVALVNEVKAALGRALAMAESTEKHARLQAV
MCIISTIMESCPSTSSFYSSATAKTQHNGMNNIIRLFLKKGLVNDLARVPHSLDLSSPNM
ANTVNAALKPLETLSRIVNQPSSLFGSKSASSKNKSEQDAQGASQDSSSNQQDPGEPGE
AEVQEEDHDVTQTEVADGDIMDGEAETDSVVIAGQPEVLSSQEMQVENELEDLIDELL
ERDGGSGNSTIIVSRSGEDESQEDVLMDEAPSNLSQASTLQANREDSMNILDPEDEEEHT
QEEDSSGSNEDEDDSQDEEEEEEEDEEDDQEDDEGEEGDEDDDDDGSEMELDEDYPD
MNASPLVRFERFDREDDLIIEFDNMFSSATDIPPSPGNIPTTHPLMVRHADHSSLTLGSGS
STTRLTQGIGRSQRTLRQLTANTGHTIHVHYPGNRQPNPPLILQRLLGPSAAADILQLSSS
LPLQSRGRARLLVGNDDVHIIARSDDELLDDFFHDQSTATSQAGTLSSIPTALTRWTEEC
KVLDAESMHDCVSVVKVSIVNHLEFLRDEELEERREKRRKQLAEEETKITDKGKEDKE
NRDQSAQCTASKSNDSTEQNLSDGTPMPDSYPTTPSSTDAATSESKETLGTLQSSQQQP
TLPTPPALGEVPQELQSPAGEGGSSTQLLMPVEPEELGPTRPSGEAETTQMELSPAPTITS
LSPERAEDSDALTAVSSQLEGSPMDTSSLASCTLEEAVGDTSAAGSSEQPRAGSSTPGD
APPAVAEVQGRSDGSGESAQPPEDSSPPASSESSSTRDSAVAISGADSRGILEEPLPSTSSE
EEDPLAGISLPEGVDPSFLAALPDDIRREVLQNQLGIRPPTRTAPSTNSSAPAVVGNPGVT
EVSPEFLAALPPAIQEEVLAQQRAEQQRRELAQNASSDTPMDPVTFIQTLPSDLRRSVLE
DMEDSVLAVMPPDIAAEAQALRREQEARQRQLMHERLFGHSSTSALSAILRSPAFTSRL
SGNRGVQYTRLAVQRGGTFQMGGSSSHNRPSGSNVDTLLRLRGRLLLDHEALSCLLVL
LFVDEPKLNTSRLHRVLRNLCYHAQTRHWVIRSLLSILQRSSESELCIETPKLTTSEEKGK
KSSKSCGSSSHENRPLDLLHKMESKSSNQLSWLSVSMDAALGCRTNIFQIQRSGGRKHT
EKHASGGSTVHIHPQAAPVVCRHVLDTLIQLAKVFPSHFTQQRTKETNCESDRERGNKA
CSPCSSQSSSSGICTDFWDLLVKLDNMNVSRKGKNSVKSVPVSAGGEGETSPYSLEASP
LGQLMNMLSHPVIRRSSLLTEKLLRLLSLISIALPENKVSEAQANSGSGASSTTTATSTTS
TTTTTAASTTPTPPTAPTPVTSAPALVAATAISTIVVAASTTVTTPTTATTTVSISPTTKGS
KSPAKVSDGGSSSTDFKMVSSGLTENQLQLSVEVLTSHSCSEEGLEDAANVLLQLSRGD
SGTRDTVLKLLLNGARHLGYTLCKQIGTLLAELREYNLEQQRRAQCETLSPDGLPEEQP
QTTKLKGKMQSRFDMAENVVIVASQKRPLGGRELQLPSMSMLTSKTSTQKFFLRVLQV
IIQLRDDTRRANKKAKQTGRLGSSGLGSASSIQAAVRQLEAEADAIIQMVREGQRARRQ
QQAATSESSQSEASVRREESPMDVDQPSPSAQDTQSIASDGTPQGEKEKEERPPELPLLS
EQLSLDELWDMLGECLKELEESHDQHAVLVLQPAVEAFFLVHATERESKPPVRDTRES
QLAHIKDEPPPLSPAPLTPATPSSLDPFFSREPSSMHISSSLPPDTQKFLRFAETHRTVLNQI
LRQSTTHLADGPFAVLVDYIRVLDFDVKRKYFRQELERLDEGLRKEDMAVHVRRDHV
FEDSYRELHRKSPEEMKNRLYIVFEGEEGQDAGGLLREWYMIISREMFNPMYALFRTSP
GDRVTYTINPSSHCNPNHLSYFKFVGRIVAKAVYDNRLLECYFTRSFYKHILGKSVRYT
DMESEDYHFYQGLVYLLENDVSTLGYDLTFSTEVQEFGVCEVRDLKPNGANILVTEEN
KKEYVHLVCQMRMTGAIRKQLAAFLEGFYEIIPKRLISIFTEQELELLISGLPTIDIDDLKS
NTEYHKYQSNSIQIQWFWRALRSFDQADRAKFLQFVTGTSKVPLQGFAALEGMNGIQK
FQIHRDDRSTDRLPSAHTCFNQLDLPAYESFEKLRHMLLLAIQECSEGFGLA;

Matrix (aka M) encoded by the M gene and having e.g., the following amino acid sequence as set forth in UniProt Accession No. Q9WBL3:

(SEQ ID NO: 60)
MDSSRAIGLYFDSALPSSSLLAFPIVLQDTGDGKKQITPQYRIQRLDSWT
DSKEDSVFITTYGFIFQVGNEEVTVGMINDNPGHELLSSAMLCLGSVPND
GDLVELARACLTMVVTCKKSATNTERIVFSVVQAPRVLQSCMVVANRYSS
VNAVNHVKAPEKIPGSGTLEYKVNFVSLTVVPRKDVYRIPTAALKSIWLK
PVQSCAQCHYDCGGGPEEPVSQIPFQVDSWILCNSFLAYRGMVHCRKEGK
ESDIDQARGEDKETQSICRAQDCARTFRACEGERCTDKAVGTFLLSQWDS
LLSYSKCLSPGGKILWSQTAHLRSVKIVIQAGTQRAVAVTADHEVTSTKI
EKRHTIAKYNPFKK;

Large (aka L) encoded by the L gene and having e.g., the following amino acid sequence as set forth in UniProt Accession No. Q6X1B6:

(SEQ ID NO: 61)
MASSGPERAEHQIILPESHLSSPLVKHKLLYYWKLTGLPLPDECDFDHLI
VSRQWKKILESAAPDTERMIKLGRAVHQTLNHNSKITGVLHPRCLEKLAS
IEVPDSTNKFRKIEKKIQIHNTRYGELFTRLCTNVEKKLLGSSWSNNVSR
SEEFNSIRTDPAFWFHSKWSKAKFGWAHFKQVPRHLIVAARTRSAVTKFV
TLTHKLGQVFGTPELVMGNHTNENKSPCLTQELVLMYADMMEGRDMVNII
SSTATHLRSLSEKIDDILRLVDALAKDLGNQVYDVVALMEGFAYGAVQLL
EPSGTFAGDFFAFNLQELKDTLIELLPNDVAELVTHAIATVFSGLEQNQA
AEMLCLLRLWGHPLLESRIAAKAVRSQMCAPKMIDFDMILQVLSFFKGTI
INGYRKKNSGVWPHVKMDTIYGKVIGQLHADSAEISHDVMLREYKSLSAL
EFEPCIEYDPVTNLSMFLKDKAIAHPRDNWLASFRRNLLSEDQKRHIKEA
TSTNRLLIEFLESNDFDPYKEMEYLTTLEYLRDDNVAVSYSLKEKEVKVN
GRIFAKLTKKLRNCQVMAEGILADQIAPFFQGNGVIQDSISLTKSMLAMS
QLSFNSNKKRITDCKERVSSNRNHDPKSKNRRRVATFITTDLQKYCLNWR
YQTVKLFAHAINQLMGLPHFFEWIHLRLMDTTMFVGDPFNPPSDPTDCDL
SRVPNDDIYIVSARGGIEGLCQKLWTMISIAAIQLAAARSHCRVACMVQG
DNQVIAVTREVRSDDSPEMVWTQWLQASDNFFKEMIHVNHLNGHNLKDRE
TIRSDTFFLYSKRIFKDGAILSQVLKNSSKLVLISGDLSENTVMSCANIA
STIARLCENGLPKDFCYYLNYIMSCVQTYFDSEFSITHSSQPDSNQSWFE
DISFVHSYVLTPAQLGGLSNLQYSRLYTRNIGDPGTTAFAEVKRLEAVGL
LSPSIMTNILTRPPGNGDWASLCNDPYSFNFETVASPNIVLKKHTQKVLF
ETCSNPLLSGVHTEDNEAEEKALVEFLLNQEVVHPRVAHAIMESSSVGRR
KQIQGLVDTTNTVINIALTRRPLGIKRLMRIINYSSMHAMLFTDDVFLSN
RPNHPLVSSNMCSLTLADYARNRSWSPLTGGRKILGVSNPDTIEPVEGEI
LSVSGGCKKCDSGDEQFTWFHLPSNIQLTDDTSKNPPMRVPYLGSKTQER
RAASLAKIAHMSPHVKAALRASSVLIWAYGDNEVNWTAALKIARSRCNIS
SEYLRLLSPLPTAGNLQHRLDDGITQMTFTPASLYRVSPYIHISNDSQRL
FTEEGIKEGNVVYQQIMLLGLSLIESLFPMTTTKTYDEITLHLHSKFSCC
IREAPVAVPFELLGLAPELRAVTSNKFMYDPSPVSERDFARLDLAIFKSY
ELNLESYPTIELMNILSISSGKLIGQSVVSYDEDTSIKNDAIIVYDNTRN
WISEAQNSDVVRLFEYAALEVLLDCSYQLYYLRVRGLNNIVLYMSDLYKN
MPGILLSNIAATISHPIIHSRLNAVGLVNHDGSHQLADTDFIEMSAKLLV
SCTRRVVSGLYAGNKYDLLFPSVLDDNLSEKMLQLISRLCCLYTVLFATT
REIPKIRGLSAEEKCSVLTEYLLSDAVKPLLRSEQVSCIMSPNIITFPAN
LYYMSRKSLNLIREREDRDAILALLFPQEPLLEFRPVQDIGVRVKDPFTR
QPAALLQELDLSAPARYDAFTLNEVRSEHTLPNPEEDYLVRYLFRGIGTA
SSSWYKASHLLSVPEVRCARYGNSLYLAEGSGAIMSLLELHVPHETIYYN
TLFSNEMNPPQRHFGPTPTQFLNSVVYRNLQAEVPCKDGFIQEFRPLWRE
NAEESDLTSDKAVGYITSAVPYRSVSLLHCDIEIPPGSNQSLLDQLAINL
SLIAMHSVREGGVVIIKVLYAMGYYFHLLMNLFTPCSTKGYILSNGYACR
GDMECYLIFVMGYLGGPTFVHEVVRMAKTLVQRHGTLLSKSDEITLTRLF
TSQQHRVTDILSSPLPRLMKFLRENIDAALIEAGGQPVRPFCAESLVSTL
TDMTQMTQIIASHIDTVIRSVIYMEAEGDLADTVFLFTPYNLSTDGKKRT
SLKQCTRQILEVTILGLRVKDLNKVGDVIGLVLRGMVSLEDLIPLRTYLR
RSTCPKYLKAVLGITKLKEMFTDTSLLYLTLAQQKFYMKTIGNAAKGYYS
NCDS;

Fusion (aka F) encoded by the F gene and having e.g., the following amino acid sequence as set forth in UniProt Accession No. Q9DLD4:

(SEQ ID NO: 62)
MGSRPFTKNPAPMMLTIRVALVLSCICPANSIDGRPFAAAGIVVTGDKAV
NIYTSSQTGSIIVKLLPNLPKDKEACAKAPLDAYNRTLTTLLTPLGDSIR
RIQESVTTSGGGRQGRLIGAIIGGVALGVATAAQITAAAALIQAKQNAAN
ILRLKESIAATNEAVHEVTDGLSQLAVAVGKMQQFVNDQFNKTAQELDCI
KIAQQVGVELNLYLTELTTVFGPQITSPALNKLTIQALYNLAGGNMDYLL
TKLGIGNNQLSSLIGSGLITGNPILYDSQTQLLGIQVTLPSVGNLNNMRA
TYLETLSVSTTRGFASALVPKVVTQVGSVIEELDTSYCIETDLDLYCTRI
VTFPMSPGIYSCLSGNTSACMYSKTEGALTTPYMTIKGSVIANCKMTTCR
CVNPPGIISQNYGEAVSLIDKQSCNVLSLGGITLRLSGEFDVTYQKNISI
QDSQVIITGNLDISTELGNVNNSISNALNKLEESNRKLDKVNVKLTSTSA
LITYIVLTIISLVFGILSLILACYLMYKQKAQQKTLLWLGNNTLDQMRAT
TKM;

Beclin-1 (aka GT197, ATG6, VPS30) encoded by the BECN1 gene and having e.g., the following amino acid sequence as set forth in UniProt Accession No. Q14457:

(SEQ ID NO: 63)
MEGSKTSNNSTMQVSFVCQRCSQPLKLDTSFKILDRVTIQELTAPLLTTA
QAKPGETQEEETNSGEEPFIETPRQDGVSRRFIPPARMMSTESANSFTLI
GEASDGGTMENLSRRLKVTGDLFDIMSGQTDVDHPLCEECTDTLLDQLDT
QLNVTENECQNYKRCLEILEQMNEDDSEQLQMELKELALEEERLIQELED
VEKNRKIVAENLEKVQAEAERLDQEEAQYQREYSEFKRQQLELDDELKSV
ENQMRYAQTQLDKLKKTNVFNATFHIWHSGQFGTINNFRLGRLPSVPVEW
NEINAAWGQTVLLLHALANKMGLKFQRYRLVPYGNHSYLESLTDKSKELP
LYCSGGLRFFWDNKFDHAMVAFLDCVQQFKEEVEKGETRFCLPYRMDVEK
GKIEDTGGSGGSYSIKTQFNSEEQWTKALKFMLTNLKWGLAWVSSQFYNK;

Bnip1 (aka SEC20, Nip1) encoded by the BNIP1 gene and having e.g., the following amino acid sequence as set forth in UniProt Accession No. Q12981:

(SEQ ID NO: 64)
MAAPQDVHVRICNQEIVKFDLEVKALIQDIRDCSGPLSALTELNTKVKEK
FQQLRHRIQDLEQLAKEQDKESEKQLLLQEVENHKKQMLSNQASWRKANL
TCKIAIDNLEKAELLQGGDLLRQRKTTKESLAQTSSTITESLMGISRMMA
QQVQQSEEAMQSLVTSSRTILDANEEFKSMSGTIQLGRKLITKYNRRELT
DKLLIFLALALFLATVLYIVKKRLFPFL;

Bnip2 (aka Nip2) encoded by the BNIP2 gene and having e.g., the following amino acid sequence as set forth in UniProt Accession No. Q12982:

(SEQ ID NO: 65)
MEGVELKEEWQDEDFPIPLPEDDSIEADILAITGPEDQPGSLEVNGNKVR
KKLMAPDISLTLDPSDGSVLSDDLDESGEIDLDGLDTPSENSNEFEWEDD
LPKPKTTEVIRKGSITEYTAAEEKEDGRRWRMFRIGEQDHRVDMKAIEPY
KKVISHGGYYGDGLNAIVVFAVCFMPESSQPNYRYLMDNLFKYVIGTLEL
LVAENYMIVYLNGATTRRKMPSLGWLRKCYQQIDRRLRKNLKSLIIVHPS
WFIRTLLAVTRPFISSKFSQKIRYVFNLAELAELVPMEYVGIPECIKQVD
QELNGKQDEPKNEQ;

Bnip3 (aka Nip3) encoded by the BNIP3 gene and having e.g., the following amino acid sequence as set forth in UniProt Accession No. Q12983:

(SEQ ID NO: 66)
MGDAAADPPGPALPCEFLRPGCGAPLSPGAQLGRGAPTSAFPPPAAEAHP
AARRGLRSPQLPSGAMSQNGAPGMQEESLQGSWVELHFSNNGNGGSVPAS
VSIYNGDMEKILLDAQHESGRSSSKSSHCDSPPRSQTPQDTNRASETDTH
SIGEKNSSQSEEDDIERRKEVESILKKNSDWIWDWSSRPENIPPKEFLFK
HPKRTATLSMRNTSVMKKGGIFSAEFLKVFLPSLLLSHLLAIGLGIYIGR
RLTTSTSTF;

Bnip31 (aka Nix, BNIP3a) encoded by the BNIP3L gene and having e.g., the following amino acid sequence as set forth in UniProt Accession No. 060238:

(SEQ ID NO: 67)
MSSHLVEPPPPLHNNNNNCEENEQSLPPPAGLNSSWVELPMNSSNGNDNG
NGKNGGLEHVPSSSSIHNGDMEKILLDAQHESGQSSSRGSSHCDSPSPQE
DGQIMFDVEMHTSRDHSSQSEEEVVEGEKEVEALKKSADWVSDWSSRPEN
IPPKEFHFRHPKRSVSLSMRKSGAMKKGGIFSAEFLKVFIPSLFLSHVLA
LGLGIYIGKRLSTPSASTY;

Brcc2 (aka Blid) encoded by the BLID gene and having e.g., the following amino acid sequence as set forth in UniProt Accession No. Q8IZY5:

(SEQ ID No: 68)
MVTLLPIEGQEIHFFEILESECVLYTGWIERASGSSIYPEAKARLPLEAL
LGSNKEPMLPKETVLSLKRYNLGSSAMKRNVPGHVLQRPSYLTRIQVTLL
CNSSAEAL;

ceBNIP3 (aka Dct-1) encoded by the CEBNIP3 gene and having e.g., the following amino acid sequence as set forth in UniProt Accession No. Q09969:

(SEQ ID NO: 69)
MSSFLEFAKPKMLDIKRKINFASGEKTDESVQPQQQTEQSSAQQTTPSAK
AVSNPFITPLTESTPGMSESWVELAPSRTSLCSSVDINMVIIDEKDKDSR
LSPVSIAQSPHVEFESLEQVKYKLVREMLPPGKNTDWIWDWSSRPENTPP
KTVRMVQYGSNLTTPPNSPEPELYQYLPCESDSLFNVRVVFGFLVTNIFS
FVVGAAVGFAVCRKLIKHHRQ;

Clusterin (aka Apo-J, CLU1, CLU2, KUB1, SGP-2, SP-40, TRPM-2) encoded by the CLU gene and having e.g., the following amino acid sequence as set forth in UniProt Accession No. P10909:

(SEQ ID NO: 70)
MMKTLLLFVGLLLTWESGQVLGDQTVSDNELQEMSNQGSKYVNKEIQNAV
NGVKQIKTLIEKTNEERKTLLSNLEEAKKKKEDALNETRESETKLKELPG
VCNETMMALWEECKPCLKQTCMKFYARVCRSGSGLVGRQLEEFLNQSSPF
YFWMNGDRIDSLLENDRQQTHMLDVMQDHFSRASSIIDELFQDRFFTREP
QDTYHYLPFSLPHRRPHFFFPKSRIVRSLMPFSPYEPLNFHAMFQPFLEM
IHEAQQAMDIHFHSPAFQHPPTEFIREGDDDRTVCREIRHNSTGCLRMKD
QCDKCREILSVDCSTNNPSQAKLRRELDESLQVAERLTRKYNELLKSYQW
KMLNTSSLLEQLNEQFNWVSRLANLTQGEDQYYLRVTTVASHTSDSDVPS
GVTEVVVKLFDSDPITVTVPVEVSRKNPKFMETVAEKALQEYRKKHREE;

Core (aka Capsid, Nucleocapsid, C) encoded by the CORE gene and having e.g., the following amino acid sequence as set forth in UniProt Accession No. Q00269:

(SEQ ID NO: 71)
MSTNPKPQRKTKRNTYRRPQDVKFPGGGQIVGGVYVLPRRGPTLGVRATR
KTSERSQPRGRRQPIPKARRPEGRAWAQPGYPWPLYGNEGLGWAGWLLSP
RGSRPSWGPTDPRRRSRNLGKVIDTLTCGFADLMGYIPLVGAPLGGAARA
LAHGVRVLEDGVNYATGNLPGCSFSIFLLALLSCLTIPASAYQVRNASGL
YHVTNDCSNSSIVYEAAGMIMHTPGCVPCVRENNASRCWVALTPTLAARN
TSIPTTTIRRHVDLLVGAAAFCSAMYVGDLCGSVFLVSQLFTFSPRRYET
VQDCNCSIYPGHVSGHRMAWDMMMNWSPTTALVVSQLLRIPQAVVDMVAG
AHWGVLAGLAYYSMVGNWAKVLIVMLLFAGVDGVTYTTGGSQARHTQSVT
SFFTQGPAQRIQLINTNGSWHINRTALNCNESLNTGFFAALFYAHKFNSS
GCPERMASCSSIDKFAQGWGPITYTEPRDLDQRPYCWHYAPRQCGIVPAS
QVCGPVYCFTPSPVVVGTTDRSGAPTYNWGANETDVLLLNNTRPPQGNWF
GCTWMNSTGFTKTCGGPPCNIGGVGNLTLTCPTDCFRKHPEATYTKCGSG
PWLTPRCIVDYPYRLWHYPCTVNFTIFKVRMYVGGVEHRLSAACNWTRGE
RCDLEDRDRSELSPLLLSTTEWQTLPCSFTTLPALSTGLIHLHQNIVDVQ
YLYGIGSAVVSFVIKWEYIVLLFLLLADARVCACLWMMLLIAQAEAALEN
LVVLNAASLAGADGILSFLVFFCAAWYIKGRLVPGAAYALYGVWPLLLLL
LALPPRAYAMDREMAASCGGVVFVGLILLTLSPHYKVFLARLIWWLQYFI
TRAEAHLCVWVPPLNVRGGRDAIILLTCAAHPELIFDITKLLLAILGPLM
VLQAAITAMPYFVRAQGLIRACMLVRKVAGGHYVQMAFMKLAALTGTYVY
DHLTPLQDWAHAGLRDLAVAVEPVVFSDMETKIITWGADTAACGDIILGL
PVSARRGREILLGPADSIEGQGWRLLAPITAYAQQTRGLLGCIVTSLTGR
DKNQVEGEVQVVSTATQSFLATCVNGVCWTVFHGAGSKTLAGPKGPITQM
YTNVDQDLVGWHAPPGARSLTPCTCGSSDLYLVTRHADVIPVRRRGDGRG
SLLSPRPVSYLKGSSGGPLLCPSGHAVGIFRAAVCTRGVAKAVDFIPVES
METTMRSPVFTDNSSPPAVPQTFQVAHLHAPTGSGKSTKVPAAYAAQGYK
VLVLNPSVAATLGFGAYMSKAHGTDPNIRTGVRTITTGAPITYSTYGKFL
ADGGCSGGAYDIIICDECHSTDSTTILGIGTVLDQAETAGARLVVLATAT
PPGSVTVPHPNIEEVALSNTGEIPFYGKAIPLEAIKGGRHLIFCHSKKKC
DELAAKLSGLGINAVAYYRGLDVSVIPTSGDVVIVATDALMTGYTGDFDS
VIDCNTCVTQTVDFSLDPTFTIETTTVPQDAVSRSQRRGRTGRGRGGIYR
FVTPGERPSGMFDSSVLCECYDAGCAWYELTPAETTVRLRAYLNTPGLPV
CQDHLEFWESVFTGLTHIDAHFLSQTKQAGDNFPYLVAYQATVCARAQAP
PPSWDQMWKCLIRLKPTLHGPTPLLYRLGAVQNEITLTHPITKFIMACMS
ADLEVVTSTWVLVGGVLAALAAYCLTTGSVVIVGRIILSGRPAVVPDREV
LYREFDEMEECASHLPYIEQGMQLAEQFKQKALGLLQTATKQAEAAAPVV
ESRWRALEAFWAKHMWNFISGIQYLAGLSTLPGNPAIASLMAFTASITSP
LTTQNTLLFNILGGWVAAQLAPPSAASAFVGAGIAGAAIGSIGLGKVLVD
ILAGYGAGVAGALVAFKVMSGEAPSAEDLVNLLPAILSPGALVVGVVCAA
ILRRHVGPGEGAVQWMNRLIAFASRGNHVSPTHYVPESDAAARVTQILSS
LTITQLLKRLHQWINEDCSTPCSGSWLKDVWDWICTVLTDFKTWLQSKLL
PKLPGVPFFSCQRGYKGVWRGDGIMQTTCPCGAQITGHVKNGSMRIVGPK
TCSNTWHGTFPINAYTTGPCTPSPAPNYSRALWRVAAEEYVEITRVGDFH
YVTGMTTDNVKCPCQVPAPEFFTELDGVRLHRYAPACRPLLREDVTFQVG
LNQYLVGSQLPCEPEPDVAVLTSMLTDPSHITAETAKRRLARGSPPSLAS
SSASQLSAPSLKATCTTHHDSPDADLIEANLLWRQEMGGNITRVESENKV
VILDSFDPLRAEEDEREVSVAAEILRKSKKFPPALPIWARPDYNPPLLES
WKSPDYVPPAVHGCPLPPTTGPPIPPPRKKRTVVLTESTVSSALAELATK
TFGSSGSSAVDSGTATAPPDQTSDDGDKESDVESYSSMPPLEGEPGDPDL
SDGSWSTVSGEASDDIVCCSMSYTWTGALITPCAAEESKLPINALSNSLL
RHHNMVYATTSRSASLRQKKVTFDRLQVLDDHYRDVLKEMKAKASTVKAK
LLSVEEACKLTPPHSAKSKFGYGAKDVRNLSSKAINHIRSVWKDLLEDTE
TPIDTTIMAKSEVFCVQPEKGGRKPARLIVFPDLGVRVCEKMALYDVVST
LPQAVMGSSYGFQYSPGQRVEFLVNAWKSKKSPMGFSYDTRCFDSTVTES
DIRVEESIYQCCDLAPEARQAIKSLTERLYIGGPLTNSKGQNCGYRRCRA
SGVLTTSCGNTLTCYLKATAACRAAKLQDCTMLVNGDDLVVICESAGTQE
DAASLRVFTEAMTRYSAPPGDPPQPEYDLELITSCSSNVSVAHDASGKRV
YYLTRDPTTPLARAAWETARHTPVNSWLGNIIMYAPTLWARMILMTHFFS
ILLAQEQLEKALDCQIYGACYSIEPLDLPQIIQRLHGLSAFSLHSYSPGE
INRVASCLRKLGVPPLRVWRHRARSVRARLLSQGGRAATCGKYLFNWAVR
TKLKLTPIPAASQLDLSSWFVAGYSGGDIYHSLSRARPRWFMWCLLLLSV
GVGIYLLPNR;

Cullin-7 (aka p193, dJ2007.5) encoded by the CULLIN-7 gene and having e.g., the following amino acid sequence as set forth in UniProt Accession No. Q8VE73:

(SEQ ID NO: 72)
MVGELRYREFRVPLGPGLHAYPDELIRQRVGHNGHPEYQIRWLILRRGDD
GDRDSTVDCKAEHILLWMSDDEIYANCHKMLGENGQVIAPSRESTEAGAL
DKSVLGEMETDVKSLIQRALRQLEECVGTVPPAPLLHTVHVLSAYASIEP
LTGIFKDRRVVNLLMHMLSSPDYQIRWSAGRMIQALSSHDAGTRTQILLS
LSQQEAIEKHLDFDSRCALLALFAQATLTEHPMSFEGVQLPQVPGRLLFS
LVKRYLHVTFLLDRLNGDAGDQGAQNNFIPEELNVGRGRLELEFSMAMGT
LISELVQAMRWDGASSRPESSSSSTFQPRPAQFRPYTQRFRRSRRFRPRA
SFASFNTYALYVRDTLRPGMRVRMLENYEEIAAGDEGQFRQSNDGVPPAQ
VLWDSTGHTYWVHWHMLEILGFEEDIEDVIDIEELQELGANGALSIVPPS
QRWKPITQLFAEPYVVPEEEDREESENLTQAEWWELLFFIRQLSEAERLH
IVDLLQDHLEEERVLDYDMLPELTVPVDLAQDLLLSLPQQLEDSALRDLF
SCSVYRKYGPEVLVGHLSYPFVPGAQPNLFGANEESEAKDPPLQSASPAL
QRLVESLGPEGEVLVELEQALGSEAPQETEVKSCLLQLQEQPQPFLALMR
SLDTSASNKTLHLTVLRILMQLVNFPEALLLPWHEAMDACVTCLRSPNTD
REVLQELIFFLHRLTTTSRDYAVILNQLGARDAISKVLEKHRGKLELAQE
LRDMVSKCEKHAHLYRKLTTNILGGCIQMVLGQIEDHRRTHRPIQIPFFD
VFLRYLCQGSSEEMKKNRYWEKVEVSSNPQRASRLTDRNPKTYWESSGRA
GSHFITLHMRPGVIIRQLTLLVAGEDSSYMPAWVVVCGGNSIKSVNKELN
TVNVMPSASRVTLLENLTRFWPIIQIRIKRCQQGGINTRIRGLEVLGPKP
TFWPVFREQLCRHTRLFYMVRAQAWSQDIAEDRRSLLHLSSRLNGALRHE
QNFAERFLPDMEAAQALSKTCWEALVSPLVQNITSPDEDSTSSLGWLLDQ
YLGCREAAYNPQSRAAAFSSRVRRLTHLLVHVEPREAAPPVVAIPRSKGR
NRIHDWSYLITRGLPSSIMKNLTRCWRSVVEEQMNKFLTASWKDDDFVPR
YCERYYVLQKSSSELFGPRAAFLLAMRNGCADAVLRLPFLRAAHVSEQFA
RHIDQRIQGSRMGGARGMEMLAQLQRCLESVLIFSPLEIATTFEHYYQHY
MADRLLSVGSSWLEGAVLEQIGPCFPSRLPQQMLQSLNVSEELQRQFHVY
QLQQLDQELLKLEDTEKKIQVAHEDSGREDKSKKEEAIGEAAAVAMAEEE
DQGKKEEGEEEGEGEDEEEERYYKGTMPEVCVLVVTPRFWPVASVCQMLN
PATCLPAYLRGTINHYTNFYSKSQSRSSLEKEPQRRLQWTWQGRAEVQFG
GQILHVSTVQMWLLLHLNNQKEVSVESLQAISELPPDVLHRAIGPLTSSR
GPLDLQEQKNVPGGVLKIRDDSEEPRPRRGNVWLIPPQTYLQAEAEEGRN
MEKRRNLLNCLVVRILKAHGDEGLHVDRLVYLVLEAWEKGPCPARGLVSS
LGRGATCRSSDVLSCILHLLVKGTLRRHDDRPQVLYYAVPVTVMEPHMES
LNPGSAGPNPPLTFHTLQIRSRGVPYASCTDNHTFSTFR

E (aka Envelope, sM) encoded by the E gene and having e.g., the following amino acid sequence as set forth in UniProt Accession No. P59637:

(SEQ ID NO: 73)
MYSFVSEETGTLIVNSVLLFLAFVVFLLVTLAILTALRLCAYCCNIVNVS
LVKPTVYVYSRVKNLNSSEGVPDLLV;

Erbb2 (aka Her2,Neu) encoded by the ERBB2 gene and having e.g., the following amino acid sequence as set forth in UniProt Accession No. P04626:

(SEQ ID NO: 74)
MELAALCRWGLLLALLPPGAASTQVCTGTDMKLRLPASPETHLDMLRHLY
QGCQVVQGNLELTYLPTNASLSFLQDIQEVQGYVLIAHNQVRQVPLQRLR
IVRGTQLFEDNYALAVLDNGDPLNNTTPVTGASPGGLRELQLRSLTEILK
GGVLIQRNPQLCYQDTILWKDIFHKNNQLALTLIDTNRSRACHPCSPMCK
GSRCWGESSEDCQSLTRTVCAGGCARCKGPLPTDCCHEQCAAGCTGPKHS
DCLACLHFNHSGICELHCPALVTYNTDTFESMPNPEGRYTFGASCVTACP
YNYLSTDVGSCTLVCPLHNQEVTAEDGTQRCEKCSKPCARVCYGLGMEHL
REVRAVTSANIQEFAGCKKIFGSLAFLPESFDGDPASNTAPLQPEQLQVF
ETLEEITGYLYISAWPDSLPDLSVFQNLQVIRGRILHNGAYSLTLQGLGI
SWLGLRSLRELGSGLALIHHNTHLCFVHTVPWDQLFRNPHQALLHTANRP
EDECVGEGLACHQLCARGHCWGPGPTQCVNCSQFLRGQECVEECRVLQGL
PREYVNARHCLPCHPECQPQNGSVTCFGPEADQCVACAHYKDPPFCVARC
PSGVKPDLSYMPIWKFPDEEGACQPCPINCTHSCVDLDDKGCPAEQRASP
LTSIISAVVGILLVVVLGVVFGILIKRRQQKIRKYTMRRLLQETELVEPL
TPSGAMPNQAQMRILKETELRKVKVLGSGAFGTVYKGIWIPDGENVKIPV
AIKVLRENTSPKANKEILDEAYVMAGVGSPYVSRLLGICLTSTVQLVTQL
MPYGCLLDHVRENRGRLGSQDLLNWCMQIAKGMSYLEDVRLVHRDLAARN
VLVKSPNHVKITDFGLARLLDIDETEYHADGGKVPIKWMALESILRRRFT
HQSDVWSYGVTVWELMTFGAKPYDGIPAREIPDLLEKGERLPQPPICTID
VYMIMVKCWMIDSECRPRFRELVSEFSRMARDPQRFVVIQNEDLGPASPL
DSTFYRSLLEDDDMGDLVDAEEYLVPQQGFFCPDPAPGAGGMVHHRHRSS
STRSGGGDLTLGLEPSEEEAPRSPLAPSEGAGSDVFDGDLGMGAAKGLQS
LPTHDPSPLQRYSEDPTVPLPSETDGYVAPLTCSPQPEYVNQPDVRPQPP
SPREGPLPAARPAGATLERPKTLSPGKNGVVKDVFAFGGAVENPEYLTPQ
GGAAPQPHPPPAFSPAFDNLYYWDQDPPERGAPPSTFKGTPTAENPEYLG
LDVPV;

Erbb4 (aka Her4) encoded by the ERBB4 gene and having e.g., the following amino acid sequence as set forth in UniProt Accession No. Q15303:

(SEQ ID NO: 75)
MKPATGLWVWVSLLVAAGTVQPSDSQSVCAGTENK
LSSLSDLEQQYRALRKYYENCEVVMGNLEITSIEH
NRDLSFLRSVREVTGYVLVALNQFRYLPLENLRII
RGTKLYEDRYALAIFLNYRKDGNFGLQELGLKNLT
EILNGGVYVDQNKFLCYADTIHWQDIVRNPWPSNL
TLVSTNGSSGCGRCHKSCTGRCWGPTENHCQTLTR
TVCAEQCDGRCYGPYVSDCCHRECAGGCSGPKDTD
CFACMNFNDSGACVTQCPQTFVYNPTTFQLEHNFN
AKYTYGAFCVKKCPHNFVVDSSSCVRACPSSKMEV
EENGIKMCKPCTDICPKACDGIGTGSLMSAQTVDS
SNIDKFINCTKINGNLIFLVTGIHGDPYNAIEAID
PEKLNVFRTVREITGFLNIQSWPPNMTDFSVFSNL
VTIGGRVLYSGLSLLILKQQGITSLQFQSLKEISA
GNIYITDNSNLCYYHTINWTTLFSTINQRIVIRDN
RKAENCTAEGMVCNHLCSSDGCWGPGPDQCLSCRR
FSRGRICIESCNLYDGEFREFENGSICVECDPQCE
KMEDGLLTCHGPGPDNCTKCSHFKDGPNCVEKCPD
GLQGANSFIFKYADPDRECHPCHPNCTQGCNGPTS
HDCIYYPWTGHSTLPQHARTPLIAAGVIGGLFILV
IVGLTFAVYVRRKSIKKKRALRRFLETELVEPLTP
SGTAPNQAQLRILKETELKRVKVLGSGAFGTVYKG
IWVPEGETVKIPVAIKILNETTGPKANVEFMDEAL
IMASMDHPHLVRLLGVCLSPTIQLVTQLMPHGCLL
EYVHEHKDNIGSQLLLNWCVQIAKGMMYLEERRLV
HRDLAARNVLVKSPNHVKITDFGLARLLEGDEKEY
NADGGKMPIKWMALECIHYRKFTHQSDVWSYGVTI
WELMTFGGKPYDGIPTREIPDLLEKGERLPQPPIC
TIDVYMVMVKCWMIDADSRPKFKELAAEFSRMARD
PQRYLVIQGDDRMKLPSPNDSKFFQNLLDEEDLED
MMDAEEYLVPQAFNIPPPIYTSRARIDSNRSEIGH
SPPPAYTPMSGNQFVYRDGGFAAEQGVSVPYRAPT
STIPEAPVAQGATAEIFDDSCCNGTLRKPVAPHVQ
EDSSTQRYSADPTVFAPERSPRGELDEEGYMTPMR
DKPKQEYLNPVEENPFVSRRKNGDLQALDNPEYHN
ASNGPPKAEDEYVNEPLYLNTFANTLGKAEYLKNN
ILSMPEKAKKAFDNPDYWNHSLPPRSTLQHPDYLQ
EYSTKYFYKQNGRIRPIVAENPEYLSEFSLKPGTV
LPPPPYRHRNTVV;

Itm2bs (aka Bri) encoded by the ITM2B gene and having e.g., the following amino acid sequence as set forth in UniProt Accession No. Q9Y287:

(SEQ ID NO: 76)
MVKVTFNSALAQKEAKKDEPKSGEEALIIPPDAVA
VDCKDPDDVVPVGQRRAWCWCMCFGLAFMLAGVIL
GGAYLYKYFALQPDDVYYCGIKYIKDDVILNEPSA
DAPAALYQTIEENIKIFEEEEVEFISVPVPEFADS
DPANIVHDFNKKLTAYLDLNLDKCYVIPLNTSIVM
PPRNLLELLINIKAGTYLPQSYLIHEHMVITDRIE
NIDHLGFFIYRLCHDKETYKLQRRETIKGIQKREA
SNCFAIRHFENKFAVETLICS;

Map-1 (aka PNMA4) encoded by the MOAP-1 gene and having e.g., the following amino acid sequence as set forth in UniProt Accession No. Q96BY2:

(SEQ ID NO: 77)
MTLRLLEDWCRGMDMNPRKALLIAGISQSCSVAEI
EEALQAGLAPLGEYRLLGRMFRRDENRKVALVGLT
AETSHALVPKEIPGKGGIWRVIFKPPDPDNTFLSR
LNEFLAGEGMTVGELSRALGHENGSLDPEQGMIPE
MWAPMLAQALEALQPALQCLKYKKLRVFSGRESPE
PGEEEFGRWMFHTTQMIKAWQVPDVEKRRRLLESL
RGPALDVIRVLKINNPLITVDECLQALEEVFGVTD
NPRELQVKYLTTYQKDEEKLSAYVLRLEPLLQKLV
QRGAIERDAVNQARLDQVIAGAVHKTIRRELNLPE
DGPAPGFLQLLVLIKDYEAAEEEEALLQAILEGNF
T;

Mcf encoded by the MCF gene and having e.g., the following amino acid sequence as set forth in UniProt Accession No. Q8KT65:

(SEQ ID NO: 78)
MASISKDFTNLLNTLIDGQIGAASRQTEWFNMSPD
ERTDYIKQVDERLQEMQQSTLSVLAAQHFQMQDNP
VSVGDQLQTLQKRRQQMTDVPGTPAINAYKQQLDR
DILLYRRQQTAMTHFDSTWRKVLVMLGPDDSKPLN
ATTLRENAVDKQAKLDTEIKRLEQQLTIQVADSTF
SQKYVTLFSELQAYKDVNARYNALLKASATEEAAA
LGALTKVPQASDDLPVNISLLMMEERPGYIRMNVA
LVNASTDGRFKDFFLENGRLVVLTDGVLNFSFGTA
ARSLAWQQQYRLKSEPPSFRSPTYTPIRSVLVKTE
FVEKYFANYLVSESTLRGGFKAQLLGNGRKMLLTS
VDRKVPNQIGIQVSGQAPNTTITREVPLASALSDL
INQNADIASFRTIGLEGFRQSSYHPDRDGLFVNIH
ELERSVGFAGRQYLLEMPQDNDYLSATPFGVMSVD
GDKVSSSHLSKAQTDTLYQYNAAFFEKLEQLRSGG
MKASRLFEGSIERTAFVQQLVRLLERNHITPAGVL
APEYPRDNMRDIKGNNLNKVLWEQAFAASVWRSRD
NDPLLFRLATRLVKNPAVVKVLQNGYVQSDIAQAR
ELLAPLYEQWRTRAVEAETQRVASANAAQHPSNPK
VHVFDQAEVERSLDDKLLILLLTGPQSLEGTDVQL
RPMVEAALLSNEGRSLRKQILFHALRPVADSFSKA
AAPVNPHAELGVGKIMINNRLNQPDPYLILNTSSE
EQAYRDGSYLIKDDKYRSYNQFRPDFKNDATRYMN
DLDTPFVGGISGTTQTVSNVLTELFGGALSVKQYW
QFQMANAAFMIRNGYHSFFETFYVAARYEPEGADS
IGKEMLQMFDKYRVEGSKKALQGKLYDGVMARVLP
IINQGLSAADEFHPPRFTRIGPRPALLGQAVKDLE
LKAGLTSVGDGFEPRQGSADIHQFVTDPVLFAKTH
TVSAEALVRSGRLPAEGSAQLVKVGSGLYELEYTE
QSANDISSSSIPAYFLGYNGPNQANAVPAYVDIPK
RTIAGNFLFTGTLSGGSLVVTSLDANTFRVYHDGR
VNSSLLYDNVVMAVDYKDYQIAGTAEGLAAAYMQY
VNHEWQLVLQRQEYQRDGQMLRLRLRDDEEPLSIQ
VADSQVVERNQAQFVAYREQIHQQLKKVATQFEVS
ISGVSDGVYTEGEFSPDHPAIAAWAKLCAEVYDRI
NADTKQLVDKRNKLYENRRNTIRRDLINQQIKQLN
ITLEYYKAQYDTVLREAGFVEQSWLWQQIKAKNGS
AAVVRIDDTAIQGGGKQRTDSVGERYAISEAYQRG
ARGTGFSDGLRNFREIEIPGVDDKMSALEMKRLFL
EGKLTSEQQGALSGRITETSRAEYIDKVLRQTAVF
SEDFHDAGSVFDRLVPQDFYLSLVGDRSGGRCYPL
VRAMTVALASGGEAGINSLVQKLFFASADPQAGSS
TLLRNSLIKLHSNVEAVQASTELGQFGLSEVVSRL
AATTGTSMFALNTQNHSMMVGSTVTTEGRRYYFYD
PNVGIFAFDNTKSLSRAMEQHLVGRRLAVHYGSFG
SKSAPAFNLIEIDTGKMAEVPVGNGLNVADLTRFE
ELSSVIGQRRQVEQVMSAQERITEDLQLSTALQAF
DAEQWGARFEAASTRLAQEHQLDSRWLPIIATTEE
QGEGRYRVQFINRDQPEQTRWLDTDDSTFVEFRRF
VDEHMSVLNEHFTLESGRMRPRGGVGEAAPVDGLN
AGFAVQALIQWFSDKNRHDAANGMASPDLATALKV
HSYLNFVQMVHGGVQDVIKVTALVRTALRGEVVAA
QTSFKEFALSLGHTVNEGVGVLFGGAMIGLDAYEL
AHAENDVQKAVFGTQLAFDSASFVTGAAGIGAGLV
GASTAGAVLGGAGVILGGLAVGFTALAQAFGAVAE
DAKAVGRYFDTVDKAYKGNGYRYDNEKQVLVPLAG
AVIKTLDLSKNQIDFDSQYIYRTHSGSTGSGKINY
FFWVGDFPRMVHDRGQAIEVRSGIGYKDVSRPLEH
GDSNVVILPGTPKSYISYEYMLLPGATTRHDAGFD
VIRRLEEDKRFDYDFYIFPGEETIRRIHHEYVDTP
IEVVLDQRNRQLVAPELPKELHGFLCYEIKGAGGE
YLIGLNEGAKVNLTSDVASTWIIDSSQLASDSISV
SKDQLLVGEKGKEVVVKLYLAQNSQVLVVNGKGEV
RKVDFTSLTAQVISEDASKWQVPGQQIEQHLSDLA
KAHQLHGQYVVVENYRHQGRDVGRAFYDVTKDRML
FTDTTNEQAKRAQLGAVMGDYAYFYDADNAVAWRV
DIATGQVDAQFEPWFNQNAGHISRFWQEGDVVYLA
RRYRLKEREAELGYRIIGDRMELVSAVGDDALLQL
SARIGRHGDELEAILQGYRSNSTQRGTLMYTLGAR
LIQPTSAALVTVFGVDAAGVPHRYWIRTSDGTLIK
PNLAPPADQTLHFEAHEQTRSAWQIPADLVLAGSM
PLLGGKEVFFFYSKEQKTLFRQEGPGQEVLDANQP
SALRVTTPALTNVINLNGHLVVVTEDGRVARLDAL
GQLSYAAVNEHWLKGRIHWWQDLTSVTDGRATLAV
FGVKDTDGKSLLPVWYHNGQVVVASAALQDKHPQF
LGFEVDGSSARLFEPASGKLYRQPAMTADALAAAF
GTDEVLEASAQLPAANELEPELHLKAAEQVDAGLR
LTTVKGEILLRTHDGKLQLVAVDKDWQQDNLVRLS
QALAEVAGQWRVKGVLTLQGDDTQGWFDVGSGQVF
SIGGIPATDNLRFIGIAVGKKGAYVYNPTDQMLYQ
VKESGAQKLNHYADVERIGSSLLLQDGGKGDLSPM
LIAGVDSVVLHGGAGSDTYRLSQTMWSYYRTVVID
NDDPNQVLDRLIILAVDAEKIFVSRHEDDLMLTDS
VNGTVLVIRKVFGSQAVTHRHLQIDLEGSSSVISV
DHLVKGFTRLGTANIGLFELPWAI;

Pxt1 encoded by the PXT1 gene and having e.g., the following amino acid sequence as set forth in UniProt Accession No. Q8K459:

(SEQ ID NO: 79)
MQLRHIGDSVNHRVIQEHLAQEVGDVLAPFVALVF
VRGQVLLRFFWNNHLL;

Rad9a (aka hRad9) encoded by the RAD9A gene and having e.g., the following amino acid sequence as set forth in UniProt Accession No. Q99638:

(SEQ ID NO: 80)
MKCLVTGGNVKVLGKAVHSLSRIGDELYLEPLEDG
LSLRTVNSSRSAYACFLFAPLFFQQYQAATPGQDL
LRCKILMKSFLSVFRSLAMLEKTVEKCCISLNGRS
SRLVVQLHCKFGVRKTHNLSFQDCESLQAVFDPAS
CPHMLRAPARVLGEAVLPFSPALAEVTLGIGRGRR
VILRSYHEEEADSTAKAMVTEMCLGEEDFQQLQAQ
EGVAITFCLKEFRGLLSFAESANLNLSIHFDAPGR
PAIFTIKDSLLDGHFVLATLSDTDSHSQDLGSPER
HQPVPQLQAHSTPHPDDFANDDIDSYMIAMETTIG
NEGSRVLPSISLSPGPQPPKSPGPHSEEEDEAEPS
TVPGTPPPKKFRSLFFGSILAPVRSPQGPSPVLAE
DSEGEG;

SpRad9 encoded by the RAD9 gene and having e.g., the following amino acid sequence as set forth in UniProt Accession No. P26306:

(SEQ ID NO: 81)
MEFTVSNVNLRDLARIFTNLSRIDDAVNWEINKNQ
IEITCLNSSRSGFSMVTLKKAFFDKYIFQPDSVLL
TGLMTPTIRIRTQVKPILSVFRNKIFDFIPTVVTT
NSKNGYGSESASRKDVIVENVQISISTGSECRIIF
KFLCKHGVIKTYKISYEQTQTLHAVFDKSLSHNNF
QINSKILKDLTEHFGQRTEELTIQPLQERVLLTSF
TEEVVHNRDILKQPTQTTVSIDGKEFERVALNEGV
SVTLSLREFRAAVILAEALGSSICAYYGVPGKPIL
LTFAKGKNSEIEAQFILATVVGSDEQEVSSMMGNR
WQHSSTPASLFNSVERNNSLTAVAHNPPGSIGWQT
DQSDSSRMFNSALDRSDETNGIKEPSTTNDAGQSL
FLDGIPNESELAAFNNDVNDDAEFGPTQAEQSYHG
IFSQED;

Soul (aka PP23, Hepb2) encoded by the HEPB2 gene and having e.g., the following amino acid sequence as set forth in UniProt Accession No. Q9Y5Z4:

(SEQ ID NO: 82)
MAEPLQPDPGAAEDAAAQAVETPGWKAPEDAGPQP
GSYEIRHYGPAKWVSTSVESMDWDSAIQTGFTKLN
SYIQGKNEKEMKIKMTAPVTSYVEPGSGPFSESTI
TISLYIPSEQQFDPPRPLESDVFIEDRAEMTVFVR
SFDGFSSAQKNQEQLLTLASILREDGKVFDEKVYY
TAGYNSPVKLLNRNNEVWLIQKNEPTKENE;

Sphk2 encoded by the SPHK2 gene and having e.g., the following amino acid sequence as set forth in UniProt Accession No. Q9NRA0:

(SEQ ID NO: 83)
MNGHLEAEEQQDQRPDQELTGSWGHGPRSTLVRAK
AMAPPPPPLAASTPLLHGEFGSYPARGPRFALTLT
SQALHIQRLRPKPEARPRGGLVPLAEVSGCCTLRS
RSPSDSAAYFCIYTYPRGRRGARRRATRTFRADGA
ATYEENRAEAQRWATALTCLLRGLPLPGDGEITPD
LLPRPPRLLLLVNPFGGRGLAWQWCKNHVLPMISE
AGLSFNLIQTERQNHARELVQGLSLSEWDGIVTVS
GDGLLHEVLNGLLDRPDWEEAVKMPVGILPCGSGN
ALAGAVNQHGGFEPALGLDLLLNCSLLLCRGGGHP
LDLLSVTLASGSRCFSFLSVAWGFVSDVDIQSERF
RALGSARFTLGTVLGLATLHTYRGRLSYLPATVEP
ASPTPAHSLPRAKSELTLTPDPAPPMAHSPLHRSV
SDLPLPLPQPALASPGSPEPLPILSLNGGGPELAG
DWGGAGDAPLSPDPLLSSPPGSPKAALHSPVSEGA
PVIPPSSGLPLPTPDARVGASTCGPPDHLLPPLGT
PLPPDWVTLEGDFVLMLAISPSHLGADLVAAPHAR
FDDGLVHLCWVRSGISRAALLRLFLAMERGSHFSL
GCPQLGYAAARAFRLEPLTPRGVLTVDGEQVEYGP
LQAQMHPGIGTLLTGPPGCPGREP;

Spike (aka Chmp5, Vps60) encoded by the CHMP5 gene and having e.g., the following amino acid sequence as set forth in UniProt Accession No. Q9NZZ3:

(SEQ ID NO: 84)
MNRLFGKAKPKAPPPSLTDCIGTVDSRAESIDKKI
SRLDAELVKYKDQIKKMREGPAKNMVKQKALRVLK
QKRMYEQQRDNLAQQSFNMEQANYTIQSLKDTKTT
VDAMKLGVKEMKKAYKQVKIDQIEDLQDQLEDMME
DANEIQEALSRSYGTPELDEDDLEAELDALGDELL
ADEDSSYLDEAASAPAIPEGVPTDTKNKDGVLVDE
FGLPQIPAS;

Tg2 (aka Tgase, TGC, Tgm2) encoded by the TGM2 gene and having e.g., the following amino acid sequence as set forth in UniProt Accession No. P21980:

(SEQ ID NO: 85)
MAEELVLERCDLELETNGRDHHTADLCREKLVVRR
GQPFWLTLHFEGRNYEASVDSLTFSVVTGPAPSQE
AGTKARFPLRDAVEEGDWTATVVDQQDCTLSLQLT
TPANAPIGLYRLSLEASTGYQGSSFVLGHFILLFN
AWCPADAVYLDSEEERQEYVLTQQGFIYQGSAKFI
KNIPWNFGQFEDGILDICLILLDVNPKFLKNAGRD
CSRRSSPVYVGRVVSGMVNCNDDQGVLLGRWDNNY
GDGVSPMSWIGSVDILRRWKNHGCQRVKYGQCWVF
AAVACTVLRCLGIPTRVVTNYNSAHDQNSNLLIEY
FRNEFGEIQGDKSEMIWNFHCWVESWMTRPDLQPG
YEGWQALDPTPQEKSEGTYCCGPVPVRAIKEGDLS
TKYDAPFVFAEVNADVVDWIQQDDGSVHKSINRSL
IVGLKISTKSVGRDEREDITHTYKYPEGSSEEREA
FTRANHLNKLAEKEETGMAMRIRVGQSMNMGSDFD
VFAHITNNTAEEYVCRLLLCARTVSYNGILGPECG
TKYLLNLNLEPFSEKSVPLCILYEKYRDCLTESNL
IKVRALLVEPVINSYLLAERDLYLENPEIKIRILG
EPKQKRKLVAEVSLQNPLPVALEGCTFTVEGAGLT
EEQKTVEIPDPVEAGEEVKVRMDLLPLHMGLHKLV
VNFESDKLKAVKGFRNVIIGPA;

Yn1305cp (aka Ybh3p, Bxi1p) encoded by the YNL305C gene and having e.g., the following amino acid sequence as set forth in UniProt Accession No. C8ZGL9:

(SEQ ID NO: 86)
MSGPPPPYEEQSSHLYGQPASSQDGNAFIPEDFKY
STVVISCEPIIRQRFMHKVYSLLSCQLLASLSFCY
WASVSTSLQNFIMSHIALFYICMVVSLVSCIWLAV
SPRPEDYEASVPEPLLTGSSEEPAQEQRRLPWYVL
SSYKQKLTLLSIFTLSEAYCLSLVTLAYDKDTVLS
ALLITTIVVVGVSLTALSERFENVLNSATSIYYWL
NWGLWIMIGMGLTALLFGWNTHSSKFNLLYGWLGA
ILFTAYLFIDTQLIFRKVYPDEEVRCAMMLYLDIV
NLFLSILRILANSNDDN;

HBSP encoded by the SP gene and having e.g., the following amino acid sequence as set forth in UniProt Accession No. Q9QBF1:

(SEQ ID NO: 87)
MPLSYQHFRKLLLLDDEAGPLEEELPRLADEGLNR
RVAEDLNLGNPNVSIPWTHKVGNFTGLYSSTVPVF
NPEWQTPSFPDIHLQEDIVDRCKQFVGPLTVNENR
RLKLIMPARFYPNVTKYLPLDKGIKPYYPEHVVNH
YFQARHYLHTLWKAGILYKRESTHSASFCGSPYSW
EQDLQHGRLVFQTSKRHGDKSFCPQSPGILPRSSV
GPCIQSQLRKSRLGPQPPQGQLAGRPQGGSGSIRA
RVHPSPWGTVGVEPSGSGHTHICASSSSSCLHQSA
VRKAAYSLISTSKGHSSSGRAVELHHFPPNSSRSQ
SQGSVPSCWWLQFRNSKPCSEYCLCHIVNLIDDWG
PCAEHGEHRIRTPRTPARVTGGVFLVDKNPHNTTE
SRLVVDFSQFSRGNTRVSWPKFAVPNLQSLTNLLS
SNLSWLSLDVSAAFYHLPLHPAAMPHLLVGSSGLS
RYVARLSSNSRIINTHQHGTMQDLHNSCSRNLYVS
LMLLYKTYGRKLHLYSHPIILGFRKIPMGVGLSPF
LLAQFTSAICSVVRRAFPHCLAFSYMDDVVLGAKS
VQHLESLYAAVTNFLVSLGIHVNPHKTKRWGYSLN
FMGYVIGSWGTLPQEHIRQKIKLCFRKLPVNRPID
WKVCQRIVGLLGFAAPFTQCGYPALMPLYACISAK
QAFTFSPTYKAFLSQQYLNLYPVARQRSGLCQVFA
DATPTGWGLAIGHQRMRGTFVSPLPIHTAELLAAC
FARSRSGAKLIGTDNSVVLSRKYTSFPWLLGCAAN
WILRGTSFVYVPSALNPADDPSRGRLGLYRPLLRL
PYRPTTGRTSLYADSPSVPSHLPDRVHFASPLHVA
WRPP;

HBx (aka X) encoded by the X gene and having e.g., the following amino acid sequence as set forth in UniProt Accession No. Q05499:

(SEQ ID NO: 88)
MAARMCCKLDPARDVLCLRPIGAESRGRPLPGPLG
AVPPSSPSAVPADDGSHLSLRGLPVCSFSSAGPCA
LRFTSARRMETTVNAPWSLPTVLHKRTLGLSGWSM
TWIEEYIKDCVFKDWEELGEEIRLKVFVLGGCRHK
LVCSPAPCNFFTSA;

AMBRA1 (aka KIAA1736) encoded by the AMBRA1 gene and having e.g., the following amino acid sequence as set forth in UniProt Accession No. Q9C0C7:

(SEQ ID NO: 89)
MKVVPEKNAVRILWGRERGARAMGAQRLLQELVED
KTRWMKWEGKRVELPDSPRSTFLLAFSPDRTLLAS
THVNHNIYITEVKTGKCVHSLIGHRRTPWCVTFHP
TISGLIASGCLDGEVRIWDLHGGSESWFTDSNNAI
ASLAFHPTAQLLLIATANEIHFWDWSRREPFAVVK
TASEMERVRLVRFDPLGHYLLTAIVNPSNQQGDDE
PEIPIDGTELSHYRQRALLQSQPVRRTPLLHNFLH
MLSSRSSGIQVGEQSTVQDSATPSPPPPPPQPSTE
RPRTSAYIRLRQRVSYPTAECCQHLGILCLCSRCS
GTRVPSLLPHQDSVPPASARATTPSFSFVQTEPFH
PPEQASSTQQDQGLLNRPSAFSTVQSSTAGNTLRN
LSLGPTRRSLGGPLSSHPSRYHREIAPGLTGSEWT
RTVLSLNSRSEAESMPPPRTSASSVSLLSVLRQQE
GGSQASVYTSATEGRGFPASGLATESDGGNGSSQN
NSGSIRHELQCDLRRFFLEYDRLQELDQSLSGEAP
QTQQAQEMLNNNIESERPGPSHQPTPHSSENNSNL
SRGHLNRCRACHNLLTFNNDTLRWERTTPNYSSGE
ASSSWQVPSSFESVPSSGSQLPPLERTEGQTPSSS
RLELSSSASPQEERTVGVAFNQETGHWERIYTQSS
RSGTVSQEALHQDMPEESSEEDSLRRRLLESSLIS
LSRYDGAGSREHPIYPDPARLSPAAYYAQRMIQYL
SRRDSIRQRSMRYQQNRLRSSTSSSSSDNQGPSVE
GTDLEFEDFEDNGDRSRHRAPRNARMSAPSLGRFV
PRRFLLPEYLPYAGIFHERGQPGLATHSSVNRVLA
GAVIGDGQSAVASNIANTTYRLQWWDFTKFDLPEI
SNASVNVLVQNCKIYNDASCDISADGQLLAAFIPS
SQRGFPDEGILAVYSLAPHNLGEMLYTKRFGPNAI
SVSLSPMGRYVMVGLASRRILLHPSTEHMVAQVFR
LQQAHGGETSMRRVFNVLYPMPADQRRHVSMSARW
LPEPGLGLAYGTNKGDLVICRPEALNSGVEYYWDQ
LNETVFTVHSNSRSSERPGTSRATWRTDRDMGLMN
AIGLQPRNPATSVTSQGTQTLALQLQNAETQTERE
VPEPGTAASGPGEGEGSEYGASGEDALSRIQRLMA
EGGMTAVVQREQSTTMASMGGFGNNIIVSHRIHRS
SQTGTEPGAAHTSSPQPSTSRGLLPEAGQLAERGL
SPRTASWDQPGTPGREPTQPTLPSSSPVPIPVSLP
SAEGPTLHCELTNNNHLLDGGSSRGDAAGPRGEPR
NR;

BOP (aka BOP, C22orf29) encoded by the RTL10 gene and having e.g., the following amino acid sequence as set forth in UniProt Accession No. Q7L3V2:

(SEQ ID NO: 90)
MPRGRCRQQGPRIPIWAAANYANAHPWQQMDKASPGVAYTPLVDPWIERP
CCGDTVCVRTTMEQKSTASGTCGGKPAERGPLAGHMPSSRPHRVDFCWVP
GSDPGTFDGSPWLLDRFLAQLGDYMSFHFEHYQDNISRVCEILRRLTGRA
QAWAAPYLDGDLPLPDDYELFCQDLKEVVQDPNSFAEYHAVVTCPLPLAS
SQLPVAPQLPVVRQYLARFLEGLALDMGTAPRSLPAAMATPAVSGSNSVS
RSALFEQQLTKESTPGPKEPPVLPSSTCSSKPGPVEPASSQPEEAAPTPV
PRLSESANPPAQRPDPAHPGGPKPQKTEEEVLETEGDQEVSLGTPQEVVE
APETPGEPPLSPGF;

BLM-s (aka Ccdc132) encoded by the Vps50 gene and having e.g., the following amino acid sequence as set forth in UniProt Accession No. QOGET0:

(SEQ ID NO: 91)
MLLLMMNVKWDVKEIMSQHNIYVDALLKEFEQFNKRLNEVSKRVRIPLPV
SNILWEHCIRLANRTIVEGYANVKKCSNEGRALMQLDFQQFLMKLEKLTD
IRPIPDKEFVETYIKAYYLTENDMERWIKEHREYSTKQLTNLVNVCLGSH
INKKARQKLLAAIDEIDRPKR;

and

InsP3R1 (aka INSP3R1) encoded by the ITPR1 gene and having e.g., the following amino acid sequence as set forth in UniProt Accession No. Q14643:

(SEQ ID NO: 92)
MSDKMSSFLHIGDICSLYAEGSTNGFISTLGLVDDRCVVQPETGDLNNPPKKFRDCLFK
LCPMNRYSAQKQFWKAAKPGANSTTDAVLLNKLHHAADLEKKQNETENRKLLGTVIQ
YGNVIQLLHLKSNKYLTVNKRLPALLEKNAMRVTLDEAGNEGSWFYIQPFYKLRSIGD
SVVIGDKVVLNPVNAGQPLHASSHQLVDNPGCNEVNSVNCNTSWKIVLFMKWSDNKD
DILKGGDVVRLFHAEQEKFLTCDEHRKKQHVFLRTTGRQSATSATSSKALWEVEVVQH
DPCRGGAGYWNSLFRFKHLATGHYLAAEVDPDFEEECLEFQPSVDPDQDASRSRLRNA
QEKMVYSLVSVPEGNDISSIFELDPTTLRGGDSLVPRNSYVRLRHLCTNTWVHSTNIPID
KEEEKPVMLKIGTSPVKEDKEAFAIVPVSPAEVRDLDFANDASKVLGSIAGKLEKGTITQ
NERRSVTKLLEDLVYFVTGGTNSGQDVLEVVFSKPNRERQKLMREQNILKQIFKLLQAP
FTDCGDGPMLRLEELGDQRHAPFRHICRLCYRVLRHSQQDYRKNQEYIAKQFGFMQK
QIGYDVLAEDTITALLHNNRKLLEKHITAAEIDTFVSLVRKNREPRFLDYLSDLCVSMN
KSIPVTQELICKAVLNPTNADILIETKLVLSRFEFEGVSSTGENALEAGEDEEEVWLFWR
DSNKEIRSKSVRELAQDAKEGQKEDRDVLSYYRYQLNLFARMCLDRQYLAINEISGQL
DVDLILRCMSDENLPYDLRASFCRLMLHMHVDRDPQEQVTPVKYARLWSEIPSEIAIDD
YDSSGASKDEIKERFAQTMEFVEEYLRDVVCQRFPFSDKEKNKLTFEVVNLARNLIYFG
FYNFSDLLRLTKILLAILDCVHVTTIFPISKMAKGEENKGNNDVEKLKSSNVMRSIHGVG
ELMTQVVLRGGGFLPMTPMAAAPEGNVKQAEPEKEDIMVMDTKLKIIEILQFILNVRLD
YRISCLLCIFKREFDESNSQTSETSSGNSSQEGPSNVPGALDFEHIEEQAEGIFGGSEENTP
LDLDDHGGRTFLRVLLHLTMHDYPPLVSGALQLLFRHFSQRQEVLQAFKQVQLLVTSQ
DVDNYKQIKQDLDQLRSIVEKSELWVYKGQGPDETMDGASGENEHKKTEEGNNKPQK
HESTSSYNYRVVKEILIRLSKLCVQESASVRKSRKQQQRLLRNMGAHAVVLELLQIPYE
KAEDTKMQEIMRLAHEFLQNFCAGNQQNQALLHKHINLFLNPGILEAVTMQHIFMNNF
QLCSEINERVVQHFVHCIETHGRNVQYIKFLQTIVKAEGKFIKKCQDMVMAELVNSGED
VLVFYNDRASFQTLIQMMRSERDRMDENSPLMYHIHLVELLAVCTEGKNVYTEIKCNS
LLPLDDIVRVVTHEDCIPEVKIAYINFLNHCYVDTEVEMKEIYTSNHMWKLFENFLVDIC
RACNNTSDRKHADSILEKYVTEIVMSIVTTFFSSPFSDQSTTLQTRQPVFVQLLQGVFRV
YHCNWLMPSQKASVESCIRVLSDVAKSRAIAIPVDLDSQVNNLFLKSHSIVQKTAMNW
RLSARNAARRDSVLAASRDYRNIIERLQDIVSALEDRLRPLVQAELSVLVDVLHRPELLF
PENTDARRKCESGGFICKLIKHTKQLLEENEEKLCIKVLQTLREMMTKDRGYGEKLISID
ELDNAELPPAPDSENATEELEPSPPLRQLEDHKRGEALRQVLVNRYYGNVRPSGRRESL
TSFGNGPLSAGGPGKPGGGGGGSGSSSMSRGEMSLAEVQCHLDKEGASNLVIDLIMNA
SSDRVFHESILLAIALLEGGNTTIQHSFFCRLTEDKKSEKFFKVFYDRMKVAQQEIKATV
TVNTSDLGNKKKDDEVDRDAPSRKKAKEPTTQITEEVRDQLLEASAATRKAFTTFRRE
ADPDDHYQPGEGTQATADKAKDDLEMSAVITIMQPILRFLQLLCENHNRDLQNFLRCQ
NNKTNYNLVCETLQFLDCICGSTTGGLGLLGLYINEKNVALINQTLESLTEYCQGPCHE
NQNCIATHESNGIDIITALILNDINPLGKKRMDLVLELKNNASKLLLAIMESRHDSENAE
RILYNMRPKELVEVIKKAYMQGEVEFEDGENGEDGAASPRNVGHNIYILAHQLARHNK
ELQSMLKPGGQVDGDEALEFYAKHTAQIEIVRLDRTMEQIVFPVPSICEFLTKESKLRIY
YTTERDEQGSKINDFFLRSEDLFNEMNWQKKLRAQPVLYWCARNMSFWSSISFNLAVL
MNLLVAFFYPFKGVRGGTLEPHWSGLLWTAMLISLAIVIALPKPHGRALIASTILRLIFS
VGLQPTLFLLGAFNVCNKIIFLMSFVGNCGTFTRGYRAMVLDVEFLYHLLYLVICAMGL
FVHEFFYSLLLFDLVYREETLLNVIKSVTRNGRSIILTAVLALILVYLFSIVGYLFFKDDFI
LEVDRLPNETAVPETGESLASEFLFSDVCRVESGENCSSPAPREELVPAEETEQDKEHTC
ETLLMCIVTVLSHGLRSGGGVGDVLRKPSKEEPLFAARVIYDLLFFFMVIIIVLNLIFGVII
DTFADLRSEKQKKEEILKTTCFICGLERDKFDNKTVTFEEHIKEEHNMWHYLCFIVLVK
VKDSTEYTGPESYVAEMIKERNLDWFPRMRAMSLVSSDSEGEQNELRNLQEKLESTMK
LVTNLSGQLSELKDQMTEQRKQKQRIGLLGHPPHMNVNPQQPA.

Non-limiting examples of other BH-3-containing proteins that are anti-apoptotic, include:

Aven (aka PDCD12) encoded by the AVEN gene and having e.g., the following amino acid sequence as set forth in UniProt Accession No. Q9NQS1:

(SEQ ID NO: 93)
MQAERGARGGRGRRPGRGRPGGDRHSERPGAAAAVARGGGGGGGGDGGGR
RGRGRGRGFRGARGGRGGGGAPRGSRREPGGWGAGASAPVEDDSDAETYG
EENDEQGNYSKRKIVSNWDRYQDIEKEVNNESGESQRGTDFSVLLSSAGD
SFSQFRFAEEKEWDSEASCPKQNSAFYVDSELLVRALQELPLCLRLNVAA
ELVQGTVPLEVPQVKPKRTDDGKGLGMQLKGPLGPGGRGPIFELKSVAAG
CPVLLGKDNPSPGPSRDSQKPTSPLQSAGDHLEEELDLLLNLDAPIKEGD
NILPDQTSQDLKSKEDGEVVQEEEVCAKPSVTEEKNMEPEQPSTSKNVTE
EELEDWLDSMIS;

vIRF-1 (aka K9) encoded by the vIRF gene and having e.g., the following amino acid sequence as set forth in UniProt Accession No. P88946:

(SEQ ID NO: 94)
MDPGQRPNPFGAPGAIPKKPCLSQGSPGTSGSGAPCDEPSRSESPGEGPS
DITRQAVVAAITEWSRTRQLRISTGASEGKASIKDWIVCQVNSGKFPGVE
GTGGSAAAGWEDEERTRFRIPVTPLADPCFEWRRDGELGVVYIRERGNMP
VDASFKGTRGRRRMLAALRRTRGLQEIGKGISQDGHHFLVFRVRKPEEEQ
CVECGVVAGAVHDFNNMARLLQEGFFSPGQCLPGEIVTPVPSCTTAEGQE
AVIDWGRLFIRMYYNGEQVHELLTTSQSGCRISSALRRDPAVHYCAVGSP
GQVWLPNVPNLACEIAKRELCDTLDACAKGILLTSSCNGIFCVCYHNGPV
HFIGNTVPPDSGPLLLPQGKPTRIFNPNTFLVGLANSPLPAPSHVTCPLV
KLWLGKPVAVGKLEPHAPSPRDFAARCSNFSDACVVLEIMPKPLWDAMQ;

ATR (aka FRP1) encoded by the ATR gene and having e.g., the following amino acid sequence as set forth in UniProt Accession No. Q13535:

(SEQ ID NO: 95)
MGEHGLELASMIPALRELGSATPEEYNTVVQKPRQILCQFIDRILTDVNVVAVELVKKT
DSQPTSVMLLDFIQHIMKSSPLMFVNVSGSHEAKGSCIEFSNWIITRLLRIAATPSCHLLH
KKICEVICSLLFLFKSKSPAIFGVLTKELLQLFEDLVYLHRRNVMGHAVEWPVVMSRFL
SQLDEHMGYLQSAPLQLMSMQNLEFIEVTLLMVLTRIIAIVFFRRQELLLWQIGCVLLEY
GSPKIKSLAISFLTELFQLGGLPAQPASTFFSSFLELLKHLVEMDTDQLKLYEEPLSKLIKT
LFPFEAEAYRNIEPVYLNMLLEKLCVMFEDGVLMRLKSDLLKAALCHLLQYFLKFVPA
GYESALQVRKVYVRNICKALLDVLGIEVDAEYLLGPLYAALKMESMEIIEEIQCQTQQE
NLSSNSDGISPKRRRLSSSLNPSKRAPKQTEEIKHVDMNQKSILWSALKQKAESLQISLE
YSGLKNPVIEMLEGIAVVLQLTALCTVHCSHQNMNCRTFKDCQHKSKKKPSvvrrwmS
LDFYTKVLKSCRSLLESVQKLDLEATIDKVVKIYDALIYMQVNSSFEDHILEDLCGMLS
LPWIYSHSDDGCLKLTTFAANLLTLSCRISDSYSPQAQSRCVFLLTLFPRRIFLEWRTAV
YNWALQSSHEVIRASCVSGFFILLQQQNSCNRVPKILIDKVKDDSDIVKKEFASILGQLV
CTLHGMFYLTSSLTEPFSEHGHVDLFCRNLKATSQHECSSSQLKASVCKPFLFLLKKKIP
SPVKLAFIDNLHHLCKHLDFREDETDVKAVLGTLLNLMEDPDKDVRVAFSGNIKHILES
LDSEDGFIKELFVLRMKEAYTHAQISRNNELKDTLILTTGDIGRAAKGDLVPFALLHLLH
CLLSKSASVSGAAYTEIRALVAAKSVKLQSFFSQYKKPICQFLVESLHSSQMTALPNTPC
QNADVRKQDVAHQREMALNTLSEIANVFDFPDLNRFLTRTLQVLLPDLAAKASPAASA
LIRTLGKQLNVNRREILINNFKYIFSHLVCSCSKDELERALHYLKNETEIELGSLLRQDFQ
GLHNELLLRIGEHYQQVFNGLSILASFASSDDPYQGPRDIISPELMADYLQPKLLGILAFF
NMQLLSSSVGIEDKKMALNSLMSLMKLMGPKHVSSVRVKMMTTLRTGLRFKDDFPEL
CCRAWDCFVRCLDHACLGSLLSHVIVALLPLIHIQPKETAAIFHYLIIENRDAVQDFLHEI
YFLPDHPELKKIKAVLQEYRKETSESTDLQTTLQLSMKAIQHENVDVRIHALTSLKETL
YKNQEKLIKYATDSETVEPIISQLVTVLLKGCQDANSQARLLCGECLGELGAIDPGRLDF
STTETQGKDFTFVTGVEDSSFAYGLLMELTRAYLAYADNSRAQDSAAYAIQELLSIYDC
REMETNGPGHQLWRRFPEHVREILEPHLNTRYKSSQKSTDWSGVKKPIYLSKLGSNFAE
WSASWAGYLITKVRHDLASKIFTCCSIMMKHDFKVTIYLLPHILVYVLLGCNQEDQQE
VYAEIMAVLKHDDQHTINTQDIASDLCQLSTQTVFSMLDHLTQWARHKFQALKAEKCP
HSKSNRNKVDSMVSTVDYEDYQSVTRFLDLIPQDTLAVASFRSKAYTRAVMHFESFITE
KKQNIQEHLGFLQKLYAAMHEPDGVAGVSAIRKAEPSLKEQILEHESLGLLRDATACY
DRAIQLEPDQIIHYHGVVKSMLGLGQLSTVITQVNGVHANRSEWTDELNTYRVEAAWK
LSQWDLVENYLAADGKSTTWSVRLGQLLLSAKKRDITAFYDSLKLVRAEQIVPLSAAS
FERGSYQRGYEYIVRLHMLCELEHSIKPLFQHSPGDSSQEDSLNWVARLEMTQNSYRA
KEPILALRRALLSLNKRPDYNEMVGECWLQSARVARKAGHHQTAYNALLNAGESRLA
ELYVERAKWLWSKGDVHQALIVLQKGVELCFPENETPPEGKNMLIHGRAMLLVGRFM
EETANFESNAIMKKYKDVTACLPEWEDGHFYLAKYYDKLMPMVTDNKMEKQGDLIR
YIVLHFGRSLQYGNQFIYQSMPRMLTLWLDYGTKAYEWEKAGRSDRVQMRNDLGKIN
KVITEHTNYLAPYQFLTAFSQLISRICHSHDEVFVVLMEIIAKVFLAYPQQAMWMMTAV
SKSSYPMRVNRCKEILNKAIHMKKSLEKFVGDATRLTDKLLELCNKPVDGSSSTLSMST
HFKMLKKLVEEATFSEILIPLQSVMIPTLPSILGTHANHASHEPFPGHWAYIAGFDDMVE
ILASLQKPKKISLKGSDGKFYIMMCKPKDDLRKDCRLMEFNSLINKCLRKDAESRRREL
HIRTYAVIPLNDECGIIEWVNNTAGLRPILTKLYKEKGVYMTGKELRQCMLPKSAALSE
KLKVFREFLLPRHPPIFHEWFLRTFPDPTSWYSSRSAYCRSTAVMSMVGYILGLGDRHG
ENILFDSLTGECVHVDFNCLFNKGETFEVPEIVPFRLTHNMVNGMGPMGTEGLFRRACE
VTMRLMRDQREPLMSVLKTFLHDPLVEWSKPVKGHSKAPLNETGEVVNEKAKTHVLD
IEQRLQGVIKTRNRVTGLPLSIEGHVHYLIQEATDENLLCQMYLGWTPYM;

and

TCTP (aka Fortilin, HRF, p23) encoded by the TPT1 gene and having e.g., the following amino acid sequence as set forth in UniProt Accession No. P13693:

(SEQ ID NO: 96)
MIIYRDLISHDEMFSDIYKIREIADGLCLEVEGKMVSRTEGNIDDSLIGG
NASAEGPEGEGTESTVITGVDIVMNHHLQETSFTKEAYKKYIKDYMKSIK
GKLEEQRPERVKPFMTGAAEQIKHILANFKNYQFFIGENMNPDGMVALLD
YREDGVTPYMIFFKDGLEMEKC.

Non-limiting examples of other BH-3-containing proteins also include:

Apol1 (aka ApoL-I) encoded by the APOL1 gene and having e.g., the following amino acid sequence as set forth in UniProt Accession No. 014791:

(SEQ ID NO: 97)
MEGAALLRVSVLCIWMSALFLGVGVRAEEAGARVQQNVPSGTDTGDPQSK
PLGDWAAGTMDPESSIFIEDAIKYFKEKVSTQNLLLLLTDNEAWNGFVAA
AELPRNEADELRKALDNLARQMIMKDKNWHDKGQQYRNWFLKEFPRLKSE
LEDNIRRLRALADGVQKVHKGTTIANVVSGSLSISSGILTLVGMGLAPFT
EGGSLVLLEPGMELGITAALTGITSSTMDYGKKWWTQAQAHDLVIKSLDK
LKEVREFLGENISNFLSLAGNTYQLTRGIGKDIRALRRARANLQSVPHAS
ASRPRVTEPISAESGEQVERVNEPSILEMSRGVKLTDVAPVSFFLVLDVV
YLVYESKHLHEGAKSETAEELKKVAQELEEKLNILNNNYKILQADQEL;

APOL2 (aka ApoL-II) encoded by the APOL2 gene and having e.g., the following amino acid sequence as set forth in UniProt Accession No. Q9BQE5:

(SEQ ID NO: 98)
MNPESSIFIEDYLKYFQDQVSRENLLQLLTDDEAWNGFVAAAELPRDEAD
ELRKALNKLASHMVMKDKNRHDKDQQHRQWFLKEFPRLKRELEDHIRKLR
ALAEEVEQVHRGTTIANVVSNSVGTTSGILTLLGLGLAPFTEGISFVLLD
TGMGLGAAAAVAGITCSVVELVNKLRARAQARNLDQSGTNVAKVMKEFVG
GNTPNVLTLVDNWYQVTQGIGRNIRAIRRARANPQLGAYAPPPHIIGRIS
AEGGEQVERVVEGPAQAMSRGTMIVGAATGGILLLLDVVSLAYESKHLLE
GAKSESAEELKKRAQELEGKLNFLTKIHEMLQPGQDQ.

Methods of the present disclosure may employ heterologous apoptosis modulators that are BCL-2 family pro-apoptotic proteins or nucleic acid sequences encoding such BCL-2 family pro-apoptotic proteins, including but not limited to e.g., where the BCL-2 family pro-apoptotic protein is a protein identified above or is derived from a protein identified above.

Pro-apoptotic Bcl-2 family members may be divided into effector proteins and BH3-only proteins. Pro-apoptotic proteins generally contain a BH3 domain for dimerization with other proteins of Bcl-2 family and which may provide for killing activity. Some pro-apoptotic Bcl-2 family members also contain BH1 and BH2 domains, such as Bax and Bak. Some pro-apoptotic Bcl-2 family members contain an additional N-terminal BH4 domain; however, this domain is also present in some anti-apoptotic BCL-2 family members. The BH3-only subset of the Bcl-2 family of proteins may contain only a single BH3-domain. The BH3-only members play a role in promoting apoptosis. The BH3-only family members include Bim, Bid, BAD and others.

In some instances, a BCL-2 family pro-apoptotic protein, or encoding sequence thereof, employed in a method of the present disclosure may share 100% sequence identity or less to a protein identified above, including but not limited to e.g., where the protein shares at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to one or more of the amino acid sequences provided above.

In some embodiments, a method of the present disclosure may employ a pro-apoptotic BIM protein, or a portion thereof, or a nucleic acid sequence encoding a pro-apoptotic BIM protein, or a portion thereof. In some instances, useful BIM proteins may include a polypeptide having a sequence sharing 100% amino acid sequence identity or less (including e.g., at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity) with the human BIM amino acid sequence (UniProt Accession No.: 043521):

(SEQ ID NO: 99)
MAKQPSDVSSECDREGRQLQPAERPPQLRPGAPTSLQTEPQGNPEGNHGG
EGDSCPHGSPQGPLAPPASPGPFATRSPLFIFMRRSSLLSRSSSGYFSFD
TDRSPAPMSCDKSTQTPSPPCQAFNHYLSAMASMRQAEPADMRPEIWIAQ
ELRRIGDEFNAYYARRVFLNNYQAAEDHPRMVILRLLRYIVRLVWRMH.

In some instances, a modified BIM polypeptide may be employed. Useful modified BIM polypeptides include, but are not necessarily limited to, truncated and/or mutated forms of BIM. Useful truncated forms include N-terminal truncations, including wherein 1 or more amino acid residues, including e.g., 1, 2, 3, 4, 5, or more residues, are deleted from the N-terminus. Useful truncated forms include C-terminal truncations, including wherein 1 or more amino acid residues, including e.g., 1, 2, 3, 4, 5, or more residues, are deleted from the C-terminus. In some instances, a truncated form may include both an N-terminal and a C-terminal truncation. Useful modified forms may also include modified BIM polypeptides having one or more amino acid substitutions, including e.g., 1, 2, 3, 4, 5, 6, 7, 8, 9, 10 or more amino acid substitutions. In some instances, one or more substitutions may be present in a modified BIM polypeptide in the absence of any truncation. In some instances, a modified BIM polypeptide may be both truncated, at one or both termini, and include one or more amino acid substitutions.

In some instances, embodiments of the methods of the present disclosure may exclude the use of a pro-apoptotic BIM or a portion thereof, or a nucleic acid sequence encoding a pro-apoptotic BIM protein, or a portion thereof. For example, in some instances, due to the strength and/or leakiness of pro-apoptotic signaling of an uninduced inducible BIM, BIM may not be employed as a pro-apoptotic agent in certain methods, circuits, cells, nucleic acids, etc. In some instances, BIM may be employed in a method, circuit, cell, nucleic acid, etc., including e.g., where the BIM is buffered by an anti-apoptotic agent. For example, in some instances, due to the strength and/or leakiness of pro-apoptotic signaling of an uninduced inducible BIM, BIM may be employed as a pro-apoptotic agent in combination with an anti-apoptotic agent that buffers the pro-apoptotic signaling of BIM.

In some embodiments, a method of the present disclosure may employ a pro-apoptotic BID protein, or a portion thereof, or a nucleic acid sequence encoding a pro-apoptotic BID protein, or a portion thereof. In some instances, useful BID proteins may include a polypeptide having a sequence sharing 100% amino acid sequence identity or less (including e.g., at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity) with the human BID amino acid sequence (UniProt Accession No.: P55957) or a portion thereof: MDCEVNNGSSLRDECITNLLVFGFLQSCSDNSFRRELDALGHELPVLAPQWEGYDELQ TDGNRSSHSRLGRIEADSESQEDIIRNIARHLAQVGDSMDRSIPPGLVNGLALQLRNTSR SEEDRNRDLATALEQLLQAYPRDMEKEKTMLVLALLLAKKVASHTPSLLRDVFHTTVN FINQNLRTYVRSLARNGMD (SEQ ID NO: 100). In some instances, embodiments may exclude a full-length BID. For example, some embodiments may employ a modified BID, including where the BID is mutated and/or truncated at one or both ends, that is not a full-length BID and, e.g., does not include the sequence provided above.

In some instances, a truncated BID may be employed. Useful truncated BID polypeptides include tBID, which refers to the C-terminal truncated fragment of BID which results from the enzymatic cleavage of cytosolic BID (e.g., by active caspase). In some instances, a tBID polypeptide employed may have the following amino acid sequence, or may share at least at least 80%, at least 85%, at least 90%, at least 91%, at least 92%, at least 93%, at least 94%, at least 95%, at least 96%, at least 97%, at least 98%, at least 99%, or 100% sequence identity, with the following amino acid sequence:

(SEQ ID NO: 101)
GNRSSHSRLGRIEADSESQEDIIRNIARHLAQVGDSMDRSIPPGLVNGLA
LQLRNTSRSEEDRNRDLATALEQLLQAYPRDMEKEKTMLVLALLLAKKVA
SHTPSLLRDVFHTTVNFINQNLRTYVRSLARNGMD.

Truncated BID polypeptides may vary and may include C-terminal truncated fragments of BID and variants thereof. In some instances, useful truncated BID polypeptides may include modified versions of tBID. For example, modified tBID polypeptides may include a tBID polypeptide, such as the polypeptide of the amino acid sequence provided above, that includes one or more, including 1, 2, 3, 4, or more additional amino acid deletions from the N-terminal end (i.e., N-terminal truncations). In some instances, modified tBID polypeptides may include a tBID polypeptide, such as the polypeptide of the amino acid sequence provided above, that includes one or more, including 1, 2, 3, 4, or more amino acid deletions from the C-terminal end (i.e., C-terminal truncations). In some instances, a truncated BID may include both N- and C-terminal truncations. In some instances, a truncated BID may include only N-terminal truncations. In some instances, a modified truncated BID polypeptide, with or without one or more C-terminal truncations and/or one or more additional N-terminal truncations, may include one or more amino acid substitutions, including but not limited to e.g., 1, 2, 3, 4, 5, 6, 7, 8, 9, 10, or more amino acid substitutions.

In some embodiments, a method of the present disclosure may employ a pro-apoptotic PUMA protein, or a portion thereof, or a nucleic acid sequence encoding a pro-apoptotic PUMA protein, or a portion thereof. In some instances, useful PUMA proteins may include a polypeptide having a sequence sharing 100% amino acid sequence identity or less (including e.g., at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity) with the human PUMA amino acid sequence (UniProt Accession No.: Q9BXH1) or a portion thereof:

(SEQ ID NO: 102)
MARARQEGSSPEPVEGLARDGPRPFPLGRLVPSAVSCGLCEPGLAAAPAA
PTLLPAAYLCAPTAPPAVTAALGGSRWPGGPRSRPRGPRPDGPQPSLSLA
EQHLESPVPSAPGALAGGPTQAAPGVRGEEEQWAREIGAQLRRMADDLNA
QYERRRQEEQQRHRPSPWRVLYNLIMGLLPLPRGHRAPEMEPN.

In some instances, a modified PUMA polypeptide may be employed. Useful modified PUMA polypeptides include, but are not necessarily limited to, truncated and/or mutated forms of PUMA. Useful truncated forms include N-terminal truncations, including wherein 1 or more amino acid residues, including e.g., 1, 2, 3, 4, 5, or more residues, are deleted from the N-terminus. Useful truncated forms include C-terminal truncations, including wherein 1 or more amino acid residues, including e.g., 1, 2, 3, 4, 5, or more residues, are deleted from the C-terminus. In some instances, a truncated form may include both an N-terminal and a C-terminal truncation. Useful modified forms may also include modified PUMA polypeptides having one or more amino acid substitutions, including e.g., 1, 2, 3, 4, 5, 6, 7, 8, 9, 10 or more amino acid substitutions. In some instances, one or more substitutions may be present in a modified PUMA polypeptide in the absence of any truncation. In some instances, a modified PUMA polypeptide may be both truncated, at one or both termini, and include one or more amino acid substitutions.

In some embodiments, a method of the present disclosure may employ a pro-apoptotic BMF protein, or a portion thereof, or a nucleic acid sequence encoding a pro-apoptotic BMF protein, or a portion thereof. In some instances, useful BMF proteins may include a polypeptide having a sequence sharing 100% amino acid sequence identity or less (including e.g., at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity) with the human BMF amino acid sequence (UniProt Accession No.: Q96LC9) or a portion thereof:

(SEQ ID NO: 102)
MEPSQCVEELEDDVFQPEDGEPVTQPGSLLSADLFAQSLLDCPLSRLQLF
PLTHCCGPGLRPTSQEDKATQTLSPASPSQGVMLPCGVTEEPQRLFYGNA
GYRLPLPASFPAVLPIGEQPPEGQWQHQAEVQIARKLQCIADQFHRLHVQ
QHQQNQNRVWWQILLFLHNLALNGEENRNGAGPR.

In some instances, a modified BMF polypeptide may be employed. Useful modified BMF polypeptides include, but are not necessarily limited to, truncated and/or mutated forms of BMF. Useful truncated forms include N-terminal truncations, including wherein 1 or more amino acid residues, including e.g., 1, 2, 3, 4, 5, or more residues, are deleted from the N-terminus. Useful truncated forms include C-terminal truncations, including wherein 1 or more amino acid residues, including e.g., 1, 2, 3, 4, 5, or more residues, are deleted from the C-terminus. In some instances, a truncated form may include both an N-terminal and a C-terminal truncation. Useful modified forms may also include modified BMF polypeptides having one or more amino acid substitutions, including e.g., 1, 2, 3, 4, 5, 6, 7, 8, 9, 10 or more amino acid substitutions. In some instances, one or more substitutions may be present in a modified BMF polypeptide in the absence of any truncation. In some instances, a modified BMF polypeptide may be both truncated, at one or both termini, and include one or more amino acid substitutions.

In some embodiments, a method of the present disclosure may employ a pro-apoptotic HRK protein, or a portion thereof, or a nucleic acid sequence encoding a pro-apoptotic HRK protein, or a portion thereof. In some instances, useful HRK proteins may include a polypeptide having a sequence sharing 100% amino acid sequence identity or less (including e.g., at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity) with the human HRK amino acid sequence (UniProt Accession No.: 000198) or a portion thereof:

(SEQ ID NO: 103)
MCPCPLHRGRGPPAVCACSAGRLGLRSSAAQLTAARLKALGDELHQRTMW
RRRARSRRAPAPGALPTYWPWLCAAAQVAALAAWLLGRRNL.

In some instances, a modified HRK polypeptide may be employed. Useful modified HRK polypeptides include, but are not necessarily limited to, truncated and/or mutated forms of HRK. Useful truncated forms include N-terminal truncations, including wherein 1 or more amino acid residues, including e.g., 1, 2, 3, 4, 5, or more residues, are deleted from the N-terminus. Useful truncated forms include C-terminal truncations, including wherein 1 or more amino acid residues, including e.g., 1, 2, 3, 4, 5, or more residues, are deleted from the C-terminus. In some instances, a truncated form may include both an N-terminal and a C-terminal truncation. Useful modified forms may also include modified HRK polypeptides having one or more amino acid substitutions, including e.g., 1, 2, 3, 4, 5, 6, 7, 8, 9, 10 or more amino acid substitutions. In some instances, one or more substitutions may be present in a modified HRK polypeptide in the absence of any truncation. In some instances, a modified HRK polypeptide may be both truncated, at one or both termini, and include one or more amino acid substitutions.

In some embodiments, a method of the present disclosure may employ a pro-apoptotic BIK protein, or a portion thereof, or a nucleic acid sequence encoding a pro-apoptotic BIK protein, or a portion thereof. In some instances, useful BIK proteins may include a polypeptide having a sequence sharing 100% amino acid sequence identity or less (including e.g., at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity) with the human BIK amino acid sequence (UniProt Accession No.: Q13323) or a portion thereof:

(SEQ ID NO: 104)
MSEVRPLSRDILMETLLYEQLLEPPTMEVLGMTDSEEDLDPMEDFDSLEC
MEGSDALALRLACIGDEMDVSLRAPRLAQLSEVAMHSLGLAFIYDQTEDI
RDVLRSFMDGFTTLKENIMRFWRSPNPGSWVSCEQVLLALLLLLALLLPL
LSGGLHLLLK.

In some instances, a modified BIK polypeptide may be employed. Useful modified BIK polypeptides include, but are not necessarily limited to, truncated and/or mutated forms of BIK. Useful truncated forms include N-terminal truncations, including wherein 1 or more amino acid residues, including e.g., 1, 2, 3, 4, 5, or more residues, are deleted from the N-terminus. Useful truncated forms include C-terminal truncations, including wherein 1 or more amino acid residues, including e.g., 1, 2, 3, 4, 5, or more residues, are deleted from the C-terminus. In some instances, a truncated form may include both an N-terminal and a C-terminal truncation. Useful modified forms may also include modified BIK polypeptides having one or more amino acid substitutions, including e.g., 1, 2, 3, 4, 5, 6, 7, 8, 9, 10 or more amino acid substitutions. In some instances, one or more substitutions may be present in a modified BIK polypeptide in the absence of any truncation. In some instances, a modified BIK polypeptide may be both truncated, at one or both termini, and include one or more amino acid substitutions.

Methods of the present disclosure may employ heterologous apoptosis modulators that are BCL-2 family anti-apoptotic proteins or nucleic acid sequences encoding such BCL-2 family anti-apoptotic proteins, including but not limited to e.g., where the BCL-2 family anti-apoptotic protein is a protein identified above or is derived from a protein identified above.

Anti-apoptotic BCL-2 family proteins may contain BH1 and BH2 domains. Some anti-apoptotic BCL-2 family proteins contain an additional N-terminal BH4 domain; however, this domain is also present in some pro-apoptotic BCL-2 family members. The prominent anti-apoptotic proteins include the proteins or gene products of Bcl-2-related gene A1 (A1), Bcl-2, Bcl-2-related gene, long isoform (Bcl-xL), Bcl-w, and myeloid cell leukemia 1 (MCL-1). A number of anti-apoptotic BCL-2 family member proteins preserve outer mitochondrial membrane integrity by directly inhibiting the pro-apoptotic Bcl-2 proteins.

In some instances, a BCL-2 family anti-apoptotic protein, or encoding sequence thereof, employed in a method of the present disclosure may share 100% sequence identity or less to a protein identified above, including but not limited to e.g., where the protein shares at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to one or more of the amino acid sequences provided above.

In some embodiments, a method of the present disclosure may employ an anti-apoptotic BCL-2 protein, or a portion thereof, or a nucleic acid sequence encoding an anti-apoptotic BCL-2 protein, or a portion thereof. In some instances, useful BCL-2 proteins may include a polypeptide having a sequence sharing 100% amino acid sequence identity or less (including e.g., at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity) with the human BCL-2 amino acid sequence (UniProt Accession No.: P10415):

(SEQ ID NO: 105)
MAHAGRTGYDNREIVMKYIHYKLSQRGYEWDAGDVGAAPPGAAPAPGIFS
SQPGHTPHPAASRDPVARTSPLQTPAAPGAAAGPALSPVPPVVHLTLRQA
GDDFSRRYRRDFAEMSSQLHLTPFTARGRFATVVEELFRDGVNWGRIVAF
FEFGGVMCVESVNREMSPLVDNIALWMTEYLNRHLHTWIQDNGGWDAFVE
LYGPSMRPLFDFSWLSLKTLLSLALVGACITLGAYLGHK.

In some instances, methods of the present disclosure may employ a single heterologous BCL-2 family protein, e.g., in a cell, in a circuit, in a nucleic acid, in an expression vector, etc. In some instances, methods of the present disclosure may employ multiple heterologous BCL-2 family proteins e.g., in the same cell, in the same circuit, in the same nucleic acid, in the same expression vector, etc. Where multiple heterologous BCL-2 family proteins are employed the total number of heterologous BCL-2 family proteins may vary and may range from 2 to 6 or more, including but not limited to e.g., 2 or more, 3 or more, 4 or more, 5 or more, 6 or more, 2, 3, 4, 5, 6, etc.

Various combinations of multiple heterologous BCL-2 family proteins may be employed including where the proteins are individually selected from any category of BCL-2 family proteins described above or multiple proteins from one category are employed. In some embodiments, multiple pro-apoptotic proteins and/or multiple anti-apoptotic proteins may be employed. In some instances, a single pro-apoptotic protein and multiple anti-apoptotic proteins may be employed. In some instances, a single anti-apoptotic protein and multiple pro-apoptotic proteins may be employed. In some instances, a single pro-apoptotic protein and a single anti-apoptotic protein may be employed.

Therapeutic Proteins of Interest (POI)

Therapeutic POIs that may vary and may include but are not limited to a therapeutic polypeptide for treating various conditions, including e.g., a neoplasia such as e.g., a tumor, a cancer, etc. In some instances, a therapeutic POI for treating a neoplasia may be a POI used in immunotherapy for cancer, where in some instances, useful POIs include antigen-specific therapeutics (e.g., CARs, TCRs, antibodies, etc.). In some instances, a therapeutic POI may be a CAR. In some instances, a therapeutic POI may be a TCR. In some instances, a therapeutic POI may be an antibody. In some instances, a therapeutic POI may be a chimeric bispecific binding member. In some instances, a therapeutic POI may be an innate-immune response inducer. In some instances, a therapeutic POI may be an immune suppression factor.

The terms “chimeric antigen receptor” and “CAR”, used interchangeably herein, refer to artificial multi-module molecules capable of triggering or inhibiting the activation of an immune cell which generally but not exclusively comprise an extracellular domain (e.g., a ligand/antigen binding domain), a transmembrane domain and one or more intracellular signaling domains. The term CAR is not limited specifically to CAR molecules but also includes CAR variants. CAR variants include split CARs wherein the extracellular portion (e.g., the ligand binding portion) and the intracellular portion (e.g., the intracellular signaling portion) of a CAR are present on two separate molecules. CAR variants also include ON-switch CARs which are conditionally activatable CARs, e.g., comprising a split CAR wherein conditional heterodimerization of the two portions of the split CAR is pharmacologically controlled. CAR variants also include bispecific CARs, which include a secondary CAR binding domain that can either amplify or inhibit the activity of a primary CAR. CAR variants also include inhibitory chimeric antigen receptors (iCARs) which may, e.g., be used as a component of a bispecific CAR system, where binding of a secondary CAR binding domain results in inhibition of primary CAR activation. CAR molecules and derivatives thereof (i.e., CAR variants) are described, e.g., in PCT Application No. US2014/016527; Fedorov et al. Sci Transl Med (2013); 5(215):215ra172; Glienke et al. Front Pharmacol (2015) 6:21; Kakarla & Gottschalk 52 Cancer J (2014) 20(2):151-5; Riddell et al. Cancer J (2014) 20(2):141-4; Pegram et al. Cancer J (2014) 20(2):127-33; Cheadle et al. Immunol Rev (2014) 257(1):91-106; Barrett et al. Annu Rev Med (2014) 65:333-47; Sadelain et al. Cancer Discov (2013) 3(4):388-98; Cartellieri et al., J Biomed Biotechnol (2010) 956304; the disclosures of which are incorporated herein by reference in their entirety.

Non-limiting examples of CARs that may be employed include those used in commercialized CAR T cell (CART) therapies including e.g., the anti-CD19-4-1BB-CD3ζ CAR expressed by lentivirus loaded CTL019 (Tisagenlecleucel-T) CAR-T cells, also referred to as Kymriah™(tisagenlecleucel) as commercialized by Novartis (Basel, Switzerland) and the anti-BCMA-4-1BB-CD3ζ CAR expressed by lentivirus loaded CAR-T cells called “bb2121” as commercialized by bluebird bio, Inc. (Cambridge, Mass.) and Celgene Corporation (Summit, N.J.).

Useful CARs or useful domains thereof may, in some instances, include those described in U.S. Pat. Nos. 9,914,909; 9,821,012; 9,815,901; 9,777,061; 9,662,405; 9,657,105; 9,629,877; 9,624,276; 9,598,489; 9,587,020; 9,574,014; 9,573,988; 9,499,629; 9,446,105; 9,394,368; 9,328,156; 9,233,125; 9,175,308 and 8,822,647; the disclosures of which are incorporated herein by reference in their entirety. In some instances, useful CARs may include or exclude heterodimeric, also referred to as dimerizable or switchable, CARs and/or include or exclude one or more of the domains thereof. Useful heterodimeric CARs and/or useful domains thereof may, in some instances, include those described in U.S. Pat. Nos. 9,587,020 and 9,821,012 as well as U.S. Pub. Nos. US20170081411A1, US20160311901A1, US20160311907A1, US20150266973A1 and PCT Pub. Nos. WO2014127261A1, WO2015142661A1, WO2015090229A1 and WO2015017214A1; the disclosures of which are incorporated herein by reference in their entirety.

Useful CARs and/or useful domains thereof may, in some instances, include those that have been or are currently being investigated in one or more clinical trials, including but not limited to the CARs directed to the following antigens (listed with an exemplary corresponding clinical trial number, further information pertaining to which may be retrieved by visiting www(dot)clinicaltrials(dot)gov): AFP, e.g., in NCT03349255; BCMA, e.g., in NCT03288493; CD10, e.g., in NCT03291444; CD117, e.g., in NCT03291444; CD123, e.g., in NCT03114670; CD133, e.g., in NCT02541370; CD138, e.g., in NCT01886976; CD171, e.g., in NCT02311621; CD19, e.g., in NCT02813252; CD20, e.g., in NCT03277729; CD22, e.g., in NCT03244306; CD30, e.g., in NCT02917083; CD33, e.g., in NCT03126864; CD34, e.g., in NCT03291444; CD38, e.g., in NCT03291444; CD5, e.g., in NCT03081910; CD56, e.g., in NCT03291444; CD7, e.g., in NCT02742727; CD70, e.g., in NCT02830724; CD80, e.g., in NCT03356808; CD86, e.g., in NCT03356808; CEA, e.g., in NCT02850536; CLD18, e.g., in NCT03159819; CLL-1, e.g., in NCT03312205; cMet, e.g., in NCT01837602; EGFR, e.g., in NCT03182816; EGFRvIII, e.g., in NCT02664363; EpCAM, e.g., in NCT03013712; EphA2, e.g., in NCT02575261; GD-2, e.g., in NCT01822652; Glypican 3, e.g., in NCT02905188; GPC3, e.g., in NCT02723942; HER-2, e.g., in NCT02547961; kappa immunoglobulin, e.g., in NCT00881920; LeY, e.g., in NCT02958384; LMP1, e.g., in NCT02980315; mesothelin, e.g., in NCT02930993; MG7, e.g., in NCT02862704; MUC1, e.g., in NCT02587689; NKG2D-ligands, e.g., in NCT02203825; PD-L1, e.g., in NCT03330834; PSCA, e.g., in NCT02744287; PSMA, e.g., in NCT03356795; ROR1, e.g., in NCT02706392; ROR1R, e.g., in NCT02194374; TACI, e.g., in NCT03287804; and VEGFR2, e.g., in NCT01218867.

In some instances, a therapeutic POI may be an anti-Fc CAR. An anti-Fc CAR generally includes the extracellular domain of an Fc receptor, an intracellular signaling domain and optionally a co-stimulatory domain. Depending on the therapeutic context, an anti-Fc CAR may include an extracellular domain of any Fc receptor including e.g., a Fc-gamma receptor (e.g., FcγRI (CD64), FcγRIIA (CD32), FcγRIIB (CD32), FcγRIIIA (CD16a), FcγRIIIB (CD16b)), a Fc-alpha receptor (e.g., FcγRI (CD89)) or a Fc-epsilon receptor (e.g., FcεRI, FcεRII (CD23)). For example, in some instances, an anti-Fc CAR may include the extracellular domain of the CD16 Fc receptor. In some instances, an anti-Fc CAR may include the extracellular domain of the CD16 Fc receptor, a CD3-zeta intracellular signaling domain and a 4-1BB co-stimulatory domain. In some instances, an anti-Fc CAR may be an Antibody-Coupled T-cell Receptor (ACTR), e.g., as available from (Unum Therapeutics Inc.; Cambridge, Mass.).

In some instances, a therapeutic POI may be a chimeric bispecific binding member. As used herein, by “chimeric bispecific binding member” is meant a chimeric polypeptide having dual specificity to two different binding partners (e.g., two different antigens). Non-limiting examples of chimeric bispecific binding members include bispecific antibodies, bispecific conjugated monoclonal antibodies (mab)₂, bispecific antibody fragments (e.g., F(ab)₂, bispecific scFv, bispecific diabodies, single chain bispecific diabodies, etc.), bispecific T cell engagers (BiTE), bispecific conjugated single domain antibodies, micabodies and mutants thereof, and the like. Non-limiting examples of chimeric bispecific binding members also include those chimeric bispecific agents described in Kontermann. MAbs. (2012) 4(2): 182-197; Stamova et al. Antibodies 2012, 1(2), 172-198; Farhadfar et al. Leuk Res. (2016) 49:13-21; Benjamin et al. Ther Adv Hematol. (2016) 7(3):142-56; Kiefer et al. Immunol Rev. (2016) 270(1):178-92; Fan et al. J Hematol Oncol. (2015) 8:130; May et al. Am J Health Syst Pharm. (2016) 73(1):e6-e13; the disclosures of which are incorporated herein by reference in their entirety.

Non-limiting examples of bispecific binding member therapeutic POIs include e.g., bsAb MDX-210 (targeting Her2 and CD64), MDX-H210 (targeting Her2 and CD64), MDX-447 (targeting EGFR and CD64), HRS-3/A9 (a bispecific F(ab′)2 antibody targeting the CD30 antigen and receptor FcγRIII (CD16)), an anti-CD3×anti-EpCAM TriomAb/bsAb, Catumaxomab, Ertumaxomab, Bi20 (Lymphomun or fBTA05), an anti-CD19×CD3 diabody, an anti-CD19×CD16 diabody, an anti-EGFR×CD3 diabody, an anti-PSMA×CD3 diabody, a diabody targeting rM28 and NG2, an anti-CD28×CD20 bispecific tandem scFv, or the like.

In some instances, a chimeric bispecific binding member may be a bispecific T cell engager (BiTE). A BiTE is generally made by fusing a specific binding member (e.g., a scFv) that binds an immune cell antigen to a specific binding member (e.g., a scFv) that binds a cancer antigen (e.g., a tumor associated antigen, a tumor specific antigen, etc.). For example, an exemplary BiTE includes an anti-CD3 scFv fused to an anti-tumor associated antigen (e.g., EpCAM, CD19, etc.) scFv via a short peptide linker (e.g., a five amino acid linker).

Non-limiting examples of BiTEs suitable for use as herein described include e.g., an anti-CD3×anti-CD19 BiTE (e.g., Blinatumomab), an anti-EpCAM×anti-CD3 BiTE (e.g., MT110), an anti-CEA×anti-CD3 BiTE (e.g., MT111/MEDI-565), an anti-CD33×anti-CD3 BiTE, an anti-HER2 BiTE, an anti-EGFR BiTE, an anti-IgE BiTE, and the like.

In some instances, a chimeric bispecific binding member may be a Micabody or mutant thereof. A Micabody generally includes an antigen-specific binding portion linked to at least one domain that specifically binds a NKG2D receptor. In some instances, a Micabody or mutant thereof includes engineered MICA α1-α2 domains that specifically bind to NKG2D receptors. Non-limiting examples of Micabodies and related components and operating principles are described in e.g., Cho et al., Cancer Res. (2010) 70(24):10121-30; Bauer et al. Science. (1999) 285(5428):727-9; Morvan et al. Nat Rev Cancer. (2016) 16(1):7-19; the disclosures of which are incorporated herein by reference in their entirety. Micabodies and mutants thereof also include those developed by AvidBiotics (South San Francisco, Calif.) and described online at (avidbiotics(dot)com).

In some instances, a chimeric bispecific binding member may be a CAR T cell adapter. As used herein, by “CAR T cell adapter” is meant an expressed bispecific polypeptide that binds the antigen recognition domain of a CAR and redirects the CAR to a second antigen. Generally, a CAR T cell adapter will have two binding regions, one specific for an epitope on the CAR to which it is directed and a second epitope directed to a binding partner which, when bound, transduces the binding signal activating the CAR. Useful CAR T cell adapters include but are not limited to e.g., those described in Kim et al. J Am Chem Soc. (2015) 137(8):2832-5; Ma et al. Proc Natl Acad Sci USA. (2016) 113(4):E450-8 and Cao et al. Angew Chem Int Ed Engl. (2016) 55(26):7520-4; the disclosures of which are incorporated herein by reference in their entirety.

In some cases, a useful therapeutic POI may be a therapeutic antibody. Suitable antibodies include, e.g., Natalizumab (Tysabri; Biogen Idec/Elan) targeting α4 subunit of α4β1 and α4β7 integrins (as used in the treatment of MS and Crohn's disease); Vedolizumab (MLN2; Millennium Pharmaceuticals/Takeda) targeting α4β7 integrin (as used in the treatment of UC and Crohn's disease); Belimumab (Benlysta; Human Genome Sciences/GlaxoSmithKline) targeting BAFF (as used in the treatment of SLE); Atacicept (TACI-Ig; Merck/Serono) targeting BAFF and APRIL (as used in the treatment of SLE); Alefacept (Amevive; Astellas) targeting CD2 (as used in the treatment of Plaque psoriasis, GVHD); Otelixizumab (TRX4; Tolerx/GlaxoSmithKline) targeting CD3 (as used in the treatment of T1D); Teplizumab (MGA031; MacroGenics/Eli Lilly) targeting CD3 (as used in the treatment of T1D); Rituximab (Rituxan/Mabthera; Genentech/Roche/Biogen Idec) targeting CD20 (as used in the treatment of Non-Hodgkin's lymphoma, RA (in patients with inadequate responses to TNF blockade) and CLL); Ofatumumab (Arzerra; Genmab/GlaxoSmithKline) targeting CD20 (as used in the treatment of CLL, RA); Ocrelizumab (2H7; Genentech/Roche/Biogen Idec) targeting CD20 (as used in the treatment of RA and SLE); Epratuzumab (hLL2; Immunomedics/UCB) targeting CD22 (as used in the treatment of SLE and non-Hodgkin's lymphoma); Alemtuzumab (Campath/MabCampath; Genzyme/Bayer) targeting CD52 (as used in the treatment of CLL, MS); Abatacept (Orencia; Bristol-Myers Squibb) targeting CD80 and CD86 (as used in the treatment of RA and JIA, UC and Crohn's disease, SLE); Eculizumab (Soliris; Alexion pharmaceuticals) targeting C5 complement protein (as used in the treatment of Paroxysmal nocturnal haemoglobinuria); Omalizumab (Xolair; Genentech/Roche/Novartis) targeting IgE (as used in the treatment of Moderate to severe persistent allergic asthma); Canakinumab (Ilaris; Novartis) targeting IL-1β (as used in the treatment of Cryopyrin-associated periodic syndromes, Systemic JIA, neonatal-onset multisystem inflammatory disease and acute gout); Mepolizumab (Bosatria; GlaxoSmithKline) targeting IL-5 (as used in the treatment of Hyper-eosinophilic syndrome); Reslizumab (SCH55700; Ception Therapeutics) targeting IL-5 (as used in the treatment of Eosinophilic oesophagitis); Tocilizumab (Actemra/RoActemra; Chugai/Roche) targeting IL-6R (as used in the treatment of RA, JIA); Ustekinumab (Stelara; Centocor) targeting IL-12 and IL-23 (as used in the treatment of Plaque psoriasis, Psoriatic arthritis, Crohn's disease); Briakinumab (ABT-874; Abbott) targeting IL-12 and IL-23 (as used in the treatment of Psoriasis and plaque psoriasis); Etanercept (Enbrel; Amgen/Pfizer) targeting TNF (as used in the treatment of RA, JIA, psoriatic arthritis, AS and plaque psoriasis); Infliximab (Remicade; Centocor/Merck) targeting TNF (as used in the treatment of Crohn's disease, RA, psoriatic arthritis, UC, AS and plaque psoriasis); Adalimumab (Humira/Trudexa; Abbott) targeting TNF (as used in the treatment of RA, JIA, psoriatic arthritis, Crohn's disease, AS and plaque psoriasis); Certolizumab pegol (Cimzia; UCB) targeting TNF (as used in the treatment of Crohn's disease and RA); Golimumab (Simponi; Centocor) targeting TNF (as used in the treatment of RA, psoriatic arthritis and AS); and the like.

Further examples of suitable antibodies include, e.g., Ipilimumab targeting CTLA-4 (as used in the treatment of Melanoma, Prostate Cancer, RCC); Tremelimumab targeting CTLA-4 (as used in the treatment of CRC, Gastric, Melanoma, NSCLC); Nivolumab targeting PD-1 (as used in the treatment of Melanoma, NSCLC, RCC); MK-3475 targeting PD-1 (as used in the treatment of Melanoma); Pidilizumab targeting PD-1 (as used in the treatment of Hematologic Malignancies); BMS-936559 targeting PD-L1 (as used in the treatment of Melanoma, NSCLC, Ovarian, RCC); MEDI4736 targeting PD-L1; MPDL33280A targeting PD-L1 (as used in the treatment of Melanoma); Rituximab targeting CD20 (as used in the treatment of Non-Hodgkin's lymphoma); Ibritumomab tiuxetan and tositumomab (as used in the treatment of Lymphoma); Brentuximab vedotin targeting CD30 (as used in the treatment of Hodgkin's lymphoma); Gemtuzumab ozogamicin targeting CD33 (as used in the treatment of Acute myelogenous leukaemia); Alemtuzumab targeting CD52 (as used in the treatment of Chronic lymphocytic leukaemia); IGN101 and adecatumumab targeting EpCAM (as used in the treatment of Epithelial tumors (breast, colon and lung)); Labetuzumab targeting CEA (as used in the treatment of Breast, colon and lung tumors); huA33 targeting gpA33 (as used in the treatment of Colorectal carcinoma); Pemtumomab and oregovomab targeting Mucins (as used in the treatment of Breast, colon, lung and ovarian tumors); CC49 (minretumomab) targeting TAG-72 (as used in the treatment of Breast, colon and lung tumors); cG250 targeting CAIX (as used in the treatment of Renal cell carcinoma); J591 targeting PSMA (as used in the treatment of Prostate carcinoma); MOv18 and MORAb-003 (farletuzumab) targeting Folate-binding protein (as used in the treatment of Ovarian tumors); 3F8, ch14.18 and KW-2871 targeting Gangliosides (such as GD2, GD3 and GM2) (as used in the treatment of Neuroectodermal tumors and some epithelial tumors); hu3S193 and IgN311 targeting Le y (as used in the treatment of Breast, colon, lung and prostate tumors); Bevacizumab targeting VEGF (as used in the treatment of Tumor vasculature); IM-2C6 and CDP791 targeting VEGFR (as used in the treatment of Epithelium-derived solid tumors); Etaracizumab targeting Integrin_V_3 (as used in the treatment of Tumor vasculature); Volociximab targeting Integrin_5_1 (as used in the treatment of Tumor vasculature); Cetuximab, panitumumab, nimotuzumab and 806 targeting EGFR (as used in the treatment of Glioma, lung, breast, colon, and head and neck tumors); Trastuzumab and pertuzumab targeting ERBB2 (as used in the treatment of Breast, colon, lung, ovarian and prostate tumors); MM-121 targeting ERBB3 (as used in the treatment of Breast, colon, lung, ovarian and prostate, tumors); AMG 102, METMAB and SCH 900105 targeting MET (as used in the treatment of Breast, ovary and lung tumors); AVE1642, IMC-A12, MK-0646, R1507 and CP 751871 targeting IGF1R (as used in the treatment of Glioma, lung, breast, head and neck, prostate and thyroid cancer); KB004 and IIIA4 targeting EPHA3 (as used in the treatment of Lung, kidney and colon tumors, melanoma, glioma and haematological malignancies); Mapatumumab (HGS-ETR1) targeting TRAILR1 (as used in the treatment of Colon, lung and pancreas tumors and haematological malignancies); HGS-ETR2 and CS-1008 targeting TRAILR2; Denosumab targeting RANKL (as used in the treatment of Prostate cancer and bone metastases); Sibrotuzumab and F19 targeting FAP (as used in the treatment of Colon, breast, lung, pancreas, and head and neck tumors); 8106 targeting Tenascin (as used in the treatment of Glioma, breast and prostate tumors); Blinatumomab (Blincyto; Amgen) targeting CD3 (as used in the treatment of ALL); pembrolizumab targeting PD-1 as used in cancer immunotherapy; 9E10 antibody targeting c-Myc; and the like.

Further examples of suitable antibodies include but are not limited to 8H9, Abagovomab, Abciximab, Abituzumab, Abrilumab, Actoxumab, Aducanumab, Afelimomab, Afutuzumab, Alacizumab pegol, ALD518, Alirocumab, Altumomab pentetate, Amatuximab, Anatumomab mafenatox, Anetumab ravtansine, Anifrolumab, Anrukinzumab, Apolizumab, Arcitumomab, Ascrinvacumab, Aselizumab, Atezolizumab, Atinumab, Atlizumab/tocilizumab, Atorolimumab, Bapineuzumab, Basiliximab, Bavituximab, Bectumomab, Begelomab, Benralizumab, Bertilimumab, Besilesomab, Bevacizumab/Ranibizumab, Bezlotoxumab, Biciromab, Bimagrumab, Bimekizumab, Bivatuzumab mertansine, Blosozumab, Bococizumab, Brentuximabvedotin, Brodalumab, Brolucizumab, Brontictuzumab, Cantuzumab mertansine, Cantuzumab ravtansine, Caplacizumab, Capromab pendetide, Carlumab, Catumaxomab, cBR96-doxorubicin immunoconjugate, Cedelizumab, Ch.14.18, Citatuzumab bogatox, Cixutumumab, Clazakizumab, Clenoliximab, Clivatuzumab tetraxetan, Codrituzumab, Coltuximab ravtansine, Conatumumab, Concizumab, CR6261, Crenezumab, Dacetuzumab, Daclizumab, Dalotuzumab, Dapirolizumab pegol, Daratumumab, Dectrekumab, Demcizumab, Denintuzumab mafodotin, Derlotuximab biotin, Detumomab, Dinutuximab, Diridavumab, Dorlimomab aritox, Drozitumab, Duligotumab, Dupilumab, Durvalumab, Dusigitumab, Ecromeximab, Edobacomab, Edrecolomab, Efalizumab, Efungumab, Eldelumab, Elgemtumab, Elotuzumab, Elsilimomab, Emactuzumab, Emibetuzumab, Enavatuzumab, Enfortumab vedotin, Enlimomab pegol, Enoblituzumab, Enokizumab, Enoticumab, Ensituximab, Epitumomab cituxetan, Erlizumab, Ertumaxomab, Etrolizumab, Evinacumab, Evolocumab, Exbivirumab, Fanolesomab, Faralimomab, Farletuzumab, Fasinumab, FBTA05, Felvizumab, Fezakinumab, Ficlatuzumab, Figitumumab, Firivumab, Flanvotumab, Fletikumab, Fontolizumab, Foralumab, Foravirumab, Fresolimumab, Fulranumab, Futuximab, Galiximab, Ganitumab, Gantenerumab, Gavilimomab, Gevokizumab, Girentuximab, Glembatumumab vedotin, Gomiliximab, Guselkumab, Ibalizumab, Ibalizumab, Icrucumab, Idarucizumab, Igovomab, IMAB362, Imalumab, Imciromab, Imgatuzumab, Inclacumab, Indatuximab ravtansine, Indusatumab vedotin, Inolimomab, Inotuzumab ozogamicin, Intetumumab, Iratumumab, Isatuximab, Itolizumab, Ixekizumab, Keliximab, Lambrolizumab, Lampalizumab, Lebrikizumab, Lemalesomab, Lenzilumab, Lerdelimumab, Lexatumumab, Libivirumab, Lifastuzumab vedotin, Ligelizumab, Lilotomab satetraxetan, Lintuzumab, Lirilumab, Lodelcizumab, Lokivetmab, Lorvotuzumab mertansine, Lucatumumab, Lulizumab pegol, Lumiliximab, Lumretuzumab, Margetuximab, Maslimomab, Matuzumab, Mavrilimumab, Metelimumab, Milatuzumab, Minretumomab, Mirvetuximab soravtansine, Mitumomab, Mogamulizumab, Morolimumab, Morolimumab immune, Motavizumab, Moxetumomab pasudotox, Muromonab-CD3, Nacolomab tafenatox, Namilumab, Naptumomab estafenatox, Narnatumab, Nebacumab, Necitumumab, Nemolizumab, Nerelimomab, Nesvacumab, Nofetumomab merpentan, Obiltoxaximab, Obinutuzumab, Ocaratuzumab, Odulimomab, Olaratumab, Olokizumab, Onartuzumab, Ontuxizumab, Opicinumab, Oportuzumab monatox, Orticumab, Otlertuzumab, Oxelumab, Ozanezumab, Ozoralizumab, Pagibaximab, Palivizumab, Pankomab, Panobacumab, Parsatuzumab, Pascolizumab, Pasotuxizumab, Pateclizumab, Patritumab, Perakizumab, Pexelizumab, Pinatuzumab vedotin, Pintumomab, Placulumab, Polatuzumab vedotin, Ponezumab, Priliximab, Pritoxaximab, Pritumumab, PRO 140, Quilizumab, Racotumomab, Radretumab, Rafivirumab, Ralpancizumab, Ramucirumab, Ranibizumab, Raxibacumab, Refanezumab, Regavirumab, Rilotumumab, Rinucumab, Robatumumab, Roledumab, Romosozumab, Rontalizumab, Rovelizumab, Ruplizumab, Sacituzumab govitecan, Samalizumab, Sarilumab, Satumomab pendetide, Secukinumab, Seribantumab, Setoxaximab, Sevirumab, SGN-CD19A, SGN-CD33A, Sifalimumab, Siltuximab, Simtuzumab, Siplizumab, Sirukumab, Sofituzumab vedotin, Solanezumab, Solitomab, Sonepcizumab, Sontuzumab, Stamulumab, Sulesomab, Suvizumab, Tabalumab, Tacatuzumab tetraxetan, Tadocizumab, Talizumab, Tanezumab, Taplitumomab paptox, Tarextumab, Tefibazumab, Telimomab aritox, Tenatumomab, Teneliximab, Teprotumumab, Tesidolumab, Tetulomab, TGN1412, Ticilimumab/tremelimumab, Tigatuzumab, Tildrakizumab, TNX-650, Toralizumab, Tosatoxumab, Tovetumab, Tralokinumab, TRBS07, Tregalizumab, Trevogrumab, Tucotuzumab celmoleukin, Tuvirumab, Ublituximab, Ulocuplumab, Urelumab, Urtoxazumab, Vandortuzumab vedotin, Vantictumab, Vanucizumab, Vapaliximab, Varlilumab, Vatelizumab, Veltuzumab, Vepalimomab, Vesencumab, Visilizumab, Vorsetuzumab mafodotin, Votumumab, Zalutumumab, Zanolimumab, Zatuximab, Ziralimumab, Zolimomab aritox, and the like.

Useful therapeutic POIs also include TCRs. A TCR generally includes an alpha chain and a beta chain; and recognizes antigen when presented by a major histocompatibility complex. In some cases, the TCR is an engineered TCR. Any engineered TCR having immune cell activation function can be induced using a method of the present disclosure. Such TCRs include, e.g., antigen-specific TCRs, Monoclonal TCRs (MTCRs), Single chain MTCRs, High Affinity CDR2 Mutant TCRs, CD1-binding MTCRs, High Affinity NY-ESO TCRs, VYG HLA-A24 Telomerase TCRs, including e.g., those described in PCT Pub Nos. WO 2003/020763, WO 2004/033685, WO 2004/044004, WO 2005/114215, WO 2006/000830, WO 2008/038002, WO 2008/039818, WO 2004/074322, WO 2005/113595, WO 2006/125962; Strommes et al. Immunol Rev. 2014; 257(1):145-64; Schmitt et al. Blood. 2013; 122(3):348-56; Chapuls et al. Sci Transl Med. 2013; 5(174):174ra27; Thaxton et al. Hum Vaccin Immunother. 2014; 10(11):3313-21 (PMID:25483644); Gschweng et al. Immunol Rev. 2014; 257(1):237-49 (PMID:24329801); Hinrichs et al. Immunol Rev. 2014; 257(1):56-71 (PMID:24329789); Zoete et al. Front Immunol. 2013; 4:268 (PMID:24062738); Marr et al. Clin Exp Immunol. 2012; 167(2):216-25 (PMID:22235997); Zhang et al. Adv Drug Deliv Rev. 2012; 64(8):756-62 (PMID:22178904); Chhabra et al. Scientific World Journal. 2011; 11:121-9 (PMID:21218269); Boulter et al. Clin Exp Immunol. 2005; 142(3):454-60 (PMID:16297157); Sami et al. Protein Eng Des Sel. 2007; 20(8):397-403; Boulter et al. Protein Eng. 2003; 16(9):707-11; Ashfield et al. IDrugs. 2006; 9(8):554-9; Li et al. Nat Biotechnol. 2005; 23(3):349-54; Dunn et al. Protein Sci. 2006; 15(4):710-21; Liddy et al. Mol Biotechnol. 2010; 45(2); Liddy et al. Nat Med. 2012; 18(6):980-7; Oates, et al. Oncoimmunology. 2013; 2(2):e22891; McCormack, et al. Cancer Immunol Immunother. 2013 April; 62(4):773-85; Bossi et al. Cancer Immunol Immunother. 2014; 63(5):437-48 and Oates, et al. Mol Immunol. 2015 October; 67(2 Pt A):67-74; the disclosures of which are incorporated herein by reference in their entirety.

In some instances, an expressed TCR targeting a particular antigen may be described as an anti-[antigen] TCR. Accordingly, in some instances, exemplary TCRs that may be induced to be expressed by a chimeric polypeptide of the instant disclosure include but are not limited to e.g., an anti-NY-ESO-1 TCR; an anti-MART-1 TCR; an anti-MAGE-A3 TCR; an anti-MAGE-A3 TCR; an anti-CEA TCR; an anti-gp100 TCR; an anti-WT1 TCR; an anti-HBV TCR; an anti-gag (WT and/or a/6) TCR; an anti-P53 TCR; an anti-TRAIL bound to DR4 TCR; an anti-HPV-16 (E6 and/or E7) TCR; an anti-Survivin TCR; an anti-KRAS mutants TCR; an anti-SSX2 TCR; an anti-MAGE-A10 TCR; an anti-MAGE-A4 TCR; an anti-AFP TCR; and the like. Examples include those described in J Immunol. (2008) 180(9):6116-31; J Immunol. (2008) 180(9):6116-31; Blood. (2009) 114(3):535-46; J Immunother. (2013) 36(2):133-51; Blood. (2013) 122(6):863-71; Mol Ther. (2011) 19(3):620-626; Blood. (2009) 114(3):535-46; Blood. (2011) 118(6):1495-503; J Hepatol. (2011) 55(1):103-10; Nat Med. (2008) 14(12):1390-5; Hum Gene Ther. (2008) 19(11):1219-32; J Immunol. (2008) 181(6):3769-76; Clin Cancer Res. (2015) 21(19):4431-9; J Clin Invest. (2015) 125(1):157-68; Cancer Immunol Res. (2016) 4(3):204-14; PLoS One. (2014) 9(3):e93321; J ImmunoTherapy Cancer. (2015) 3(Supp12):P14; Clin Cancer Res. (2015) 21(10):2268-77; J ImmunoTherapy Cancer. (2013) 1(Supp11):P10; and the like.

Useful TCRs include essentially any TCR useful in the treatment of cancer, including single-chain and multi-chain TCRs, directed to a targeting antigen. A TCR used in the instant methods will generally include, at a minimum, an antigen binding domain and a modified or unmodified TCR chain, or portion thereof, including but not limited to e.g., a modified or unmodified α-chain, a modified or unmodified β-chain, etc. An employed TCR may further include one or more costimulatory domains. In some instances, a TCR employed herein will include an alpha chain and a beta chain and recognize antigen when presented by a major histocompatibility complex.

In some instances, a therapeutic POI may be an innate-immune response inducer. As used herein, by “innate-immune response inducer” is meant any protein that when expressed within a mammal induces an innate immune response. Innate immune inducers include but are not limited to e.g., proteins or fragments thereof derived from bacteria, proteins or fragments thereof derived from virus, proteins or fragments thereof derived from fungus, proteins or fragments thereof derived from a mammalian parasite, including e.g., human parasites. Any protein that induces an innate immune response when expressed by a mammalian cell may find use as an innate-immune inducer of the instant disclosure. In some instances, an innate immune response inducer may be a flagellin protein.

In some instances, a therapeutic POI may be an immune suppression factor. As used herein, by “immune suppression factor” is meant any protein that when expressed within a mammal suppresses an immune response. Immune suppression factors include but are not limited to e.g., immunosuppressive cytokines (e.g., IL-10), immunosuppressive cell-to-cell signaling ligands (e.g., PD-L1), immunosuppressive secreted proteins (e.g., TGF-beta), immunosuppressive antibodies (e.g., anti-CD3 antibodies (e.g., Orthoclone OKT3 (also known as Muromonab-CD3), etc.), anti-CD25 antibodies, (e.g., Basiliximab, Daclizumab, etc.) anti-CD52 antibodies (e.g., Campath-1H (also known as alemtuzumab), etc.), and the like. Any protein that suppresses an immune response when expressed by a mammalian cell may find use as an immune suppression factor of the instant disclosure. In some instances, an immune suppression factor may be IL-10. In some instances, an immune suppression factor may be PD-L1. In some instances, an immune suppression factor may be TGF-beta. In some instances, an immune suppression factor may be an immunosuppressive antibody (e.g., (e.g., an anti-CD3 antibody (e.g., Orthoclone OKT3 (also known as Muromonab-CD3), etc.), an anti-CD25 antibody, (e.g., Basiliximab, Daclizumab, etc.) anti-CD52 antibody (e.g., Campath-1H (also known as alemtuzumab), etc.).

In some instances, a therapeutic POI may be chemokine. An expressed chemokine may affect one or more cellular behaviors including but not limited to cell migration. Examples of suitable chemokines include, e.g., MIP-1, MIP-1(3, MCP-1, RANTES, IP10, and the like. Additional examples of suitable chemokines include, but are not limited to, chemokine (C-C motif) ligand-2 (CCL2; also referred to as monocyte chemotactic protein-1 or MCP1); chemokine (C-C motif) ligand-3 (CCL3; also known as macrophage inflammatory protein-1A or MINA); chemokine (C-C motif) ligand-5 (CCL5; also known as RANTES); chemokine (C-C motif) ligand-17 (CCL17; also known as thymus and activation regulated chemokine or TARC); chemokine (C-C motif) ligand-19 (CCL19; also known as EBI1 ligand chemokine or ELC); chemokine (C-C motif) ligand-21 (CCL21; also known as 6Ckine); C-C chemokine receptor type 7 (CCR7); chemokine (C-X-C motif) ligand 9 (CXCL9; also known as monokine induced by gamma interferon or MIG); chemokine (C-X-C motif) ligand 10 (CXCL10; also known as interferon gamma-induced protein 10 or IP-10); chemokine (C-X-C motif) ligand 11 (CXCL11; also called interferon-inducible T-cell alpha chemoattractant or I-TAC); chemokine (C-X-C motif) ligand 16 (CXCL16; chemokine (C motif) ligand (XCL1; also known as lymphotactin); and macrophage colony-stimulating factor (MCSF).

In some instances, a therapeutic POI may be cytokine. Non-limiting examples of cytokines, the expression/secretion of which may be controlled by expression of a heterologous coding sequence, include but are not limited to e.g., Interleukins and related (e.g., IL-1-like, IL-1a, IL-1(3, IL-1RA, IL-18, IL-2, IL-4, IL-7, IL-9, IL-13, IL-15, IL-3, IL-5, GM-CSF, IL-6-like, IL-6, IL-11, G-CSF, IL-12, LIF, OSM, IL-10-like, IL-10, IL-20, IL-14, IL-16, IL-17, etc.), Interferons (e.g., IFN-α, IFN-β, IFN-γ, etc.), TNF family (e.g., CD154, LT-β, TNF-α, TNF-β, 4-1BBL, APRIL, CD70, CD153, CD178, GITRL, LIGHT, OX40L, TALL-1, TRAIL, TWEAK, TRANCE, etc.), TGF-β family (e.g., TGF-β1, TGF-β2, TGF-β3, etc.) and the like.

The above description of therapeutic agents that may be included in a therapeutic cell which is configured for survival modulation as described herein is not intended to be limiting and, in some instances, other therapeutic agents may be employed in conjunction with the methods for modulating therapeutic cell survival as described herein.

Binding-Triggered Transcriptional Switches

As used herein, a “binding-triggered transcriptional switch” (BTTS) generally refers to a synthetic modular polypeptide or system of interacting polypeptides having an extracellular domain that includes a first member of a specific binding pair that binds a binding partner (i.e., the second member of the specific binding pair; e.g., an antigen), a binding-transducer and an intracellular domain. Upon binding of the second member of the specific binding pair to the BTTS the binding signal is transduced to the intracellular domain such that the intracellular domain becomes activated and performs some function within the cell that it does not perform in the absence of the binding signal. Certain BTTS's are described in e.g., PCT Pub. No. WO 2016/138034 as well as U.S. Pat. Nos. 9,670,281 and 9,834,608; the disclosures of which are incorporated herein by reference in their entirety.

The specific binding member of the extracellular domain generally determines the specificity of the BTTS. In some instances, a BTTS may be referred according to its specificity as determined based on its specific binding member. For example, a specific binding member having binding partner “X” may be referred to as an X-BTTS or an anti-X BTTS.

A BTTS useful in the cells, systems, methods, etc., of the present disclosure may make use of a member of a specific binding pair, i.e., specific binding member, and thus, the BTTS may be specific for an antigen as described herein. Useful specific binding members include but not limited to e.g., antigen-antibody pairs, ligand receptor pairs, scaffold protein pairs, etc., including those specific for an antigen described herein.

In some instances, the specific binding member may be an antibody and its binding partner may be an antigen to which the antibody specifically binds. In some instances, the specific binding member may be a receptor and its binding partner may be a ligand to which the receptor specifically binds. In some instances, the specific binding member may be a scaffold protein and its binding partner may be a protein to which the scaffold protein specifically binds.

Useful specific binding pairs include those specific for an antigen, including those antigens described herein. For simplicity, regardless of the actual nature of the binding pair (i.e., antigen/antibody, receptor/ligand, etc.), the member of the binding pair attached to the BTTS will be referred to herein as an antigen binding domain and the member to which it binds will be referred to as an antigen herein (i.e., regardless of whether such a molecule would otherwise be considered an “antigen” in the conventional sense). However, one of ordinary skill will readily understand that descriptions of antigen binding domain-antigen interactions can be substituted with ligand/receptor, scaffold/binding partner pair where desired as appropriate.

In some cases, the specific binding member is an antibody. The antibody can be any antigen-binding antibody-based polypeptide, a wide variety of which are known in the art. In some instances, the specific binding member is or includes a monoclonal antibody, a single chain Fv (scFv), a Fab, etc. Other antibody-based recognition domains (cAb VHH (camelid antibody variable domains) and humanized versions, IgNAR VH (shark antibody variable domains) and humanized versions, sdAb VH (single domain antibody variable domains) and “camelized” antibody variable domains are suitable for use. In some instances, T-cell receptor (TCR) based recognition domains such as single chain TCR (scTv, single chain two-domain TCR containing VαVβ) are also suitable for use.

Where the specific binding member is an antibody-based binding member, the BTTS can be activated in the presence of a binding partner to the antibody-based binding member, including e.g., an antigen specifically bound by the antibody-based binding member. In some instances, antibody-based binding member may be defined, as is commonly done in the relevant art, based on the antigen bound by the antibody-based binding member, including e.g., where the antibody-based binding member is described as an “anti-” antigen antibody, e.g., an anti-CD19 antibody. Accordingly, antibody-based binding members suitable for inclusion in a BTTS or an antigen-specific therapeutic of the present methods can have a variety of antigen-binding specificities.

The components of BTTS's, employed in the described cells, systems, methods, etc., and the arrangement of the components of the switch relative to one another will vary depending on many factors including but not limited to e.g., the desired antigen, the activity of the intracellular domain, the overall function of the BTTS, the broader arrangement of a system comprising the BTTS, etc. The first binding member may include but is not limited to e.g., those agents that bind an antigen described herein. The intracellular domain may include but is not limited e.g., those intracellular domains that activate or repress transcription at a regulatory sequence, e.g., to induce or inhibit expression of a downstream component such as an antigen-triggered polypeptide (e.g., a second antigen-triggered polypeptide).

The binding transducer of BTTS's will also vary depending on the desired method of transduction of the binding signal. Generally, binding transducers may include those polypeptides and/or domains of polypeptides that transduce an extracellular signal to intracellular signaling e.g., as performed by the receptors of various signal transduction pathways. Transduction of a binding signal may be achieved through various mechanisms including but not limited to e.g., binding-induced proteolytic cleavage, binding-induced phosphorylation, binding-induced conformational change, etc. In some instances, a binding-transducer may contain a ligand-inducible proteolytic cleavage site such that upon binding the binding-signal is transduced by cleavage of the BTTS, e.g., to liberate an intracellular domain. For example, in some instances, a BTTS may include a Notch derived cleavable binding transducer, such as, e.g., a chimeric notch receptor polypeptide (e.g., a synNotch polypeptide) as described herein.

In other instances, the binding signal may be transduced in the absence of inducible proteolytic cleavage. Any signal transduction component or components of a signaling transduction pathway may find use in a BTTS whether or not proteolytic cleavage is necessary for signal propagation. For example, in some instances, a phosphorylation-based binding transducer, including but not limited to e.g., one or more signal transduction components of the Jak-Stat pathway, may find use in a non-proteolytic BTTS.

For simplicity, BTTS's, including but not limited to chimeric notch receptor polypeptides, are described primarily as single polypeptide chains. However, BTTS's, including chimeric notch receptor polypeptides, may be divided or split across two or more separate polypeptide chains where the joining of the two or more polypeptide chains to form a functional BTTS, e.g., a chimeric notch receptor polypeptide, may be constitutive or conditionally controlled. For example, constitutive joining of two portions of a split BTTS may be achieved by inserting a constitutive heterodimerization domain between the first and second portions of the split polypeptide such that upon heterodimerization the split portions are functionally joined.

Useful BTTS's that may be employed in the subject methods include, but are not limited to modular extracellular sensor architecture (MESA) polypeptides. A MESA polypeptide comprises: a) a ligand binding domain; b) a transmembrane domain; c) a protease cleavage site; and d) a functional domain. The functional domain can be a transcription regulator (e.g., a transcription activator, a transcription repressor). In some cases, a MESA receptor comprises two polypeptide chains. In some cases, a MESA receptor comprises a single polypeptide chain. Non-limiting examples of MESA polypeptides are described in, e.g., U.S. Patent Publication No. 2014/0234851; the disclosure of which is incorporated herein by reference in its entirety.

Useful BTTS's that may be employed in the subject methods include, but are not limited to polypeptides employed in the TANGO assay. The subject TANGO assay employs a TANGO polypeptide that is a heterodimer in which a first polypeptide comprises a tobacco etch virus (Tev) protease and a second polypeptide comprises a Tev proteolytic cleavage site (PCS) fused to a transcription factor. When the two polypeptides are in proximity to one another, which proximity is mediated by a native protein-protein interaction, Tev cleaves the PCS to release the transcription factor. Non-limiting examples of TANGO polypeptides are described in, e.g., Barnea et al. (Proc Natl Acad Sci USA. 2008 Jan. 8; 105(1):64-9); the disclosure of which is incorporated herein by reference in its entirety.

Useful BTTS's that may be employed in the subject methods include, but are not limited to von Willebrand Factor (vWF) cleavage domain-based BTTS's, such as but not limited to e.g., those containing an unmodified or modified vWF A2 domain. A subject vWF cleavage domain-based BTTS will generally include: an extracellular domain comprising a first member of a binding pair; a von Willebrand Factor (vWF) cleavage domain comprising a proteolytic cleavage site; a cleavable transmembrane domain and an intracellular domain. Non-limiting examples of vWF cleavage domains and vWF cleavage domain-based BTTS's are described in Langridge & Struhl (Cell (2017) 171(6):1383-1396); the disclosure of which is incorporated herein by reference in its entirety.

Useful BTTS's that may be employed in the subject methods include, but are not limited to chimeric Notch receptor polypeptides, such as but not limited to e.g., synNotch polypeptides (also referred to as “synNotch receptors”), non-limiting examples of which are described in PCT Pub. No. WO 2016/138034, U.S. Pat. Nos. 9,670,281, 9,834,608, Roybal et al. Cell (2016) 167(2):419-432, Roybal et al. Cell (2016) 164(4):770-9, and Morsut et al. Cell (2016) 164(4):780-91; the disclosures of which are incorporated herein by reference in their entirety.

SynNotch polypeptides are generally proteolytically cleavable chimeric polypeptides that generally include: a) an extracellular domain comprising a specific binding member; b) a proteolytically cleavable Notch receptor polypeptide comprising one or more proteolytic cleavage sites; and c) an intracellular domain. Binding of the specific binding member by its binding partner generally induces cleavage of the synNotch at the one or more proteolytic cleavage sites, thereby releasing the intracellular domain. In some instances, the instant methods may include where release of the intracellular domain triggers (i.e., induces) the production of an encoded payload, the encoding nucleic acid sequence of which is contained within the cell. Depending on the particular context, the produced payload is then generally expressed on the cell surface or secreted. SynNotch polypeptides generally include at least one sequence that is heterologous to the Notch receptor polypeptide (i.e., is not derived from a Notch receptor), including e.g., where the extracellular domain is heterologous, where the intracellular domain is heterologous, where both the extracellular domain and the intracellular domain are heterologous to the Notch receptor, etc.

Useful synNotch BTTS's will vary in the domains employed and the architecture of such domains. SynNotch polypeptides will generally include a Notch receptor polypeptide that includes one or more ligand-inducible proteolytic cleavage sites. The length of Notch receptor polypeptides will vary and may range in length from about 50 amino acids or less to about 1000 amino acids or more.

In some cases, the Notch receptor polypeptide present in a synNotch polypeptide has a length of from 50 amino acids (aa) to 1000 aa, e.g., from 50 aa to 75 aa, from 75 aa to 100 aa, from 100 aa to 150 aa, from 150 aa to 200 aa, from 200 aa to 250 aa, from 250 a to 300 aa, from 300 aa to 350 aa, from 350 aa to 400 aa, from 400 aa to 450 aa, from 450 aa to 500 aa, from 500 aa to 550 aa, from 550 aa to 600 aa, from 600 aa to 650 aa, from 650 aa to 700 aa, from 700 aa to 750 aa, from 750 aa to 800 aa, from 800 aa to 850 aa, from 850 aa to 900 aa, from 900 aa to 950 aa, or from 950 aa to 1000 aa. In some cases, the Notch receptor polypeptide present in a synNotch polypeptide has a length of from 300 aa to 400 aa, from 300 aa to 350 aa, from 300 aa to 325 aa, from 350 aa to 400 aa, from 750 aa to 850 aa, from 50 aa to 75 aa. In some cases, the Notch receptor polypeptide has a length of from 310 aa to 320 aa, e.g., 310 aa, 311 aa, 312 aa, 313 aa, 314 aa, 315 aa, 316 aa, 317 aa, 318 aa, 319 aa, or 320 aa. In some cases, the Notch receptor polypeptide has a length of 315 aa. In some cases, the Notch receptor polypeptide has a length of from 360 aa to 370 aa, e.g., 360 aa, 361 aa, 362 aa, 363 aa 364 aa, 365 aa, 366 aa, 367 aa, 368 aa, 369 aa, or 370 aa. In some cases, the Notch receptor polypeptide has a length of 367 aa.

In some cases, a Notch receptor polypeptide comprises an amino acid sequence having at least 50%, at least 55%, at least 60%, at least 65%, at least 70%, at least 75%, at least 80%, at least 85%, at least 90%, at least 95%, at least 98%, at least 99%, or 100%, amino acid sequence identity to the amino acid sequence of a Notch receptor. In some instances, the Notch regulatory region of a Notch receptor polypeptide is a mammalian Notch regulatory region, including but not limited to e.g., a mouse Notch (e.g., mouse Notch1, mouse Notch2, mouse Notch3 or mouse Notch4) regulatory region, a rat Notch regulatory region (e.g., rat Notch1, rat Notch2 or rat Notch3), a human Notch regulatory region (e.g., human Notch1, human Notch2, human Notch3 or human Notch4), and the like or a Notch regulatory region derived from a mammalian Notch regulatory region and having at least 50%, at least 55%, at least 60%, at least 65%, at least 70%, at least 75%, at least 80%, at least 85%, at least 90%, at least 95%, at least 98%, at least 99%, or 100%, amino acid sequence identity to the amino acid sequence of a mammalian Notch regulatory region of a mammalian Notch receptor amino acid sequence.

Subject Notch regulatory regions may include or exclude various components (e.g., domains, cleavage sites, etc.) thereof. Examples of such components of Notch regulatory regions that may be present or absent in whole or in part, as appropriate, include e.g., one or more EGF-like repeat domains, one or more Lin12/Notch repeat domains, one or more heterodimerization domains (e.g., HD-N or HD-C), a transmembrane domain, one or more proteolytic cleavage sites (e.g., a furin-like protease site (e.g., an 51 site), an ADAM-family protease site (e.g., an S2 site) and/or a gamma-secretase protease site (e.g., an S3 site)), and the like. Notch receptor polypeptides may, in some instances, exclude all or a portion of one or more Notch extracellular domains, including e.g., Notch-ligand binding domains such as Delta-binding domains. Notch receptor polypeptides may, in some instances, include one or more non-functional versions of one or more Notch extracellular domains, including e.g., Notch-ligand binding domains such as Delta-binding domains. Notch receptor polypeptides may, in some instances, exclude all or a portion of one or more Notch intracellular domains, including e.g., Notch Rbp-associated molecule domains (i.e., RAM domains), Notch Ankyrin repeat domains, Notch transactivation domains, Notch PEST domains, and the like. Notch receptor polypeptides may, in some instances, include one or more non-functional versions of one or more Notch intracellular domains, including e.g., non-functional Notch Rbp-associated molecule domains (i.e., RAM domains), non-functional Notch Ankyrin repeat domains, non-functional Notch transactivation domains, non-functional Notch PEST domains, and the like.

Non-limiting examples of particular synNotch BTTS's, the domains thereof, and suitable domain arrangements are described in PCT Pub. Nos. WO 2016/138034, WO 2017/193059, WO 2018/039247 and U.S. Pat. Nos. 9,670,281 and 9,834,608; the disclosures of which are incorporated herein by reference in their entirety.

Domains of a useful BTTS, e.g., the extracellular domain, the binding-transducer domain, the intracellular domain, etc., may be joined directly, i.e., with no intervening amino acid residues or may include a peptide linker that joins two domains. Peptide linkers may be synthetic or naturally derived including e.g., a fragment of a naturally occurring polypeptide.

A peptide linker can vary in length of from about 3 amino acids (aa) or less to about 200 aa or more, including but not limited to e.g., from 3 aa to 10 aa, from 5 aa to 15 aa, from 10 aa to 25 aa, from 25 aa to 50 aa, from 50 aa to 75 aa, from 75 aa to 100 aa, from 100 aa to 125 aa, from 125 aa to 150 aa, from 150 aa to 175 aa, or from 175 aa to 200 aa. A peptide linker can have a length of from 3 aa to 30 aa, e.g., 3, 4, 5, 6, 7, 8, 9, 10, 11, 12, 13, 14, 15, 16, 17, 18, 19, 20, 21, 22, 23, 24, 25, 26, 27, 28, 29, or 30 aa. A peptide linker can have a length of from 5 aa to 50 aa, e.g., from 5 aa to 40 aa, from 5 aa to 35 aa, from 5 aa to 30 aa, from 5 aa to 25 aa, from 5 aa to 20 aa, from 5 aa to 15 aa or from 5 aa to 10 aa.

In some instances, a BTTS may have an extracellular domain that includes a first member of a specific binding pair that binds a second member of the specific binding pair, wherein the extracellular domain does not include any additional first or second member of a second specific binding pair. For example, in some instances, a BTTS may have an extracellular domain that includes a first antigen-binding domain that binds an antigen, wherein the extracellular domain does not include any additional antigen-binding domains and does not bind any other antigens. A subject BTTS may, in some instances, include only a single extracellular domain. Accordingly, an employed BTTS may be specific for a single antigen and only specific for the single antigen. Such, BTTS's may be referred to as a “single antigen BTTS”.

BTTS's specific for a single antigen may be monovalent or multivalent (e.g., bivalent, trivalent, etc.) for the antigen. For example, in some instances, a monovalent BTTS may be employed that includes an antigen binding domain (e.g., a single antigen binding domain) for binding a single molecule of antigen. In some instances, a multivalent BTTS may be employed that includes an antigen binding domain or multiple antigen binding domains (e.g., 1, 2, 3, 4, 5, 6, etc. antigen binding domains) for binding multiple molecules of antigen.

In some instances, a BTTS may have an extracellular domain that includes the first or second members of two or more specific binding pairs. For example, in some instances, a BTTS may have an extracellular domain that includes a first antigen-binding domain and a second antigen-binding domain that are different such that the extracellular domain is specific for two different antigens. In some instances, a BTTS may have two or more extracellular domains that each includes the first or second members of two different specific binding pairs. For example, in some instances, a BTTS may have a first extracellular domain that includes a first antigen-binding domain and a second extracellular domain that includes a second antigen-binding domain where the two different antigen binding domains are each specific for a different antigen. As such, the BTTS may be specific for two different antigens.

A BTTS specific for two or more different antigens, containing either two extracellular domains or one extracellular domain specific for two different antigens, may be configured such that the binding of either antigen to the BTTS is sufficient to trigger activation of the BTTS, e.g., proteolytic cleavage of a cleavage domain of the BTTS, e.g., releasing an intracellular domain of the BTTS. Such a BTTS, capable of being triggered by any of two or more antigens, may find use as a component of a logic gate containing OR functionality. In some instances, a BTTS specific for two different antigens may be referred to as a “two-headed BTTS”. BTTS's specific for multiple antigens will not be limited to only two antigens and may, e.g., be specific for and/or triggered by more than two antigens, including e.g., three or more, four or more, five or more, etc.

As summarized above, antigen binding domains of BTTS's may be substituted, amended or exchanged as desired. For example, an antigen binding domain of any antigen specific molecule, such as an antibody, may be employed as the antigen binding domain of a BTTS described herein. Correspondingly, an antigen binding domain described above as used in a CAR may be employed in other contexts, such as e.g., in a BTTS as described. As such, disclosure of any agent targeted to a specific antigen in any context herein would be understood to constitute a disclosure of the use of an antigen binding domain in any other antigen-specific polypeptide described herein as well.

In some instances, a BTTS employed in method, cell and/or system of the present disclosure may employ a synthetic transcription factor. Accordingly, in some instances, an encoded heterologous apoptosis modulating agent of the present disclosure may be operably linked to a regulatory element responsive to a synthetic transcription factor.

Synthetic transcription factors, and regulatory elements responsive thereto, will vary and may include but are not limited to e.g., estradiol ligand binding domain (LBD) based synthetic transcription factors, progesterone LBD based synthetic transcription factors, zinc-finger based synthetic transcription factors, and the like. Synthetic transcription factors may by chimeric and may include various domains, e.g., a DNA binding domain (DBD), activation domain, zinc-finger domain(s), and the like. Useful domains, e.g., LBDs, DBDs, activation domains, etc., will vary and may include but are not limited to e.g., the Gal4p DBD, the Zif268 transcription factor DBD, viral activation domains (e.g., VP16, VP64, etc.), Msn2p activation domains, and the like. Non-limiting examples of useful synthetic transcription factors include but are not limited to e.g., GEM (Gal4 DNA binding domain-Estradiol hormone binding domain-Msn2 activation domain), Z3PM (Z3 zinc finger-Progesterone hormone binding domain-Msn2 activation domain), and the like. Correspondingly, useful regulatory elements will vary and may include promoters responsive to synthetic transcription factors, including but not limited to e.g., pZ promoters, pZ3 promoters, pGAL1 promoters, and the like. Examples of suitable promoters and synthetic transcription factors include, but are not limited to e.g., those described herein, those described in Aranda-Diaz et al. ACS Synth Biol. (2017) 6(3): 545-554; the disclosure of which is incorporated herein by reference in its entirety, and the like.

As described herein, methods and systems of the present disclosure may, in some instances, employ one or more BTTS's. For example, in some instances, a method or system of the present disclosure may include a single BTTS that, when triggered, induces expression of an encoded polypeptide, such as but not limited to e.g., a heterologous apoptosis modulating agents, an encoded therapeutic, or the like. In some instances, a method or system of the present disclosure may include multiple BTTS's, including e.g., 2 or more, 3 or more, 4 or more, 5 or more, 6 or more, etc., BTTS's. In some instances, multiple BTTS's may be employed that are triggered by different antigens and induce expression of different encoded polypeptides in response to the presence of each antigen. For example, a method or system of the present may include separate BTTS's that, when triggered by their respective antigens, separately induce expression of an anti-apoptotic BCL-2 family protein, a pro-apoptotic BCL-2 family protein, a therapeutic polypeptide, or the like, or a combination thereof.

Cells

As summarized above, the present disclosure includes cells, including therapeutic cells, that include one or more heterologous apoptosis modulating agents. The cells of the present disclosure may be configured to perform functions set forth in the methods described above. Accordingly, a cell of the present disclosure may be configured to include any of the agents described herein, including e.g., where such agents are arranged according to the arrangements described herein, e.g., where such arrangement performs a function as described herein or such arrangements provide for a molecular circuit providing the functions as described herein. For example, a cell may be configured such that survival of the cell is modulated, e.g., according to a method as described above. Essentially any appropriate cell may be employed or configured as described, where useful cell types include but are not limited to e.g., therapeutic cells, immune cells (e.g., therapeutic immune cells), stem cells (e.g., therapeutic stem cells), and the like.

Therapeutic cells of the present disclosure may include a heterologous nucleic acid encoding a therapeutic agent. Useful therapeutic agents that may be encoded in the subject therapeutic cells include but are not limited to e.g., a therapeutic antibody, a chimeric antigen receptor, an engineered T cell receptor, and the like. An encoded therapeutic agent of a therapeutic cell may be expressed constitutively or may be inducible, including e.g., where expression of the therapeutic agent is regulated, e.g., by an inducible promoter, by a binding-triggered transcriptional switch, or the like.

Suitable cells include immune cells. As used herein, the term “immune cells” generally includes white blood cells (leukocytes) which are derived from hematopoietic stem cells (HSC) produced in the bone marrow. “Immune cells” includes, e.g., lymphoid cells, i.e., lymphocytes (T cells, B cells, natural killer (NK) cells), and myeloid-derived cells (neutrophil, eosinophil, basophil, monocyte, macrophage, dendritic cells). “T cell” includes all types of immune cells expressing CD3 including T-helper cells (CD4+ cells), cytotoxic T-cells (CD8+ cells), T-regulatory cells (Treg) and gamma-delta T cells. A “cytotoxic cell” includes CD8+ T cells, natural-killer (NK) cells, and neutrophils, which cells are capable of mediating cytotoxicity responses. “B cell” includes mature and immature cells of the B cell lineage including e.g., cells that express CD19 such as Pre B cells, Immature B cells, Mature B cells, Memory B cells and plasmablasts. Immune cells also include B cell progenitors such as Pro B cells and B cell lineage derivatives such as plasma cells.

Suitable cells include a stem cells, progenitor cells, and the progeny thereof, including e.g., embryonic stem (ES) cells, induced pluripotent stem (iPS) cells; human embryonic stem cells, fetal cardiomyocytes, myofibroblasts, mesenchymal stem cells, autotransplated expanded cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogenic cells, allogenic cells, and post-natal stem cells. Suitable therapeutic stem cells include autologous and allogenic stem cells.

Suitable cells include primary cells and immortalized cell lines. Suitable cell lines include mammalian cell lines, e.g., human cell lines, non-human primate cell lines, rodent (e.g., mouse, rat) cell lines, and the like. In some instances, the cell is not an immortalized cell line, but is instead a cell (e.g., a primary cell) obtained from an individual. For example, in some cases, the cell is an immune cell, immune cell progenitor or immune stem cell obtained from an individual. As an example, the cell is a lymphoid cell, e.g., a lymphocyte, or progenitor thereof, obtained from an individual. As another example, the cell is a cytotoxic cell, or progenitor thereof, obtained from an individual. As another example, the cell is a stem cell or progenitor cell obtained from an individual.

Cells of the present disclosure include those that contain one or more of the described nucleic acids, expression vectors, etc. Cells of the present disclosure include cells that are genetically modified to produce one or more of the encoded components of the present disclosure or to which a nucleic acid, as described above, has been otherwise introduced. In some instances, the subject cells have been transduced with one or more nucleic acids and/or expression vectors to express one or more components of a molecular circuit of the present disclosure.

Cells encoding a heterologous apoptosis modulating agent of the present disclosure may be generated by any convenient method. Nucleic acids encoding one or more encoding components may be stably or transiently introduced into the subject cell, including where the subject nucleic acids are present only temporarily, maintained extrachromosomally, or integrated into the host genome. Introduction of the subject nucleic acids and/or genetic modification of the subject immune cell can be carried out in vivo, in vitro, or ex vivo.

In some cases, the introduction of the subject nucleic acids and/or genetic modification is carried out ex vivo. For example, a T lymphocyte, a stem cell, or an NK cell is obtained from an individual; and the cell obtained from the individual is modified to express one or more heterologous apoptosis modulating agents and, in some instances, additional polypeptides (e.g., a therapeutic polypeptide). The modified cell can thus be configured to produce the one or more heterologous apoptosis modulating agents and, optionally, additional polypeptides, including e.g., where the introduced heterologous apoptosis modulating agents and the additional polypeptides, where present, are independently constitutive or inducible. In some cases, the modified cell is modulated ex vivo. In other cases, the cell is introduced into (e.g., the individual from whom the cell was obtained) and/or already present in an individual; and the cell is modulated in vivo, e.g., by administering a nucleic acid or vector to the individual in vivo.

Nucleic Acids

As summarized above, the present disclosure provides nucleic acids encoding heterologous apoptosis modulating agents, including e.g., where such nucleic acids are configured to encode a molecular circuit for modulating survival of a cell, e.g., a therapeutic cell, into which the nucleic acid is introduced. Useful nucleic acids include isolated nucleic acids, nucleic acids introduced into the genome of a cell, nucleic acids present extrachromosomally in a cell, expression cassettes, vectors, and the like.

Useful nucleic acids include therapeutic encoding nucleic acids, i.e., nucleic acid encoding one or more therapeutic polypeptides. In some embodiments, one or more nucleic acids are provided where the nucleic acid, or nucleic acids collectively, include a first sequence encoding a therapeutic polypeptide and a second sequence encoding an apoptotic modulating agent. In some instances, the encoded therapeutic agent is responsive to a target antigen. Useful therapeutic agents responsive to target antigens include but are not limited to e.g., therapeutic antibodies, chimeric antigen receptors, engineered T cell receptors, and the like. In some instances, the encoded apoptotic modulating agent may be inducible.

Nucleic acids encoding apoptotic modulating agents include nucleic acids encoding pro- and/or anti-apoptotic BCL-2 family proteins. In some instances, a nucleic acid encoding an apoptotic modulating agent may include a sequence encoding an anti-apoptotic agent comprising a BCL-2 family anti-apoptotic protein. In some instances, a nucleic acid encoding an apoptotic modulating agent may include a sequence encoding a pro-apoptotic agent comprising a BCL-2 family pro-apoptotic protein.

Provided are nucleic acids encoding essentially any component, or combination thereof, for modulating survival of cells as describe herein, including but not limited to those combinations of encoded polypeptides and circuits specifically described herein. Encompassed are isolated nucleic acids encoding the subject components and/or circuits as well as various configurations containing such nucleic acids, such as vectors, e.g., expression cassettes, recombinant expression vectors, viral vectors, and the like.

Recombinant expression vectors of the present disclosure include those comprising one or more of the described nucleic acids. A nucleic acid comprising a nucleotide sequence encoding all or a portion of the components of a circuit of the present disclosure will in some embodiments be DNA, including, e.g., a recombinant expression vector. A nucleic acid comprising a nucleotide sequence encoding all or a portion of the components of a circuit of the present disclosure will in some embodiments be RNA, e.g., in vitro synthesized RNA.

As summarized above, in some instances, the subject encoding nucleic acid may include one or more regulatory nucleic acids, including where one or more encoding nucleic acids are operably linked to one or more regulatory sequences such as a transcriptional control element (e.g., a promoter; an enhancer; etc.). In some cases, the transcriptional control element is inducible. In some cases, the transcriptional control element is constitutive. In some cases, the promoters are functional in eukaryotic cells. In some cases, the promoters are cell type-specific promoters. In some cases, the promoters are tissue-specific promoters.

Depending on the host/vector system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544).

A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular temperature, compound, or protein.), it may be a spatially restricted promoter (i.e., transcriptional control element, enhancer, etc.)(e.g., tissue specific promoter, cell type specific promoter, etc.), and it may be a temporally restricted promoter (i.e., the promoter is in the “ON” state or “OFF” state during specific stages of embryonic development or during specific stages of a biological process, e.g., hair follicle cycle in mice).

Suitable promoter and enhancer elements are known in the art. For expression in a bacterial cell, suitable promoters include, but are not limited to, lacI, lacZ, T3, T7, gpt, lambda P and trc. For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue specific promoters.

In some instances, a transcriptional control element of a herein described nucleic acid may include a cis-acting regulatory sequence. Any suitable cis-acting regulatory sequence may find use in the herein described nucleic acids. For example, in some instances a cis-acting regulatory sequence may be or include an upstream activating sequence or upstream activation sequence (UAS). In some instances, a UAS of a herein described nucleic acid may be a Gal4 responsive UAS.

Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

Inducible promoters suitable for use include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline-responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

In some cases, the promoter is an immune cell promoter such as a CD8 cell-specific promoter, a CD4 cell-specific promoter, a neutrophil-specific promoter, or an NK-specific promoter. For example, a CD4 gene promoter can be used; see, e.g., Salmon et al. (1993) Proc. Natl. Acad. Sci. USA 90: 7739; and Marodon et al. (2003) Blood 101:3416. As another example, a CD8 gene promoter can be used. NK cell-specific expression can be achieved by use of an Ncrl (p46) promoter; see, e.g., Eckelhart et al. (2011) Blood 117:1565.

In some instances, an immune cell specific promoter of a nucleic acid of the present disclosure may be a promoter of a B29 gene promoter, a CD14 gene promoter, a CD43 gene promoter, a CD45 gene promoter, a CD68 gene promoter, a IFN-β gene promoter, a WASP gene promoter, a T-cell receptor β-chain gene promoter, a V9 γ (TRGV9) gene promoter, a V2 δ (TRDV2) gene promoter, and the like.

In some cases, a nucleic acid comprising a nucleotide sequence encoding a circuit of the present disclosure, or one or more components thereof, is a recombinant expression vector or is included in a recombinant expression vector. In some embodiments, the recombinant expression vector is a viral construct, e.g., a recombinant adeno-associated virus (AAV) construct, a recombinant adenoviral construct, a recombinant lentiviral construct, a recombinant retroviral construct, etc. In some cases, a nucleic acid comprising a nucleotide sequence encoding a circuit of the present disclosure, or one or more components thereof, is a recombinant lentivirus vector. In some cases, a nucleic acid comprising a nucleotide sequence encoding a circuit of the present disclosure, or one or more components thereof, is a recombinant AAV vector.

Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., Hum Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. In some cases, the vector is a lentivirus vector. Also suitable are transposon-mediated vectors, such as piggyback and sleeping beauty vectors.

In some instances, nucleic acids of the present disclosure may have a single sequence encoding two or more polypeptides where expression of the two or more polypeptides is made possible by the presence of a sequence element between the individual coding regions that facilitates separate expression of the individual polypeptides. Such sequence elements, may be referred to herein as bicistronic-facilitating sequences, where the presence of a bicistronic-facilitating sequence between two coding regions makes possible the expression of a separate polypeptide from each coding region present in a single nucleic acid sequence. In some instances, a nucleic acid may contain two coding regions encoding two polypeptides present in a single nucleic acid with a bicistronic-facilitating sequence between the coding regions. Any suitable method for separate expression of multiple individual polypeptides from a single nucleic acid sequence may be employed and, similarly, any suitable method of bicistronic expression may be employed.

In some instances, a bicistronic-facilitating sequence may allow for the expression of two polypeptides from a single nucleic acid sequence that are temporarily joined by a cleavable linking polypeptide. In such instances, a bicistronic-facilitating sequence may include one or more encoded peptide cleavage sites. Suitable peptide cleavage sites include those of self-cleaving peptides as well as those cleaved by a separate enzyme. In some instances, a peptide cleavage site of a bicistronic-facilitating sequence may include a furin cleavage site (i.e., the bicistronic-facilitating sequence may encode a furin cleavage site).

In some instances, the bicistronic-facilitating sequence may encode a self-cleaving peptide sequence. Useful self-cleaving peptide sequences include but are not limited to e.g., peptide 2A sequences, including but not limited to e.g., the T2A sequence

In some instances, a bicistronic-facilitating sequence may include one or more spacer encoding sequences. Spacer encoding sequences generally encode an amino acid spacer, also referred to in some instances as a peptide tag. Useful spacer encoding sequences include but are not limited to e.g., V5 peptide encoding sequences, including those sequences encoding a V5 peptide tag.

Multi- or bicistronic expression of multiple coding sequences from a single nucleic acid sequence may make use of but is not limited to those methods employing furin cleavage, T2A, and V5 peptide tag sequences. For example, in some instances, an internal ribosome entry site (IRES) based system may be employed. Any suitable method of bicistronic expression may be employed including but not limited to e.g., those described in Yang et al. (2008) Gene Therapy. 15(21):1411-1423; Martin et al. (2006) BMC Biotechnology. 6:4; the disclosures of which are incorporated herein by reference in their entirety.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-106 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. A therapeutic cell comprising a heterologous inducible pro-apoptotic agent comprising an inducible BCL-2 family pro-apoptotic protein.
2. The cell according to aspect 1, wherein the BCL-2 family pro-apoptotic protein is selected from the group consisting of: an inducible truncated BID, an inducible PUMA, an inducible BMF, an inducible HRK, and an inducible BIK.
3. The cell according to aspect 1 or 2, further comprising a heterologous anti-apoptotic agent comprising a BCL-2 family anti-apoptotic protein.
4. The cell accordingly to aspect 3, wherein the BCL-2 family anti-apoptotic protein is BCL-2.
5. The cell according to aspect 3 or 4, wherein the heterologous anti-apoptotic agent is constitutive.
6. The cell according to aspect 3 or 4, wherein the heterologous anti-apoptotic agent is inducible.
7. The cell according to any of the preceding aspects, wherein the cell is a therapeutic immune cell.
8. The cell according to any of the preceding aspects, wherein the cell comprises a heterologous nucleic acid encoding a therapeutic agent selected from the group consisting of: a therapeutic antibody, a chimeric antigen receptor, and an engineered T cell receptor.
9. The cell according to aspect 8, wherein expression of the therapeutic agent is regulatable.
10. The cell according to aspect 9, wherein the regulatable system comprises an inducible promoter controlling expression of the therapeutic agent.
11. The cell according to aspect 9 or 10, wherein expression of the therapeutic agent is regulated by a binding-triggered transcriptional switch.
12. The cell according to any of the preceding aspects, wherein the inducible pro-apoptotic agent is ligand inducible.
13. The cell according to aspect 12, wherein the ligand inducible pro-apoptotic agent comprises a sequence encoding a BCL-2 family pro-apoptotic protein operably linked to a regulatory sequence responsive to binding of a binding-triggered transcriptional switch to a ligand.
14. The cell according to aspect 13, wherein the BCL-2 family pro-apoptotic protein is a BIM, a truncated BID, a PUMA, a BMF, a HRK, or a BIK.
15. The cell according to aspect 13 or 14, wherein the ligand is expressed by non-target cells.
16. The cell according to aspect 15, wherein the non-target cells are non-cancer cells.
17. The cell according to aspect 13 or 14, wherein the ligand is present on a solid support.
18. The cell according to aspect 17, wherein the solid support is a polymer particle.
19. The cell according to any of aspects 1 to 11, wherein the inducible pro-apoptotic agent is small molecule inducible.
20. The cell according to any of aspects 1 to 11, wherein the inducible pro-apoptotic agent is stimuli inducible.
21. The cell according to aspect 20, wherein the stimuli inducible pro-apoptotic agent is induced by light, ultrasound or hypoxia.
22. A method comprising administering a therapeutic cell according to any of aspects 1 to 21 to a subject in need thereof.
23. A method of treating a subject for an adverse reaction to a therapeutic cell of any of aspects 1 to 21, the method comprising inducing the heterologous inducible pro-apoptotic agent.
24. The method according to aspect 23, wherein the inducible pro-apoptotic agent is small molecule inducible and the method comprises administering to the subject an amount of a small molecule effective to induce the pro-apoptotic agent.
25. The method according to aspect 24, wherein the inducible pro-apoptotic agent comprises a sequence encoding a BCL-2 family pro-apoptotic protein operably linked to a regulatory sequence.
26. The method according to aspect 25, wherein the BCL-2 family pro-apoptotic protein is selected from the group consisting of: a truncated BID, a PUMA, a BMF, a HRK, and a BIK.
27. The method according to aspect 25 or 26, wherein the small molecule binds a transcriptional activator of the regulatory sequence thereby inducing expression of the BCL-2 family pro-apoptotic protein.
28. The method according to aspect 25 or 26, wherein the small molecule competitively binds a transcriptional repressor of the regulatory sequence thereby inducing expression of the BCL-2 family pro-apoptotic protein.
29. The method according to aspect 24, wherein the inducible pro-apoptotic agent comprises a split BCL-2 family pro-apoptotic protein dimerized by the small molecule.
30. The method according to aspect 29, wherein the split BCL-2 family pro-apoptotic protein is selected from the group consisting of: a split tBID, a split PUMA, a split BMF, a split HRK, and a split BIK.
31. The method according to aspect 23, wherein the inducible pro-apoptotic agent is stimuli inducible and the method comprises stimulating at least a portion of the subject with an amount of a stimuli effective to induce the pro-apoptotic agent.
32. The method according to aspect 23, wherein the inducible pro-apoptotic agent is ligand inducible and the method comprises contacting the subject with an amount of a ligand effective to induce the pro-apoptotic agent.
33. One or more nucleic acids comprising:
a first sequence encoding a therapeutic polypeptide responsive to a target antigen; and
a second sequence encoding an inducible pro-apoptotic agent comprising an inducible BCL-2 family pro-apoptotic protein.
34. The one or more nucleic acids according to aspect 33, wherein the inducible BCL-2 family pro-apoptotic protein is selected from the group consisting of: an inducible truncated BID, an inducible PUMA, an inducible BMF, an inducible HRK, and an inducible BIK.
35. The one or more nucleic acids according to aspect 33 or 34, wherein the therapeutic polypeptide is selected from the group consisting of: a therapeutic antibody, a chimeric antigen receptor, and an engineered T cell receptor.
36. The one or more nucleic acids according to any of aspects 33 to 35, wherein the target antigen is a cancer antigen.
37. The one or more nucleic acids according to any of aspects 33 to 35, wherein the target antigen is a non-natural bioorthogonal ligand.
38. The one or more nucleic acids according to any of aspects 33 to 37, wherein the inducible pro-apoptotic agent is small molecule inducible.
39. The one or more nucleic acids according to any of aspects 33 to 37, wherein the inducible pro-apoptotic agent is stimuli inducible.
40. The one or more nucleic acids according to aspect 39, wherein the stimuli inducible pro-apoptotic agent is induced by light, ultrasound or hypoxia.
41. The one or more nucleic acids according to any of aspects 33 to 37, wherein the inducible pro-apoptotic agent is ligand inducible.
42. The one or more nucleic acids according to aspect 41, wherein the ligand inducible pro-apoptotic agent comprises a sequence encoding a BCL-2 family pro-apoptotic protein operably linked to a regulatory sequence responsive to binding of a binding-triggered transcriptional switch to a ligand.
43. The one or more nucleic acids according to aspect 42, wherein the BCL-2 family pro-apoptotic protein is selected from the group consisting of: a tBID, a PUMA, a BMF, a HRK, and a BIK.
44. The one or more nucleic acids according to aspect 42 or 43, wherein the ligand is expressed by non-target cells.
45. The one or more nucleic acids according to aspect 44, wherein the non-target cells are non-cancer cells.
46. The one or more nucleic acids according to any of aspects 33 to 45, further comprising a third sequence encoding a heterologous anti-apoptotic agent comprising a BCL-2 family anti-apoptotic protein.
47. The one or more nucleic acids according to aspect 46, wherein the BCL-2 family anti-apoptotic protein is a BCL-2.
48. The one or more nucleic acids according to aspect 46 or 47, wherein the heterologous anti-apoptotic agent is constitutive.
49. The one or more nucleic acids according to aspect 46 or 47, wherein the heterologous anti-apoptotic agent is inducible.
50. A vector comprising the one or more nucleic acids according to any of aspects 33 to 49.
51. A cell comprising the vector of aspect 50.
52. A therapeutic cell comprising a heterologous constitutive or inducible anti-apoptotic agent comprising a BCL-2 family anti-apoptotic protein.
53. The cell according to aspect 52, wherein the BCL-2 family anti-apoptotic protein is a BCL-2.
54. The cell according to aspect 52 or 53, wherein the anti-apoptotic agent is ligand inducible.
55. The cell according to aspect 54, wherein the ligand inducible anti-apoptotic agent comprises a sequence encoding a BCL-2 family anti-apoptotic protein operably linked to a regulatory sequence responsive to binding of a binding-triggered transcriptional switch to a ligand.
56. The cell according to aspect 55, wherein the ligand is expressed by a target cell.
57. The cell according to aspect 56, wherein the target cell is a cancer cell.
58. The cell according to aspect 56, wherein the ligand is expressed tissue specifically.
59. The cell according to aspect 55, wherein the ligand is present on a solid support.
60. The cell according to aspect 59, wherein the solid support is a polymer particle.
61. The cell according to any of aspects 55 to 60, wherein the ligand is a non-natural bioorthogonal ligand.
62. The cell according to aspect 52 or 53, wherein the anti-apoptotic agent is small molecule inducible.
63. The cell according to aspect 52 or 53, wherein the anti-apoptotic agent is stimuli inducible.
64. The cell according to aspect 63, wherein the stimuli inducible anti-apoptotic agent is induced by light, ultrasound or hypoxia.
65. The cell according to any of aspects 52 to 64, wherein the therapeutic cell is a therapeutic immune cell.
66. The cell according to any of aspects 52 to 65, wherein the therapeutic cell comprises a heterologous nucleic acid encoding a therapeutic agent selected from the group consisting of: a therapeutic antibody, a chimeric antigen receptor, and an engineered T cell receptor.
67. The cell according to aspect 66, wherein expression of the therapeutic agent is regulated by a binding-triggered transcriptional switch.
68. The cell according to any of clams 52 to 66, further comprising a heterologous inducible pro-apoptotic agent comprising an inducible BCL-2 family pro-apoptotic protein.
69. The cell according to aspect 68, wherein the BCL-2 family pro-apoptotic protein is selected from the group consisting of: an inducible BIM, an inducible truncated BID, an inducible PUMA, an inducible BMF, an inducible HRK, and an inducible BIK.
70. A method comprising administering a therapeutic cell according to any of aspects 52 to 69 to a subject in need thereof.
71. A method of enhancing a cellular therapy, the method comprising:
administering or having administered a therapeutic cell comprising an inducible heterologous anti-apoptotic agent comprising a BCL-2 family anti-apoptotic protein to a subject; and inducing the inducible heterologous anti-apoptotic agent.
72. The method according to aspect 71, wherein the BCL-2 family anti-apoptotic protein is a BCL-2.
73. The method according to aspect 71 or 72, wherein the therapeutic cell comprises a therapeutic polypeptide, or an encoding sequence thereof, responsive to a target antigen.
74. The method according to aspect 73, wherein the therapeutic polypeptide is selected from the group consisting of: a therapeutic antibody, a chimeric antigen receptor, and an engineered T cell receptor.
75. The method according to aspect 74, wherein the target antigen is a cancer antigen.
76. The method according to any of aspects 71 to 75, wherein the inducible anti-apoptotic agent is small molecule inducible and the method comprises administering to the subject an amount of a small molecule effective to induce the anti-apoptotic agent.
77. The method according to aspect 76, wherein the inducible anti-apoptotic agent comprises a sequence encoding a BCL-2 family anti-apoptotic protein operably linked to a regulatory sequence.
78. The method according to aspect 77, wherein the small molecule binds a transcriptional activator of the regulatory sequence thereby inducing expression of the BCL-2 family anti-apoptotic protein.
79. The method according to aspect 77, wherein the small molecule competitively binds a transcriptional repressor of the regulatory sequence thereby inducing expression of the BCL-2 family anti-apoptotic protein.
80. The method according to aspect 76, wherein the inducible anti-apoptotic agent comprises a split BCL-2 family anti-apoptotic protein dimerized by the small molecule.
81. The method according to aspect 80, wherein the split BCL-2 family anti-apoptotic protein is a split BCL-2.
82. The method according to any of aspects 71 to 75, wherein the inducible anti-apoptotic agent is stimuli inducible and the method comprises stimulating at least a portion of the subject with an amount of a stimuli effective to induce the anti-apoptotic agent.
83. The method according to any of aspects 71 to 75, wherein the inducible anti-apoptotic agent is ligand inducible and the method comprises contacting the subject with an amount of a ligand effective to induce the anti-apoptotic agent.
84. One or more nucleic acids comprising:
a first sequence encoding a therapeutic polypeptide responsive to a target antigen; and
a second sequence encoding an anti-apoptotic agent comprising a BCL-2 family anti-apoptotic protein.
85. The one or more nucleic acids according to aspect 84, wherein the BCL-2 family anti-apoptotic protein is a BCL-2.
86. The one or more nucleic acids according to aspect 84 or 85, further comprising a third sequence encoding a heterologous inducible pro-apoptotic agent comprising an inducible BCL-2 family pro-apoptotic protein.
87. The one or more nucleic acids according to aspect 85 or 86, wherein the BCL-2 family pro-apoptotic protein is selected from the group consisting of: an inducible BIM, an inducible truncated BID, an inducible PUMA, an inducible BMF, an inducible HRK, and an inducible BIK.
88. The one or more nucleic acids according to aspect 85 or 86, wherein the heterologous inducible pro-apoptotic agent is small molecule inducible.
89. The one or more nucleic acids according to aspect 85 or 86, wherein the heterologous inducible pro-apoptotic agent is stimuli inducible.
90. The one or more nucleic acids according to aspect 89, wherein the stimuli inducible pro-apoptotic agent is induced by light, ultrasound or hypoxia.
91. The one or more nucleic acids according to aspect 85 or 86, wherein the heterologous inducible pro-apoptotic agent is ligand inducible.
92. The one or more nucleic acids according to aspect 91, wherein the ligand inducible pro-apoptotic agent comprises a sequence encoding a BCL-2 family pro-apoptotic protein operably linked to a regulatory sequence responsive to binding of a binding-triggered transcriptional switch to a ligand.
93. The one or more nucleic acids according to any of aspects 84 or 92, wherein the therapeutic polypeptide is selected from the group consisting of: a therapeutic antibody, a chimeric antigen receptor, and an engineered T cell receptor.
94. The one or more nucleic acids according to any of aspects 84 to 93, wherein the target antigen is a cancer antigen.
95. The one or more nucleic acids according to any of aspects 84 to 94, wherein the anti-apoptotic agent is constitutive.
96. The one or more nucleic acids according to any of aspects 84 to 94, wherein the anti-apoptotic agent is inducible.
97. The one or more nucleic acids according to aspect 96, wherein the anti-apoptotic agent is small molecule inducible.
98. The one or more nucleic acids according to aspect 96, wherein the inducible anti-apoptotic agent is stimuli inducible.
99. The one or more nucleic acids according to aspect 98, wherein the stimuli inducible anti-apoptotic agent is induced by light, ultrasound or hypoxia.
100. The one or more nucleic acids according to aspect 96, wherein the inducible anti-apoptotic agent is ligand inducible.
101. The one or more nucleic acids according to aspect 100, wherein the ligand inducible anti-apoptotic agent comprises a sequence encoding a BCL-2 family anti-apoptotic protein operably linked to a regulatory sequence responsive to binding of a binding-triggered transcriptional switch to a ligand.
102. The one or more nucleic acids according to aspect 101, wherein the ligand is expressed by a target cell.
103. The one or more nucleic acids according to aspect 102, wherein the target cell is a cancer cell.
104. The one or more nucleic acids according to aspect 102, wherein the ligand is expressed tissue specifically.
105. A vector comprising the one or more nucleic acids according to any of aspects 84 to 105.
106. A cell comprising the vector of aspect 105.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1: BCL-2 Family Protein Modulation of Therapeutic Cell Survival

One major feature lacking from current clinical engineered cell therapies, including CAR and engineered TCR therapies, is a means to prevent activation of the engineered cells in an antigen-dependent manner. CAR and TCR T cell therapies have caused severe and sometimes fatal reactions by killing cells in healthy tissues. Engineering of T cells to recognize an antigen expressed by healthy tissues and transduce this recognition into inhibition of activation and target killing, would prevent toxic cross-reactivities due to killing of healthy tissues.

Additionally, although cellular therapies have been shown to be very effective, there are concerns about toxic side effects that can be mediated by these engineered cells. One approach to prevent adverse outcomes is to incorporate a safety switch gene in the therapeutic cells. In this strategy, a drug can be administered to active the safety switch and eliminate the therapeutic cells in the case of an adverse event. While there have been multiple safety switch designs reported in the literature, none of them have demonstrated robust efficacy in the clinic. In addition to a lack of efficacy (e.g., a lack of complete elimination of engineered therapeutic cells), some of these safety switch systems may cause toxicity themselves by targeting normal cells in addition to the engineered therapeutic cells. Regulated BCL-2 family member activity as described herein provides both enhanced efficacy and reduced toxicity relative to currently available cell therapy safety switches.

Members of the BCL-2 protein family have been identified that can be synthetically expressed in T cells to control apoptosis of these cells. The BCL-2 family is an evolutionarily-conserved set of proteins known as important regulators of apoptotic signaling at the mitochondrion. Each of the BCL-2 proteins contains at least one BCL-2 homology (BH) domain, which provide the regulatory function of these proteins. The BCL-2 protein family comprises both pro-apoptotic (e.g., BIM) and anti-apoptotic proteins (e.g., BCL-2) that interact with one another to regulate apoptosis. The dynamic balance between these pro-apoptotic and anti-apoptotic proteins helps determine whether a cell undergoes apoptosis. There are at least 25 members of the BCL-2 family, and the exact identity of individual proteins in interacting pro-apoptotic and anti-apoptotic pairs is an important parameter regulating the initiation of apoptosis.

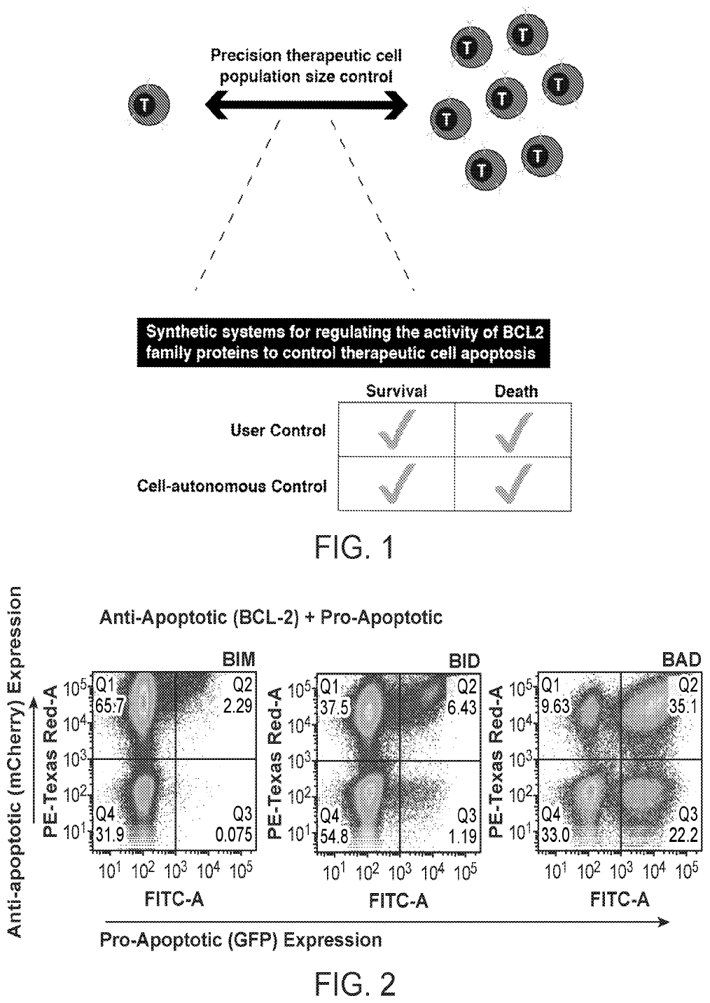

This example pertains, at least in part, to the screening of the BCL-2 proteins for their ability to regulate apoptosis when synthetically expressed in primary human T cells. This example demonstrates the ability to control the population size of engineered therapeutic cells by synthetically regulating the activity of pro- and anti-apoptotic BCL2 family members. For example, as schematically depicted in FIG. 1, precision therapeutic cell population size control is achieved through the implementation of synthetic systems for regulating the activity of BCL2 family proteins to control therapeutic cell apoptosis. Depending on the configuration employed, such systems can achieve user and/or cell-autonomous control of both therapeutic cell survival and death.

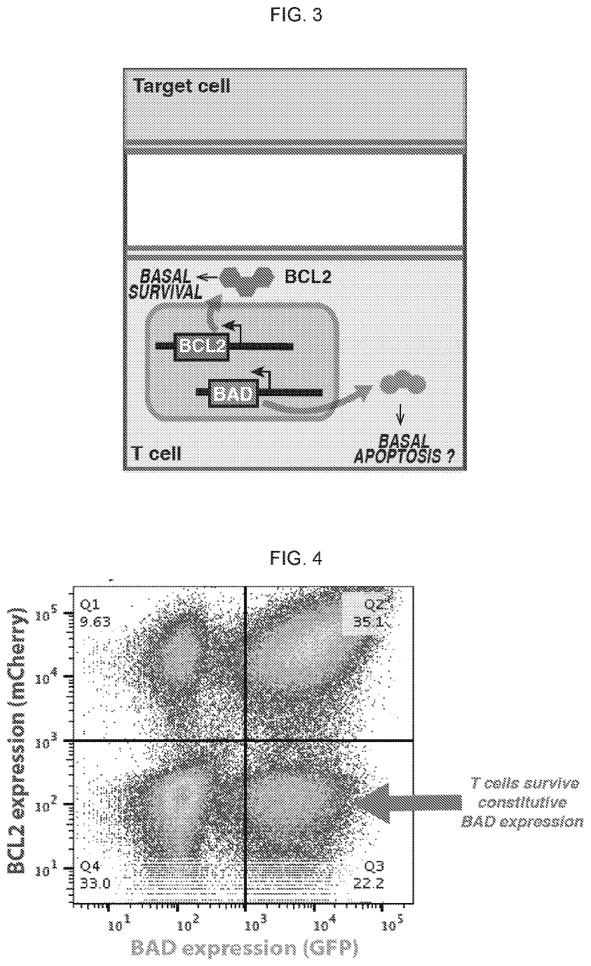

The abilities of several pro-apoptotic proteins (e.g., BIM, truncated Bid (tBID), and BAD) to induce T cell apoptosis when synthetically overexpressed from a constitutive promoter were tested. The tested T cells were also engineered to constitutively overexpress anti-apoptotic BCL-2. While BIM, tBID, and BAD are all pro-apoptotic BCL-2 family members generally, significant differences were observed in the ability of each protein to induce apoptosis of primary human T cells in this context (see FIG. 2, showing levels of anti- and pro-apoptotic reporter expression at culture day 12 as measured by FACS). In particular, it was found that constitutive expression of BAD in the presence of constitutive expression of BCL2 (as schematically depicted in FIG. 3) was insufficient to drive T cell death as numerous T cells survived constitutive BAD expression (FIG. 4). Further experiments demonstrated that, even in the absence of overexpressed BCL-2, BAD was insufficient to drive T cell death.

In contrast, constitutive expression of tBID or BIM drove significant apoptosis, as demonstrated by the fact that few live T cells expressing these proteins were present at day 12 of culture (FIG. 2, left and middle panels). However, constitutive co-expression of BCL-2 was able to at least partially buffer against the apoptotic effects of the induced pro-apoptotic factors tBID and BIM. It was further found that, in the absence of co-expression with anti-apoptotic BCL-2, uninduced BIM leads to significant levels of cell death. Without being bound by theory, these effects were likely due to leaky expression of BIM, the high effectiveness of BIM in inducing apoptosis, or a combination thereof. These results contraindicate the use of BIM as the inducible pro-apoptotic agent in unbuffered circuits for controlling therapeutic cell survival. However, the results also demonstrate that BIM may be effectively used as the pro-apoptotic agent in circuits that include anti-apoptotic buffering, such as those circuits that include co-expression with anti-apoptotic BCL-2.

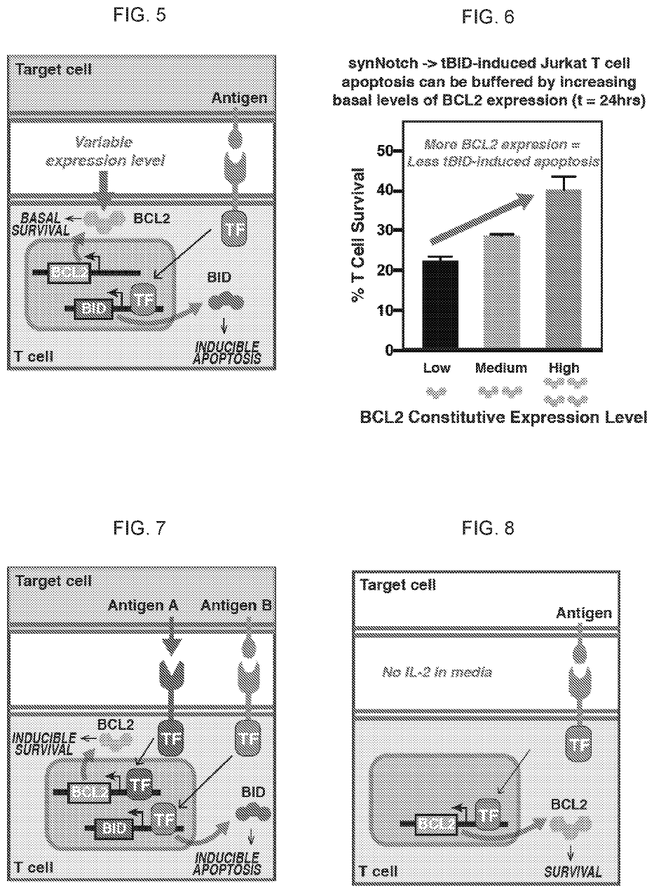

The ability of anti-apoptotic BCL-2 to buffer a pro-apoptotic protein was further tested in an inducible tBID circuit (described in more detail below) with varied levels of constitutive BCL-2 expression (schematically depicted in FIG. 5). In this experiment, constant levels of tBID expression were induced by the presence of antigen expressing target cells, BCL-2 expression levels were varied, and the amount of T cell survival at different levels of BCL-2 expression was quantitated (FIG. 6). Higher levels of T cell survival were seen in populations with higher expression of BCL2. Thus, these data demonstrate that expression of anti-apoptotic BCL-2 titratably buffers against apoptosis, including where such apoptosis is induced by a pro-apoptotic BCL-2 family protein such as tBID. These results show that expression of anti-apoptotic BCL-2 family proteins can be used to regulate apoptosis driven by pro-apoptotic BCL-2 family proteins in the context of engineered T cells.

While the preceding employed constitutive expression to drive the anti-apoptotic BCL-2 family protein, inducible expression of anti-apoptotic proteins may similarly be employed. For example, as schematized in FIG. 7, T cell survival may be inducibly promoted through antigen-dependent expression of BCL2 using, e.g., a binding triggered transcriptional switch responsive to “antigen A”. Any useful antigen and binding-triggered transcriptional switch may be employed, including e.g., where an orthogonal synNotch is employed to induce expression of BCL-2 or other BCL-2 family anti-apoptotic protein.

The use of expressed anti-apoptotic proteins, whether constitutive or inducible, in therapeutic cells is not limited to the buffering of the effects of pro-apoptotic proteins. For example, in some instances, expressed anti-apoptotic proteins may be employed generally to promote survival and/or expansion of therapeutic cells and/or to prevent therapeutic cell death in context where therapeutic cell survival would low or not otherwise expected. For example, as schematized in FIG. 8, an inducible BCL-2 circuit may be employed in result in induced expression of BCL-2 in response to the presence of an antigen. Thus, in situations where T cell survival and/or expansion would be otherwise limited (e.g., during IL-2 withdrawal), induced expression of BCL-2 serves as an antigen-dependent pro-survival switch that protect T cells from cytokine withdrawal-induced apoptosis.

The finding that anti-apoptotic BCL-2 proteins can buffer against the effects of induced pro-apoptotic BCL-2 family proteins in engineered T cells provides an advantage for the building of circuits to control inducible (e.g., ligand-responsive, small molecule-responsive, etc.) T cell death. Inducible systems to regulate protein expression/function are generally limited by some level of basal activity of the inducible construct as exemplified above. Such basal activity is expected to be especially problematic in systems configured to have an output of induction of apoptosis. Having a means to buffer against pro-apoptotic signals “leaking” basally from inducible systems provides an advantage in building inducible death circuits, while also improving the maximum dynamic range by e.g., providing essentially no basal “leaky” cell death and/or complete cell death upon induction.

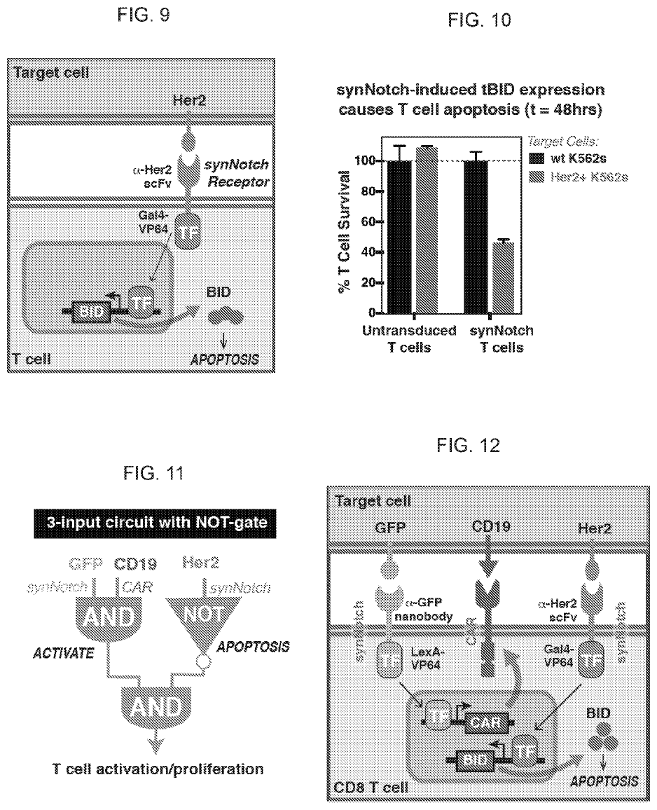

After finding that constitutive expression of BCL-2 family members could regulate apoptosis of engineered T cells, whether inducible expression of pro-apoptotic tBID could be used to induce T cell death was tested. BID is a member of the BCL-2 protein family whose cleavage by caspase-8 releases tBID (truncated Bid); tBID then translocates to the mitochondria to initiate apoptosis. To test the ability of synNotch-induced tBID expression to drive apoptosis, human primary CD8+ T cells were engineered with an anti-Her2 Gal4-VP64 synNotch controlling expression of tBID (schematically depicted in FIG. 9). It was found that these engineered T cells selectively die in the presence of Her2 expressing K562 target cells, demonstrating that synNotch-induced tBID expression can drive T cell death (FIG. 10).

Whether synNotch-induced apoptosis could act as a NOT-gate when integrated into a higher order AND-gate with 3-input antigen recognition circuit (FIG. 11) was next evaluated. A critical component of a therapeutic T cell response is the explosive cytokine-driven cell proliferation that results from initial activation. It was reasoned that if a NOT input induced apoptosis, then the T cells might be locally blocked from undergoing this critical expansion. Human primary CD8+ T cells were engineered with a composite circuit: for negative selection the cells contained an anti-Her2 synNotch driving tBID expression; for positive selection the cells contained a two input AND gate-anti-GFP LexA-VP64 synNotch controlling expression of the anti-CD19 CAR (FIG. 12). Based on previous experience with this AND-gate module, it was known that these T cells should activate and kill only target cells expressing both GFP and CD19. It was therefore tested whether anti-Her2 synNotch-driven tBID expression could then override this AND-gate to prevent killing of Her2/GFP/CD19 triple antigen positive cells (i.e., Her2 is an overriding negative input).

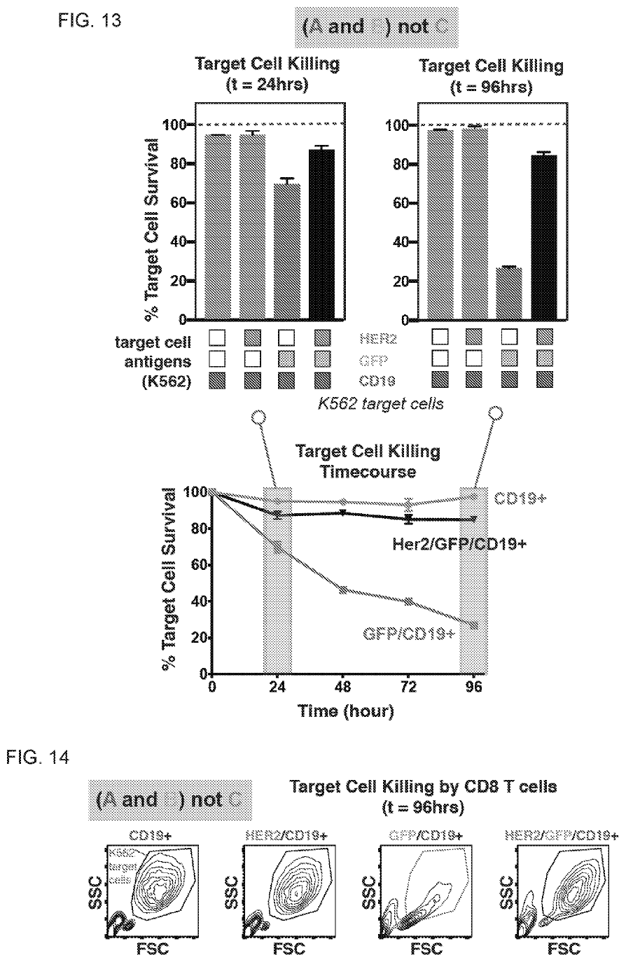

To test the this positive/negative 3 input gating circuit, a time-course was performed coculturing these engineered T cells with K562 target cells expressing either CD19, Her2/CD19, GFP/CD19, or Her2/GFP/CD19. It was found that after 24 hours there was largely selective killing of GFP/CD19 positive cells, with some minor killing of Her2/GFP/CD19 positive cells (FIG. 13). From 24 to 96 hours, however, the engineered T cells continued to kill GFP/CD19 positive cells, but stopped killing Her2/GFP/CD19 positive cells as shown by the flat line of target cell survival (FIG. 13 and FIG. 14). Thus, activation of the new NOT module indeed led to sustained blocking of T cell killing in response to the presence of the respective NOT antigen.

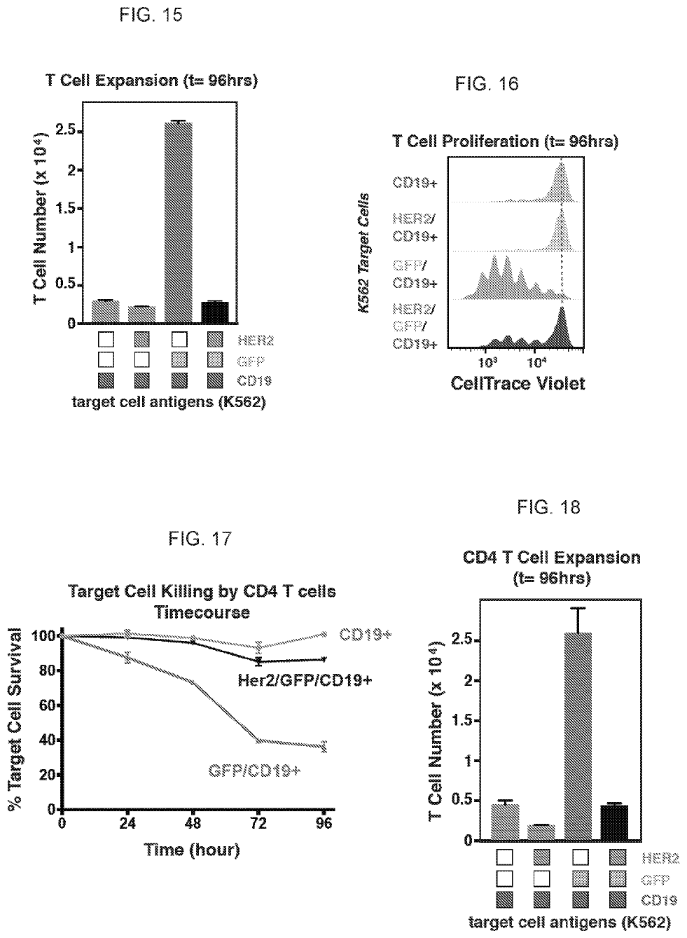

To more precisely monitor the effect of synNotch-driven tBID expression on the engineered T cells, T cell population expansion and single cell proliferation were measured at the end of the time-course co-cultures. Engineered CD8+ T cells were found to selectively expand and proliferate in the presence of GFP/CD19 positive cells, while anti-Her2 synNotch prevented T cell expansion and eliminated proliferation in response to Her2/GFP/CD19 positive cells (FIG. 15 and FIG. 16). Human primary CD4+ T cells engineered with this same circuit showed similar patterns of controlled target cell killing and T cell expansion, (FIG. 17 and FIG. 18). These results show that the synNotch NOT module (synNotch→tBID apoptotic inducer) can block T cell expansion and survival that is essential for a strong T cell response, and that this system can be modularly incorporated into combinatorial antigen recognition circuits that include both positive and negative antigen selection. Thus, this data shows that synNotch-induced tBID expression can be used as a NOT-gate in combinatorial antigen sensing circuits to prevent killing of potential target cells expressing the synNotch antigen by causing T cell apoptosis and inhibiting T cell population expansion. The ability to incorporate this type of modular negative selection on a therapeutic T cell provides powerful new tools for shaping the types of antigen patterns can be recognized and discriminated against.

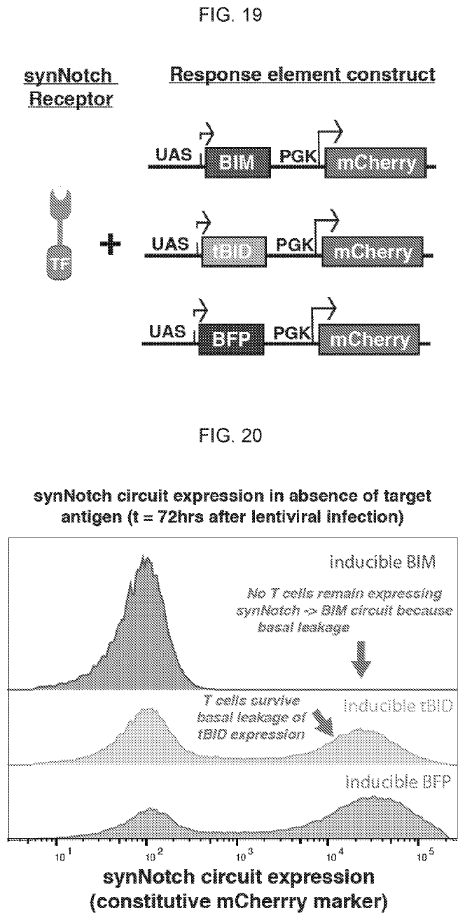

Differences in the potency of various BCL-2 family members in inducible systems for therapeutic cell population control were also investigated and differences in potency were observed. For example, molecular circuits were designed and tested that included a response element construct including a UAS, inducible by the released transcription factor (TF) domain of a synNotch receptor, driving expression of various different BCL-2 family members (e.g., BIM, tBID, etc.) and a constitutive reporter (mCherry) (see schematic depiction provided in FIG. 19). Upon binding its target antigen, the synNotch receptor releases the TF domain which activates the UAS thereby driving expression of the respective BCL-2 family protein and the mCherry reporter is used to observe the size of the cell population thereafter. However, as shown in FIG. 20, even in the absence of the synNotch target antigen, leaky expression from the inducible pro-apoptotic factor BIM construct was sufficient to cause widespread T cell death (top line). In contrast, a T cell population comparable in size to that of the inducible BFP negative control (bottom line) survived any basal leaky expression of the tBID construct (middle line). These results demonstrate a difference in potency between BIM and tBID pro-apoptotic proteins, which also demonstrates that apoptotic modulating factors should be carefully chosen when configuring user and cell-autonomously controllable molecular circuits.

While inducible BCL-2 family member expression for regulating T cell apoptosis was tested as described above in the context of synNotch-induced expression, any appropriate system that can induce protein expression/activity could be employed to regulate survival of therapeutically-relevant cells such as stem cells, engineered T cells, or the like.

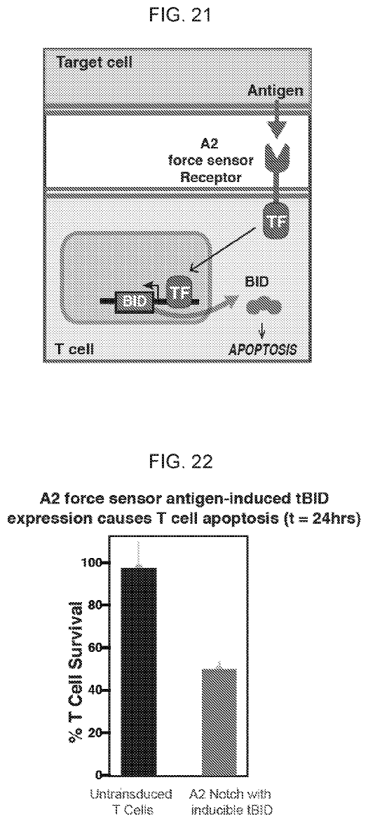

For example, a circuit employing a hybrid Notch and von Willebrand A2 domain-containing force sensor in place of the above described synNotch receptor was designed and tested. The subject circuit is schematically depicted in FIG. 21, where a Jurkat T cell was engineered with a tBID encoding sequence operably linked to a regulatory sequence responsive to the released transcription factor (TF) domain of the hybrid Notch/A2 force sensor receptor specific for an antigen. Correspondingly, upon binding its antigen, the TF domain of the hybrid Notch/A2 force sensor is released thereby driving expression of tBID and promoting apoptosis of the engineered T cell. Quantification of T cell survival at 24 hours is shown in FIG. 22, demonstrating that T cells transduced with the Notch/A2/tBID circuit express tBID in an antigen dependent (i.e., inducible) manner and such tBID expression causes T cell apoptosis. These results demonstrate that various binding-triggered transcriptional switches (BTTSs) can be used to regulate expression and activity of apoptosis modulators, including pro-apoptotic factor tBID, to control death of engineered T cells.

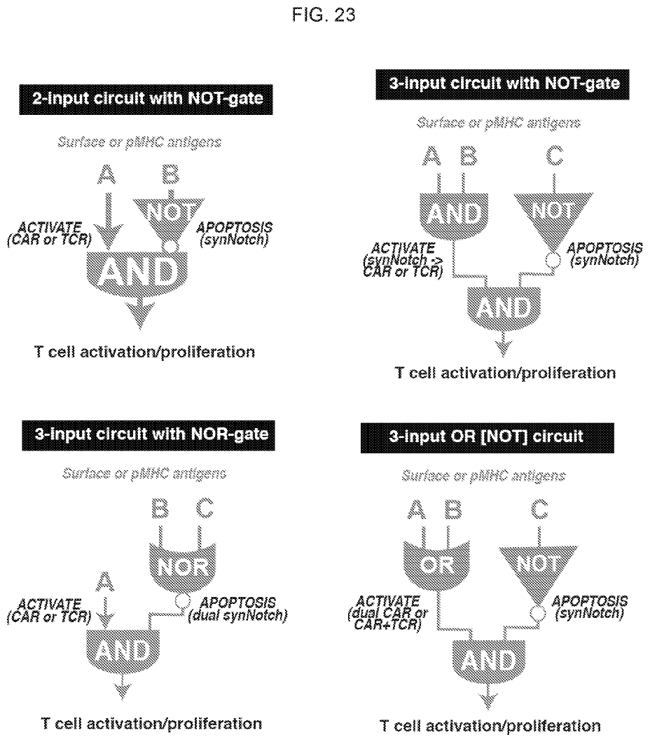

Accordingly, a variety of different antigen-input driven circuits may employ the apoptosis modulating strategies described herein to result combinatorial antigen sensing therapeutic cell population control. Non-limiting examples of such circuits are depicted in FIG. 23, which include two- and three-input NOT, NOR and OR[NOT] gates employing different combinations of a BTTS (depicted as a “synNotch”) and effectors (depicted as chimeric T cell receptors (CAR) or T cell receptors (TCR). Depending on the configuration and the particular components employed, such circuits may or may not employ buffering with a constitutive or inducible anti-apoptotic BCL-2 family protein.

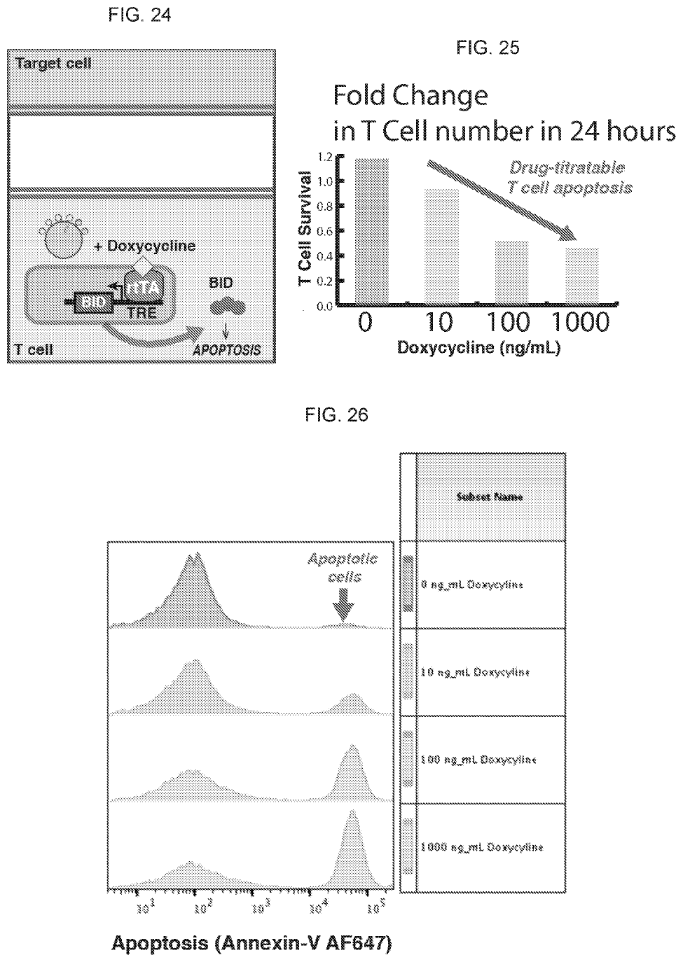

To test the use of other, non-synNotch, systems to regulate BCL-2 family member expression and activity, primary human T cells were engineered with a Tet-On system for doxycycline-inducible tBID expression (schematized in FIG. 24). Briefly, a T cell was engineered to include a sequence encoding tBID operably linked to a Tet Response Element (TRE) such that, in the presence of doxycycline, reverse tetracycline-controlled transactivator (rtTA) binds the TRE and tBID is expressed. Using this system it was demonstrated that doxycycline is able to titratably regulate apoptosis of T cells engineered with a Tet-On controlling tBID expression (FIG. 25). In addition, raw flow cytometry data (quantified in FIG.T) clearly shows the doxycycline-titratable primary T cell apoptosis of the system (FIG. 26). The result of this doxycycline controlled tBID and T cell apoptosis system demonstrate that a diversity of control systems, including drug-controllable systems, may be employed to regulate BCL-2 family member activity in engineered therapeutic cells.

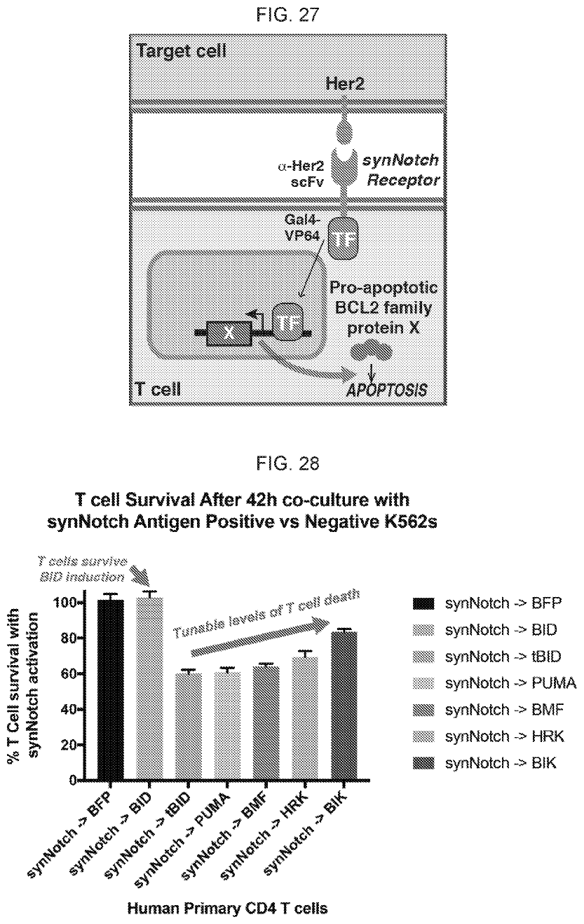

Examples of pro-apoptotic factors, of the various tested, that were found to be effective for controlling therapeutic cell survival include tBID, PUMA, BMF, HRK and BIK. For example, a circuit was designed, as schematically depicted in FIG. 27, with an anti-Her2 synNotch driving expression of a pro-apoptotic BCL-2 family protein “X”, where X stands for the various pro-apoptotic BCL-2 family members were tested in the depicted circuit. As shown, upon the anti-Her2 scFv portion of the synNotch receptor binding Her2 expressed by a target cell, the transcription factor (TF) portion of the synNotch, containing Gal4-VP64, is released and inducing expression of the pro-apoptotic BCL-2 family protein “X”. The level of induced apoptosis in the presence of cells expressing the target antigen, as compared to control cells not expressing the target antigen, was then assessed.

Results of testing BID, tBID, PUMA, BMF, HRK and BIK as pro-apoptotic BCL-2 family protein “X” in this system are shown in FIG. 28, along with a negative control employing BFP in place of the pro-apoptotic BCL-2 family protein “X”. As shown, tBID, PUMA, BMF, HRK and BIK employed in the above described circuit were all found to induce apoptosis of human primary CD4 T cells, leading to a reduction in therapeutic cell survival, in the presence of antigen-expressing target cells. Moreover, due to the observed differences in T cell survival seen between tBID, PUMA, BMF, HRK and BIK, these data demonstrate the ability to tune the level of induced therapeutic cell death as desired, e.g., by employing a pro-apoptotic factor with a higher or lower effect on apoptosis. For example, these results show that, where higher levels of induced therapeutic cell killing are desired, a pro-apoptotic factor such as tBID may be employed. Correspondingly, where a lower level of induced therapeutic cell killing is desired, a pro-apoptotic factor such as BIK may be employed. As such, these results demonstrate the ability of the herein described circuits to provide tunable levels of inducible therapeutic cell death.

In addition, the data provided in FIG. 28 further demonstrates the varied levels of effectiveness of different BCL-2 family members in the herein described circuits, methods, cells, etc. For example, when BID was employed as the apoptotic BCL-2 family protein “X” in the system depicted in FIG. 27, the level of T cell survival in the presence of target cells expressing the Her2 antigen was similar to the BFP negative control. Thus, the therapeutic cells encoding the BID circuit were able to survive BID induction.

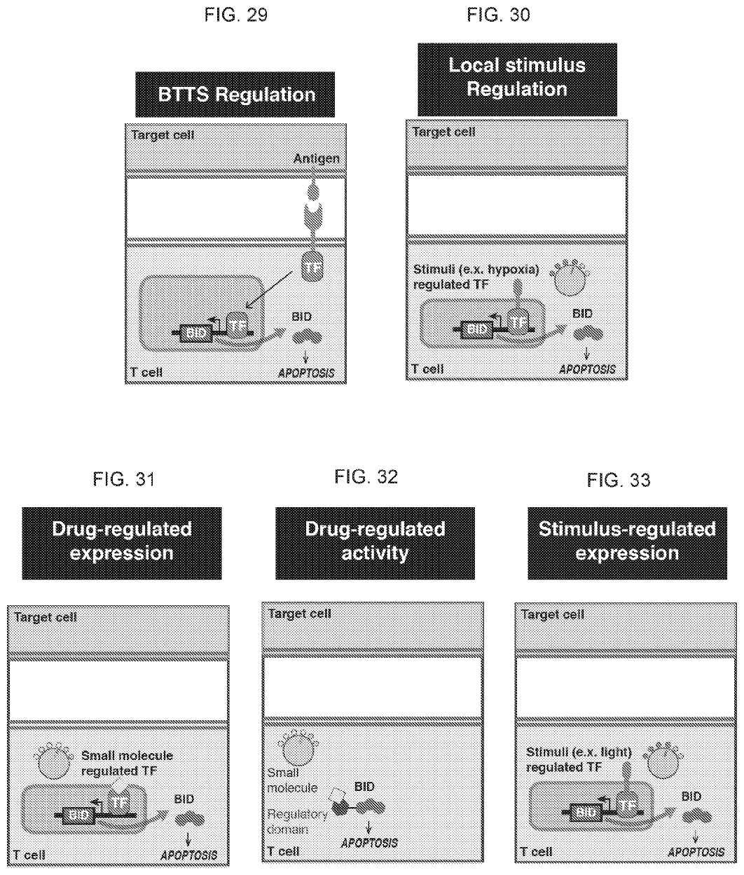

Collectively, the examples provided above demonstrate that various circuits employing pro- and/or anti-apoptotic BCL-2 family member proteins may be configured and employed to modulate therapeutic cell survival in a controlled, inducible, and tunable manner. Various different circuits may be employed, alone or in combination with other components and/or circuits, including for cell autonomous antigen-dependent expression regulation (e.g., as in the exemplary circuit schematized in FIG. 29, using tBID merely as a non-limiting example of a BCL-2 family member that may be employed), for stimuli-dependent expression regulation (e.g., as in the exemplary circuit schematized in FIG. 30, using tBID merely as a non-limiting example of a BCL-2 family member that may be employed), or user-controlled drug dependent or stimuli-dependent expression/activity regulation (e.g., as in the exemplary circuits schematized in FIG. 31, FIG. 32 and FIG. 33, using tBID merely as a non-limiting example of a BCL-2 family member that may be employed). These exemplary circuits depict the diversity of inducible systems that can be used to regulate the expression/activity of different BCL-2 family members to create death and survival switches with varied behaviors as described herein.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A therapeutic cell comprising a heterologous inducible pro-apoptotic agent comprising an inducible BCL-2 family pro-apoptotic protein.

2. The cell according to claim 1, wherein the BCL-2 family pro-apoptotic protein is selected from the group consisting of: an inducible truncated BID, an inducible PUMA, an inducible BMF, an inducible HRK, and an inducible BIK.

3. The cell according to claim 1 or 2, further comprising a heterologous anti-apoptotic agent comprising a BCL-2 family anti-apoptotic protein.

4. The cell accordingly to claim 3, wherein the BCL-2 family anti-apoptotic protein is BCL-2.

5. The cell according to claim 3 or 4, wherein the heterologous anti-apoptotic agent is constitutive.

6. The cell according to claim 3 or 4, wherein the heterologous anti-apoptotic agent is inducible.

7. The cell according to any of the preceding claims, wherein the cell is a therapeutic immune cell.

8. The cell according to any of the preceding claims, wherein the cell comprises a heterologous nucleic acid encoding a therapeutic agent selected from the group consisting of: a therapeutic antibody, a chimeric antigen receptor, and an engineered T cell receptor.

9. The cell according to claim 8, wherein expression of the therapeutic agent is regulatable.

10. The cell according to claim 9, wherein the regulatable system comprises an inducible promoter controlling expression of the therapeutic agent.

11. The cell according to claim 9 or 10, wherein expression of the therapeutic agent is regulated by a binding-triggered transcriptional switch.

12. The cell according to any of the preceding claims, wherein the inducible pro-apoptotic agent is ligand inducible.

13. The cell according to claim 12, wherein the ligand inducible pro-apoptotic agent comprises a sequence encoding a BCL-2 family pro-apoptotic protein operably linked to a regulatory sequence responsive to binding of a binding-triggered transcriptional switch to a ligand.

14. The cell according to claim 13, wherein the BCL-2 family pro-apoptotic protein is a BIM, a truncated BID, a PUMA, a BMF, a HRK, or a BIK.

15. The cell according to claim 13 or 14, wherein the ligand is expressed by non-target cells.

16. The cell according to claim 15, wherein the non-target cells are non-cancer cells.

17. The cell according to claim 13 or 14, wherein the ligand is present on a solid support.

18. The cell according to claim 17, wherein the solid support is a polymer particle.

19. The cell according to any of claims 1 to 11, wherein the inducible pro-apoptotic agent is small molecule inducible.

20. The cell according to any of claims 1 to 11, wherein the inducible pro-apoptotic agent is stimuli inducible.

21. The cell according to claim 20, wherein the stimuli inducible pro-apoptotic agent is induced by light, ultrasound or hypoxia.

22. A method comprising administering a therapeutic cell according to any of claims 1 to 21 to a subject in need thereof.

23. A method of treating a subject for an adverse reaction to a therapeutic cell of any of claims 1 to 21, the method comprising inducing the heterologous inducible pro-apoptotic agent.

24. The method according to claim 23, wherein the inducible pro-apoptotic agent is small molecule inducible and the method comprises administering to the subject an amount of a small molecule effective to induce the pro-apoptotic agent.

25. The method according to claim 24, wherein the inducible pro-apoptotic agent comprises a sequence encoding a BCL-2 family pro-apoptotic protein operably linked to a regulatory sequence.

26. The method according to claim 25, wherein the BCL-2 family pro-apoptotic protein is selected from the group consisting of: a truncated BID, a PUMA, a BMF, a HRK, and a BIK.

27. The method according to claim 25 or 26, wherein the small molecule binds a transcriptional activator of the regulatory sequence thereby inducing expression of the BCL-2 family pro-apoptotic protein.

28. The method according to claim 25 or 26, wherein the small molecule competitively binds a transcriptional repressor of the regulatory sequence thereby inducing expression of the BCL-2 family pro-apoptotic protein.

29. The method according to claim 24, wherein the inducible pro-apoptotic agent comprises a split BCL-2 family pro-apoptotic protein dimerized by the small molecule.

30. The method according to claim 29, wherein the split BCL-2 family pro-apoptotic protein is selected from the group consisting of: a split tBID, a split PUMA, a split BMF, a split HRK, and a split BIK.

31. The method according to claim 23, wherein the inducible pro-apoptotic agent is stimuli inducible and the method comprises stimulating at least a portion of the subject with an amount of a stimuli effective to induce the pro-apoptotic agent.

32. The method according to claim 23, wherein the inducible pro-apoptotic agent is ligand inducible and the method comprises contacting the subject with an amount of a ligand effective to induce the pro-apoptotic agent.

33. One or more nucleic acids comprising:
a first sequence encoding a therapeutic polypeptide responsive to a target antigen; and
a second sequence encoding an inducible pro-apoptotic agent comprising an inducible BCL-2 family pro-apoptotic protein.

34. The one or more nucleic acids according to claim 33, wherein the inducible BCL-2 family pro-apoptotic protein is selected from the group consisting of: an inducible truncated BID, an inducible PUMA, an inducible BMF, an inducible HRK, and an inducible BIK.

35. The one or more nucleic acids according to claim 33 or 34, wherein the therapeutic polypeptide is selected from the group consisting of: a therapeutic antibody, a chimeric antigen receptor, and an engineered T cell receptor.

36. The one or more nucleic acids according to any of claims 33 to 35, wherein the target antigen is a cancer antigen.

37. The one or more nucleic acids according to any of claims 33 to 35, wherein the target antigen is a non-natural bioorthogonal ligand.

38. The one or more nucleic acids according to any of claims 33 to 37, wherein the inducible pro-apoptotic agent is small molecule inducible.

39. The one or more nucleic acids according to any of claims 33 to 37, wherein the inducible pro-apoptotic agent is stimuli inducible.

40. The one or more nucleic acids according to claim 39, wherein the stimuli inducible pro-apoptotic agent is induced by light, ultrasound or hypoxia.

41. The one or more nucleic acids according to any of claims 33 to 37, wherein the inducible pro-apoptotic agent is ligand inducible.

42. The one or more nucleic acids according to claim 41, wherein the ligand inducible pro-apoptotic agent comprises a sequence encoding a BCL-2 family pro-apoptotic protein operably linked to a regulatory sequence responsive to binding of a binding-triggered transcriptional switch to a ligand.

43. The one or more nucleic acids according to claim 42, wherein the BCL-2 family pro-apoptotic protein is selected from the group consisting of: a tBID, a PUMA, a BMF, a HRK, and a BIK.

44. The one or more nucleic acids according to claim 42 or 43, wherein the ligand is expressed by non-target cells.

45. The one or more nucleic acids according to claim 44, wherein the non-target cells are non-cancer cells.

46. The one or more nucleic acids according to any of claims 33 to 45, further comprising a third sequence encoding a heterologous anti-apoptotic agent comprising a BCL-2 family anti-apoptotic protein.

47. The one or more nucleic acids according to claim 46, wherein the BCL-2 family anti-apoptotic protein is a BCL-2.

48. The one or more nucleic acids according to claim 46 or 47, wherein the heterologous anti-apoptotic agent is constitutive.

49. The one or more nucleic acids according to claim 46 or 47, wherein the heterologous anti-apoptotic agent is inducible.

50. A vector comprising the one or more nucleic acids according to any of claims 33 to 49.

51. A cell comprising the vector of claim 50.

52. A therapeutic cell comprising a heterologous constitutive or inducible anti-apoptotic agent comprising a BCL-2 family anti-apoptotic protein.

53. The cell according to claim 52, wherein the BCL-2 family anti-apoptotic protein is a BCL-2.

54. The cell according to claim 52 or 53, wherein the anti-apoptotic agent is ligand inducible.

55. The cell according to claim 54, wherein the ligand inducible anti-apoptotic agent comprises a sequence encoding a BCL-2 family anti-apoptotic protein operably linked to a regulatory sequence responsive to binding of a binding-triggered transcriptional switch to a ligand.

56. The cell according to claim 55, wherein the ligand is expressed by a target cell.

57. The cell according to claim 56, wherein the target cell is a cancer cell.

58. The cell according to claim 56, wherein the ligand is expressed tissue specifically.

59. The cell according to claim 55, wherein the ligand is present on a solid support.

60. The cell according to claim 59, wherein the solid support is a polymer particle.

61. The cell according to any of claims 55 to 60, wherein the ligand is a non-natural bioorthogonal ligand.

62. The cell according to claim 52 or 53, wherein the anti-apoptotic agent is small molecule inducible.

63. The cell according to claim 52 or 53, wherein the anti-apoptotic agent is stimuli inducible.

64. The cell according to claim 63, wherein the stimuli inducible anti-apoptotic agent is induced by light, ultrasound or hypoxia.

65. The cell according to any of claims 52 to 64, wherein the therapeutic cell is a therapeutic immune cell.

66. The cell according to any of claims 52 to 65, wherein the therapeutic cell comprises a heterologous nucleic acid encoding a therapeutic agent selected from the group consisting of: a therapeutic antibody, a chimeric antigen receptor, and an engineered T cell receptor.

67. The cell according to claim 66, wherein expression of the therapeutic agent is regulated by a binding-triggered transcriptional switch.

68. The cell according to any of claims 52 to 66, further comprising a heterologous inducible pro-apoptotic agent comprising an inducible BCL-2 family pro-apoptotic protein.

69. The cell according to claim 68, wherein the BCL-2 family pro-apoptotic protein is selected from the group consisting of: an inducible BIM, an inducible truncated BID, an inducible PUMA, an inducible BMF, an inducible HRK, and an inducible BIK.

70. A method comprising administering a therapeutic cell according to any of claims 52 to 69 to a subject in need thereof.

71. A method of enhancing a cellular therapy, the method comprising:
administering or having administered a therapeutic cell comprising an inducible heterologous anti-apoptotic agent comprising a BCL-2 family anti-apoptotic protein to a subject; and
inducing the inducible heterologous anti-apoptotic agent.

72. The method according to claim 71, wherein the BCL-2 family anti-apoptotic protein is a BCL-2.

73. The method according to claim 71 or 72, wherein the therapeutic cell comprises a therapeutic polypeptide, or an encoding sequence thereof, responsive to a target antigen.

74. The method according to claim 73, wherein the therapeutic polypeptide is selected from the group consisting of: a therapeutic antibody, a chimeric antigen receptor, and an engineered T cell receptor.

75. The method according to claim 74, wherein the target antigen is a cancer antigen.

76. The method according to any of claims 71 to 75, wherein the inducible anti-apoptotic agent is small molecule inducible and the method comprises administering to the subject an amount of a small molecule effective to induce the anti-apoptotic agent.

77. The method according to claim 76, wherein the inducible anti-apoptotic agent comprises a sequence encoding a BCL-2 family anti-apoptotic protein operably linked to a regulatory sequence.

78. The method according to claim 77, wherein the small molecule binds a transcriptional activator of the regulatory sequence thereby inducing expression of the BCL-2 family anti-apoptotic protein.

79. The method according to claim 77, wherein the small molecule competitively binds a transcriptional repressor of the regulatory sequence thereby inducing expression of the BCL-2 family anti-apoptotic protein.

80. The method according to claim 76, wherein the inducible anti-apoptotic agent comprises a split BCL-2 family anti-apoptotic protein dimerized by the small molecule.

81. The method according to claim 80, wherein the split BCL-2 family anti-apoptotic protein is a split BCL-2.

82. The method according to any of claims 71 to 75, wherein the inducible anti-apoptotic agent is stimuli inducible and the method comprises stimulating at least a portion of the subject with an amount of a stimuli effective to induce the anti-apoptotic agent.

83. The method according to any of claims 71 to 75, wherein the inducible anti-apoptotic agent is ligand inducible and the method comprises contacting the subject with an amount of a ligand effective to induce the anti-apoptotic agent.

84. One or more nucleic acids comprising:
a first sequence encoding a therapeutic polypeptide responsive to a target antigen; and
a second sequence encoding an anti-apoptotic agent comprising a BCL-2 family anti-apoptotic protein.

85. The one or more nucleic acids according to claim 84, wherein the BCL-2 family anti-apoptotic protein is a BCL-2.

86. The one or more nucleic acids according to claim 84 or 85, further comprising a third sequence encoding a heterologous inducible pro-apoptotic agent comprising an inducible BCL-2 family pro-apoptotic protein.

87. The one or more nucleic acids according to claim 85 or 86, wherein the BCL-2 family pro-apoptotic protein is selected from the group consisting of: an inducible BIM, an inducible truncated BID, an inducible PUMA, an inducible BMF, an inducible HRK, and an inducible BIK.

88. The one or more nucleic acids according to claim 85 or 86, wherein the heterologous inducible pro-apoptotic agent is small molecule inducible.

89. The one or more nucleic acids according to claim 85 or 86, wherein the heterologous inducible pro-apoptotic agent is stimuli inducible.

90. The one or more nucleic acids according to claim 89, wherein the stimuli inducible pro-apoptotic agent is induced by light, ultrasound or hypoxia.

91. The one or more nucleic acids according to claim 85 or 86, wherein the heterologous inducible pro-apoptotic agent is ligand inducible.

92. The one or more nucleic acids according to claim 91, wherein the ligand inducible pro-apoptotic agent comprises a sequence encoding a BCL-2 family pro-apoptotic protein operably linked to a regulatory sequence responsive to binding of a binding-triggered transcriptional switch to a ligand.

93. The one or more nucleic acids according to any of claim 84 or 92, wherein the therapeutic polypeptide is selected from the group consisting of: a therapeutic antibody, a chimeric antigen receptor, and an engineered T cell receptor.

94. The one or more nucleic acids according to any of claims 84 to 93, wherein the target antigen is a cancer antigen.

95. The one or more nucleic acids according to any of claims 84 to 94, wherein the anti-apoptotic agent is constitutive.

96. The one or more nucleic acids according to any of claims 84 to 94, wherein the anti-apoptotic agent is inducible.

97. The one or more nucleic acids according to claim 96, wherein the anti-apoptotic agent is small molecule inducible.

98. The one or more nucleic acids according to claim 96, wherein the inducible anti-apoptotic agent is stimuli inducible.

99. The one or more nucleic acids according to claim 98, wherein the stimuli inducible anti-apoptotic agent is induced by light, ultrasound or hypoxia.

100. The one or more nucleic acids according to claim 96, wherein the inducible anti-apoptotic agent is ligand inducible.

101. The one or more nucleic acids according to claim 100, wherein the ligand inducible anti-apoptotic agent comprises a sequence encoding a BCL-2 family anti-apoptotic protein operably linked to a regulatory sequence responsive to binding of a binding-triggered transcriptional switch to a ligand.

102. The one or more nucleic acids according to claim 101, wherein the ligand is expressed by a target cell.

103. The one or more nucleic acids according to claim 102, wherein the target cell is a cancer cell.

104. The one or more nucleic acids according to claim 102, wherein the ligand is expressed tissue specifically.

105. A vector comprising the one or more nucleic acids according to any of claims 84 to 104.

106. A cell comprising the vector of claim 105.
